SEQUENCE LISTING (36 sequences)

SEQ ID NO: 1 - DNA, 20 bp, synthetic construct
  source 1..20: mol_type = other DNA
accattaaagaaaatatcat

SEQ ID NO: 2 - DNA, 20 bp, synthetic construct
  source 1..20: mol_type = other DNA
tctgtatctatattcatcat

SEQ ID NO: 3 - DNA, 23 bp, synthetic construct
  source 1..23: mol_type = other DNA
gacaacacagttctctgagaagg

SEQ ID NO: 4 - DNA, 80 bp, synthetic construct
  source 1..80: mol_type = other DNA
agttttcctggattatgcctggcaccattaaagaaaatatcatctttggtgtttcctatgatgaatatagatacagaagc

SEQ ID NO: 5 - DNA, 80 bp, synthetic construct
  source 1..80: mol_type = other DNA
gcttctgtatctatattcatcataggaaacaccaaagatgatattttctttaatggtgccaggcataatccaggaaaact

SEQ ID NO: 6 - DNA, 120 bp, synthetic construct
  source 1..120: mol_type = other DNA
aagaatttcattctgttctcagttttcctggattatgcctggcaccattaaagaaaatatcatctttggtgtttcctatgatgaatatagatacagaagcgtcatcaaagcatgccaact

SEQ ID NO: 7 - DNA, 87 bp, synthetic construct
  source 1..87: mol_type = other DNA
ataggaaacaccaaagatgatattttctttaatggtgccaggcataatccaggaaaactgagaacagaatgaaattcttccactgtg

SEQ ID NO: 8 - DNA, 200 bp, synthetic construct
  source 1..200: mol_type = other DNA
tttcaattttggattgtgcatgctaaattttatttctggtgttatgctttggataataggacatcaccaagtttgcagaacaagacaacacagttctaggagaaggtggagtcacactgagtggaggtcagcgtgcaaggatttctttagcaaggtaaatatttaactgttggtcttgtgagcacttgctgtaaatacta

SEQ ID NO: 9 - DNA, 160 bp, synthetic construct
  source 1..160: mol_type = other DNA
tgctaaattttatttctggtgttatgctttggataataggacatcaccaagtttgcagaacaagacaacacagttctaggagaaggtggagtcacactgagtggaggtcagcgtgcaaggatttctttagcaaggtaaatatttaactgttggtcttgtg

SEQ ID NO: 10 - DNA, 120 bp, synthetic construct
  source 1..120: mol_type = other DNA
gttatgctttggataataggacatcaccaagtttgcagaacaagacaacacagttctaggagaaggtggagtcacactgagtggaggtcagcgtgcaaggatttctttagcaaggtaaat

SEQ ID NO: 11 - DNA, 80 bp, synthetic construct
  source 1..80: mol_type = other DNA
acatcaccaagtttgcagaacaagacaacacagttctaggagaaggtggagtcacactgagtggaggtcagcgtgcaagg

SEQ ID NO: 12 - DNA, 80 bp, synthetic construct
  source 1..80: mol_type = other DNA
ccttgcacgctgacctccactcagtgtgactccaccttctcctagaactgtgttgtcttgttctgcaaacttggtgatgt

SEQ ID NO: 13 - DNA, 60 bp, synthetic construct
  source 1..60: mol_type = other DNA
gtttgcagaacaagacaacacagttctaggagaaggtggagtcacactgagtggaggtca

SEQ ID NO: 14 - DNA, 4203 bp, Homo sapiens
  source 1..4203: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 15 - RNA, 20 nt, synthetic construct
  source 1..20: mol_type = other RNA
  ncRNA 1..20: ncRNA_class = sgRNA
gctgaagcactgcacgccat

SEQ ID NO: 16 - RNA, 20 nt, synthetic construct
  source 1..20: mol_type = other RNA
  ncRNA 1..20: ncRNA_class = sgRNA
gacaacacagttctctgaga

SEQ ID NO: 17 - RNA, 20 nt, synthetic construct
  source 1..20: mol_type = other RNA
  ncRNA 1..20: ncRNA_class = sgRNA
accattaaagaaaatatcat

SEQ ID NO: 18 - DNA, 127 bp, synthetic construct
  source 1..127: mol_type = other DNA
gccacctacggcaagctgaccctgaagttcatctgcaccaccggcaagctgcccgtgccctggcccaccctcgtgaccaccctgacgtacggcgtgcagtgcttcagccgctaccccgaccacatga

SEQ ID NO: 19 - DNA, 200 bp, synthetic construct; identical to SEQ ID NO: 8
  source 1..200: mol_type = other DNA

SEQ ID NO: 20 - DNA, 120 bp, synthetic construct; identical to SEQ ID NO: 6
  source 1..120: mol_type = other DNA

SEQ ID NO: 21 - DNA, 19 bp, synthetic construct
  source 1..19: mol_type = other DNA
aagcttggtaccgagctcg

SEQ ID NO: 22 - DNA, 28 bp, synthetic construct
  source 1..28: mol_type = other DNA
tgaatggcacttgagtttatatgatggg

SEQ ID NO: 23 - DNA, 22 bp, synthetic construct
  source 1..22: mol_type = other DNA
tgggggcaagtgaatcctgagc

SEQ ID NO: 24 - DNA, 23 bp, synthetic construct
  source 1..23: mol_type = other DNA
ttgtggcggatcttgaagttcac

SEQ ID NO: 25 - DNA, 27 bp, synthetic construct
  source 1..27: mol_type = other DNA
gaaattcaaatcagaggcatctgggga

SEQ ID NO: 26 - DNA, 29 bp, synthetic construct
  source 1..29: mol_type = other DNA
cattgaggacgtttgtctcactaatgagt

SEQ ID NO: 27 - DNA, 20 bp, synthetic construct
  source 1..20: mol_type = other DNA
acaagacaacacagttctct

SEQ ID NO: 28 - DNA, 22 bp, synthetic construct
  source 1..22: mol_type = other DNA
tccatgcaccataacaacaagt

SEQ ID NO: 29 - DNA, 20 bp, synthetic construct
  source 1..20: mol_type = other DNA
acaagacaacacagttcttg

SEQ ID NO: 30 - DNA, 27 bp, Mus sp.
  source 1..27: mol_type = other DNA
caagacaacacagttctctgagaaggt

SEQ ID NO: 31 - DNA, 27 bp, Mus sp.
  source 1..27: mol_type = other DNA
caagacaacacagttctaggagaaggt

SEQ ID NO: 32 - DNA, 24 bp, Homo sapiens
  source 1..24: mol_type = other DNA
accattaaagaaaatatcattggt

SEQ ID NO: 33 - DNA, 27 bp, Homo sapiens
  source 1..27: mol_type = other DNA
accattaaagaaaatatcatctttggt

SEQ ID NO: 34 - DNA, 30 bp, Homo sapiens
  source 1..30: mol_type = other DNA
ctgacccatggcgtgcagtgcttcagccgc

SEQ ID NO: 35 - DNA, 30 bp, Homo sapiens
  source 1..30: mol_type = other DNA
ctgacgtacggcgtgcagtgcttcagccgc

SEQ ID NO: 36 - DNA, 24 bp, Homo sapiens
  source 1..24: mol_type = other DNA
ctgacccatggcgtgcagtgcttc

(19) United States
(12) Patent Application Publication
(10) Pub. No.: US 2024/0261440 A1
(43) Pub. Date: Aug. 8, 2024

(54) COMPOSITIONS, METHODS AND USES FOR TREATING CYSTIC FIBROSIS AND RELATED DISORDERS

(71) Applicant: The Board of Regents of The University of Texas System, Austin, TX (US)

(72) Inventors: Tuo WEI, Dallas, TX (US); Yehui SUN, Dallas, TX (US); Qiang CHENG, Dallas, TX (US); Daniel SIEGWART, Dallas, TX (US)

(73) Assignee: The Board of Regents of The University of Texas System, Austin, TX (US)

(21) Appl. No.: 18/419,370

(22) Filed: Jan. 22, 2024

Related U.S. Application Data
(63) Continuation-in-part of application No. 18/553,975, filed on Jan. 1, 0001.
(60) Provisional application No. 63/481,166, filed on Jan. 23, 2023.
(60) Provisional application No. 63/171,071, filed on Apr. 5, 2021.

(51) Int. Cl. A61K 48/00; A61K 31/7088; A61K 38/46; A61K 47/24; A61K 47/54; C07K 14/705; C12N 9/22; C12N 15/11; C12N 15/88; C12N 15/90

(52) U.S. Cl. CPC A61K 48/0066; A61K 31/7088; A61K 38/465; A61K 47/24; A61K 47/543; C07K 14/705; C12N 9/22; C12N 15/11; C12N 15/88; C12N 15/907; C12N 2310/20; C12N 2800/80

(57) ABSTRACT

Described herein are compositions, kits, and methods for potent delivery to a cell of a subject. The cell can be of a particular cell type, such as a basal cell, a ciliated cell, or a secretory cell. In some cases, the cell can be a lung cell of a particular cell type. Also described herein are pharmaceutical compositions comprising a therapeutic or prophylactic agent assembled with a lipid composition. The lipid composition can comprise an ionizable cationic lipid, a phospholipid, and a selective organ targeting lipid. Further described herein are high-potency dosage forms of a therapeutic or prophylactic agent formulated with a lipid composition.

This application claims the benefit of priority to U.S. Provisional Application No. 63/481,166, filed on Jan. 23, 2023, the entire contents of which are hereby incorporated by reference. This application is also a continuation in part of U.S. application Ser. No. 18/553,975, filed Oct. 4, 2023, which is a national phase application under 35 U.S.C. § 371 of International Application No. PCT/US2022/023333, filed Apr. 4, 2022, which claims the benefit of priority to U.S. Provisional Application No. 63/171,071, filed Apr. 5, 2021, the entire contents of each of which are hereby incorporated by reference.

### STATEMENT AS TO FEDERALLY SPONSORED RESEARCH

This invention was made with government support under contract number RO1 EB025192-01A1 awarded by the National Institutes of Health National Institute of Biomedical Imaging and Bioengineering. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Jan. 21, 2024, is named UTSDP3895USCP1.xml and is 39,275 bytes in size.

### BACKGROUND

Cystic fibrosis (CF) is an autosomal recessive, hereditary disease caused by defects in the cystic fibrosis transmembrane conductance regulator (CFTR) gene. The CFTR gene encodes a cAMP-gated channel that is involved in chloride and bicarbonate transport. It regulates sodium transport through inhibition of the epithelial sodium channel, which is encoded by the SCNN1A gene. CFTR is expressed on the apical surface of epithelial cells in the airway, gastrointestinal tract, reproductive tract, sweat glands and submucosal glands.

Subjects having CF can present at birth or in early infancy with pancreatic insufficiency. Pancreatic dysfunction leads to malabsorption of fat and fat-soluble vitamins, which causes poor growth as well as gallstones and biliary disease. The lung manifestations of the disease can be more severe but may present slightly after pancreatic manifestations, in infancy or early childhood. Aberrant chloride and sodium transport due to decreased CFTR activity causes lowered apical surface fluid levels in the lungs, which leads to “sticky” mucous and lower airway obstruction. Subjects having CF or CF-like disease suffer from frequent infections due to inability to clear mucous. Local inflammatory mediators try to clear the infection but have difficulty. The triad of inflammation, infection and obstruction leads to progressive destruction of the lung parenchyma. Eventually, many subjects having CF or CF-like disease die in their late 30's due to respiratory failure.

Development of effective and durable therapies for cystic fibrosis (CF) patients remains an important and significant goal. Notably, most all patients with loss-of-function mutation(s) in CFTR remain untreatable with existing approaches.

Gene editing technologies, including CRISPR/Cas, represent a revolutionary approach for gene correction that if successfully developed to correct CFTR mutations would be a transformative advance resulting in long lasting therapies for CF patients, including those with loss-of-function mutations. A key bottleneck is the lack of delivery strategies required to enable targeted editing in specific cells, especially cells in the lungs. To date, successful in vivo editing has been mediated mainly by viral vectors, which present challenges for clinical translation due to potential immunogenicity, concerns about rare but dangerous integration events, and inability to re-dose. Non-viral lipid nanoparticle (LNP) delivery offers advantages in those respects, but advances have to date been limited to targets in the liver.

### SUMMARY

In an aspect, the present disclosure provides a method for enhancing an expression or activity of cystic fibrosis transmembrane conductance regulator (CFTR) protein in a cell, the method comprising: (a) contacting the cell with a nucleic acid editing system assembled with a lipid composition, which nucleic acid editing system comprises (i) a guide nucleic acid, (ii) a heterologous polypeptide comprising an endonuclease or a heterologous polynucleotide encoding the heterologous polypeptide, and (iii) a donor template nucleic acid, to yield a complex of the heterologous endonuclease with the guide nucleic acid in the cell; (b) cleaving a CFTR gene or transcript in the cell with the complex at a cleavage site to yield a cleaved CFTR gene or transcript; and (c) using the donor template nucleic acid to repair the cleaved CFTR gene or transcript to yield a repaired CFTR gene or transcript encoding a functional CFTR protein in the cell, thereby enhancing the expression or activity of CFTR protein in the cell. In some embodiments, c) is characterized by an off-target insertion or/and deletion (indel) rate of no more than about 50%, 49%, 48%, 47%, 46%, 45%, 44%, 43%, 42%, 41%, or 40%. In some embodiments, the off-target indel rate comprises a ratio of (1) a sum of test cells detected to have an incorrectly altered CFTR gene or transcript relative to (2) a sum of total test cells. In some embodiments, c) is characterized by an on-target repair rate of at least about 5%, 6%, 7%, 8%, 9%, 10%, 11%, 12%, 13%, 14%, 15%, 16%, 17%, 18%, 19%, or 20%. In some embodiments, the on-target repair rate comprises a ratio of (1) a sum of test cells detected to have the repaired CFTR gene or transcript relative to (2) a sum of total test cells. In some embodiments, the method increases an amount of a functional CFTR gene, transcript or protein in the cell (e.g., by at least about 1.1-fold) relative to a corresponding control, optionally, wherein the corresponding control is a corresponding cell absent the contacting. In some embodiments, the method yields a therapeutically effective amount of a functional of CFTR gene, transcript or protein in the cell (e.g., at least about 10%, 15%, 20%, 25%, or 30% among all detectable CFTR gene, transcript or protein). In some embodiments, the method enhances (e.g., chloride) ion transport in the cell (e.g., by at least about 1.1-fold) relative to a corresponding control, optionally, wherein the corresponding control is a corresponding cell absent the contacting. In some embodiments, (b) comprises cleaving a CFTR gene or transcript that comprises a loss-of-function mutation. In some embodiments, the method further comprises deriving a cell composition from the cell.

In some embodiments, the cell is a lung cell. In some embodiments, the cell is a lung basal cell. In some embodiments, the cell is an airway epithelial cell (e.g., a bronchial epithelial cell). In some embodiments, the cell is undifferentiated. In some embodiments, the cell is differentiated.

In some embodiments, the loss-of-function mutation comprises a mutation in an exon selected from exons 9-27 (e.g., exon 10, exon 12) of CFTR. In some embodiments, the loss-of-function mutation is F508del or G542X. In some embodiments, the loss-of-function mutation is associated with cystic fibrosis, hereditary emphysema, or chronic obstructive pulmonary disease (COPD).

In some embodiments, the contacting is ex vivo. In some embodiments, the contacting is in vitro. In some embodiments, the contacting is in vivo. In some embodiments, the contacting is repeated. In some embodiments, the contacting comprises contacting a plurality of cells that comprise the cell.

In some embodiments, the repairing yields a functional CFTR gene, transcript or protein in at least about 10%, 15%, 20%, 25%, 30%, 35%, 40%, 45%, 50%, 60%, 65%, or 70% of the plurality of cells, optionally wherein the plurality of cells are a plurality of (e.g., lung) basal cells. In some embodiments, the lipid composition comprises: an ionizable cationic lipid; and a selective organ targeting (SORT) lipid separate from the ionizable cationic lipid. In some embodiments, the lipid composition comprises a phospholipid separate from the SORT lipid.

In another aspect, the present disclosure provides an engineered cell composition comprising or derived from a cell having an expression or activity of cystic fibrosis transmembrane conductance regulator (CFTR) protein enhanced by a method described elsewhere herein.

In another aspect, the present disclosure provides a composition comprising a nucleic acid editing system assembled with a lipid composition, wherein the nucleic acid editing system comprises: (i) a guide nucleic acid comprising a targeting sequence that is complementary with a target sequence of a cystic fibrosis transmembrane conductance regulator (CFTR) gene or transcript; (ii) a polypeptide comprising an endonuclease or a polynucleotide encoding the polypeptide, which endonuclease is configured to (1) form a complex with the guide nucleic acid and (2) cleave the CFTR gene or transcript in a cell in a cleavage event; and (iii) a donor template nucleic acid configured to alter the CFTR gene or transcript, subsequent to the cleavage event, to provide a functional CFTR gene, transcript or protein in the cell. In some embodiments, (ii) is a messenger ribonucleic acid (mRNA) encoding the polypeptide comprising the endonuclease. In some embodiments, (i) and (iii) are present on two different molecules. In some embodiments, (ii) is the polypeptide comprising the endonuclease. In some embodiments, (i), (ii), and (iii) are present on three different molecules. In some embodiments, at least two of (i), (ii) and (iii) are present on one molecule. In some embodiments, the (i) and (ii) are present in the composition at a molar or weight ratio from 1:1 to 1:20. In some embodiments, (i) and (iii) are present in the composition at a molar or weight ratio from 1:1 to 1:30.

In some embodiments, the guide nucleic acid comprises a nucleotide sequence selected from those set forth in Table A (or disclosed elsewhere herein) and complementary sequences thereof. In some embodiments, the donor template nucleic acid comprises a nucleotide sequence selected from those set forth in Table B (or disclosed elsewhere herein) and complementary sequences thereof. In some embodiments, the donor template nucleic acid comprises a 5′ homology arm. In some embodiments, the donor template nucleic acid comprises a 3′ homology arm.

In some embodiments, the endonuclease is a CRISPR-associated (Cas) polypeptide or a modification thereof. In some embodiments, the endonuclease is Cas9.

In some embodiments, the composition is formulated for pharmaceutical (e.g., systemic) administration.

In another aspect, the present disclosure provides an engineered cell composition comprising or derived from a cell, which cell comprises a heterologous cystic fibrosis transmembrane conductance regulator (CFTR) gene, transcript or protein produced by a composition disclosed elsewhere herein.

In another aspect, the present disclosure provides a method for genetic correction of cystic fibrosis transmembrane conductance regulator (CFTR) in a lung basal cell, comprising: contacting the lung basal cell with a composition that comprises a nucleic acid editing system assembled with a lipid composition, thereby delivering the nucleic acid editing system to the lung basal cell.

In another aspect, the present disclosure provides a method for genetic correction of cystic fibrosis transmembrane conductance regulator (CFTR) in a cell composition, comprising: contacting the cell composition comprising a plurality of lung basal cells with a composition that comprises a nucleic acid editing system assembled with a lipid composition, thereby delivering the nucleic acid editing system to at least about 10%, 15%, 20%, 25%, 30%, 35%, 40%, 45%, 50%, 60%, 65%, or 70% of the plurality of lung basal cells.

In another aspect, the present disclosure provides a method for genetic correction of cystic fibrosis transmembrane conductance regulator (CFTR) in a cell composition, comprising: contacting the cell composition with a composition that comprises a nucleic acid editing system assembled with a lipid composition, which cell composition comprise a lung basal cell and a lung non-basal cell, thereby delivering the nucleic acid editing system to the lung basal cell in a greater amount than that delivered to the lung non-basal cell. In some embodiments, the non-basal cell is an ionocyte, a ciliated cell, or a secretory cell. In some embodiments, the lung basal cell or the plurality of lung basal cells is/are determined to exhibit a mutation in CFTR gene. In some embodiments, the lung basal cell or the plurality of lung basal cells exhibit(s) a mutation in CFTR gene.

In some embodiments, the lung basal cell or the plurality of lung basal cells is/are from a subject. In some embodiments, the subject is determined to exhibit a mutation in CFTR gene. In some embodiments, the subject exhibits a mutation in CFTR gene.

In some embodiments, the contacting is ex vivo. In some embodiments, the contacting is in vitro. In some embodiments, the contacting is in vivo.

In another aspect, the present disclosure provides a method for treating a subject having or suspected of having a cystic fibrosis transmembrane conductance regulator (CFTR)-associated condition, the method comprising administering to the subject a composition comprising a nucleic acid editing system assembled with a lipid composition. In some embodiments, the CFTR-associated condition is cystic fibrosis, hereditary emphysema, chronic obstructive pulmonary disease (COPD), or a combination thereof. In some embodiments, the subject is a mammal. In some embodiments, the subject is a human. In some embodiments, the subject is determined to exhibit a mutation (e.g., F508del or G542X) in CFTR gene. In some embodiments, the administering comprises systemic administration.

In another aspect, the present disclosure provides a composition comprising a lipid composition assembled with a nucleic acid editing system, wherein the nucleic acid editing system comprises (a) a guide nucleic acid, (b) a heterologous polypeptide comprising an endonuclease or heterologous polynucleotide encoding said heterologous polypeptide, and (c) a donor template nucleic acid. In some embodiments, the lipid composition comprises a selective organic targeting (SORT) lipid, wherein said SORT lipid has a structural formula (S-I′):

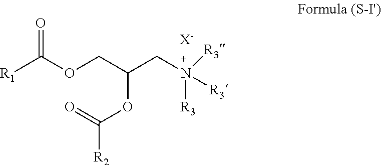

In some embodiments, R₁ and R₂ are each independently alkyl(C₈-C₂₄), alkenyl(C₈-C₂₄), or a substituted version of either group. In some embodiments, R₃, R₃′, and R₃″ are each independently alkyl(C_{≤6}) or substituted alkyl(C≤₆). In some embodiments, X⁻ is a monovalent anion. In some embodiments, the composition is configured to repair a cleaved cystic fibrosis transmembrane conductance regulator (CFTR) gene or transcript to yield a repaired CFTR gene or transcript encoding a functional CFTR protein when said composition is delivered to a cell, thereby enhancing an expression or activity of said functional CFTR protein in said cell.

In some embodiments, the SORT lipid is 1,2-dioleoyl-3-trimethylammonium-propane (DOTAP). In some embodiments, the lipid composition comprises about 10 mole percent (mol %) to about 40 mol % of said SORT lipid (e.g., DOTAP). In some embodiments, the lipid composition comprises an ionizable cationic lipid separate from said SORT lipid.

In some embodiments, the donor template nucleic acid is configured to alter a gene or transcript in a homology directed repair (HDR) pathway. In some embodiments, the endonuclease is a CRISPR-associated (Cas) polypeptide or a modification thereof. In some embodiments, the endonuclease is Cas9.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1D shows charts of indel and knockin percentages of various compositions

FIGS. 2A-2B shows charts of indel and knockin percentages of various compositions for editing and correction of G542X CFTR mutations [GACAACACAGTTCTCTGAGAAGG=SEQ ID NO: 3]

FIGS. 3A-3B shows charts of indel and knockin percentages of various compositions using different ratios of components

FIGS. 4A-4B show delivery of mRNA to mice basal cells by the lipid composition(s) described herein in the present application. FIGS. 4C-4D shows a chart of the indel and knock in percentages.

FIGS. 5A-5B show TIDER analysis of HBE cell treatment as described herein, illustrating the editing efficiency (FIG. 5A) and knock-in % (FIG. 5B) of the gene editing technology.

FIG. 6 shows SORT LNPs encapsulating three nucleic acids (Cas9 mRNA, sgRNA, and ssDNA HDR template) effectively corrected CFTR gene mutations in a G542X CF mouse model (in vivo) (SEQ ID NOs: 30 and 31) and in a patient-derived F508del HBE cell model (ex vivo) (SEQ ID NOs: 32 and 33).

FIGS. 7A-I show mRNA delivery in mice with low toxicity as described herein, mDLNP-2 was optimized by adjusting the internal molar ratio among 5A2-SC8/DOPE/Chol/PEG-DMG to 36/20/40/4 (FIG. 7A). Relative luciferase expression in mouse liver of mDLNP-2 encapsulating Luc mRNA was higher than that delivered using mDLNP-1, measured using IVIS (0.1 mg kg-1 Luc mRNA, i.v., 6h) (FIG. 7B). Quantification data further confirmed that optimization of the internal ratio enhanced mRNA delivery efficiency in mouse liver. Data are shown as mean±s.e.m. (n=3 biologically independent animals) (FIG. 7C). Permanently cationic lipid structures were screened to optimize lung targeted LNPs using IVIS imaging systems. A series of 5A2-SC8 LNPs with 20% to 50% different permanently cationic lipid (fraction of total lipids) were prepared. These LNPs encapsulating Luc mRNA were injected into mice tail vein for optimization (0.1 mg kg-1 Luc mRNA, i.v., 6h) (FIG. 7D). quantification data in mouse lungs suggested that DOTAP LNPs and DDAB LNPs showed higher luciferase expression in mouse lungs (FIG. 7E). Relative luciferase expression of Lung/Liver and Lung/Spleen were calculated to evaluate the lung targeting specificity. DOTAP40 and DDAB30 LNPs were selected by comparing lung delivery efficacy and specificity. Data are shown as mean±s.e.m. (n=3 biologically independent animals). (FIG. 7F). In vivo toxicity of DOTAP40 and DDAB30 LNPs were evaluated by measuring liver function parameters (ALT and AST) in mouse serum after 24h of treatment (1.5 mg kg⁻¹ mCherry mRNA, i.v., 40/1 (total lipid/total RNA)). PBS treatment (i.v.) was used as a negative control, and lipopolysaccharide (LPS, i.p., 5 mg kg⁻¹) treatment was used as a positive control (FIG. 7G). DOTAP40 LNPs showed lower acute toxicity compared to DDAB30 LNPs, determined by the body weight changes after each treatment. The body weights of mice with different treatments were monitored during the whole treatment period. LPS group mice were sacrificed at day 2, other groups were killed at day 5 (FIG. 7H). at sacrifice time point, all the organs (liver, spleen and lung) were collected and weighed. Liver/body weight, spleen/body weight, and lung/body weight ratio was calculated to evaluate their in vivo toxicity. DOTAP40 LNPs showed lower toxicity in vivo than DDAB30 LNPs (FIG. 7I). Data of FIG. 6G-I are shown as mean±s.e.m. (n=5 biologically independent animals). A two-tailed unpaired t-test was used to determine the significance of the comparisons of data (*P<0.05; **P<0.01; ***P<0.001).

FIG. 8 shows chemical structures of permanently cationic lipids used, including DDAB, DOTMA, EPC, MVL5, and DOTAP.

FIG. 9 shows in vivo toxicity of DOTAP40 and DDAB30 LNPs evaluated by measuring kidney function parameters. DOTAP40 showed lower toxicity than DDAB30 in vivo. In vivo toxicity of DOTAP40 and DDAB30 LNPs were evaluated by measuring kidney function parameters (BUN and CREA) and liver function parameters (ALT and AST) in mouse serum after 24h of treatment (1.5 mg kg⁻¹ mCherry mRNA, i.v., 40/1 (total lipid/total RNA)). PBS treatment (i.v.) was used as a negative control, and lipopolysaccharide (LPS, i.p., 5 mg kg⁻¹) treatment was used as a positive control. Data are presented as mean±s.e.m. (n=5 biologically independent animals). A two-tailed unpaired t-test was used to determine the significance of the comparisons of data (*P<0.05; **P<0.01; ***P<0.001).

FIG. 10 shows representative H&E staining images of mouse tissues after treatment with PBS, DDAB30, DOTAP40 and LPS. Mice were treated with DOTAP40 and DDAB30 LNPs (1.5 mg kg⁻¹ mCherry mRNA, i.v., 40/1 (total lipid/total RNA)). PBS treatment (i.v.) was used as a negative control, and lipopolysaccharide (LPS, i.p., 5 mg kg-1) treatment was used as a positive control. LPS group mice were sacrificed at day 2 after treatment and other groups were killed at day 5. At sacrifice time point, all the organs (heart, liver, spleen, lung and kidney) were collected and sectioned for H&E staining. Obvious tissue damages in heart, liver and lungs were observed when treated with DDAB30 LNPs and LPS, while DOTAP40 LNPs treatment showed low toxicity. Scale bar: 100 m.

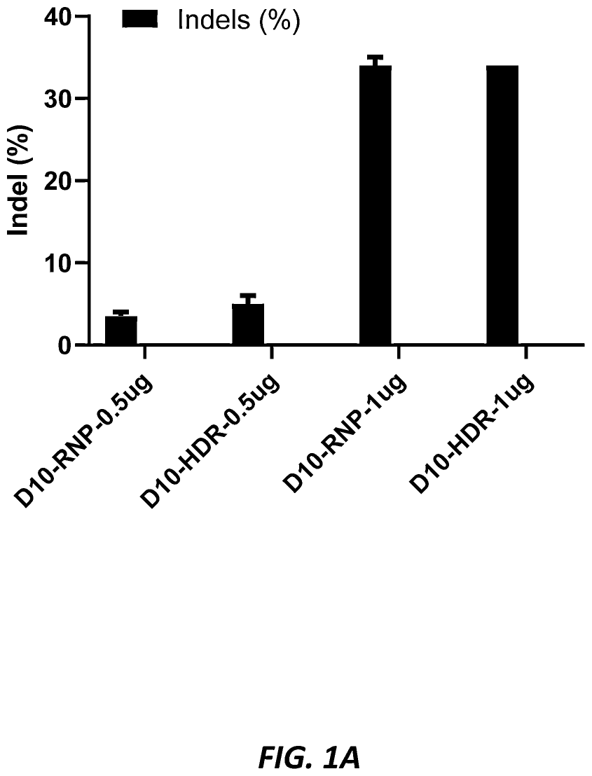

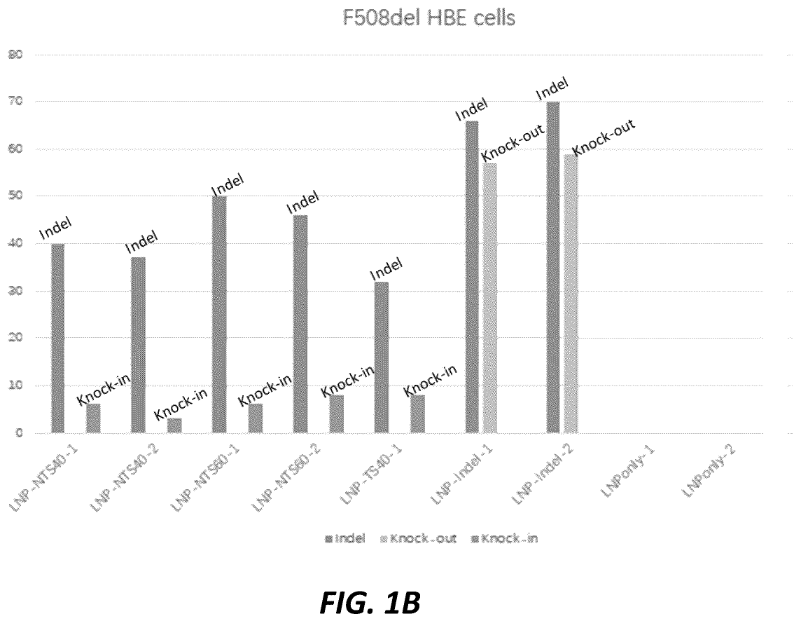

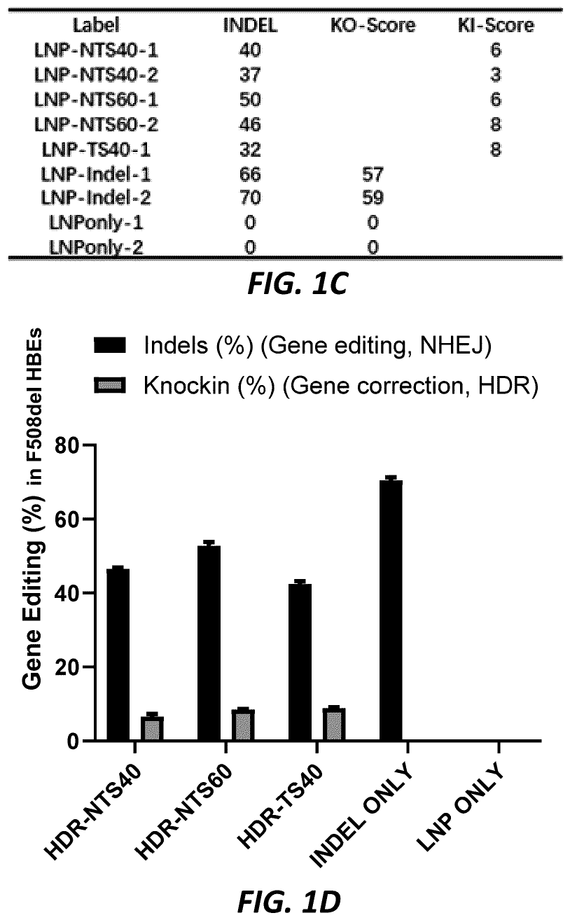

FIGS. 1A-B show the size and polymer dispersity index (PDI) of a series of mDLNPs-2 LNPs with 5%-30% DOTAP were measured by Dynamic Light Scattering (DLS) (FIG. 11A), and DOTAP10 LNPs efficiently delivered Luc mRNA to Hela cells and led to the highest luciferase expression after treatment of 24 hours with no obvious toxicity (20 ng per well of Luc mRNA) (FIG. 11B). DOTAP10 delivering Luc mRNA induced the highest luminescence intensity in Hela cells.

FIG. 12A-H show results of DOTAP10 LNPs, encapsulating Cas9 mRNA, sgRNA and ssDNA HDR template, successfully inducing HDR in HEK293 cells with Y66H GFP mutation.

FIG. 12A shows the HEK293 cells have a single amino acid mutation (Y66H) in their GFP sequencing (SEQ ID NOs: 34 and 35), which alters the fluorescence from GFP to BFP. Once the mutation is corrected, the fluorescence will turn from BFP to native GFP, the type and amount of gene editing were analyzed using TIDER analysis with DNA sequencing data obtained after transfections with a series of DOTAP10 LNPs containing Cas9 mRNA:sgRNA weight ratio fixed at 1:1 (total NA at 0.8 ng L⁻¹) (FIG. 12B). The type and amount of gene editing were analyzed using TIDER analysis with DNA sequencing data obtained after transfections with a series of DOTAP10 LNPs containing sgRNA:HDR weight ratio fixed at 1:6 (total NA at 0.8 ng L⁻¹). The optimal weight ratio among Cas9 mRNA, sgRNA and HDR was 0.5/1/6 (named as DOTAP10-HDR). Data of FIG. 12B, FIG. 12C are shown as mean±s.e.m. (n=3 biologically independent samples) (FIG. 12C). DNA sequencing result after treatment of DOTAP10-HDR demonstrated that the editing area was successfully corrected from “CCAT” to “GTAC” (SEQ ID NO: 36) with HDR correction efficiency as high as 50% (FIG. 12D). DOTAP10-HDR efficiently corrected Y55H GFP mutation in HEK293 cells and turned on bright GFP fluorescence. NC group (PBS treatment) and DOTAP 10 LNPs encapsulating Cas9 mRNA and sgRNA (named as DOTAP10-Indel) group were used as controls. The total NA was fixed at 0.4 ng L⁻¹. Before confocal imaging, cells were stained with Cell Mask Deep Red Stain solution to show cell membranes. Scale bar: 100 m (FIG. 12E). Ai14 mice were used to evaluate CRISPR/Cas gene editing. tdTOM mice were treated once a week with DOTAP40-NHEJ (Cas9 mRNA:sgTOM1=2:1, wt/wt) (FIG. 12G) and DOTAP40-HDR (Cas9 mRNA: sgTOM1: ssDNA HDR template for CF G542X=2:1:3, wt/wt/wt) (FIG. 12H) formulations respectively with a total RNA dosage of 1 mg kg⁻¹ IV. One week following the third injection, mice were sacrificed, and the lungs were collected for flow cytometry analysis to determine the proportion of tdTOM⁺ cells in each different cell type (n=3). (FIG. 12F).

FIGS. 13A-B show the Indel % and HDR % were analyzed using TIDER analysis with DNA sequencing data obtained after transfections with a series of DOTAP10 LNPs containing Cas9 mRNA:sgRNA weight ratio fixed at 1:1 (total NA at 0.8 ng L-1) (FIG. 13A), and the Indel % and HDR % were analyzed using TIDER analysis with DNA sequencing data obtained after transfections with a series of DOTAP10 LNPs containing sgRNA:HDR weight ratio fixed at 1:6 (total NA at 0.8 ng μL-1). The optimal weight ratio among Cas9 mRNA, sgRNA and HDR was 0.5/1/6 (named as DOTAP10-HDR) (FIG. 13B). Data are shown as mean±s.e.m. (n=3 biologically independent samples). DOTAP10 LNPs, encapsulating Cas9 mRNA, sgRNA and ssDNA HDR template at 0.5:1:6 (weight ratio) showed the highest HDR correction efficiency in HEK293 cells with Y66H GFP mutation.

FIGS. 14A-H show optimized DOTAP40 LNPs showed higher luciferase expression in mouse lung, compared to Lung SORT LNPs reported, and similar mRNA delivery efficiency was observed after decreasing weight ratio of total lipid/total NA from 40/1 to 20/1 (0.1 mg kg⁻¹ Luc mRNA, n=3 biologically independent animals) (FIG. 14A). DOTAP40 LNPs efficiently delivered Cre mRNA to basal cells of tdTOM mouse lung and turned on tdTOM fluorescence. tdTOM mice were treated with DOTAP40 LNPs (20:1) encapsulating Cre mRNA twice by I.V. injection (2 mg kg⁻¹ Cre mRNA). Two days after the second injection, mouse lungs were collected and lung basal cell populations were sorted using Anti-p75 NGF receptor antibody and the tdTOM positive cells in the whole basal cell populations were analyzed using flow cytometry (n=4 biologically independent animals in DOTAP40-Cre group). Nanoparticle only treatment group was used as negative control (NC) (FIG. 14B). Schematic illustration shows that DOTAP40-HDR LNPs delivering Cas9 mRNA, sgRNA and HDR template were I.V. injected into CFTR mouse model containing G542X mutation every week for total three times (2 mg kg⁻¹ total NA, 20:1). One week after the last injection, mouse lungs were collected and homogenized for following genome DNA extraction and DNA sequencing (FIG. 14C). DOTAP40-HDR treatment successfully corrected G542X mutation in mouse lung. The genome DNA of whole lung tissue after different treatment was extracted at the end time point and HDR correction efficiency at the G542X mutation site was analyzed using Sanger sequencing and TIDER analysis. NP only treatment group was used as negative control (NC). Data are shown as mean±s.e.m. (n=6 biologically independent animals) (FIG. 14D). Representative sequencing result showed the successful HDR correction at G542X mutation site was observed in mouse lung after treatment of DOTAP40-HDR LNPs, using TA clone and DNA sequencing (FIG. 14E). Schematic illustration shows the mechanism of intestinal organoid based forksolin-induced swelling (FIS) assay. The CFTR activator (Forksolin) can stimulate intracellular signal pathways and phosphorylate CFTR, finally opening the CFTR channel leading to ion/water uptake and organoid swelling. Hence, the intestinal organoid swelling will be observed with Forskolin addition if the mutated CFTR gene is corrected, while intestinal organoids with CFTR mutation will remain at the baseline volume (FIG. 14F). Intestinal organoid swelling was observed after treatment of DOTAP10-HDR, suggesting successful gene correction at G542X mutation site was achieved. No organoid swelling was detected after NC (NP only) treatment (n=8 biologically independent samples). Scale bar: 500 m (FIG. 14G). Quantitative organoid swelling (%) after administration of DOTAP10-HDR compared to the negative control (FIG. 14H). More than 20% swelled organoids were detected after treated with DOTAP10-HDR, suggesting efficient gene correction at CFTR gene. i, HDR correction in intestinal organoids were successfully detected after treatment of DOTAP10-HDR. (FIG. 14I). Data of FIG. 14H-I are shown as mean±s.e.m. (n=8 biologically independent samples). DOTAP LNPs successfully corrected CFTR gene mutation in G542X-CF mouse model and restored CFTR function in intestinal organoid cell model containing G542X mutation.

FIGS. 15A-B show luciferase expression in mouse organs after administration of lung SORT LNPs or DOTAP40 LNPs as described herein, by adjusting the internal ratio among lipid components, the optimized DOTAP40 LNPs showed better delivery efficiency in mouse lungs than Lung SORT LNPs reported previously (0.1 mg kg-1 Luc mRNA). Similar mRNA delivery efficiency was observed after decreasing weight ratio of total lipid/total NA from 40/1 to 20/1, detected using IVIS imaging system (FIG. 15A), and quantitative results after different treatment were measured from IVIS images obtained in FIG. 15A. Data are shown as mean±s.e.m. (n=3 biologically independent animals) (FIG. 15B). The optimized DOTAP40 LNPs showed much higher luciferase expression in mouse lung, compared to Lung SORT LNPs reported.

FIG. 16 shows representative flow cytometry images showing that DOTAP40 LNPs efficiently delivered Cre mRNA to basal cells of tdTOM mouse lung and turned on tdTOM fluorescence. tdTOM mice were treated with DOTAP40 LNPs (20:1) encapsulating Cre mRNA twice by I.V. injection (2 mg kg⁻¹ Cre mRNA). Two days after the second injection, mouse lungs were collected and lung basal cell populations were sorted using Anti-p75 NGF receptor antibody and the tdTOM positive cells in the whole basal cell populations were analyzed using flow cytometry (n=4 biologically independent animals in DOTAP40-Cre group). Nanoparticle only treatment group was used as negative control (NC).

FIGS. 17A-N show patient derived undifferentiated HBE cells with F508del mutation (passage 1, P1) were treated with DOTAP10-HDR LNPs at total NA concentration of 800 ng/well for 4 days before transferring into 10 cm² dish and T-75 flask for cell expansion (passage 2, P2). One week after, these cells were established on transwell inserts for cell differentiation (passage 3, P3) and CFTR function study. Untreated group was used as negative control (NC) (FIG. 17A). DOTAP10 LNPs encapsulating mCherry mRNA (DOTAP10-mCherry) were efficiently internalized into P1 HBE cells and bright mCherry fluorescence was observed after treatment of 24h (1 μg well⁻¹ of mCherry mRNA). Scale bar: 500 m (FIG. 17B). The HDR correction efficiency maintained at as high as 16% after single treatment of DOTAP10-HDR formulations. The HDR editing % in P1, P2, and differentiated (P3) F508del HBE cells were evaluated using Sanger sequencing and TIDER analysis (FIG. 17C). DOTAP10-HDR treatment successfully restored CFTR mediated Cl⁻ transport, compared to untreated group (NC) (FIG. 17D). Quantitative data from values of area under the curve (AUC) further confirmed CFTR restoration. Data are shown as mean±s.e.m. (n=6 biologically independent samples) (FIG. 17E). Expression of CFTR protein was detected after treatment of DOTAP10-HDR, while no CFTR expression was detected with NC treatment (1, 2 means two sample repeats) (FIG. 17F). Co-treatment of DOTAP10-HDR and VX-809 further enhanced Cl⁻ transport and improved CFTR function (FIG. 17G). Quantitative data from values of area under the curve (AUC) shown in FIG. 17H. Data are shown as mean±s.e.m. (n=4 biologically independent samples) (FIG. 17H). increased expression of CFTR protein was detected after co-treatment of DOTAP10-HDR+VX-809, compared to DOTAP10-HDR treatment and VX-809 treatment (FIG. 17I). DOTAP10-HDR LNPs were directly added onto the surface of differentiated HBE cells with F508del mutation in transwell inserts to correct mutated gene and restore CFTR function (total NA at 12 μg/well) (FIG. 17J). DOTAP10 LNPs encapsulating mCherry mRNA (DOTAP10-mCherry) were efficiently internalized into fully differentiated HBE cells with F508del mutation. Bright mCherry fluorescence was observed after treatment for 24h (12 g well⁻¹ of mCherry mRNA). Scale bar: 500 m (FIG. 17K). DOTAP10-HDR successfully corrected F508del mutation in differentiated HBE cells, detected by sanger sequencing and TIDER analysis. Data are shown as mean±s.e.m. (n=5 biologically independent samples) (FIG. 17L). Co-treatment of DOTAP10-HDR and VX-809 significantly enhanced Cl⁻ transport and improved CFTR function in differentiated F508del HBE cells (FIG. 17M). Quantitative data from values of area under the curve (AUC) shown in 1. Data are shown as mean±s.e.m. (n=3 biologically independent samples) (FIG. 17N).

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “N-terminal end sequence,” as used herein with respect to a polypeptide or polynucleotide sequence of interest, generally means that no other amino acid or nucleotide residues precede the N-terminal end sequence in the polypeptide or polynucleotide sequence of interest at the N-terminal end. The term “C-terminal end sequence,” as used herein with respect to a polypeptide or polynucleotide sequence of interest, generally means that no other amino acid or nucleotide residues follows the C-terminal end sequence in the polypeptide or polynucleotide sequence of interest at the C-terminal end.

The terms “non-naturally occurring” and “non-natural” are used interchangeably herein. The term “non-naturally occurring” or “non-natural,” as used herein with respect to a polypeptide or polynucleotide, generally means that the agent is not biologically derived in mammals (including but not limited to human). The term “non-naturally occurring” or “non-natural,”as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “equivalent molar dose” generally means that the amounts of materials administered to a subject have an equivalent amount of moles, based on the molecular weight of the material used in the dose.

The term “therapeutically effective and non-toxic dose,” as used herein, generally refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The terms “cancer” and “cancerous” refer to or describe the physiological condition in mammals that is typically characterized by unregulated cell growth/proliferation.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “−” means a single bond, “═” means a double bond, and “≡” means triple bond. The symbol “----” represents an optional bond, which if present is either single or double. The symbol “ [symbol] ” represents a single bond or a double bond. Thus, for example, the formula

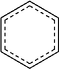

includes

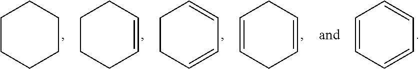

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

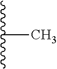

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

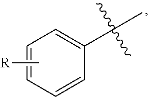

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

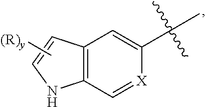

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cₙ₋ₙ′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or

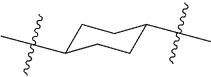 [SMILES: *C1CCC(*)CC1]

triple bonds, and no atoms other than carbon and hydrogen. The group is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

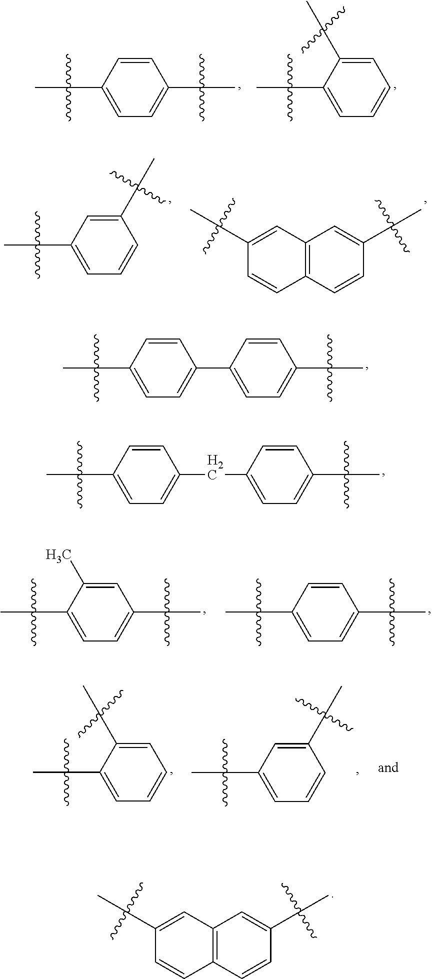

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

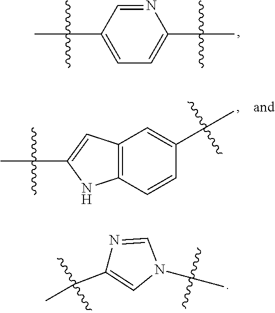

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to a divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

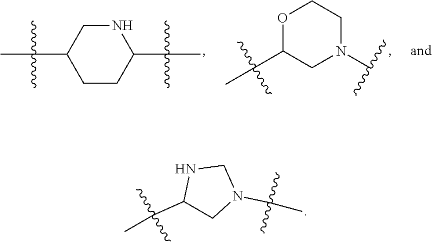 [SMILES: *C1CCC(*)NC1 ; *C1CN(*)CCO1 ; *C1CN(*)CN1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

Throughout this application, the term “about” is used to indicate that a value includes the inherent variation of error for the device, the method being employed to determine the value, or the variation that exists among the study subjects. Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

As used herein, the term “detectable” refers to an occurrence of, or a change in, a signal that is directly or indirectly detectable either by observation or by instrumentation. Typically, a detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density

The term “potent” or “potency,” as used herein in connection with delivery of nucleic acid editing composition(s), generally refers to a greater ability of a delivery system (e.g., a lipid composition) to achieve or bring about a desired amount, activity, or effect of a nucleic acid editing system (such as a desired level of translation, transcription, production, expression, or activity of a protein or gene) in cells (e.g., targeted cells) to any measurable extent, e.g., relative to a reference delivery system. For example, a lipid composition with a higher potency may achieve a desired therapeutic effect in a greater population of relevant cells, within a shorter response time, or that last a longer period of time.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer or dendron, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Off-targe indel”, as the term used herein, generally refers to an indel at or near a site other than the target sequence of the targeting domain of the gRNA molecule. Such sites may comprise, for example, 1, 2, 3, 4, 5 or more mismatch nucleotides relative to the sequence of the targeting domain of the gRNA

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### Nucleic Acid Editing Systems

Disclosed herein includes a composition comprising a nucleic acid editing system assembled with (e.g., encapsulated within) a lipid composition, wherein the nucleic acid editing system comprises: (i) a guide nucleic acid comprising a targeting sequence that is complementary with a target sequence of a (e.g., endogenous) (e.g., mutant) cystic fibrosis transmembrane conductance regulator (CFTR) gene or transcript; (ii) a (e.g., heterologous) polypeptide (such as one described herein) comprising a (e.g., heterologous) actuator moiety (such as endonuclease) or a (e.g., heterologous) polynucleotide (such as one described herein) encoding the polypeptide, which actuator moiety is configured to (1) form a complex with the guide nucleic acid and (2) cleave the (e.g., endogenous) (e.g., mutant) CFTR gene or transcript in a cell in a cleavage event; and (iii) a donor template nucleic acid configured to alter the (e.g., endogenous) (e.g., mutant) CFTR gene or transcript, subsequent to the cleavage event, to provide a functional CFTR gene, transcript or protein in the cell. The composition may be for enhancing an expression or activity of cystic fibrosis transmembrane conductance regulator (CFTR) protein in a cell that exhibits a (e.g., endogenous) mutant CFTR gene or transcript.

In some embodiments, the guide nucleic acid comprises a nucleotide sequence selected from those set forth in Table A (or disclosed elsewhere herein) and complementary sequences thereof. The targeting sequence of the guide nucleic acid may comprise a nucleotide sequence selected from those set forth in Table A (or disclosed elsewhere herein) and complementary sequences thereof. The target sequence may be within a region of the CFTR gene or transcript. For example, the target sequence may be specific to a region with a mutation of CFTR relative to a corresponding wild-type couterpart. The target sequence may be within a region coding for an ATP binding domain, an intrically disordered domain, a transmembrane domain, transporter domain, a PDZ domain, or other region, domain, or motif of the CFTR protein, gene or transcript. Exon 10 is the location of the F508del mutation in human CFTR. Exon 12 is the location of the G542X mutation in mouse CFTR gene. The (e.g., heterologous) actuator moiety (such as endonuclease) may be configured to cleave a (e.g., endogenous) CFTR gene or transcript at a cleavage site flanking a mutation of CFTR.

TABLE A
Non-limiting example sequence(s) of guide(s)

| SEQ | | |
|---|---|---|
| ID | | |
| NO: | Sequence | |
| 1 | ACCATTAAAGAAAATATCAT | SgF508Del-1 |
| 2 | TCTGTATCTATATTCATCAT | SgF508Del-2 |
| 3 | GACAACACAGTTCTCTGAGAAGG | SgCFTR-1 |

In some embodiments, the donor template nucleic acid is configured to insert one or more nucleotides into an endogenous (e.g., mutant) CFTR gene or transcript at or near a cleavage site in a cleavage event. In some embodiments, the donor template nucleic acid comprises a nucleotide sequence selected from those set forth in Table B (or disclosed elsewhere herein) and complementary sequences thereof. In some embodiments, the donor template nucleic acid comprises a 5′ homology arm. In some embodiments, the donor template nucleic acid comprises a 3′ homology arm. In some embodiments, the donor template nucleic acid comprises a 5′ homology arm and a 3′ homology arm. The donor template nucleic acid may comprise a sequence of a wild-type CFTR gene or transcript (or a fragment thereof). The donor template nucleic acid may be a (e.g., single-stranded) oligonucleotide donor (ODN). The donor template sequence may have homology to a target sequence region such that homology-based repair mechanisms may be utilized. For example, the donor template nucleic acid may have homology 3′ and 5′ of a target location or cleavage location. The homologous arms may anneal to the target sequence and allow homology based repair to occur and allow incorporation of the donor nucleic acid. In some embodiments, the donor template nucleic acid is configured to insert one or more nucleotides into said mutant CFTR gene or transcript at or near the cleavage site. The one or more nucleotides may correspond to a sequence of a wild-type CFTR. The insertion of the one or more nucleotides may cause the mutant CFTR gene or transcript to be repaired such that the gene or transcript corresponds to a wild-type CFTR (or non-mutant sequence). The donor template nucleic acid may allow sequence to be inserted into the gene, and may be used to correct a deletion. The donor template nucleic acid may be configured to alter the mutant CFTR gene or transcript in a homology directed repair (HDR) pathway or event(s) subsequent to the cleavage event.

TABLE B
Non-limiting example sequence(s) of HDR template(s)

| SEQ | | |
|---|---|---|
| ID | | |
| NO: | Sequence | |
| 4 | AGTTTTCCTGGATTATGCCTGGCACCATTAAAGAA | SgF508-1-NTS-40 |
| | AATATCATCTTTGGTGTTTCCTATGATGAATATAGA | |
| | TACAGAAGC | |
| 5 | GCTTCTGTATCTATATTCATCATAGGAAACACCAA | SgF508-1-TS-40 |
| | AGATGATATTTTCTTTAATGGTGCCAGGCATAATCC | |
| | AGGAAAACT | |
| 6 | AAGAATTTCATTCTGTTCTCAGTTTTCCTGGATTAT | SgF508-1-NTS-60 |
| | GCCTGGCACCATTAAAGAAAATATCATCTTTGGTG | |
| | TTTCCTATGATGAATATAGATACAGAAGCGTCATC | |
| | AAAGCATGCCAACT | |
| 7 | ATAGGAAACACCAAAGATGATATTTTCTTTAATGG | SgF508-1-TS |
| | TGCCAGGCATAATCCAGGAAAACTGAGAACAGAA | |
| | TGAAATTCTTCCACTGTG | |
| 8 | TTTCAATTTTGGATTGTGCATGCTAAATTTTATTTC | HDR-CFTR1- |
| | TGGTGTTATGCTTTGGATAATAGGACATCACCAAG | NTS-100 |
| | TTTGCAGAACAAGACAACACAGTTCTAGGAGAAG | |
| | GTGGAGTCACACTGAGTGGAGGTCAGCGTGCAAG | |
| | GATTTCTTTAGCAAGGTAAATATTTAACTGTTGGTC | |
| | TTGTGAGCACTTGCTGTAAATACTA | |
| 9 | TGCTAAATTTTATTTCTGGTGTTATGCTTTGGATAA | HDR-CFTR1- |
| | TAGGACATCACCAAGTTTGCAGAACAAGACAACAC | NTS-80 |
| | AGTTCTAGGAGAAGGTGGAGTCACACTGAGTGGA | |
| | GGTCAGCGTGCAAGGATTTCTTTAGCAAGGTAAAT | |
| | ATTTAACTGTTGGTCTTGTG | |
| 10 | GTTATGCTTTGGATAATAGGACATCACCAAGTTTG | HDR-CFTR1- |
| | CAGAACAAGACAACACAGTTCTAGGAGAAGGTGG | NTS-60 |
| | AGTCACACTGAGTGGAGGTCAGCGTGCAAGGATTT | |
| | CTTTAGCAAGGTAAAT | |
| 11 | ACATCACCAAGTTTGCAGAACAAGACAACACAGTT | HDR-CFTR1- |
| | CTAGGAGAAGGTGGAGTCACACTGAGTGGAGGTC | NTS-40 |
| | AGCGTGCAAGG | |
| 12 | CCTTGCACGCTGACCTCCACTCAGTGTGACTCCACC | HDR-CFTR1-TS- |
| | TTCTCCTAGAACTGTGTTGTCTTGTTCTGCAAACTT | 40 |
| | GGTGATGT | |
| 13 | GTTTGCAGAACAAGACAACACAGTTCTAGGAGAA | HDR-CFTR1- |
| | GGTGGAGTCACACTGAGTGGAGGTCA | NTS-30 |

In some embodiments of the composition or nucleic acid editing system, (ii) is a messenger ribonucleic acid (mRNA) encoding the polypeptide comprising the (e.g., heterologous) actuator moiety (such as endonuclease). In some embodiments of the composition or nucleic acid editing system, (ii) is the polypeptide comprising the (e.g., heterologous) actuator moiety (such as endonuclease). The (e.g., heterologous) actuator moiety (such as endonuclease) may be a CRISPR-associated (Cas) polypeptide or a modification thereof. The (e.g., heterologous) actuator moiety (such as endonuclease) may be a Cas9, Cas12, Cas13, Cpf1 (or Cas12a), C2C1, C2C2 (or Cas13a), Cas13b, Cas13c, Cas13d, Cas14, C2C3, Casl, CaslB, Cas2, Cas3, Cas4, Cas5, Cas5e (CasD), Cas6, Cas6e, Cas6f, Cas7, Cas8a, Cas8al, Cas8a2, Cas8b, Cas8c, Csnl, Csx12, Cas10, Cas10d, CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx1O, Csx16, CsaX, Csx3, Csxl, Csx15, Csfl, Csf2, Csf3, Csf4, or Cu1966; any derivative thereof; any variant thereof; or any fragment thereof. The (e.g., heterologous) actuator moiety (such as endonuclease) may be Cas9.

TABLE C
Non-limiting example sequence(s) of polynucleotide(s) encoding actuator moiety

| SEQ | | |
|---|---|---|
| ID | | |
| NO | Sequence | |
| 14 | ATGGCCCCAAAGAAGAAGCGGAAGGTCGGTATCCACGGAGTCCCAG | Cas9 |
| | CAGCCGACAAGAAGTACAGCATCGGCCTGGACATCGGCACCAACTC | mRNA |
| | TGTGGGCTGGGCCGTGATCACCGACGAGTACAAGGTGCCCAGCAAG | |
| | AAATTCAAGGTGCTGGGCAACACCGACCGGCACAGCATCAAGAAGA | |
| | ACCTGATCGGAGCCCTGCTGTTCGACAGCGGCGAAACAGCCGAGGC | |
| | CACCCGGCTGAAGAGAACCGCCAGAAGAAGATACACCAGACGGAA | |
| | GAACCGGATCTGCTATCTGCAAGAGATCTTCAGCAACGAGATGGCC | |
| | AAGGTGGACGACAGCTTCTTCCACAGACTGGAAGAGTCCTTCCTGGT | |
| | GGAAGAGGATAAGAAGCACGAGCGGCACCCCATCTTCGGCAACATC | |
| | GTGGACGAGGTGGCCTACCACGAGAAGTACCCCACCATCTACCACC | |
| | TGAGAAAGAAACTGGTGGACAGCACCGACAAGGCCGACCTGCGGCT | |
| | GATCTATCTGGCCCTGGCCCACATGATCAAGTTCCGGGGCCACTTCC | |
| | TGATCGAGGGCGACCTGAACCCCGACAACAGCGACGTGGACAAGCT | |
| | GTTCATCCAGCTGGTGCAGACCTACAACCAGCTGTTCGAGGAAAAC | |
| | CCCATCAACGCCAGCGGCGTGGACGCCAAGGCCATCCTGTCTGCCA | |
| | GACTGAGCAAGAGCAGACGGCTGGAAAATCTGATCGCCCAGCTGCC | |
| | CGGCGAGAAGAAGAATGGCCTGTTCGGAAACCTGATTGCCCTGAGC | |
| | CTGGGCCTGACCCCCAACTTCAAGAGCAACTTCGACCTGGCCGAGG | |
| | ATGCCAAACTGCAGCTGAGCAAGGACACCTACGACGACGACCTGGA | |
| | CAACCTGCTGGCCCAGATCGGCGACCAGTACGCCGACCTGTTTCTGG | |
| | CCGCCAAGAACCTGTCCGACGCCATCCTGCTGAGCGACATCCTGAG | |
| | AGTGAACACCGAGATCACCAAGGCCCCCCTGAGCGCCTCTATGATC | |
| | AAGAGATACGACGAGCACCACCAGGACCTGACCCTGCTGAAAGCTC | |
| | TCGTGCGGCAGCAGCTGCCTGAGAAGTACAAAGAGATTTTCTTCGA | |
| | CCAGAGCAAGAACGGCTACGCCGGCTACATTGACGGCGGAGCCAGC | |
| | CAGGAAGAGTTCTACAAGTTCATCAAGCCCATCCTGGAAAAGATGG | |
| | ACGGCACCGAGGAACTGCTCGTGAAGCTGAACAGAGAGGACCTGCT | |
| | GCGGAAGCAGCGGACCTTCGACAACGGCAGCATCCCCCACCAGATC | |
| | CACCTGGGAGAGCTGCACGCCATTCTGCGGCGGCAGGAAGATTTTT | |
| | ACCCATTCCTGAAGGACAACCGGGAAAAGATCGAGAAGATCCTGAC | |
| | CTTCCGCATCCCCTACTACGTGGGCCCTCTGGCCAGGGGAAACAGCA | |
| | GATTCGCCTGGATGACCAGAAAGAGCGAGGAAACCATCACCCCCTG | |
| | GAACTTCGAGGAAGTGGTGGACAAGGGCGCTTCCGCCCAGAGCTTC | |
| | ATCGAGCGGATGACCAACTTCGATAAGAACCTGCCCAACGAGAAGG | |
| | TGCTGCCCAAGCACAGCCTGCTGTACGAGTACTTCACCGTGTATAAC | |
| | GAGCTGACCAAAGTGAAATACGTGACCGAGGGAATGAGAAAGCCC | |
| | GCCTTCCTGAGCGGCGAGCAGAAAAAGGCCATCGTGGACCTGCTGT | |
| | TCAAGACCAACCGGAAAGTGACCGTGAAGCAGCTGAAAGAGGACT | |
| | ACTTCAAGAAAATCGAGTGCTTCGACTCCGTGGAAATCTCCGGCGTG | |
| | GAAGATCGGTTCAACGCCTCCCTGGGCACATACCACGATCTGCTGA | |
| | AAATTATCAAGGACAAGGACTTCCTGGACAATGAGGAAAACGAGGA | |
| | CATTCTGGAAGATATCGTGCTGACCCTGACACTGTTTGAGGACAGAG | |
| | AGATGATCGAGGAACGGCTGAAAACCTATGCCCACCTGTTCGACGA | |
| | CAAAGTGATGAAGCAGCTGAAGCGGCGGAGATACACCGGCTGGGG | |
| | CAGGCTGAGCCGGAAGCTGATCAACGGCATCCGGGACAAGCAGTCC | |
| | GGCAAGACAATCCTGGATTTCCTGAAGTCCGACGGCTTCGCCAACA | |
| | GAAACTTCATGCAGCTGATCCACGACGACAGCCTGACCTTTAAAGA | |
| | GGACATCCAGAAAGCCCAGGTGTCCGGCCAGGGCGATAGCCTGCAC | |
| | GAGCACATTGCCAATCTGGCCGGCAGCCCCGCCATTAAGAAGGGCA | |
| | TCCTGCAGACAGTGAAGGTGGTGGACGAGCTCGTGAAAGTGATGGG | |
| | CCGGCACAAGCCCGAGAACATCGTGATCGAAATGGCCAGAGAGAAC | |
| | CAGACCACCCAGAAGGGACAGAAGAACAGCCGCGAGAGAATGAAG | |
| | CGGATCGAAGAGGGCATCAAAGAGCTGGGCAGCCAGATCCTGAAA | |
| | GAACACCCCGTGGAAAACACCCAGCTGCAGAACGAGAAGCTGTACC | |
| | TGTACTACCTGCAGAATGGGCGGGATATGTACGTGGACCAGGAACT | |
| | GGACATCAACCGGCTGTCCGACTACGATGTGGACCATATCGTGCCTC | |
| | AGAGCTTTCTGAAGGACGACTCCATCGACAACAAGGTGCTGACCAG | |
| | AAGCGACAAGAACCGGGGCAAGAGCGACAACGTGCCCTCCGAAGA | |
| | GGTCGTGAAGAAGATGAAGAACTACTGGCGGCAGCTGCTGAACGCC | |
| | AAGCTGATTACCCAGAGAAAGTTCGACAATCTGACCAAGGCCGAGA | |
| | GAGGCGGCCTGAGCGAACTGGATAAGGCCGGCTTCATCAAGAGACA | |
| | GCTGGTGGAAACCCGGCAGATCACAAAGCACGTGGCACAGATCCTG | |
| | GACTCCCGGATGAACACTAAGTACGACGAGAATGACAAGCTGATCC | |
| | GGGAAGTGAAAGTGATCACCCTGAAGTCCAAGCTGGTGTCCGATTT | |
| | CCGGAAGGATTTCCAGTTTTACAAAGTGCGCGAGATCAACAACTAC | |
| | CACCACGCCCACGACGCCTACCTGAACGCCGTCGTGGGAACCGCCC | |
| | TGATCAAAAAGTACCCTAAGCTGGAAAGCGAGTTCGTGTACGGCGA | |
| | CTACAAGGTGTACGACGTGCGGAAGATGATCGCCAAGAGCGAGCAG | |
| | GAAATCGGCAAGGCTACCGCCAAGTACTTCTTCTACAGCAACATCAT | |
| | GAACTTTTTCAAGACCGAGATTACCCTGGCCAACGGCGAGATCCGG | |
| | AAGCGGCCTCTGATCGAGACAAACGGCGAAACCGGGGAGATCGTGT | |
| | GGGATAAGGGCCGGGATTTTGCCACCGTGCGGAAAGTGCTGAGCAT | |
| | GCCCCAAGTGAATATCGTGAAAAAGACCGAGGTGCAGACAGGCGGC | |
| | TTCAGCAAAGAGTCTATCCTGCCCAAGAGGAACAGCGATAAGCTGA | |
| | TCGCCAGAAAGAAGGACTGGGACCCTAAGAAGTACGGCGGCTTCGA | |
| | CAGCCCCACCGTGGCCTATTCTGTGCTGGTGGTGGCCAAAGTGGAA | |
| | AAGGGCAAGTCCAAGAAACTGAAGAGTGTGAAAGAGCTGCTGGGG | |
| | ATCACCATCATGGAAAGAAGCAGCTTCGAGAAGAATCCCATCGACT | |
| | TTCTGGAAGCCAAGGGCTACAAAGAAGTGAAAAAGGACCTGATCAT | |
| | CAAGCTGCCTAAGTACTCCCTGTTCGAGCTGGAAAACGGCCGGAAG | |
| | AGAATGCTGGCCTCTGCCGGCGAACTGCAGAAGGGAAACGAACTGG | |
| | CCCTGCCCTCCAAATATGTGAACTTCCTGTACCTGGCCAGCCACTAT | |
| | GAGAAGCTGAAGGGCTCCCCCGAGGATAATGAGCAGAAACAGCTGT | |
| | TTGTGGAACAGCACAAGCACTACCTGGACGAGATCATCGAGCAGAT | |
| | CAGCGAGTTCTCCAAGAGAGTGATCCTGGCCGACGCTAATCTGGAC | |
| | AAAGTGCTGTCCGCCTACAACAAGCACCGGGATAAGCCCATCAGAG | |
| | AGCAGGCCGAGAATATCATCCACCTGTTTACCCTGACCAATCTGGGA | |
| | GCCCCTGCCGCCTTCAAGTACTTTGACACCACCATCGACCGGAAGAG | |
| | GTACACCAGCACCAAAGAGGTGCTGGACGCCACCCTGATCCACCAG | |
| | AGCATCACCGGCCTGTACGAGACACGGATCGACCTGTCTCAGCTGG | |
| | GAGGCGACAAAAGGCCGGCGGCCACGAAAAAGGCCGGCCAGGCAA | |
| | AAAAGAAAAAGTAA | |

In some embodiments of the composition or nucleic acid editing system, (i) and (iii) are present on two different molecules. In some embodiments of the composition or nucleic acid editing system, (i), (ii), and (iii) are present on three different molecules. In some embodiments of the composition or nucleic acid editing system, at least two of (i), (ii) and (iii) are present on one molecule.

In some embodiments of the composition or nucleic acid editing system, (i) and (ii) are present at a molar ratio that is not 1:1. In some embodiments, (i) and (ii) are present at a molar or weight ratio less than 1:1 (indicating that the mole(s) of (i) present in the composition or nucleic acid editing system is/are less than the mole(s) of (ii) present in the composition or nucleic acid editing system). In some embodiments, (i) and (ii) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (i) and (ii) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (i) and (ii) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments of the composition or nucleic acid editing system, (ii) and (i) are present at a molar or weight ratio that is not 1:1. In some embodiments, (ii) and (i) are present at a molar or weight ratio less than 1:1 (indicating that the mole(s) of (ii) present in the composition or nucleic acid editing system is/are less than the mole(s) of (i) present in the composition or nucleic acid editing system). In some embodiments, (ii) and (i) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (ii) and (i) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (ii) and (i) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments of the composition or nucleic acid editing system, (i) and (iii) are present at a molar or weight ratio that is not 1:1. In some embodiments, (i) and (iii) are present at a molar or weight ratio less than 1:1 (indicating that the mole(s) of (i) present in the composition or nucleic acid editing system is/are less than the mole(s) of (iii) present in the composition or nucleic acid editing system). In some embodiments, (i) and (iii) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (i) and (iii) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (i) and (iii) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments of the composition or nucleic acid editing system, (iii) and (i) are present at a molar or weight ratio that is not 1:1. In some embodiments, (iii) and (i) are present at a molar or weight ratio less than 1:1 (indicating that the mole(s) of (iii) present in the composition or nucleic acid editing system is/are less than the mole(s) of (i) present in the composition or nucleic acid editing system). In some embodiments, (iii) and (i) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (iii) and (i) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (iii) and (i) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments of the composition or nucleic acid editing system, (ii) and (iii) are present at a molar or weight ratio that is not 1:1. In some embodiments, (ii) and (iii) are present at a molar or weight ratio less than 1:1 (indicating that the mole(s) of (ii) present in the composition or nucleic acid editing system is/are less than the mole(s) of (iii) present in the composition or nucleic acid editing system). In some embodiments, (ii) and (iii) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (ii) and (iii) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (ii) and (iii) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments of the composition or nucleic acid editing system, (iii) and (ii) are present at a molar or weight ratio that is not 1:1. In some embodiments, (iii) and (ii) are present at a molar or weight ratio less than 1:1 (indicating that the mole(s) of (iii) present in the composition or nucleic acid editing system is/are less than the mole(s) of (ii) present in the composition or nucleic acid editing system). In some embodiments, (iii) and (ii) are present at a molar or weight ratio of at most about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30. In some embodiments, (iii) and (ii) are present at a molar or weight ratio of at least about 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In some embodiments, (iii) and (ii) are present at a molar or weight ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, or 1:30, or a range between any two of the foregoing values.

In some embodiments, in the cleavage event, the heterologous actuator moiety (such as endonuclease) cleaves the (e.g., mutant) CFTR gene or transcript at a cleavage site flanking said mutation. The cleavage event may excise a portion of the CFTR gene comprising a mutation. For example, a mutation may be an insertion and the cleavage may remover the insertion. The cleavage event may make a cleavage to the backbone of a nuclease, without excising a nucleotide. The mutant CFTR gene or transcript may comprise a mutation relative to a corresponding wild type counterpart. The CFTR mutation may be associated with cystic fibrosis, hereditary emphysema, or chronic obstructive pulmonary disease (COPD). The mutation may be selected from the group consisting of F508del, G542X, or combinations thereof. The mutant CFTR gene or transcript may comprise a (e.g., nonsense or frameshift) mutation in one or more of exons 9-27 (e.g., exon 10, exon 12) of the CFTR gene.

### Polynucleotides

In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an actuator moiety (such as endonuclease). Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

Some embodiments of the composition or nucleic acid editing system provided herein comprise a heterologous polypeptide comprising an actuator moiety. The actuator moiety can be configured to complex with a target polynucleotide corresponding to a target gene. In some embodiments, administration of the composition or nucleic acid editing system results in a modified expression or activity of the target gene. The modified expression or activity of the target gene can be detectable, for example, in at least about 1% (e.g., at least about 2%, 5%, 10%, 15%, or 20%) cells (e.g., lung cells, such as lung basal cells) of the subject.

The composition or nucleic acid editing system may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced short palindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Casl, CaslB, Cas2, Cas3, Cas4, Cas5, Cas5e (CasD), Cas6, Cas6e, Cas6f, Cas7, Cas8a, Cas8al, Cas8a2, Cas8b, Cas8c, Csnl, Csxl2, Cas10, Cas10d, CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, or Cul966; any derivative thereof; any variant thereof; or any fragment thereof. In some embodiments, Cas13 can include, but are not limited to, Casl3a, Casl3b, Casl3c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIM5, KYP, SUVR4, Set4, Setl, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. For example, a CFTR gene may be modified by the guide polynucleotide guided nuclease.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂ CH═CH₂, O—CH₂ CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂—2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof; 4′-C(CH₃)(CH₃)—O-2′ and analogs thereof; 4′-CH₂— —N(OCH₃)-2′ and analogs thereof; 4′-CH₂—O—N(CH₃)-2′; 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group; 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each Ji and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

In some embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In certain embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides poylnucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S).

Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or (S), a or R such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

### Polypeptides

In some embodiments of the compositions or nucleic acid editing systems of the present application, the composition or nucleic acid editing system assembled with the lipid composition comprises one or more one or more polypeptides. Some polypeptide may include endonucleases such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof. In some embodiments, the nuclease enzyme may include Cas proteins such as Cas1, Cas1B, Cas2, Cas3, Cas4, Cas5, Cas6, Cas7, Cas8, Cas9 (also known as Csnl and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein. For example, the CRISPR enzyme may be directed and cleaved a genomic locus of CFTR.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce non-homologous end-joining (NHEJ) or homology directed repair (HDR).

In some embodiments, the present application provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin a, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin α, Darbepoetin α, Follicle-stimulating hormone (FSH), and Factor VIII.

In some embodiments, the polypeptide comprises a peptide sequence that is at least partially identical to any of the composition or nucleic acid editing system comprising a peptide sequence. For example, the polypeptide may comprise a peptide sequence that is at least partially identical to an antibody (e.g., a monoclonal antibody) for treating a lung disease such as lung cancer.

In some embodiments, the polypeptide comprises a peptide or protein that restores the function of a defective protein in a subject being treated by the pharmaceutical composition described herein. For example, the polynucleotide comprises a peptide or protein that restores function of cystic fibrosis transmembrane conductance regulator (CFTR) protein, which may be used to rescue a subject who is afflicted with inborn error leading to the expression of the mutated CFTR protein.

### Pharmaceutical Compositions

In some embodiments, the composition is formulated for pharmaceutical (e.g., systemic) administration. The pharmaceutical (e.g., systemic) administration may be intravenous administration. In some embodiments, the pharmaceutical composition of the present application can be administrated through any suitable routes including, for example, oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical composition of the present application can be administered in a local rather than systemic manner, for example, via injection of the pharmaceutical composition directly into a targeted tissue, preferably in a sustained release formulation. Local delivery can be affected in various ways, depending on the tissue to be targeted.

In some embodiments, aerosols containing the composition of the present application can be inhaled (for nasal, tracheal, or bronchial delivery). In some embodiments, the composition of the present application can be injected into the site of injury, disease manifestation, or pain, for example. In some embodiments, the composition of the present application can be provided in lozenges for oral, tracheal, or esophageal application. In some embodiments, the composition of the present application can be supplied in liquid, tablet or capsule form for administration to the stomach or intestines. In some embodiments, the composition of the present application can be supplied in suppository form for rectal or vaginal application. In some embodiments, the composition of the present application can even be delivered to the eye by use of creams, drops, or even injection.

The pharmaceutical composition may be formulated such that administration may be performed in such a way to target or come in contact with an organ of interest. In some embodiments, the composition is formulated for nebulization. In some embodiments, the the composition is formulated for intravenous administration. In some embodiments the composition is formulated for apical delivery

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the (e.g., pharmaceutical) composition of the present application, a molar or weight ratio of (1) the polypeptide comprising the endonuclease or the polynucleotide encoding the polypeptide to (2) total lipids of the lipid composition is no more than (about) 1:1, no more than (about) 1:5, no more than (about) 1:10, no more than (about) 1:15, no more than (about) 1:20, no more than (about) 1:25, no more than (about) 1:30, no more than (about) 1:35, no more than (about) 1:40, no more than (about) 1:45, no more than (about) 1:50, no more than (about) 1:60, no more than (about) 1:70, no more than (about) 1:80, no more than (about) 1:90, or more than (about) 1:100. In some embodiments of the (e.g., pharmaceutical) composition of the present application, a molar or weight ratio of (1) the polypeptide comprising the endonuclease or the polynucleotide encoding the polypeptide to (2) total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100. In some embodiments of the (e.g., pharmaceutical) composition of the present application, a molar or weight ratio of (1) the polypeptide comprising the endonuclease or the polynucleotide encoding the polypeptide to (2) total lipids of the lipid composition is (about) 1:1, 1:5, 1:10, 1:15, 1:20, 1:25, 1:30, 1:35, 1:40, 1:45, 1:50, 1:60, 1:70, 1:80, 1:90, or 1:100, or of a range between any two of the foregoing values.

In some embodiments of the pharmaceutical composition of the present application, a molar or weight ratio of (1) total payload molecules (including polypeptide(s) and polynucleotide(s)) to (2) total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100. In some embodiments of the pharmaceutical composition of the present application, a molar or weight ratio of (1) total payload molecules (including polypeptide(s) and polynucleotide(s)) to (2) total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100. In some embodiments of the pharmaceutical composition of the present application, a molar or weight ratio of (1) total payload molecules (including polypeptide(s) and polynucleotide(s)) to (2) total lipids of the lipid composition is (about) 1:1, 1:5, 1:10, 1:15, 1:20, 1:25, 1:30, 1:35, 1:40, 1:45, 1:50, 1:60, 1:70, 1:80, 1:90, or 1:100, or of a range between any two of the foregoing values.

In some embodiments of the pharmaceutical composition of the present application, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or (about) 100% of the nucleic acid editing system is encapsulated in particles of the lipid compositions.

In some embodiments of the pharmaceutical composition of the present application, the lipid composition comprises a plurality of particles characterized by one or more characteristics of the following: (1) a (e.g., average) size of 100 nanometers (nm) or less; (2) a polydispersity index (PDI) of no more than about 0.2; and (3) a zeta potential of −10 millivolts (mV) to 10 mV.

In some embodiments of the pharmaceutical composition of the present application, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present application, the lipid composition comprises a plurality of particles with a (e.g., average) size from about 50 nanometers (nm) to about 80 nanometers (nm). In some embodiments of the pharmaceutical composition of the present application, the lipid composition comprises a plurality of particles with a (e.g., average) size from about 60 nanometers (nm) to about 80 nanometers (nm). In some embodiments of the pharmaceutical composition of the present application, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present application, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by size exclusion chromatography (SEC). The (e.g., average) size may be determined by spectroscopic methods or image based methods, for example, dynamic light scattering, static light scattering, multi-angle light scattering, laser light scattering, or dynamic image analysis.

In some embodiments of the pharmaceutical composition of the present application, the lipid composition comprises a plurality of particles with a polydispersity index (PDI) from about 0.05 to about 0.5. In some embodiments of the pharmaceutical composition of the present application, the lipid composition comprises a plurality of particles with a polydispersity index (PDI) from about 0.1 to about 0.5. In some embodiments of the pharmaceutical composition of the present application, the lipid composition comprises a plurality of particles with a polydispersity index (PDI) from about 0.1 to about 0.3. In some embodiments of the pharmaceutical composition of the present application, the lipid composition comprises a plurality of particles with a polydispersity index (PDI) from about 0.2 to about 0.5. In some embodiments of the pharmaceutical composition of the present application, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present application, the lipid composition comprises a plurality of particles with a negative zeta potential of −5 millivolts (mV) or less. In some embodiments of the pharmaceutical composition of the present application, the lipid composition comprises a plurality of particles with a negative zeta potential of −10 millivolts (mV) or less. In some embodiments of the pharmaceutical composition of the present application, the lipid composition comprises a plurality of particles with a negative zeta potential of −15 millivolts (mV) or less. In some embodiments of the pharmaceutical composition of the present application, the lipid composition comprises a plurality of particles with a negative zeta potential of −20 millivolts (mV) or less. In some embodiments of the pharmaceutical composition of the present application, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present application, the lipid composition comprises a plurality of particles with a negative zeta potential of 15 millivolts (mV) or less. In some embodiments of the pharmaceutical composition of the present application, the lipid composition comprises a plurality of particles with a negative zeta potential of 20 millivolts (mV) or less.

In some embodiments of the pharmaceutical composition of the present application, the lipid composition comprises a plurality of particles with a negative zeta potential of −5 millivolts (mV) or more. In some embodiments of the pharmaceutical composition of the present application, the lipid composition comprises a plurality of particles with a negative zeta potential of −10 millivolts (mV) or more. In some embodiments of the pharmaceutical composition of the present application, the lipid composition comprises a plurality of particles with a negative zeta potential of −15 millivolts (mV) or more. In some embodiments of the pharmaceutical composition of the present application, the lipid composition comprises a plurality of particles with a negative zeta potential of −20 millivolts (mV) or more. In some embodiments of the pharmaceutical composition of the present application, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present application, the lipid composition comprises a plurality of particles with a zeta potential of 15 millivolts (mV) or more. In some embodiments of the pharmaceutical composition of the present application, the lipid composition comprises a plurality of particles with a zeta potential of 20 millivolts (mV) or more.

In some embodiments of the pharmaceutical composition of the present application, the lipid composition has an apparent ionization constant (pKa) outside a range of 6 to 7. In some embodiments of the pharmaceutical composition of the present application, the lipid composition has an apparent pKa of about 8 or higher, about 9 or higher, about 10 or higher, about 11 or higher, about 12 or higher, or about 13 or higher. In some embodiments of the pharmaceutical composition of the present application, the lipid composition has an apparent pKa of about 8 to about 13. In some embodiments of the pharmaceutical composition of the present application, the lipid composition has an apparent pKa of about 8 to about 10. In some embodiments of the pharmaceutical composition of the present application, the lipid composition has an apparent pKa of about 9 to about 11. In some embodiments of the pharmaceutical composition of the present application, the lipid composition has an apparent pKa of about 10 to about 13. In some embodiments of the pharmaceutical composition of the present application, the lipid composition has an apparent pKa of about 8 to about 12. In some embodiments of the pharmaceutical composition of the present application, the lipid composition has an apparent pKa of about 10 to about 12.

### Lipid Compositions

In one aspect, provided herein is a lipid composition comprising: (i) an ionizable cationic lipid (such as described herein); and (iii) a selective organ targeting (SORT) lipid (such as described herein) separate from the ionizable cationic lipid. The lipid composition may further comprise a phospholipid separate from the SORT lipid.

### Ionizable Cationic Lipids

In some embodiments of the lipid composition of the present application, the lipid composition comprises an ionizable cationic lipid. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some embodiments of the lipid composition of the present application, the ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar Rₙ Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g. molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments of the lipid composition of the present application, the dendrimers or dendrons are assembled using the differential reactivity of the acrylate and methacrylate groups with amines and thiols. The dendrimers or dendrons may include secondary or tertiary amines and thioethers formed by the reaction of an acrylate group with a primary or secondary amine and a methacrylate with a mercapto group. Additionally, the repeating units of the dendrimers or dendrons may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer or dendron may be formed by removing one or more hydrogen atoms from this core. In some embodiments, these hydrogen atoms are on a heteroatom such as a nitrogen atom. In some embodiments, the terminating group is a lipophilicgroups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments of the lipid composition of the present application, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups.

Dendrimers or dendrons of Formula (I)

In some embodiments of the lipid composition, the ionizable cationic lipid comprises at least two C₈-C₂₄ alkyl groups. In some embodiments, the ionizable cationic lipid is a dendrimer or dendron further defined by the formula:

### Core-Repeating Unit-Terminating Group (D-I)

wherein the core is linked to the repeating unit by removing one or more hydrogen atoms from
the core and replacing the atom with the repeating unit and wherein:
the core has the formula:

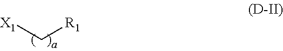

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

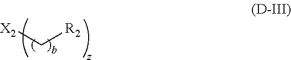

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   bis 1, 2, 3, 4, 5, or 6; and
   z is 1, 2, 3; provided that the sum of z and y is 3; or
the core has the formula:

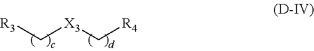

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

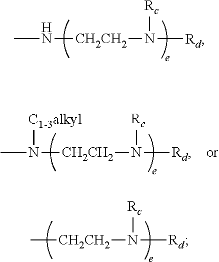

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

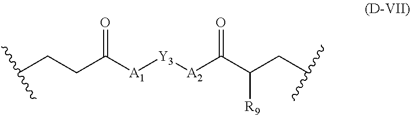

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

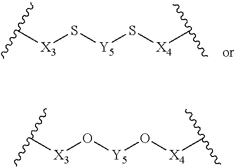

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
the linker group has the formula:

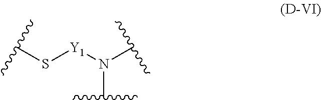

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

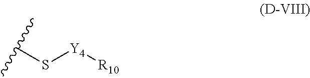

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
      or a pharmaceutically acceptable salt thereof. In some embodiments, the terminating group is further defined by the formula:

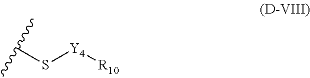

wherein:
Y₄ is alkanediyl_{(C≤18)}; and
R₁₀ is hydrogen. In some embodiments, A₁ and A₂ are each independently —O— or —NRₐ—.

In some embodiments of the dendrimer or dendron of formula (D-I), the core is further defined by the formula:

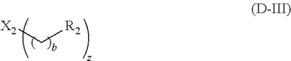

wherein:
X₂ is N(R₅)_{y};
   R₅ is hydrogen or alkyl_{(C≤8)}, or substituted alkyl_{(C≤18)}; and
   y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments of the dendrimer or dendron of formula (D-I), the core is further defined by the formula:

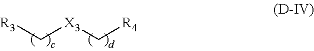

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

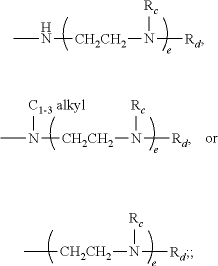

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments of the dendrimer or dendron of formula (I), the terminating group is represented by the formula:

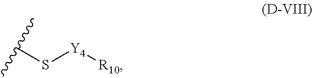

wherein:
Y₄ is alkanediyl_{(C≤18)}; and
R₁₀ is hydrogen.

In some embodiments of the dendrimer or dendron of formula (D-I), the core is further defined as:

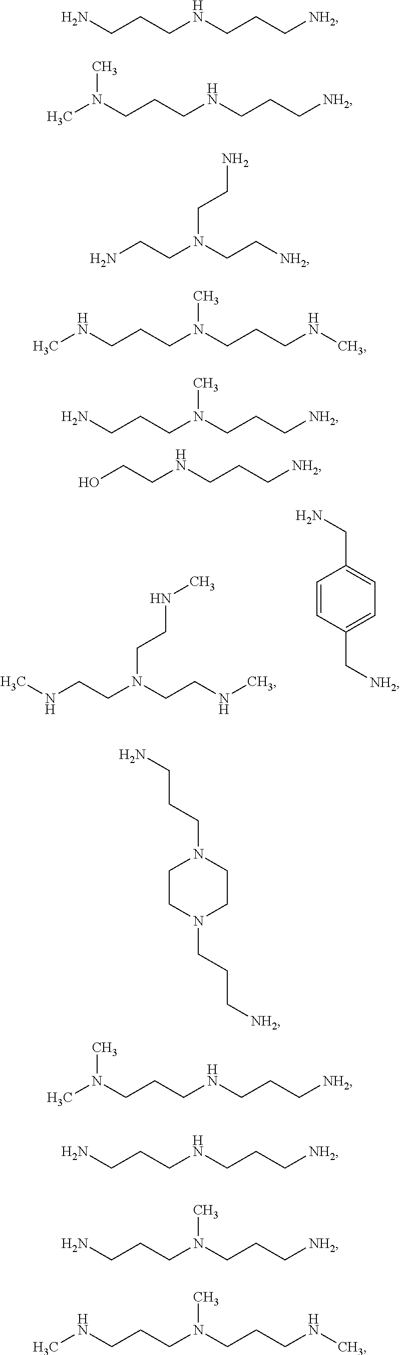

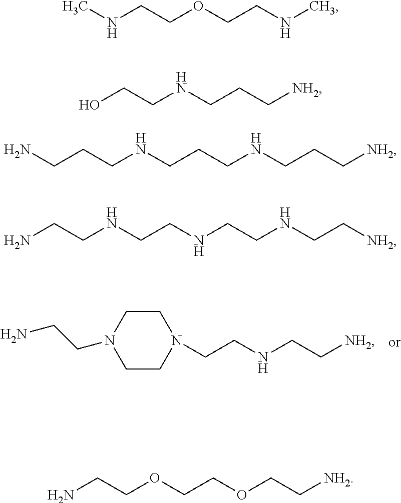

In some embodiments of the dendrimer or dendron of formula (D-I), the degradable diacyl is further defined as:

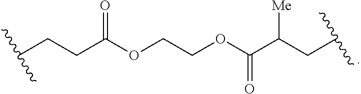 [SMILES: *CCC(=O)OCCOC(=O)C(C)C*]

In some embodiments of the dendrimer or dendron of formula (D-I), the linker is further defined as

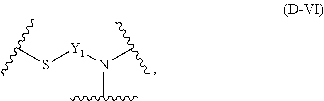

wherein Y₁ is alkanediyl_{(C≤8)} or substituted alkanediyl_{(C≤8)}.

In some embodiments of the dendrimer or dendron of formula (D-I), the dendrimer or dendron is selected from the group consisting of:

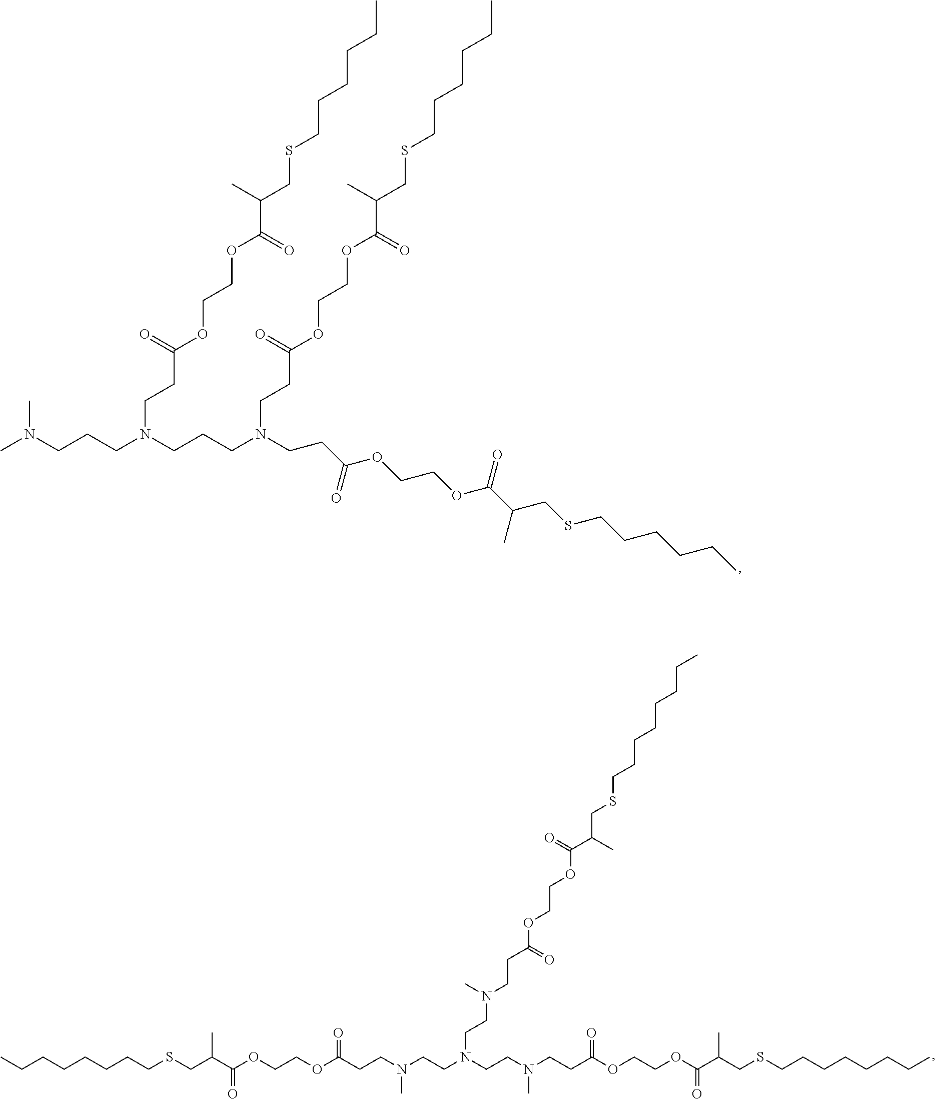

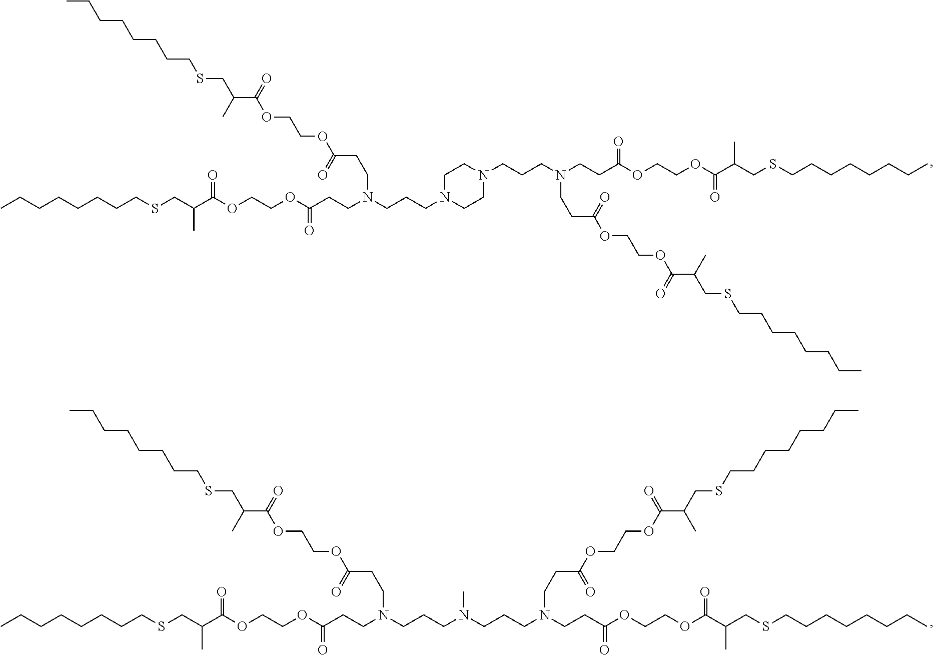

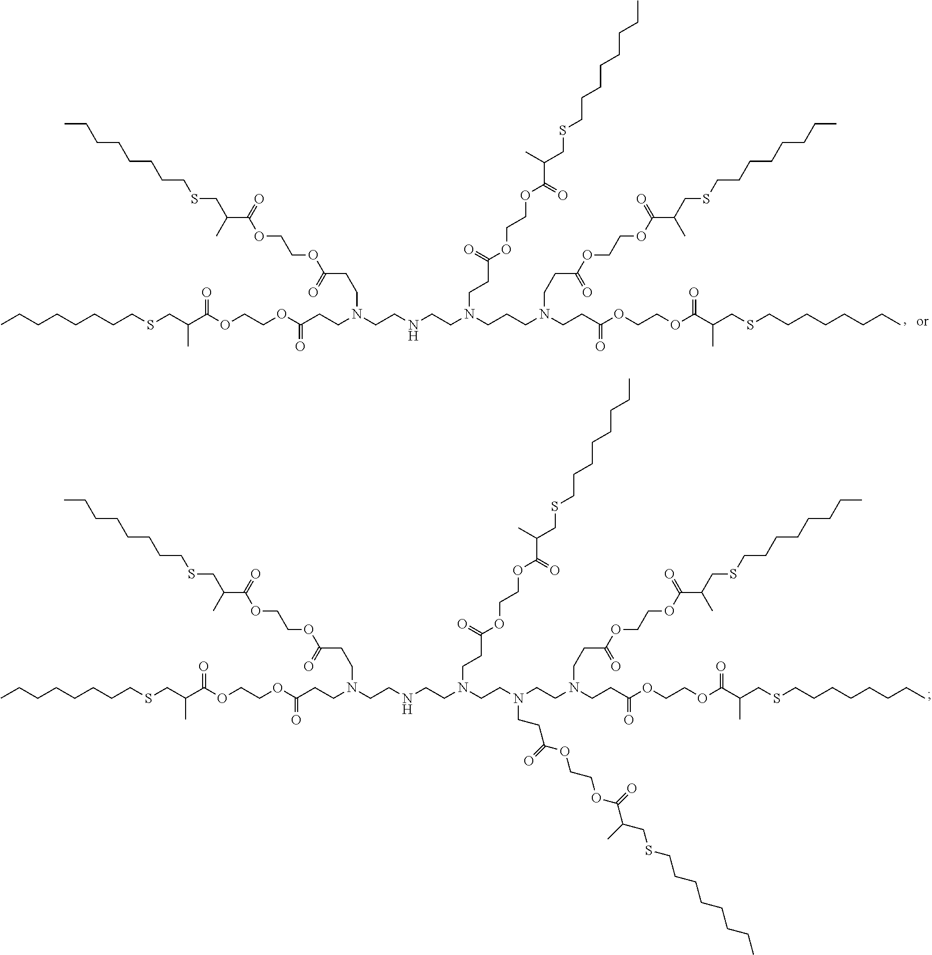

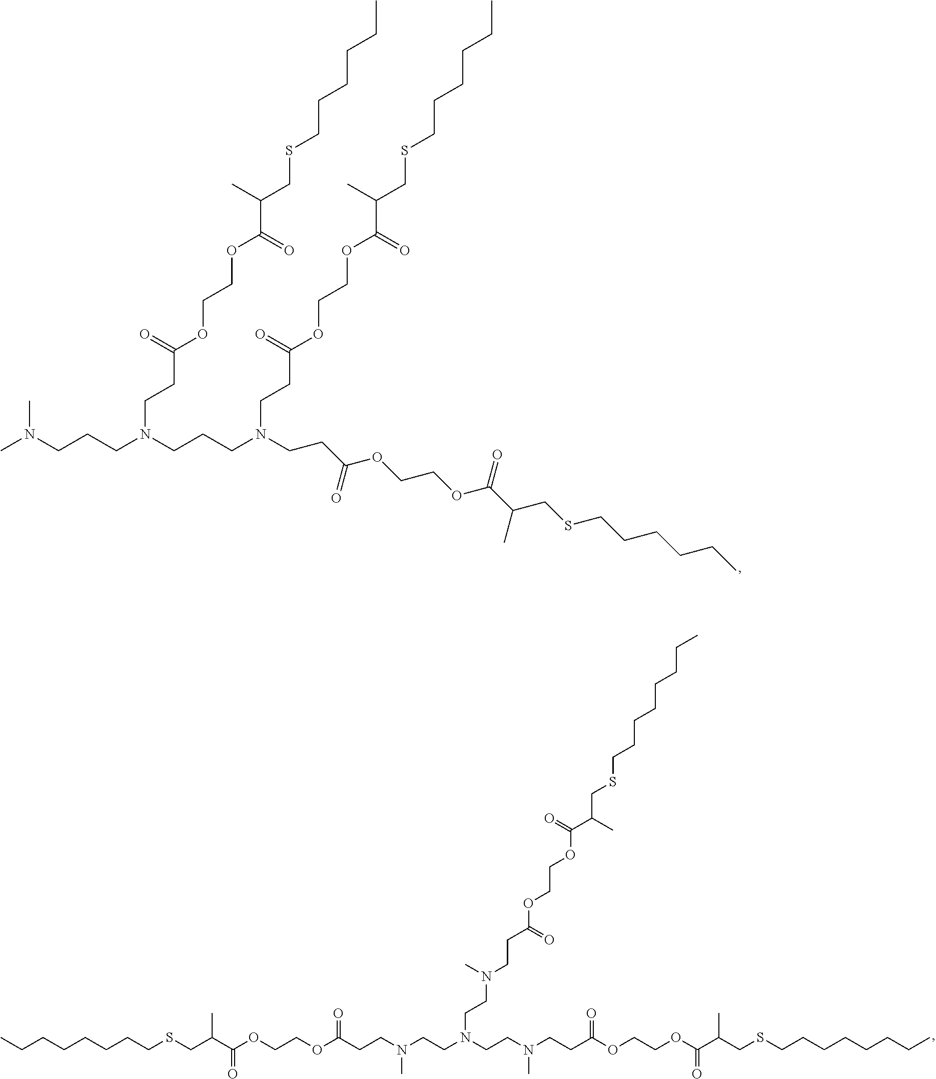

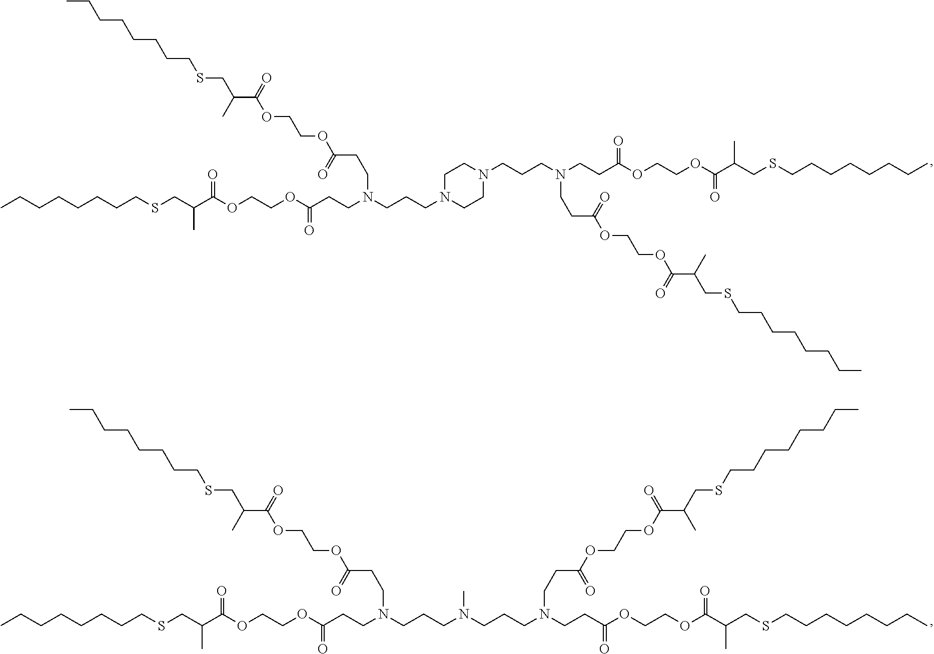

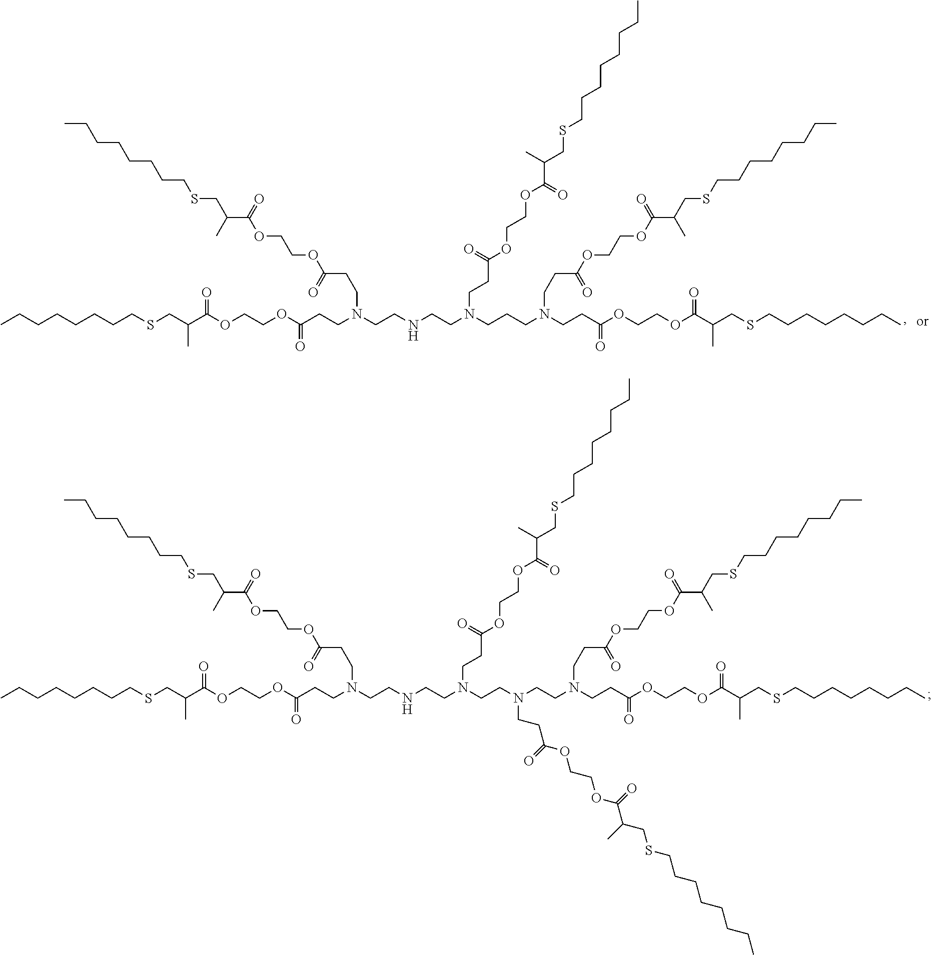

and pharmaceutically acceptable salts thereof.

### Dendrimers or Dendrons of Formula (X)

In some embodiments of the lipid composition, the ionizable cationic lipid is a dendrimer or dendron of the formula

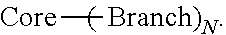

In some embodiments, the ionizable cationic lipid is a dendrimer or dendron of the formula

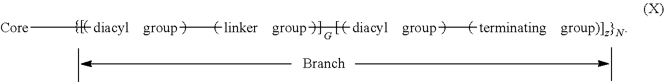

In some embodiments of the lipid composition, the ionizable cationic lipid is a dendrimer or dendron of a generation (g) having a structural formula:

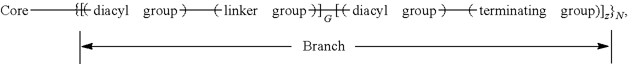

or a pharmaceutically acceptable salt thereof, wherein:
(a) the core comprises a structural formula (X_{Core}):

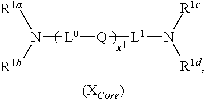

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of Li form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch})

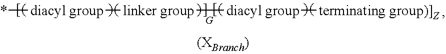

wherein:
   * indicates a point of attachment of the branch to the core;
   g is 1, 2, 3, or 4;

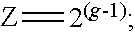

G=0, when g=1; or G=Σᵢ₌₀^{i=g-2}2ⁱ, when g≠1;
(c) each diacyl group independently comprises a structural formula

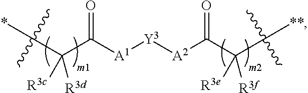

wherein:
   * indicates a point of attachment of the diacyl group at the proximal end thereof;
   ** indicates a point of attachment of the diacyl group at the distal end thereof;
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

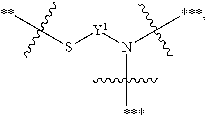

wherein:
   ** indicates a point of attachment of the linker to a proximal diacyl group;
   *** indicates a point of attachment of the linker to a distal diacyl group; and
   Y¹ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or -L²-NR^{1e}R^{1f}. In some embodiments of X_{Core} Q is independently at each occurrence a —CR^{3a}R^{3b} R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of Li form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁-₄—(CH₂CH₂)—), [(C₁-C₄) alkylene]-[(C₄-C₆) heterocycloalkyl]-[(C₁-C₄) alkylene] (e.g.,

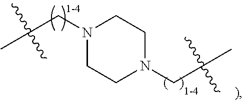

and [(C₁-C₄) alkylene]-phenylene-[(C₁-C₄) alkylene] (e.g.,

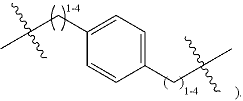

In some embodiments of X_{Core}, L⁰, L¹, and L² are each independently at each occurrence selected from C₁-C₆ alkylene (e.g., C₁-C₃ alkylene), —(C₁-C₃ alkylene-O)₁-₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments Of X_{Core}, x¹ is 1. In some embodiments Of X_{Core}, x¹ is 2. In some embodiments of X_{Core}, x¹ is 0, 3. In some embodiments of X_{Core} x¹ is 4. In some embodiments of X_{Core} x¹ is 5. In some embodiments of X_{Core}, x¹ is 6.

In some embodiments of X_{Core}, the core comprises a structural formula:

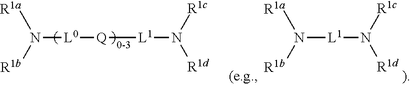

In some embodiments of X_{Core}, the core comprises a structural formula:

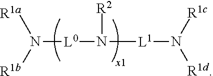

In some embodiments of X_{Core}, the core comprises a structural formula:

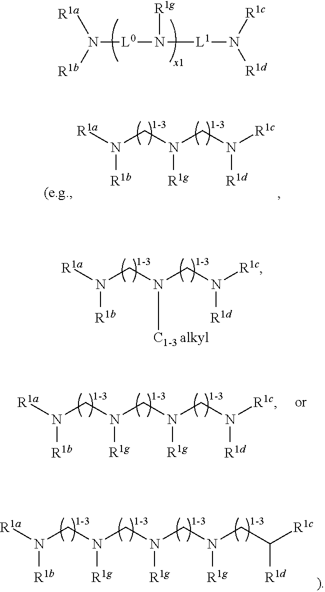

In some embodiments of X_{Core}, the core comprises a structural formula:

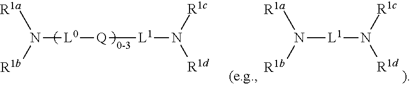

In some embodiments of X_{Core}, the core comprises a structural formula:

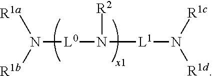

In some embodiments of X_{Core}, the core comprises a structural formula:

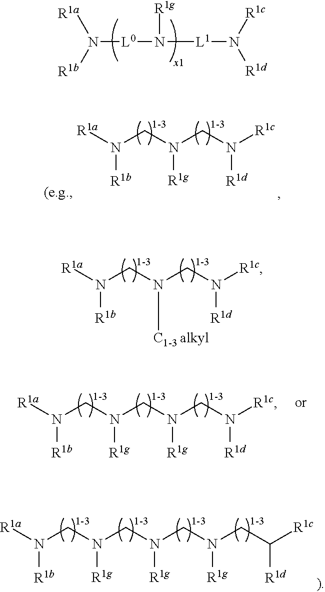

In some embodiments of X_{Core}, the core comprises a structural formula:

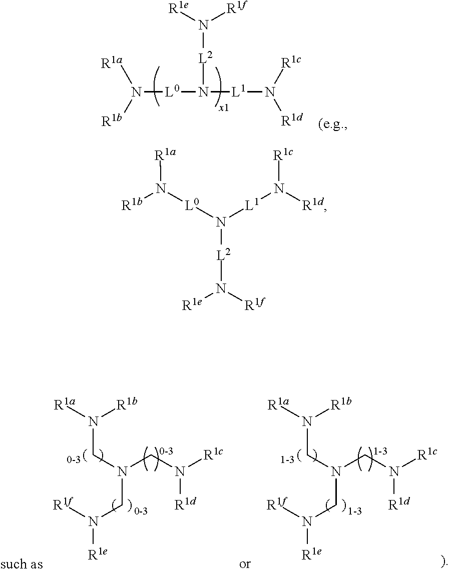

In some embodiments of X_{Core}, the core comprises a structural formula:

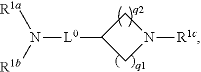

wherein Q′ is —NR²— or —CR^{3a}R^{3b}—; q¹ and q² are each independently 1 or 2. In some embodiments of X_{Core}, the core comprises a structural formula:

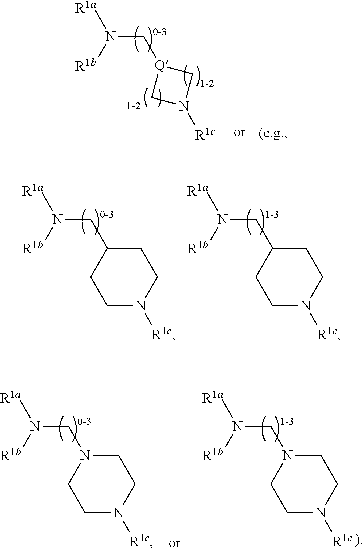

In some embodiments of X_{Core}, the core comprises a structural formula

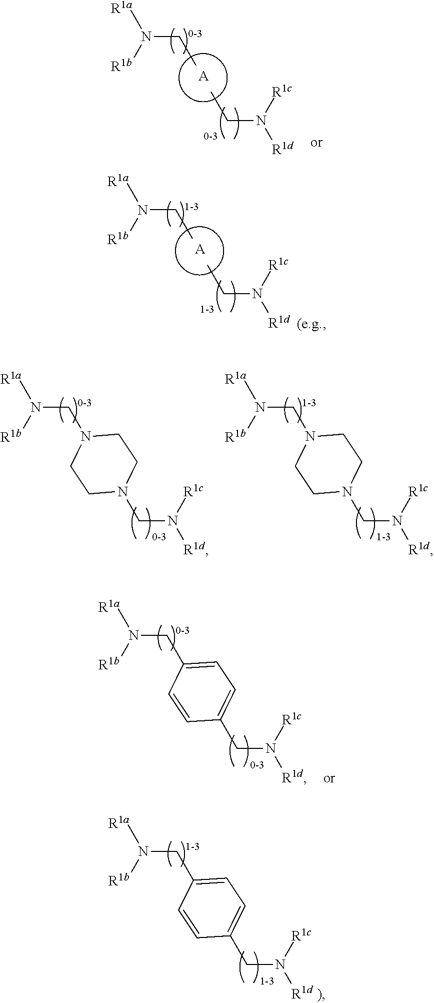

wherein ring A is an optionally substituted aryl or an optionally substituted (e.g., C₃-C₁₂, such as C₃-C₅) heteroaryl. In some embodiments of X_{Core}, the core comprises has a structural formula

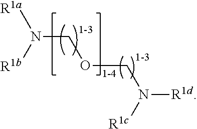

In some embodiments of X_{Core}, the core comprises a structural formula set forth in Table. 1 and pharmaceutically acceptable salts thereof, wherein * indicates a point of attachment of the core to a branch of the plurality of branches. In some embodiments, the example cores of Table. 1 are not limited to the stereoisomers (i.e. enantiomers, diastereomers) listed.

TABLE 1
Example core structures

| ID # | Structure |
|---|---|
| 1A1 | 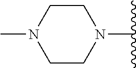 [SMILES: *N1CCN(C)CC1] |
| 1A2-1 | 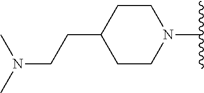 [SMILES: *N1CCC(CCN(C)C)CC1] |
| 1A2-2 | 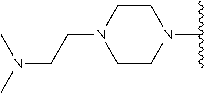 [SMILES: *N1CCN(CCN(C)C)CC1] |
| 1A3-1 | 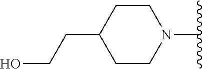 [SMILES: *N1CCC(CCO)CC1] |
| 1A3-2 | 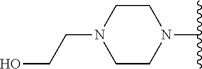 [SMILES: *N1CCN(CCO)CC1] |
| 1A4 | 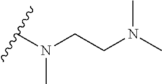 [SMILES: *N(C)CCN(C)C] |
| 1A5-1 | 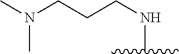 [SMILES: *NCCCN(C)C] |
| 1A5-2 | 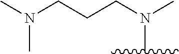 |
| 2A1-1 | 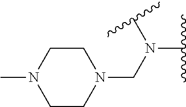 [SMILES: *N(*)CN1CCN(C)CC1] |
| 2A1-2 | 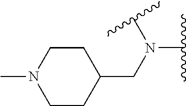 [SMILES: *N(*)CC1CCN(C)CC1] |
| 2A2-1 | 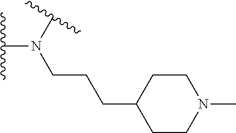 [SMILES: *N(*)CCCC1CCN(C)CC1] |
| 2A2-2 | 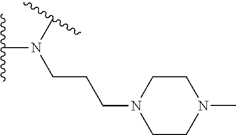 [SMILES: *N(*)CCCN1CCN(C)CC1] |
| 2A3 | 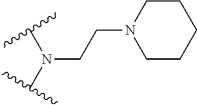 [SMILES: *N(*)CCN1CCCCC1] |
| 2A4 | 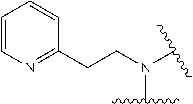 [SMILES: *N(*)CCc1ccccn1] |
| 2A5 | 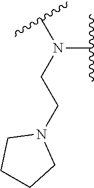 [SMILES: *N(*)CCN1CCCC1] |
| 2A6 | 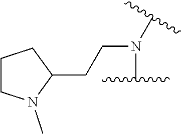 [SMILES: *N(*)CCC1CCCN1C] |
| 2A7-1 | 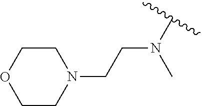 [SMILES: *N(C)CCN1CCOCC1] |
| 2A7-2 | 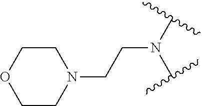 [SMILES: *N(*)CCN1CCOCC1] |
| 2A8 | 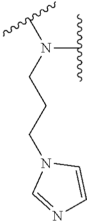 [SMILES: *N(*)CCCn1ccnc1] |
| 2A9 | 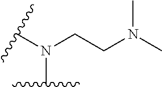 [SMILES: *N(*)CCN(C)C] |
| 2A9V | 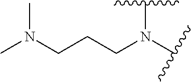 [SMILES: *N(*)CCCN(C)C] |
| 2A10 | 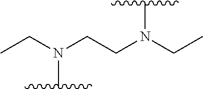 [SMILES: *N(CC)CCN(*)CC] |
| 2A11 | 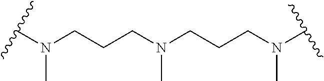 [SMILES: *N(C)CCCN(C)CCCN(*)C] |
| 2A12 | 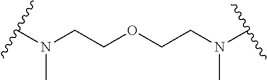 [SMILES: *N(C)CCOCCN(*)C] |
| 3A1 | 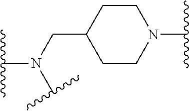 [SMILES: *N(*)CC1CCN(*)CC1] |
| 3A2 | 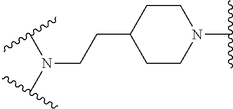 [SMILES: *N(*)CCC1CCN(*)CC1] |
| 3A3 | 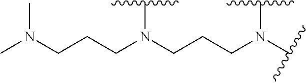 [SMILES: *N(*)CCCN(*)CCCN(C)C] |
| 3A4 | 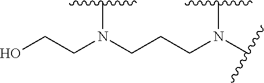 [SMILES: *N(*)CCCN(*)CCO] |
| 3A5 | 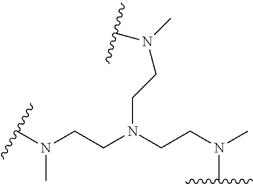 [SMILES: *N(C)CCN(CCN(*)C)CCN(*)C] |
| 3A6 | 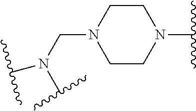 [SMILES: *N(*)CN1CCN(*)CC1] |
| 3A7 | 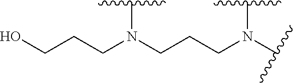 [SMILES: *N(*)CCCN(*)CCCO] |
| 4A1 | 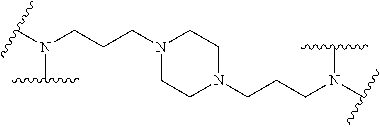 [SMILES: *N(*)CCCN1CCN(CCCN(*)*)CC1] |
| 4A2 | 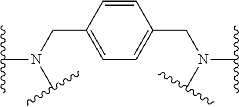 [SMILES: *N(*)Cc1ccc(CN(*)*)cc1] |
| 4A3 | 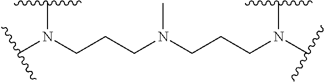 [SMILES: *N(*)CCCN(C)CCCN(*)*] |
| 4A4 | 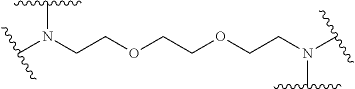 [SMILES: *N(*)CCOCCOCCN(*)*] |
| 5A1 | 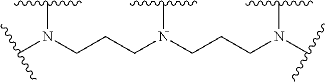 [SMILES: *N(*)CCCN(*)CCCN(*)*] |
| 5A2-1 (5-arm) | 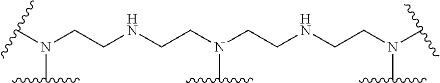 [SMILES: *N(*)CCNCCN(*)CCNCCN(*)*] |
| 5A2-2 (5-arm) | 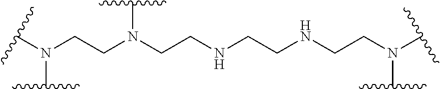 [SMILES: *N(*)CCNCCNCCN(*)CCN(*)*] |
| 5A2-3 (5-arm) | 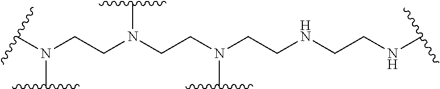 [SMILES: *NCCNCCN(*)CCN(*)CCN(*)*] |
| 5A2-4 (5-arm) | 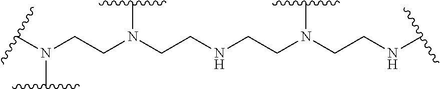 [SMILES: *NCCN(*)CCNCCN(*)CCN(*)*] |
| 5A3-1 (5-arm) | 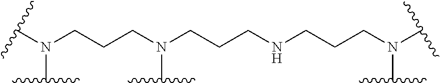 [SMILES: *N(*)CCCNCCCN(*)CCCN(*)*] |
| 5A4-1 (5-arm) | 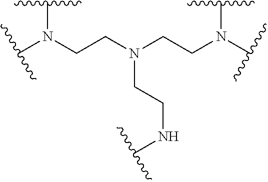 [SMILES: *NCCN(CCN(*)*)CCN(*)*] |
| 5A5 | 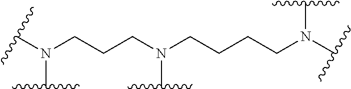 [SMILES: *N(*)CCCCN(*)CCCN(*)*] |
| 5A6 | 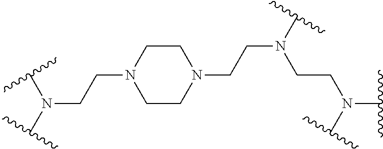 [SMILES: *N(*)CCN(*)CCN1CCN(CCN(*)*)CC1] |
| 5A2-4 (6-arm) | 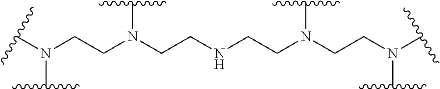 [SMILES: *N(*)CCN(*)CCNCCN(*)CCN(*)*] |
| 5A2-5 (6 arm) | 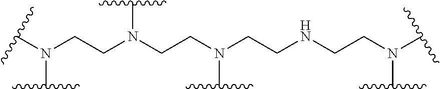 [SMILES: *N(*)CCNCCN(*)CCN(*)CCN(*)*] |
| 5A2-6 (6 arm) | 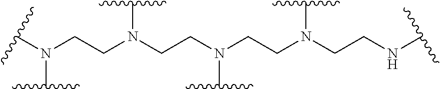 [SMILES: *NCCN(*)CCN(*)CCN(*)CCN(*)*] |
| 5A3-2 (6 arm) | 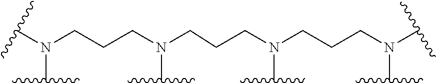 [SMILES: *N(*)CCCN(*)CCCN(*)CCCN(*)*] |
| 5A4-2 (6 arm) | 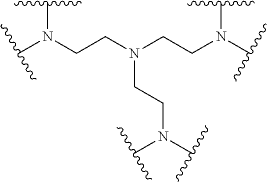 [SMILES: *N(*)CCN(CCN(*)*)CCN(*)*] |
| 6A4 | 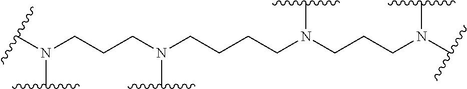 [SMILES: *N(*)CCCN(*)CCCCN(*)CCCN(*)*] |
| 1H1 | 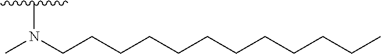 [SMILES: *N(C)CCCCCCCCCCCC] |
| 1H2 | 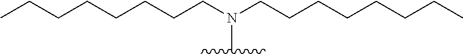 [SMILES: *N(CCCCCCCC)CCCCCCCC] |
| 1H3 | 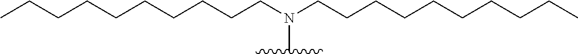 [SMILES: *N(CCCCCCCCCC)CCCCCCCCCC] |
| 2H1 | 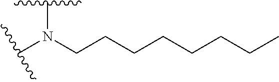 [SMILES: *N(*)CCCCCCCC] |
| 2H2 | 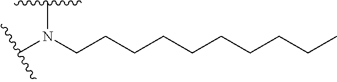 [SMILES: *N(*)CCCCCCCCCC] |
| 2H3 | 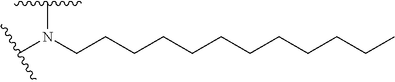 [SMILES: *N(*)CCCCCCCCCCCC] |
| 2H4 | 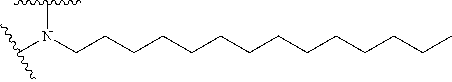 [SMILES: *N(*)CCCCCCCCCCCCCC] |
| 2H5 | 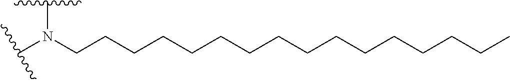 [SMILES: *N(*)CCCCCCCCCCCCCCCC] |
| 2H6 | 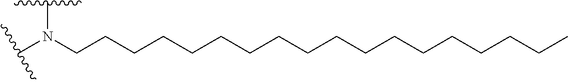 [SMILES: *N(*)CCCCCCCCCCCCCCCCCC] |

In some embodiments of X_{Core}, the core comprises a structural formula selected from the group consisting of:

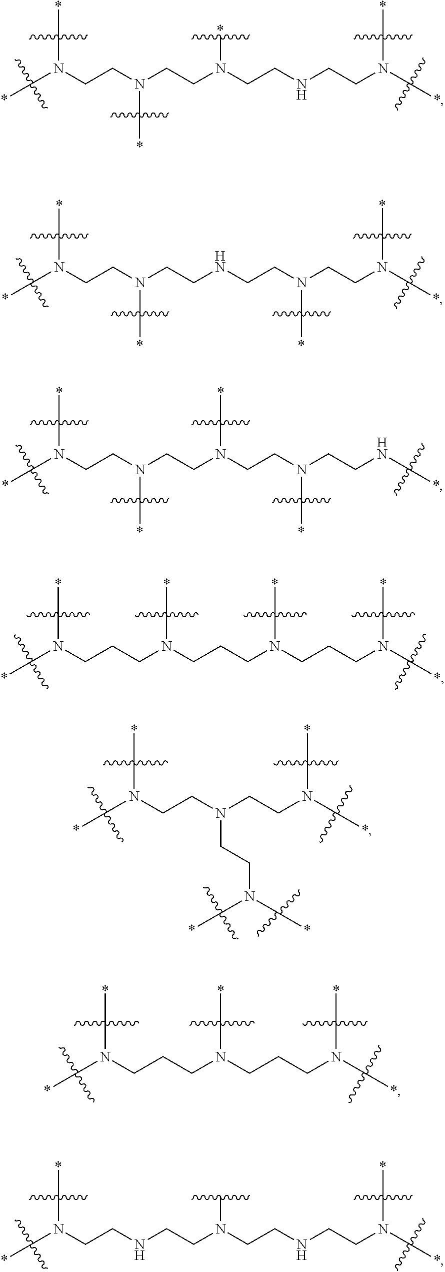

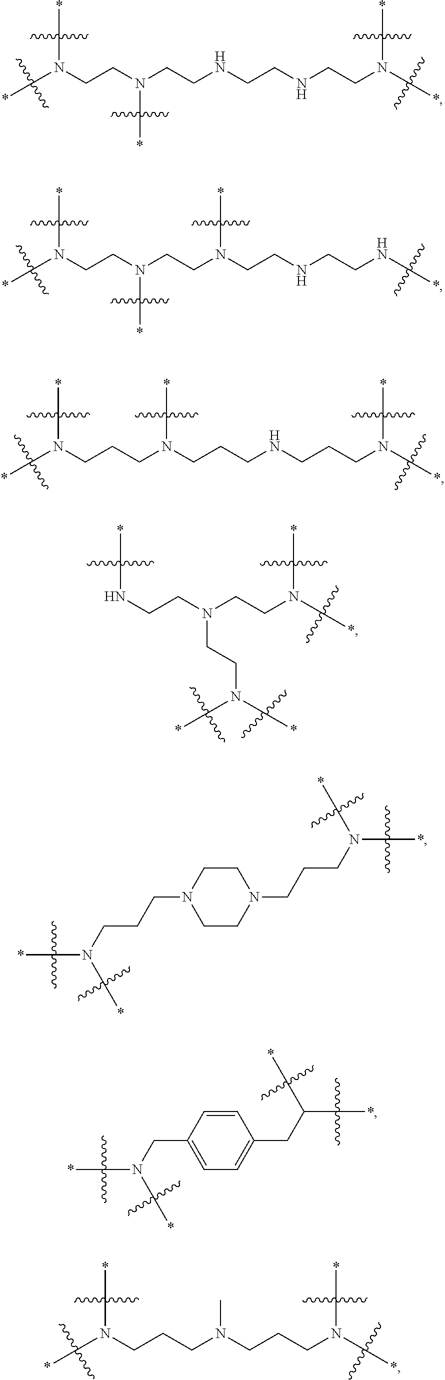

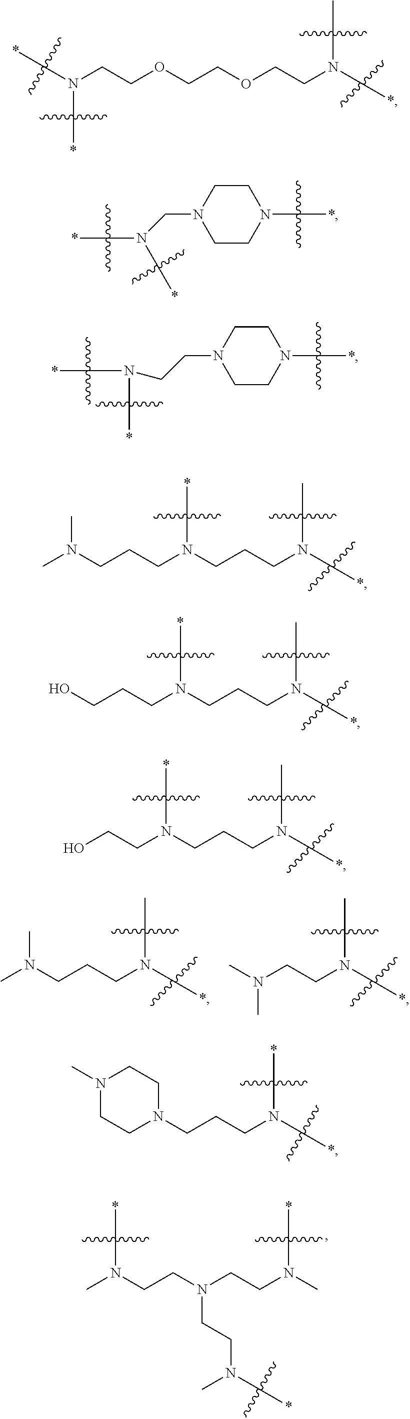

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

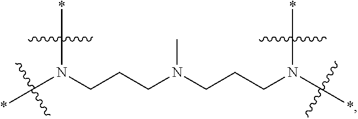

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

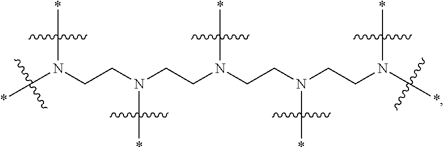

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σᵢ₌₀^{i=g-2}2ⁱ, when g≠1.

In some embodiments of X_{Branch}, g=1, G=0, Z=1, and each branch of the plurality of branches comprises a structural formula each branch of the plurality of branches comprises a structural formula

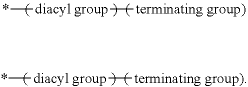

In some embodiments of X_{Branch}, g=2, G=1, Z=2, and each branch of the plurality of branches comprises a structural formula

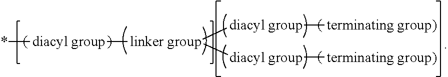

In some embodiments of X_{Branch}, g=3, G=3, Z=4, and each branch of the plurality of branches comprises a structural formula

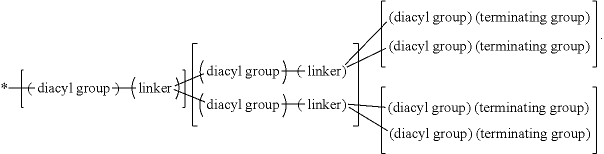

In some embodiments of X_{Branch}, g=4, G=7, Z=8, and each branch of the plurality of branches comprises a structural formula

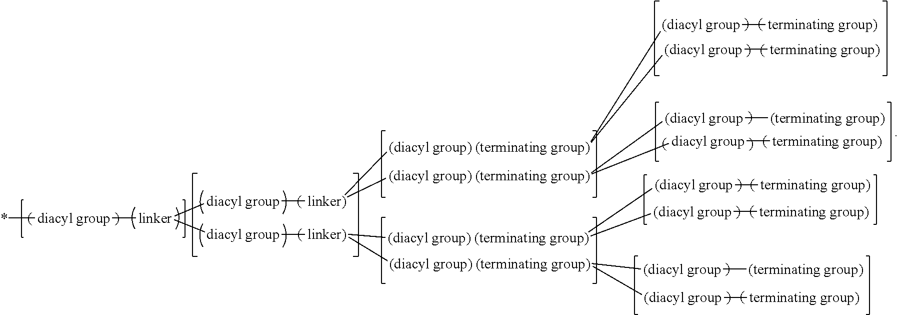

In some embodiments, the dendrimers or dendrons described herein with a generation (g)=1 has the structure:

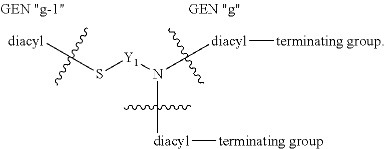

In some embodiments, the dendrimers or or dendrons described herein with a generation (g) 1 has the structure:

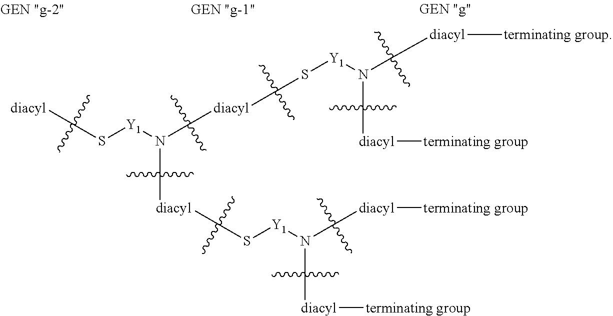

An example formulation of the dendrimers or dendrons described herein for generations 1-4 is shown in Table 2. The number of diacyl groups, linker groups, and terminating groups can be calculated based on g.

TABLE 2
Formulation of Dendrimer or Dendron Groups Based on Generation (g)

| | g = 1 | g = 2 | g = 3 | g = 4 | |
|---|---|---|---|---|---|
| # of diacyl grp | 1 | 1 + 2 = 3 | 1 + 2 + 2² = 7 | 1 + 2 + 2² + 2³ = 15 | 1 + 2 + . . . + 2^{g-1} |
| # of linker grp | 0 | 1 | 1 + 2 | 1 + 2 + 2² | 1 + 2 + . . . + 2^{g-2} |
| # of terminating | 1 | 2 | 2² | 2³ | 2(^{g-1}) |
| grp | | | | | |

In some embodiments, the diacyl group independently comprises a structural formula

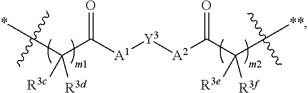

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R³¹ are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

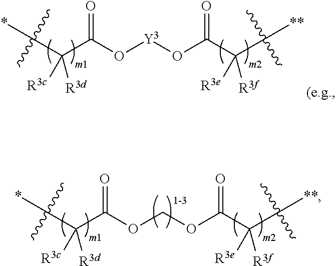

such as

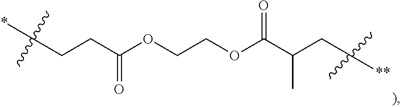

and optionally R^{3c}, R^{3d}, R^{3e}, and R^{3f} are each independently at each occurrence hydrogen or C₁-C₃ alkyl.

In some embodiments, linker group independently comprises a structural formula

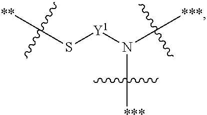

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y1 is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y1 is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

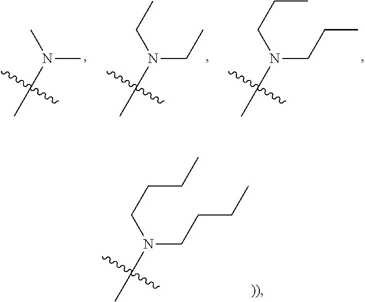

C₄-C₆ N-heterocycloalkyl (e.g., N-pyrrolidinyl

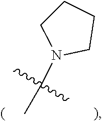 [SMILES: *N1CCCC1]

### N-piperidinyl

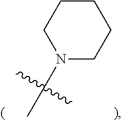 [SMILES: *N1CCCCC1]

### N-azepanyl

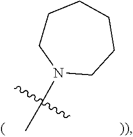 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

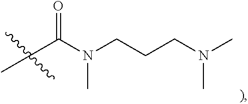 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

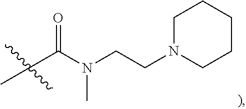 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl) (e.g.,

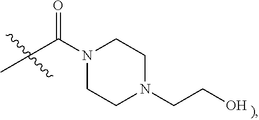 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

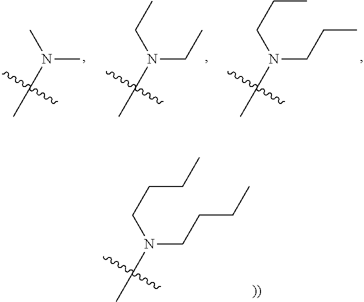

and C₄-C₆ N-heterocycloalkyl (e.g., N-pyrrolidinyl

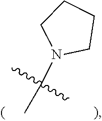 [SMILES: *N1CCCC1]

### N-piperidinyl

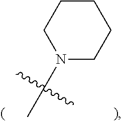 [SMILES: *N1CCCCC1]

### N-azepanyl

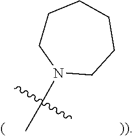 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table 3. In some embodiments, the dendrimers or dendrons described herein can comprise a terminating group or pharmaceutically acceptable salt, or thereof selected in Table 3. In some embodiments, the example terminating group of Table 3 are not limiting of the stereoisomers (i.e. enantiomers, diastereomers) listed.

TABLE 3
Example terminating group/peripheries structures

| ID # | Structure |
|---|---|
| SC1 | 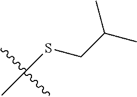 [SMILES: *SCC(C)C] |
| SC2 | 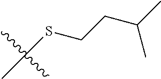 [SMILES: *SCCC(C)C] |
| SC3 | 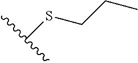 [SMILES: *SCCC] |
| SC4 | 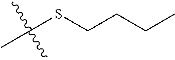 [SMILES: *SCCCC] |
| SC5 | 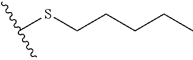 [SMILES: *SCCCCC] |
| SC6 | 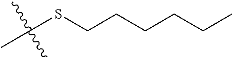 [SMILES: *SCCCCCC] |
| SC7 | 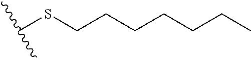 [SMILES: *SCCCCCCC] |
| SC8 | 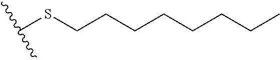 [SMILES: *SCCCCCCCC] |
| SC9 | 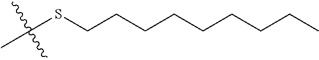 [SMILES: *SCCCCCCCCC] |
| SC10 | 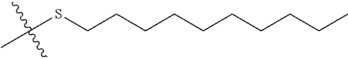 [SMILES: *SCCCCCCCCCC] |
| SC11 | 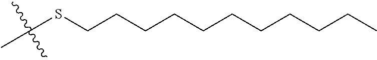 [SMILES: *SCCCCCCCCCCC] |
| SC12 | 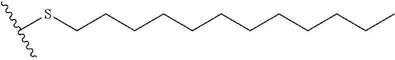 [SMILES: *SCCCCCCCCCCCC] |
| SC14 | 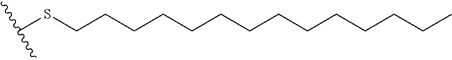 [SMILES: *SCCCCCCCCCCCCCC] |
| SC16 | 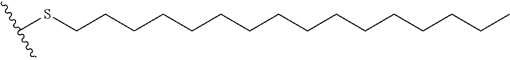 [SMILES: *SCCCCCCCCCCCCCCCC] |
| SC18 | 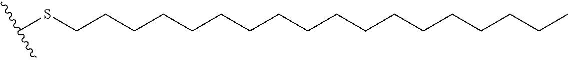 [SMILES: *SCCCCCCCCCCCCCCCCCC] |
| SC19 | 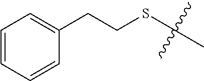 [SMILES: *SCCc1ccccc1] |
| SO1 | 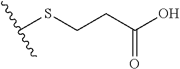 [SMILES: *SCCC(=O)O] |
| SO2 | 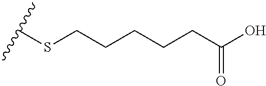 [SMILES: *SCCCCCC(=O)O] |
| SO3 | 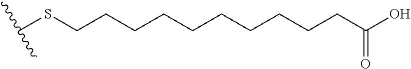 [SMILES: *SCCCCCCCCCCC(=O)O] |
| SO4 | 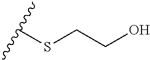 [SMILES: *SCCO] |
| SO5 | 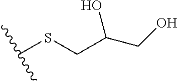 [SMILES: *SCC(O)CO] |
| SO6 | 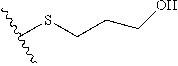 [SMILES: *SCCCO] |
| SO7 | 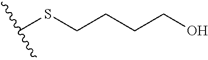 [SMILES: *SCCCCO] |
| SO8 | 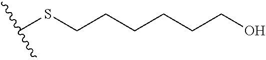 [SMILES: *SCCCCCCO] |
| SO9 | 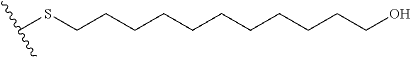 [SMILES: *SCCCCCCCCCCCO] |
| SN1 | 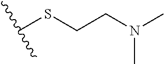 [SMILES: *SCCN(C)C] |
| SN2 | 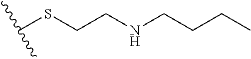 [SMILES: *SCCNCCCC] |
| SN3 | 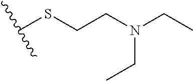 [SMILES: *SCCN(CC)CC] |
| SN4 | 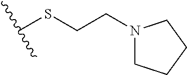 [SMILES: *SCCN1CCCC1] |
| SN5 | 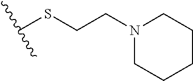 [SMILES: *SCCN1CCCCC1] |
| SN6 | 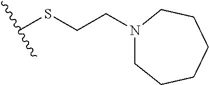 [SMILES: *SCCN1CCCCCC1] |
| SN7 | 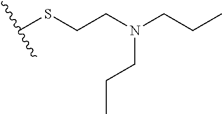 [SMILES: *SCCN(CCC)CCC] |
| SN8 | 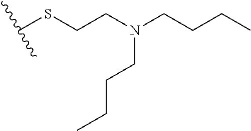 [SMILES: *SCCN(CCCC)CCCC] |
| SN9 | 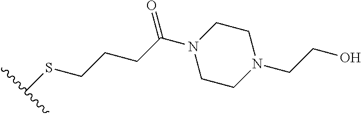 [SMILES: *SCCCC(=O)N1CCN(CCO)CC1] |
| SN10 | 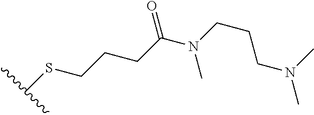 [SMILES: *SCCCC(=O)N(C)CCCN(C)C] |
| SN11 | 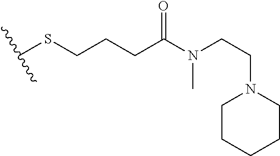 [SMILES: *SCCCC(=O)N(C)CCN1CCCCC1] |

In some embodiments, the dendrimer or dendron of Formula (X) is selected from those set forth in Table 4 and pharmaceutically acceptable salts thereof.

TABLE 4
Example ionizable cationic lipo-dendrimers

| ID # | Structure |
|---|---|
| 2A2- SC14 | 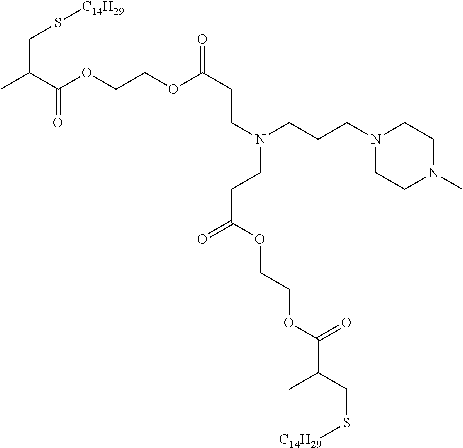 |
| 2A6- SC14 | 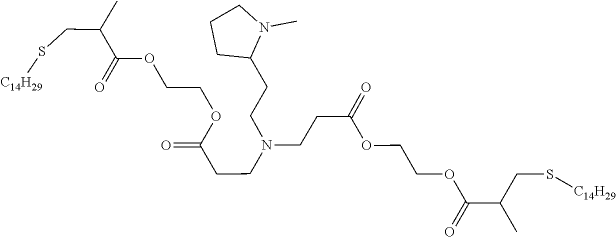 |
| 2A9- SC14 | 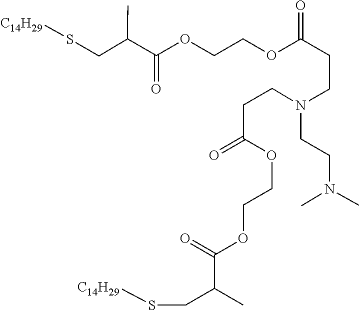 |
| 3A3- SC10 | 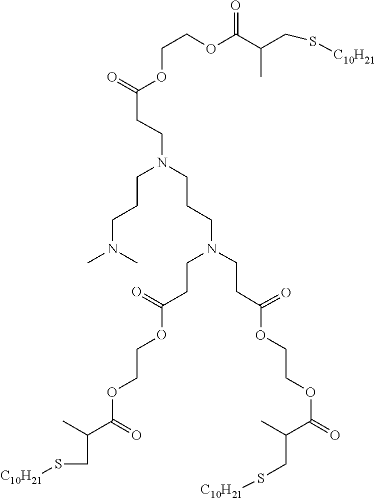 |
| 3A3- SC14 | 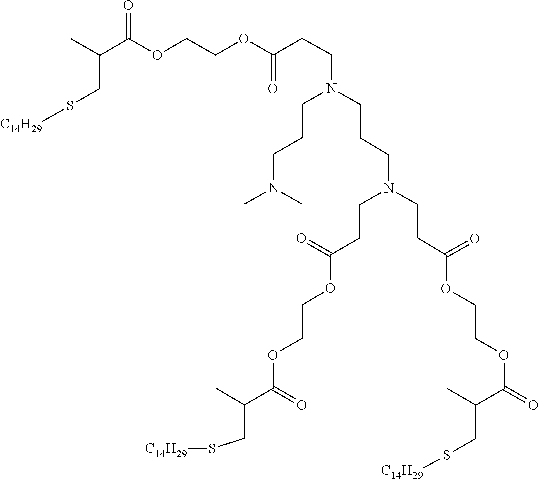 |
| 3A5- SC10 | 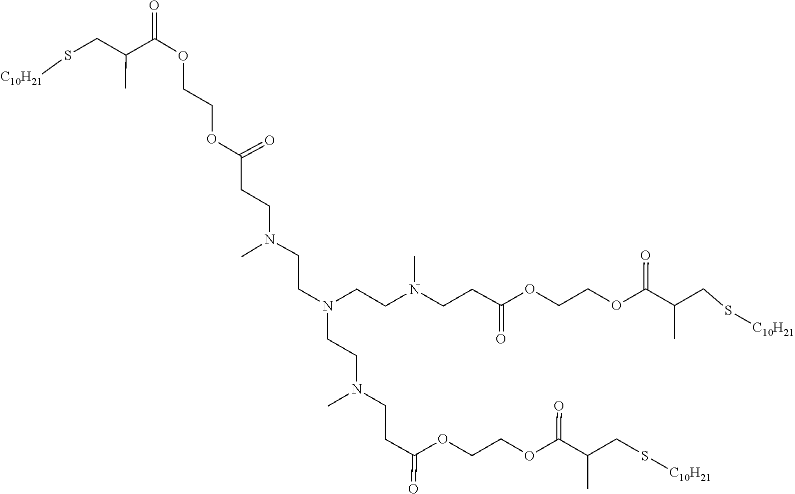 |
| 3A5- SC14 | 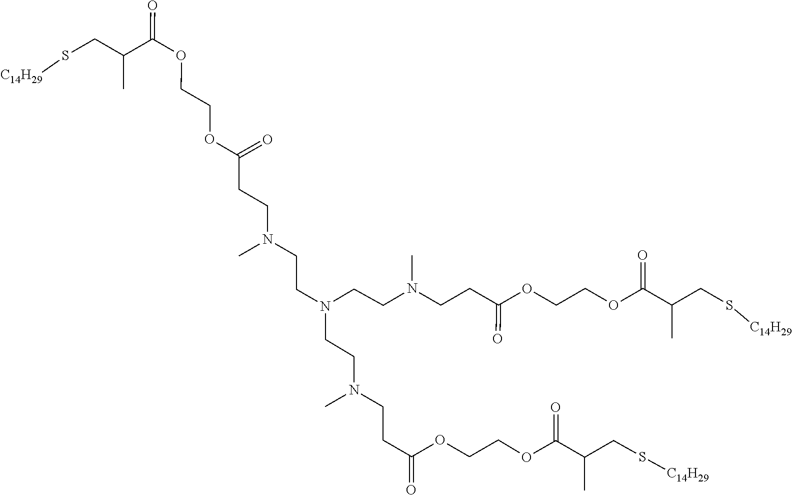 |
| 4A1- SC12 | 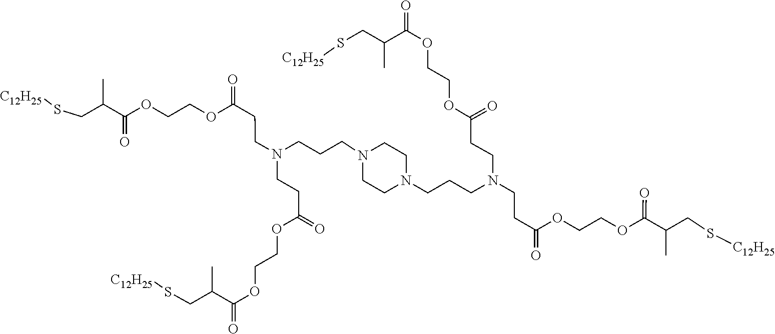 |
| 4A3- SC12 | 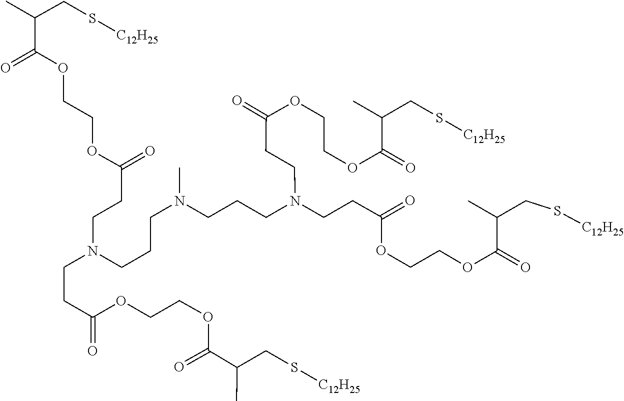 |
| 5A1- SC12 | 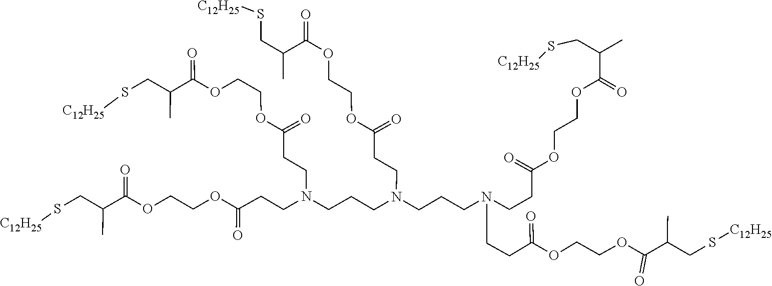 |
| 5A1- SC8 | 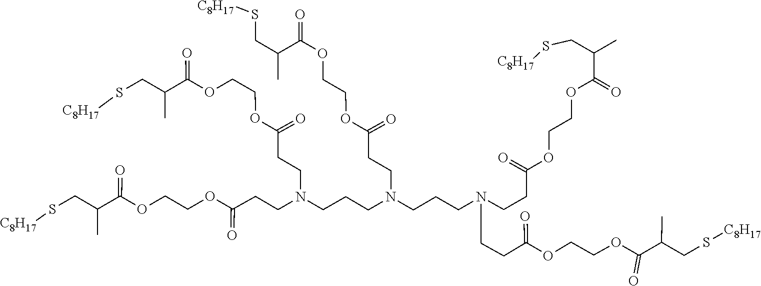 |
| 5A2-2- SC12 (5-arm) | 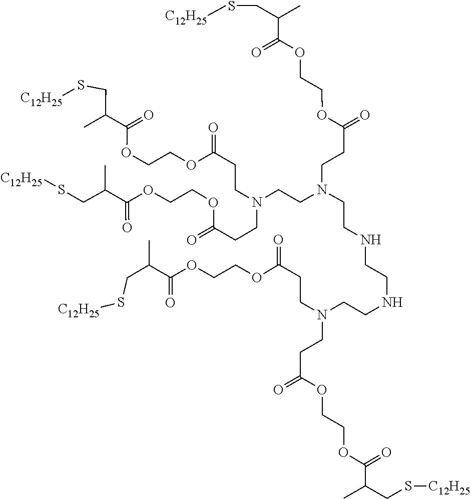 |
| 5A3-1- SC12 (5 arm) | 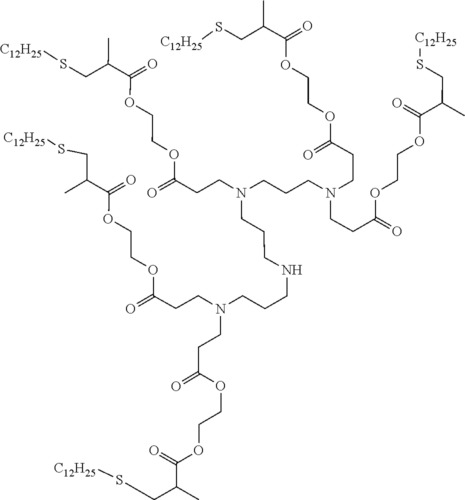 |
| 5A3-1- SC8 (5-arm) | 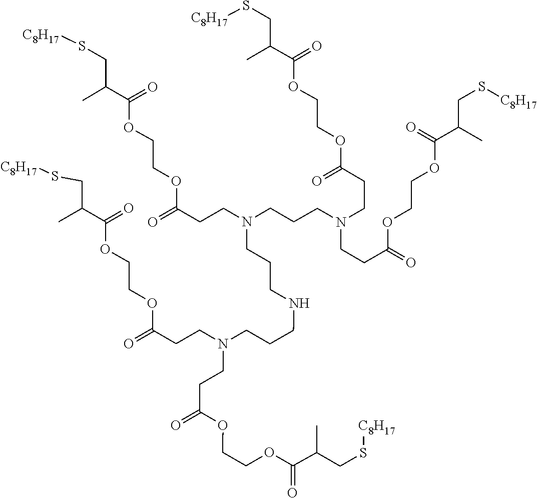 |
| 5A4-1- SC12 (5-arm) | 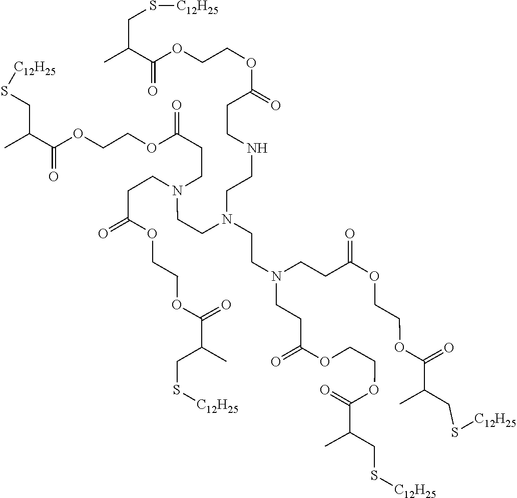 |
| 5A4-1- SC8 (5-arm) | 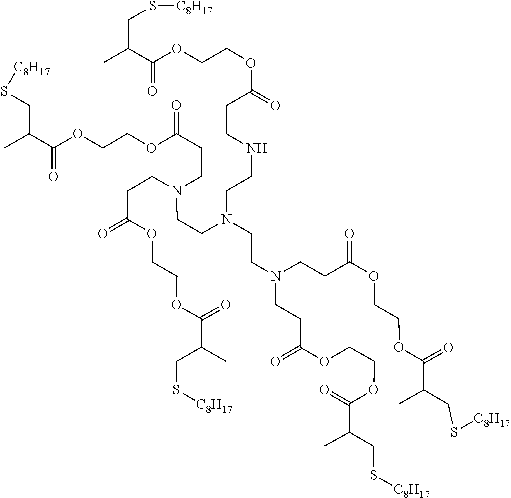 |
| 5A5- SC8 | 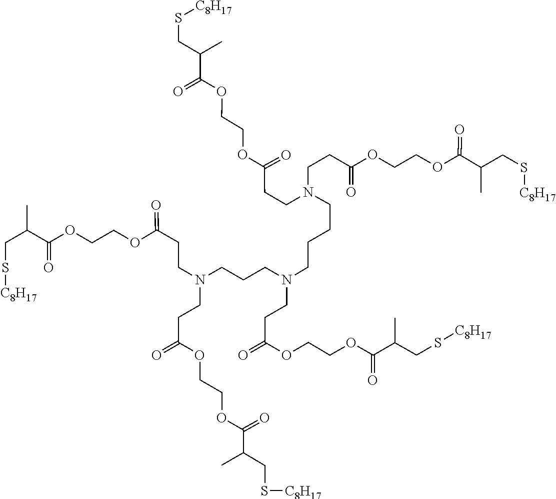 |
| 5A5- SC12 | 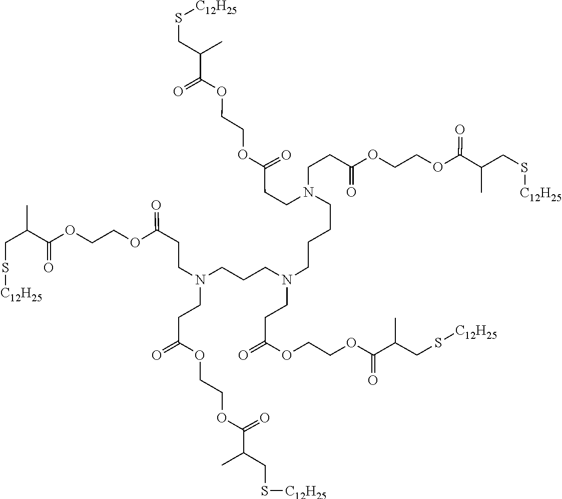 |
| 5A2-4- SC12 (6-arm) | 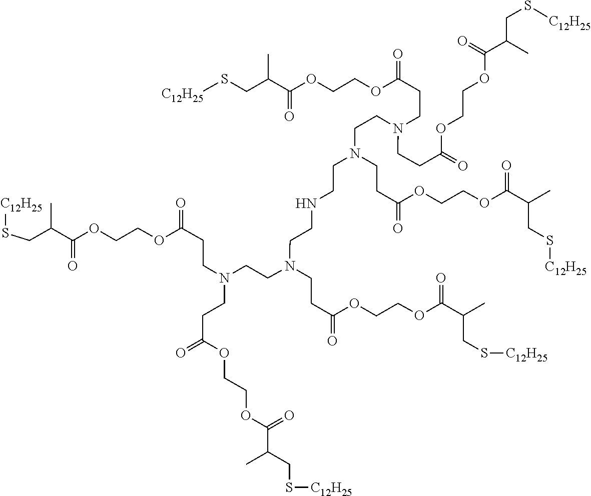 |
| 5A2-4- SC10 (6-arm) | 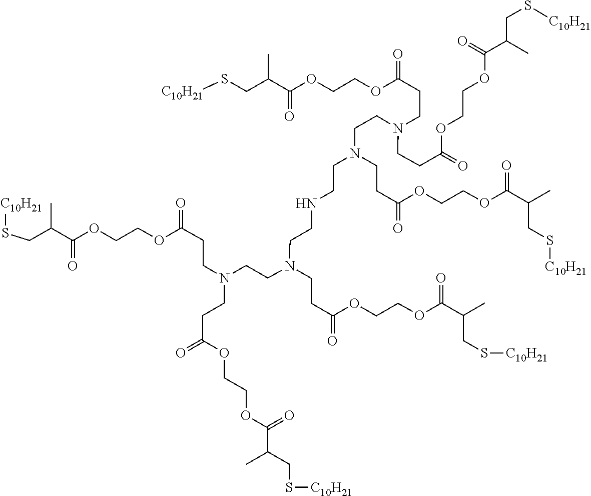 |
| 5A3- 2-- SC8 (6-arm) | 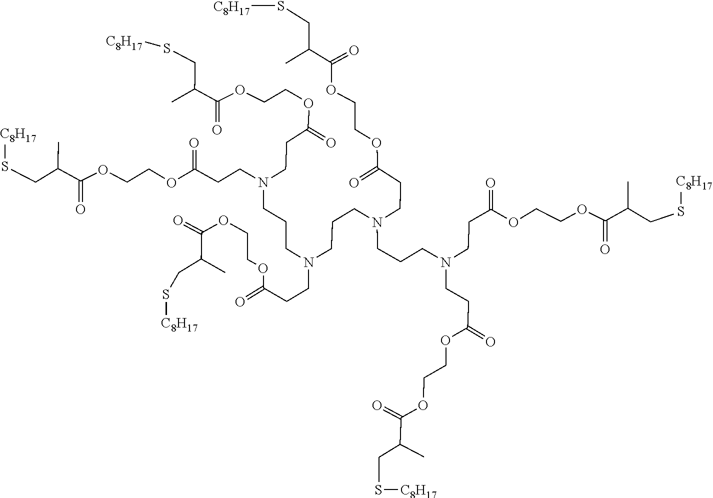 |
| 5A3-2- SC12 (6-arm) | 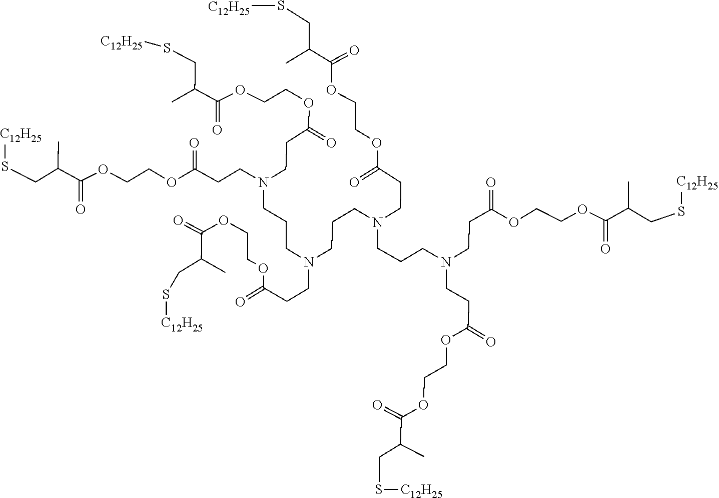 |
| 5A4-2- SC8 (6-arm) | 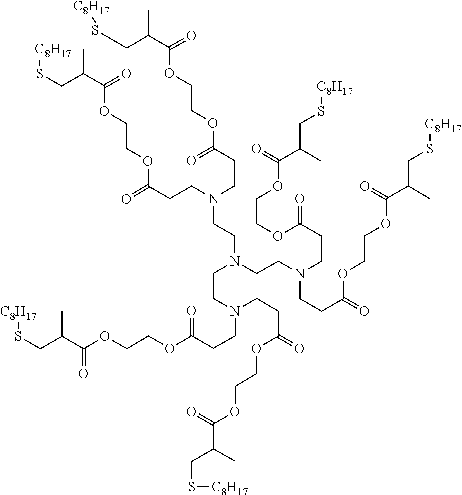 |
| 5A4-2- SC12 (6-arm) | 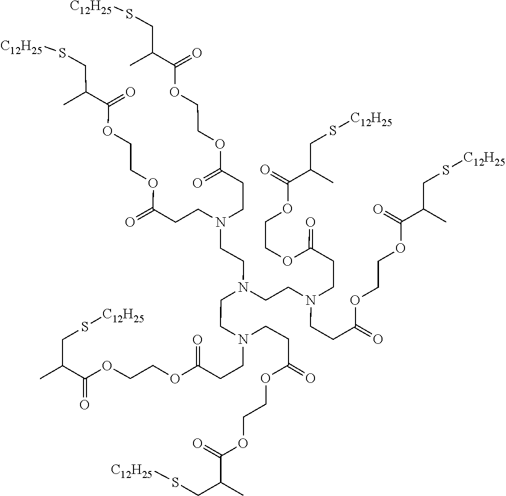 |
| 6A4- SC8 | 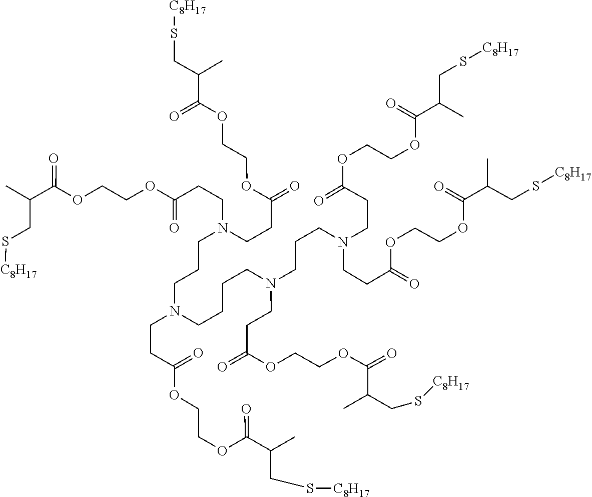 |
| 6A4- SC12 | 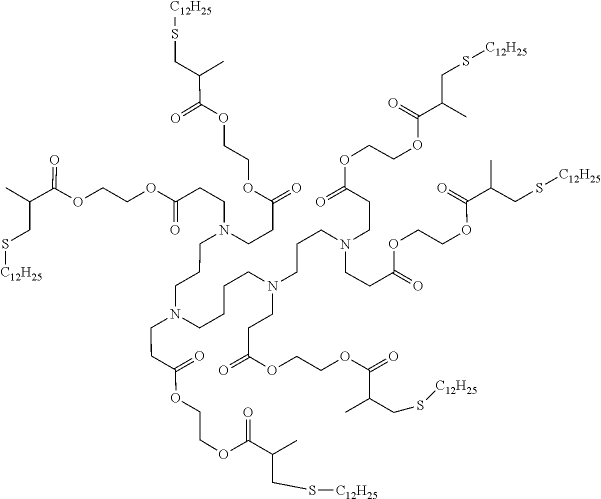 |
| 2A2- g2- SC12 | 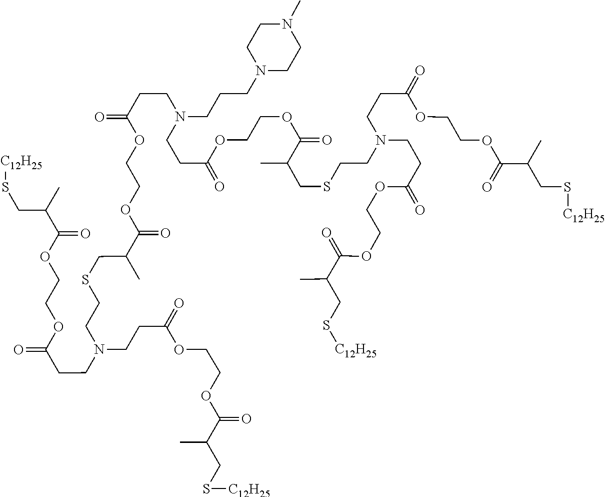 |
| 2A2- g2- SC8 | 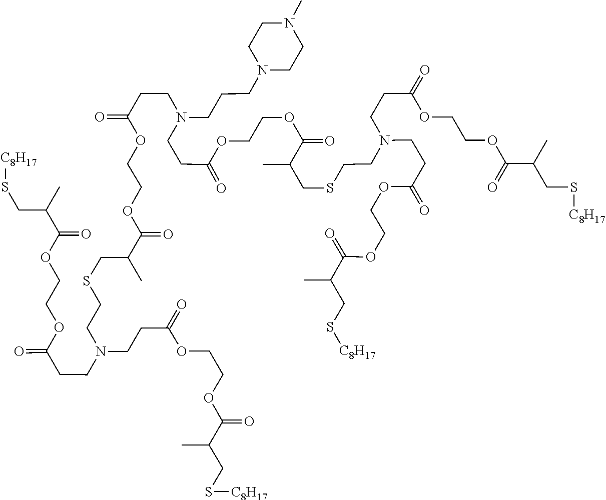 |
| 2A11- g2- SC12 | 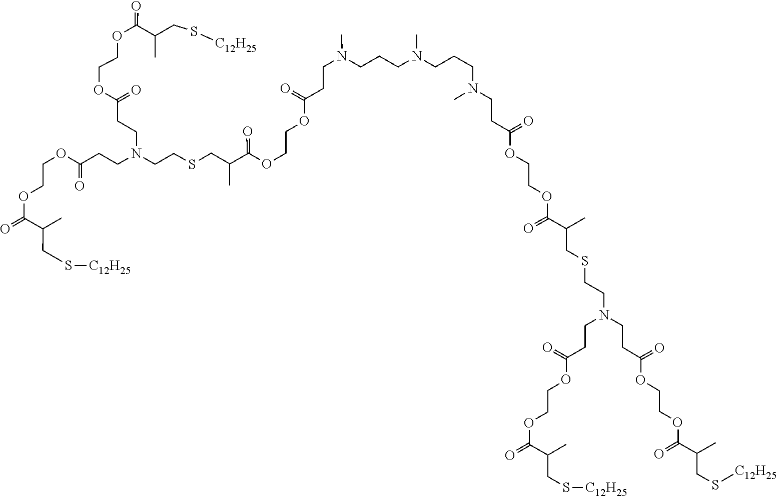 |
| 2A11- g2- SC8 | 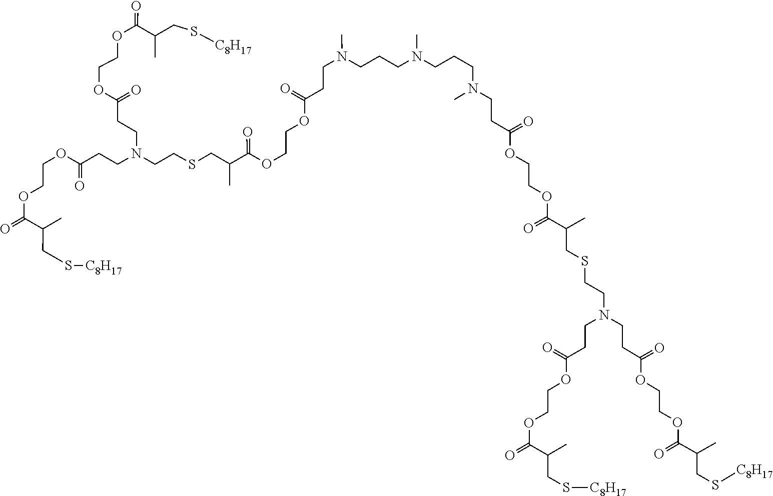 |
| 3A3- g2- SC12 | 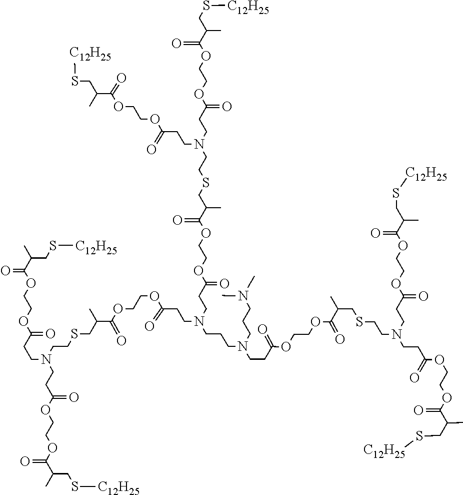 |
| 3A3- g2- SC8 | 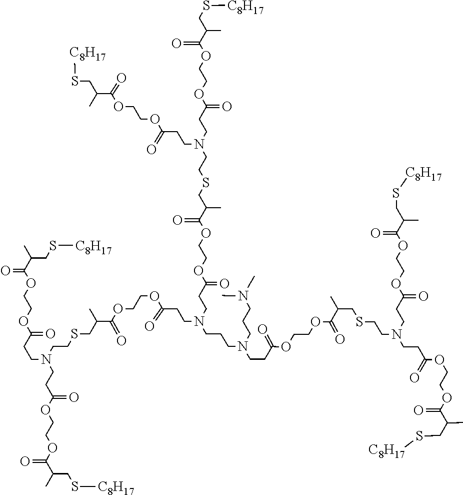 |
| 3A5- g2- SC12 | 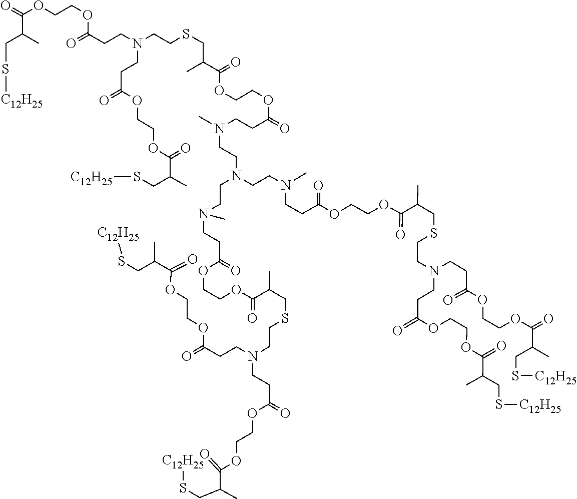 |
| 2A11- g3- SC12 | 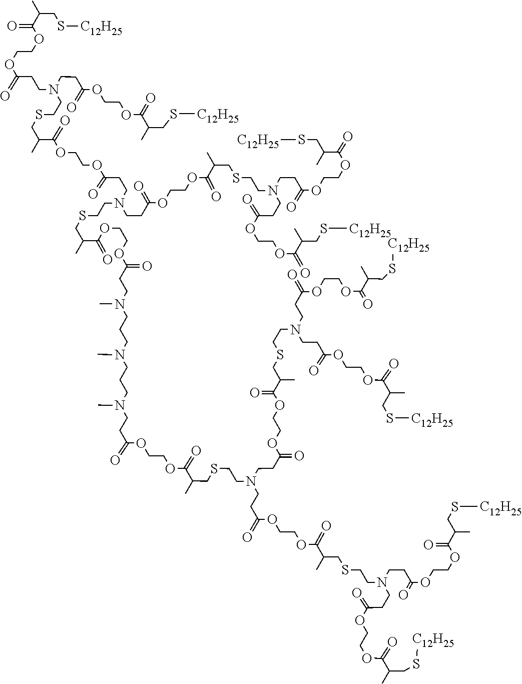 |
| 2A11- g3- SC8 | 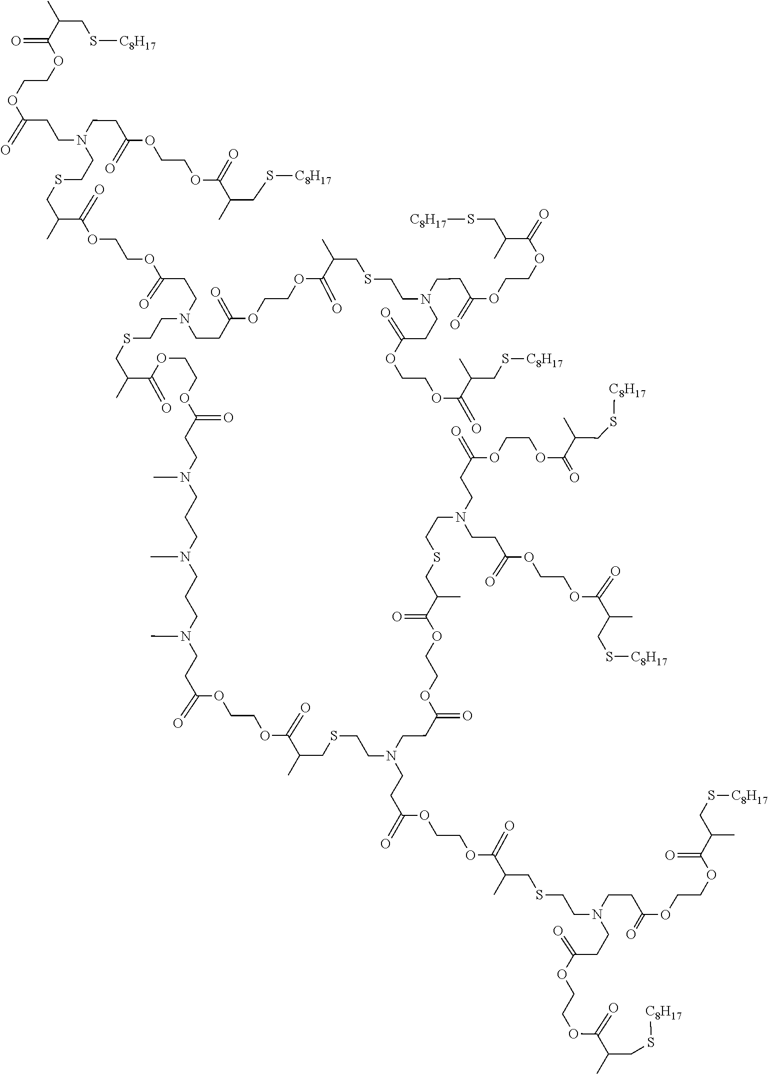 |
| 1A2- g4- SC12 | 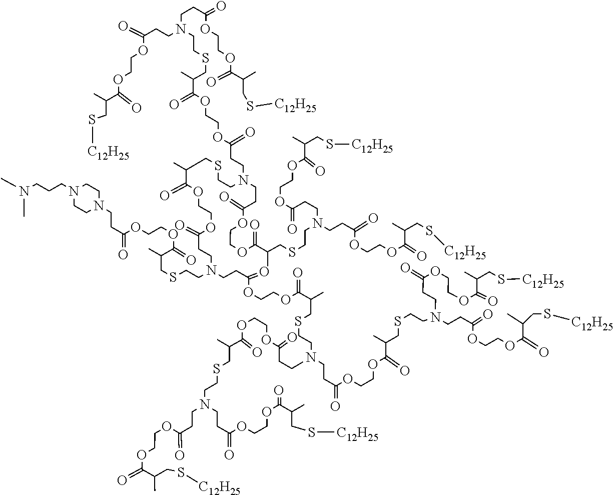 |
| 4A1- g2- SC12 | 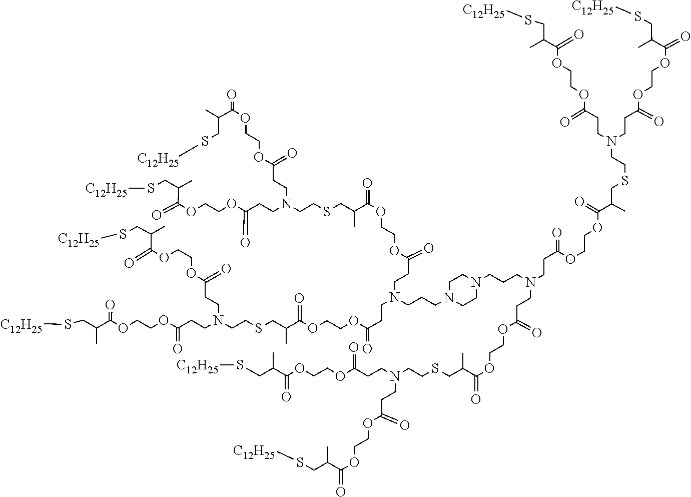 |
| 1A2- g4- SC8 | 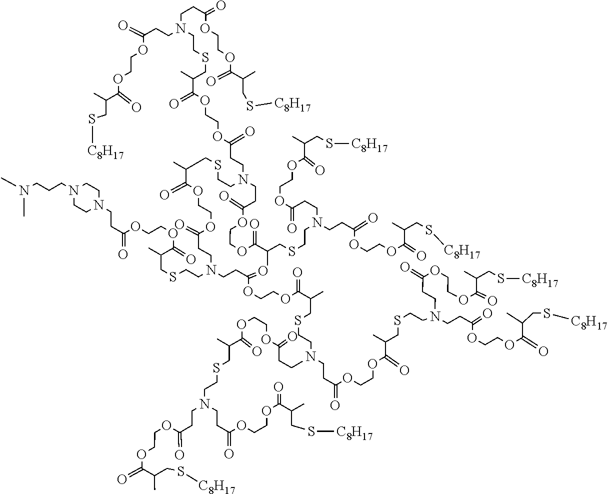 |
| 4A1- g2- SC8 | 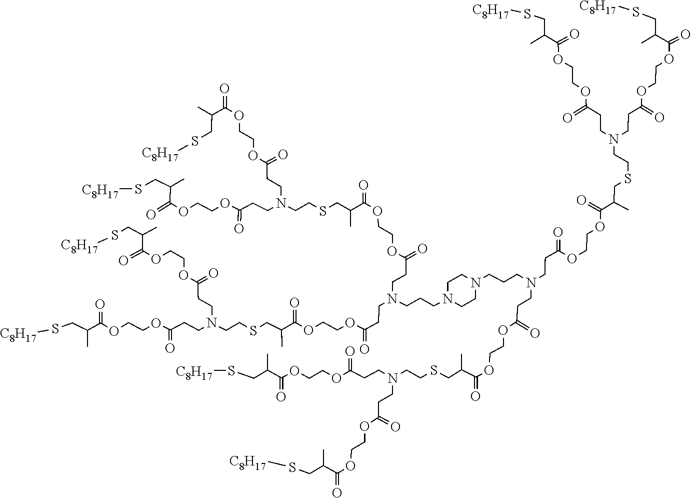 |
| 4A3- g2- SC12 | 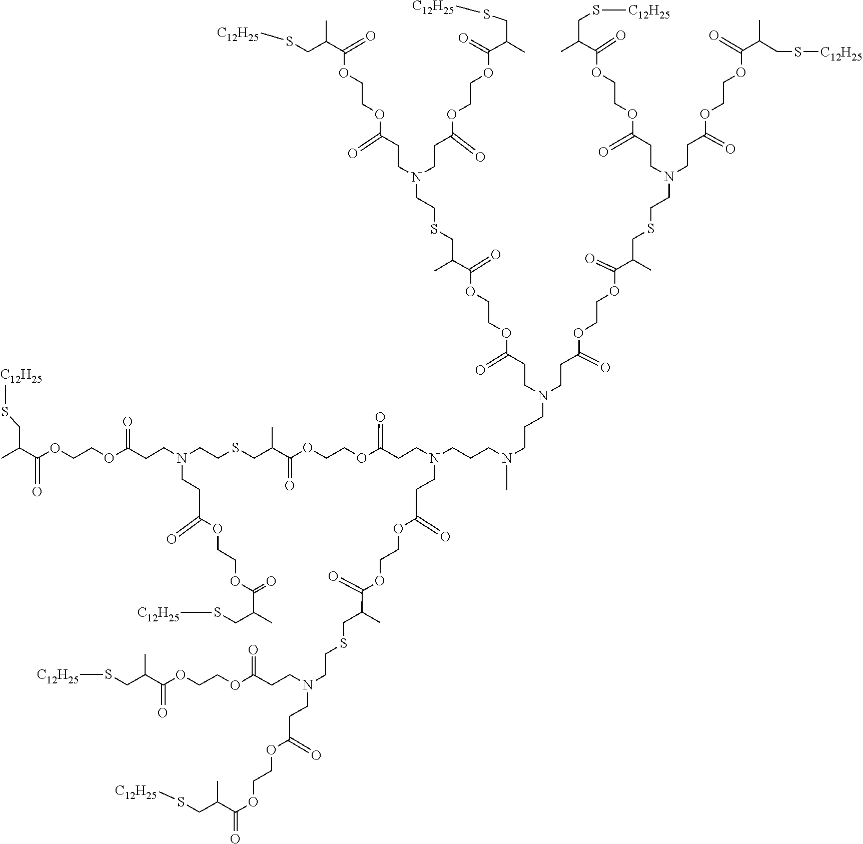 |
| 4A3- g2- SC8 | 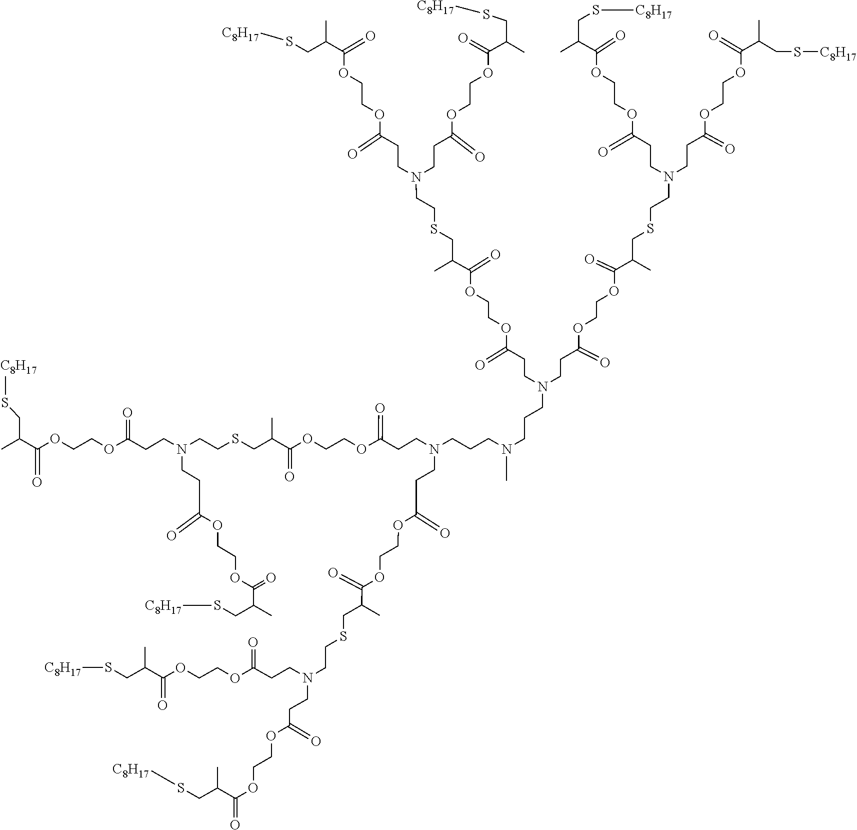 |
| 1A2- g3- SC12 | 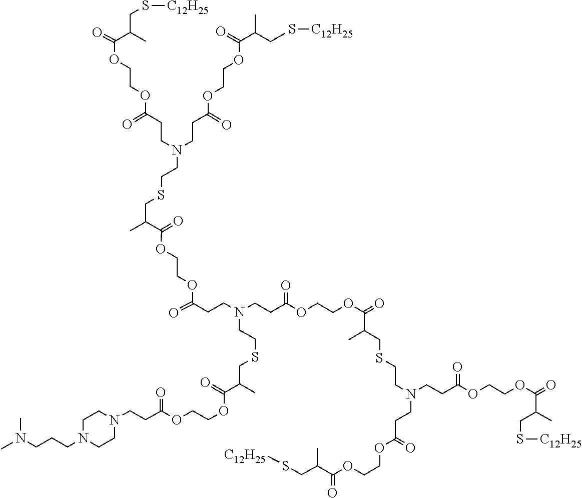 |
| 1A2- g3- SC8 | 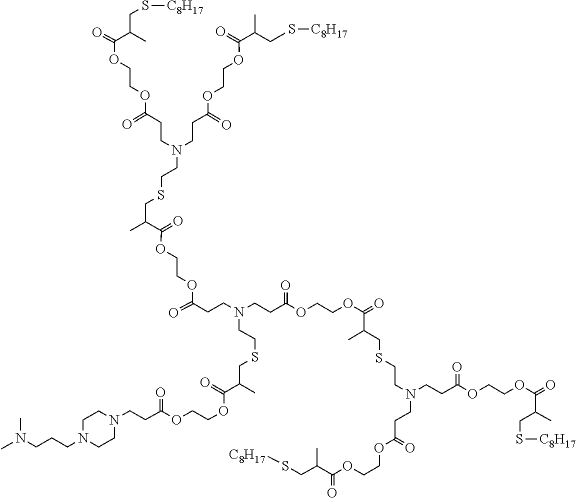 |
| 2A2- g3- SC12 | 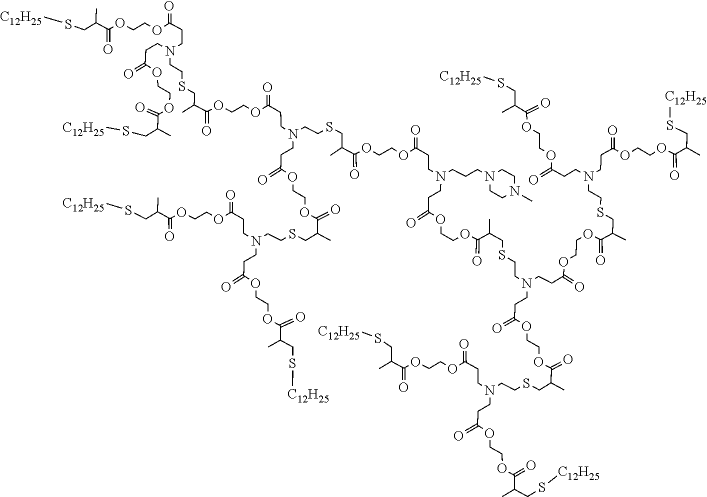 |
| 2A2- g3- SC8 | 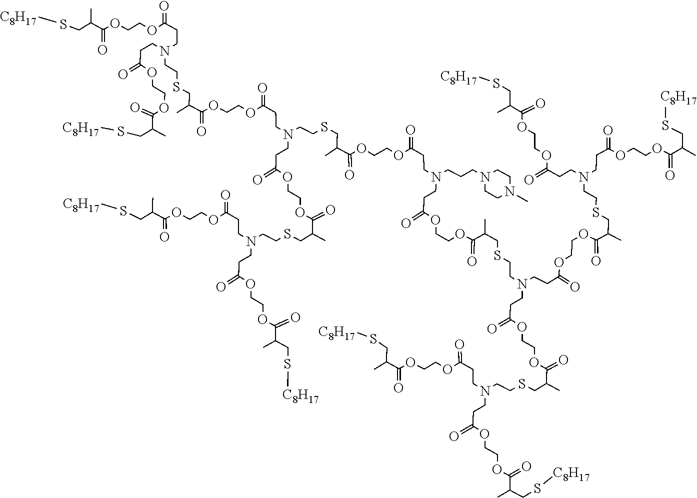 |
| 5A2- 4- SC8 (6- arm) | 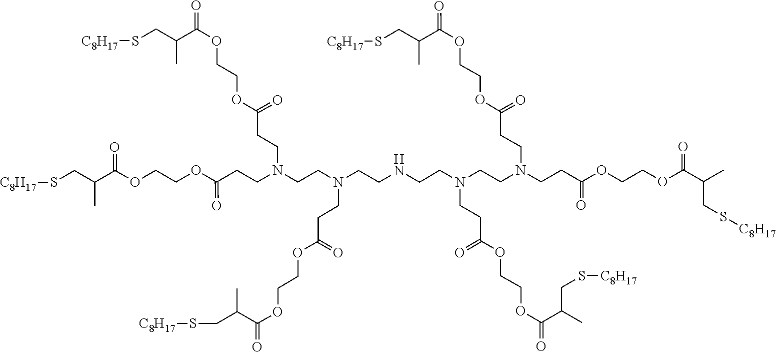 |
| 5A-5- SC8 (6 arm) | 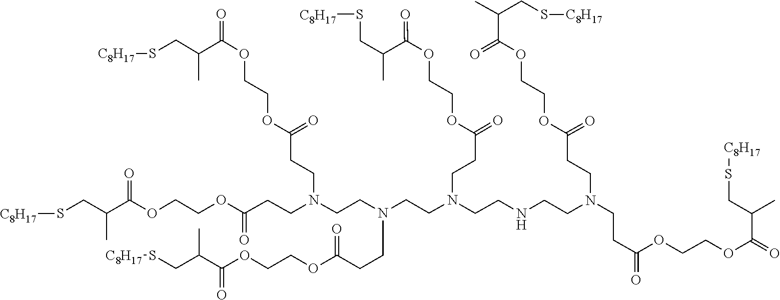 |
| 5A2- 6-SC8 (6 arm) | 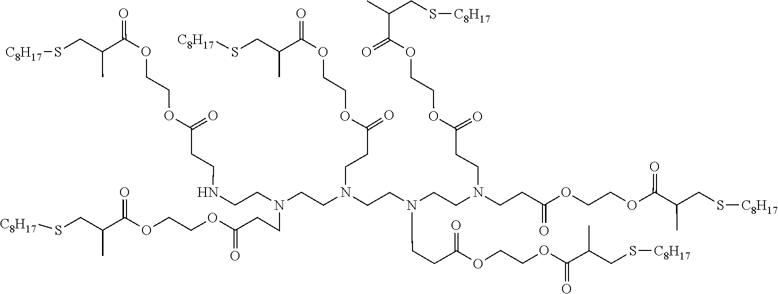 |
| 5A2-1- SC8 (5-arm) | 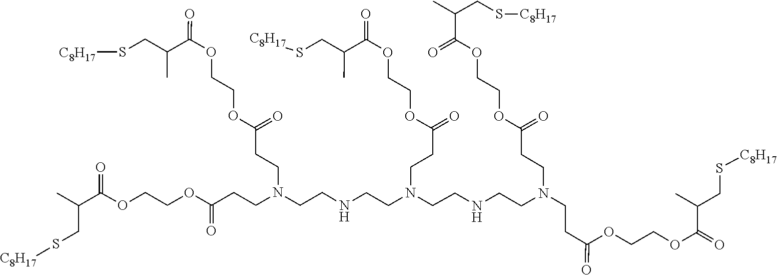 |
| 5A2-2- SC8 | 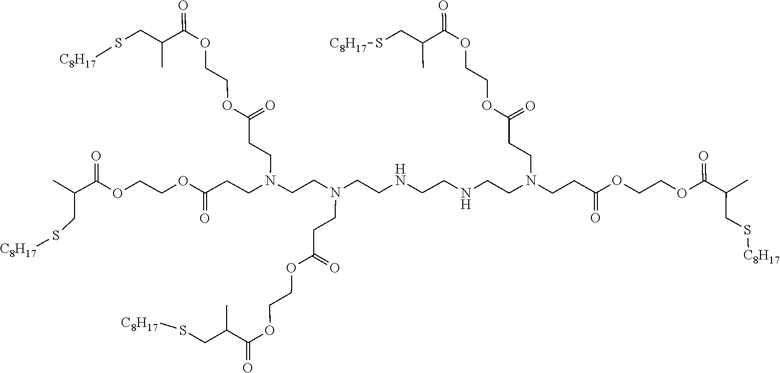 |
| 4A1- SC5 | 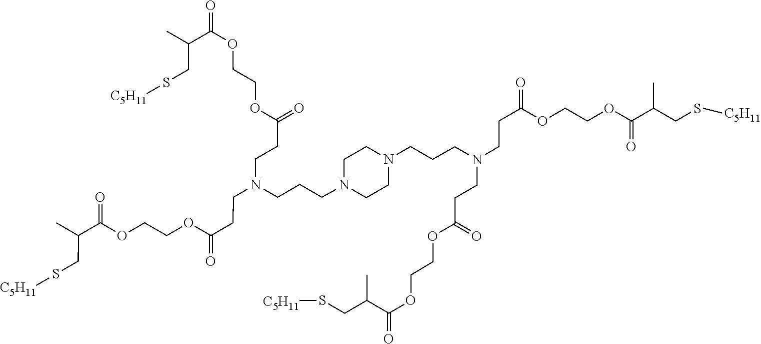 |
| 4A1- SC8 | 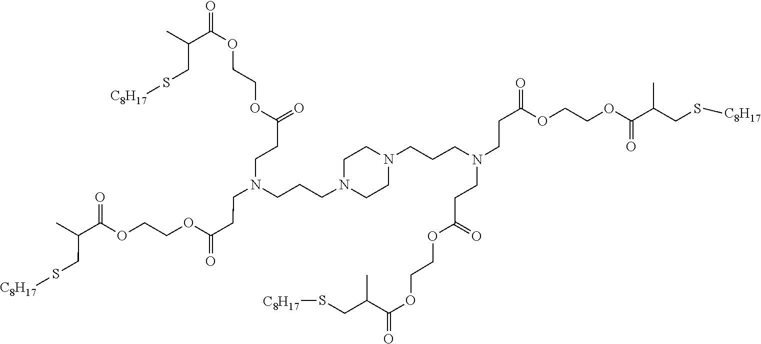 |
| 4A3- SC6 | 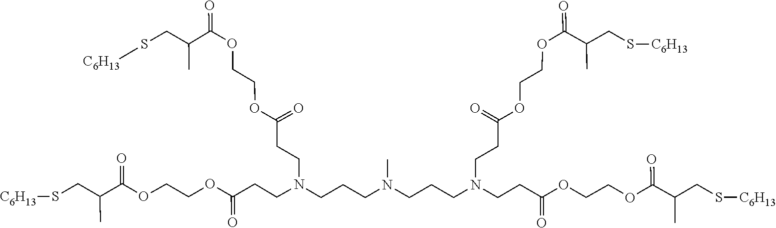 |
| 4A3- SC7 | 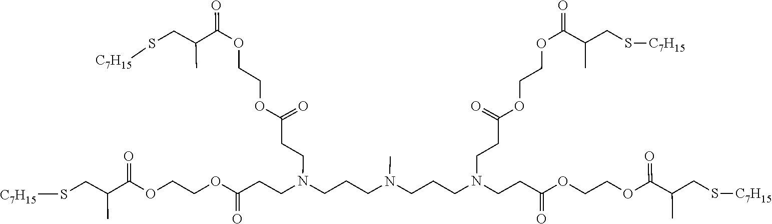 |
| 4A3- SC8 | 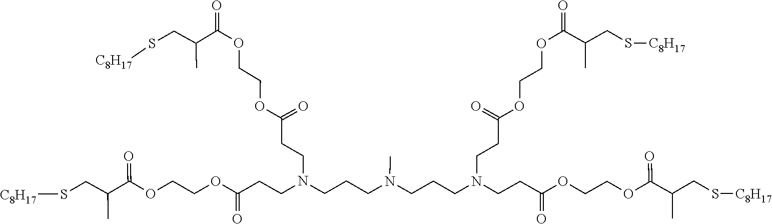 |
| 5A4-2- SC5 (6 arm) | 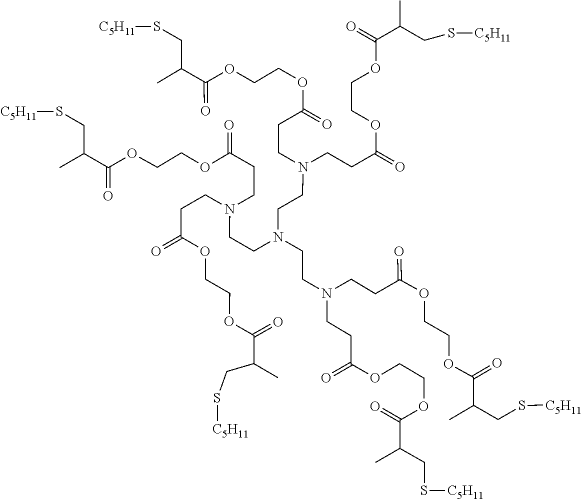 |
| 5A4-2- SC6 (6 arm) | 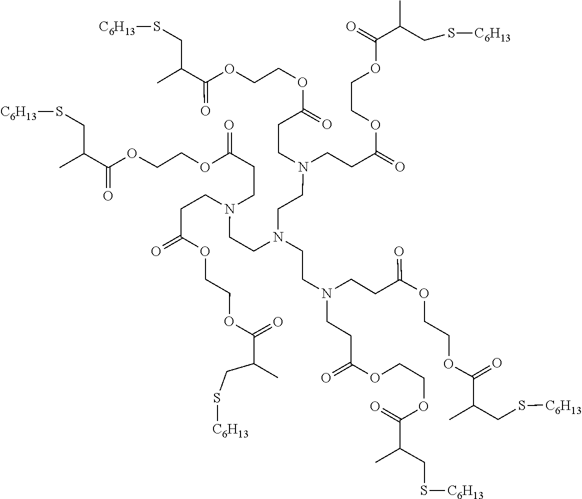 |
| 5A2-4- SC8 (5-arm) | 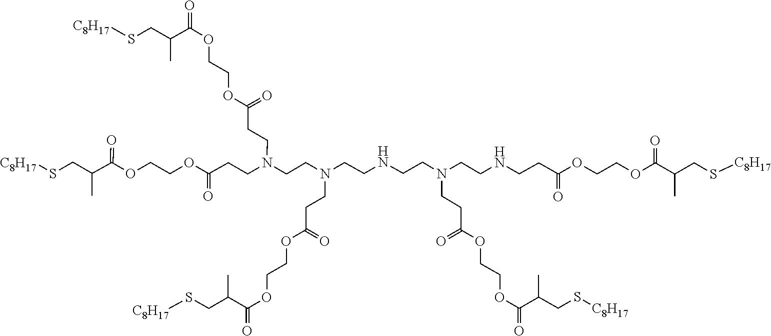 |
| 3A5- g2- SC8 | 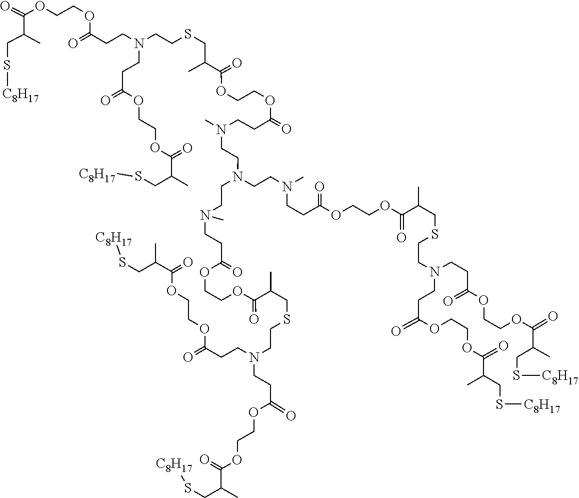 |

### Other Ionizable Cationic Lipids

In some embodiments of the lipid composition, the cationic lipid comprises a structural formula (D-I′):

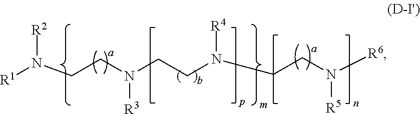

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand; provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and wherein one or more of the nitrogen atoms indicated in formula (D-I′) may be protonated to provide a cationic lipid.

In some embodiments of the cationic lipid of formula (D-I′), a is 1. In some embodiments of the cationic lipid of formula (D-I′), b is 2. In some embodiments of the cationic lipid of formula (D-I′), m is 1. In some embodiments of the cationic lipid of formula (D-I′), n is 1. In some embodiments of the cationic lipid of formula (D-I′), R¹, R², R³, R⁴, R⁵, and R⁶ are each independently H or —CH₂CH(OH)R⁷. In some embodiments of the cationic lipid of formula (D-I′), R¹, R², R³, R⁴, R⁵, and R⁶ are each independently H or

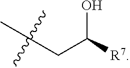

In some embodiments of the cationic lipid of formula (D-I′), R¹, R², R³, R⁴, R⁵, and R⁶ are each independently H or

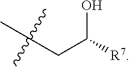

In some embodiments of the cationic lipid of formula (D-I′), R⁷ is C₃-C₁₈ alkyl (e.g., C₆-C₁₂ alkyl).

In some embodiments, the cationic lipid of formula (D-I′) is 13,16,20-tris(2-hydroxydodecyl)-13,16,20,23-tetraazapentatricontane-11,25-diol:

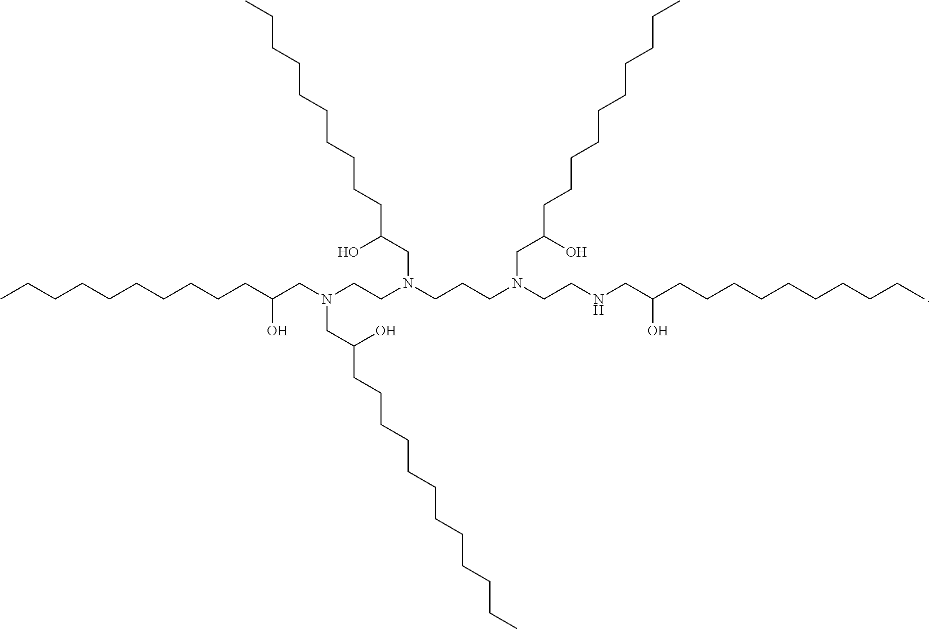 [SMILES: CCCCCCCCCCCCC(O)CN(CCN(CCCN(CCNCC(O)CCCCCCCCCC)CC(O)CCCCCCCCCC)CC(O)CCCCCCCCCC)CC(O)CCCCCCCCCC]

In some embodiments, the cationic lipid of formula (D-I′) is (11R,25R)-13,16,20-tris((R)-2-hydroxydodecyl)-13,16,20,23-tetraazapentatricontane-11,25-diol:

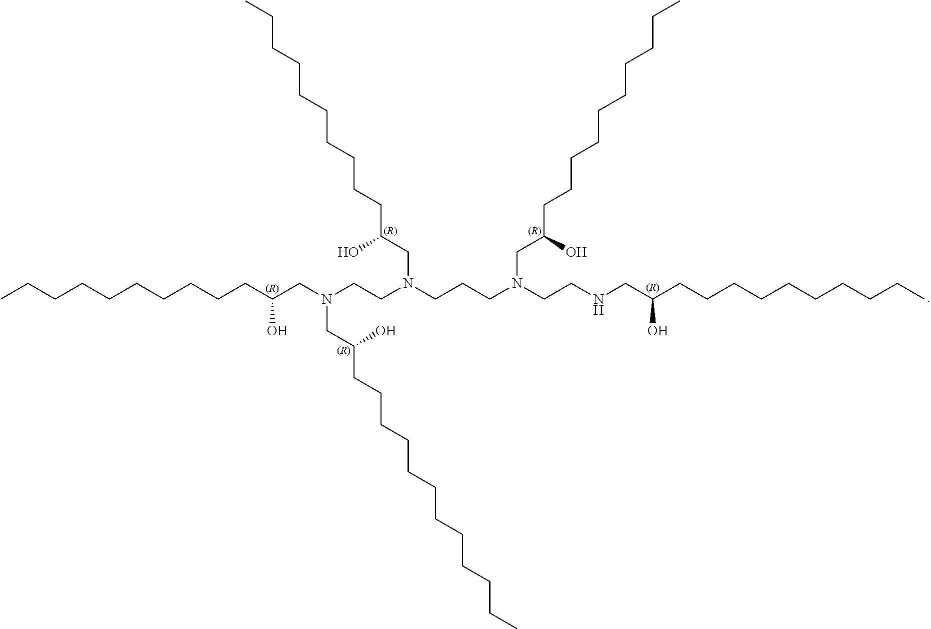

Additional cationic lipids that can be used in the compositions and methods of the present application include those cationic lipids as described in J. McClellan, M. C. King, Cell 2010, 141, 210-217, and International Patent Publication WO 2010/144740, WO 2013/149140, WO 2016/118725, WO 2016/118724, WO 2013/063468, WO 2016/205691, WO 2015/184256, WO 2016/004202, WO 2015/199952, WO 2017/004143, WO 2017/075531, WO 2017/117528, WO 2017/049245, WO 2017/173054 and WO 2015/095340, which are incorporated herein by reference for all purposes. Examples of those ionizable cationic lipids include but are not limited to those as shown in Table 5.

TABLE 5
Example ionizable cationic lipids

| # | Structure of example ionizable cationic lipid |
|---|---|
| 1 | 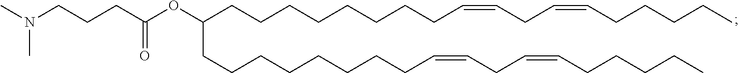 [SMILES: CCCCC/C=C\C/C=C\CCCCCCCCC(CCCCCCCC/C=C\C/C=C\CCCCC)OC(=O)CCCN(C)C] |
| 2 | 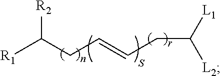 |
| 3 | 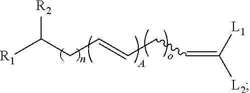 |
| 4 | 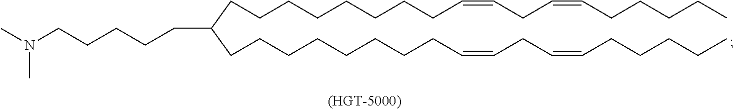 [SMILES: CCCCC/C=C\C/C=C\CCCCCCCCC(CCCCCCCC/C=C\C/C=C\CCCCC)CCCCCN(C)C] |
| 5 | 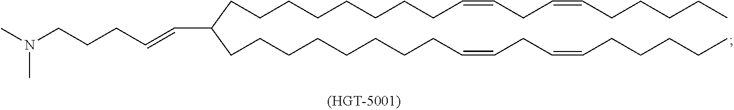 [SMILES: CCCCC/C=C\C/C=C\CCCCCCCCC(/C=C/CCCN(C)C)CCCCCCCC/C=C\C/C=C\CCCCC] |
| 6 | 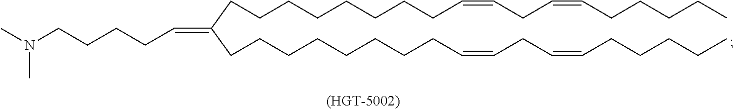 [SMILES: CCCCC/C=C\C/C=C\CCCCCCCCC(=CCCCCN(C)C)CCCCCCCC/C=C\C/C=C\CCCCC] |
| 7 | 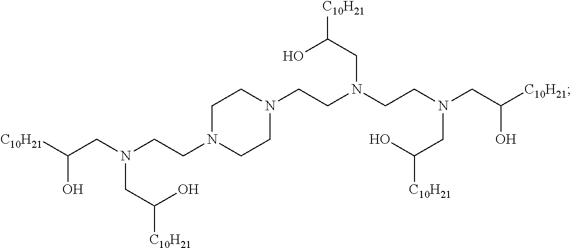 |
| 8 | 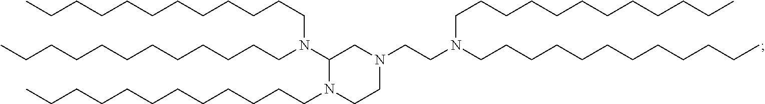 [SMILES: CCCCCCCCCCCCN(CCCCCCCCCCCC)CCN1CCN(CCCCCCCCCCCC)C(N(CCCCCCCCCCCC)CCCCCCCCCCCC)C1] |
| 9 | 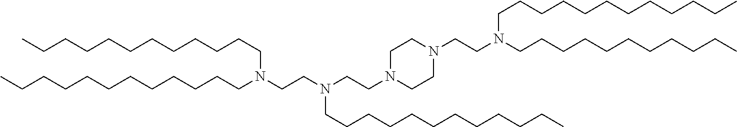 [SMILES: CCCCCCCCCCCCN(CCCCCCCCCCCC)CCN(CCCCCCCCCCCC)CCN1CCN(CCN(CCCCCCCCCCC)CCCCCCCCCCCC)CC1] |
| 10 | 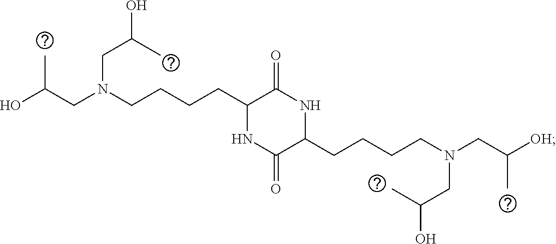 [SMILES: CC(O)CN(CCCCC1NC(=O)C(CCCCN(CC(C)O)CC(C)O)NC1=O)CC(C)O] |
| 11 | 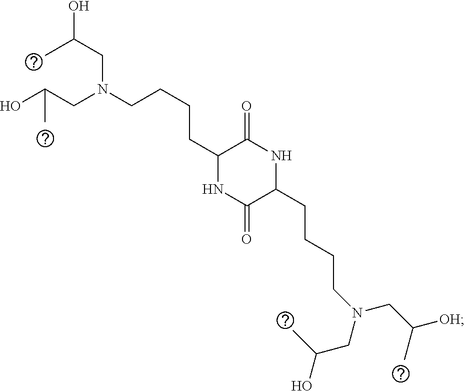 [SMILES: CC(O)CN(CCCCC1NC(=O)C(CCCCN(CC(C)O)CC(C)O)NC1=O)CC(C)O] |
| 12 | 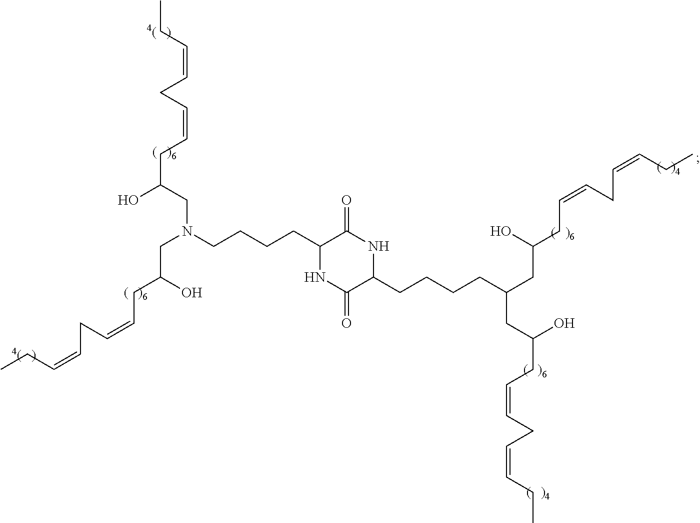 |
| 13 | 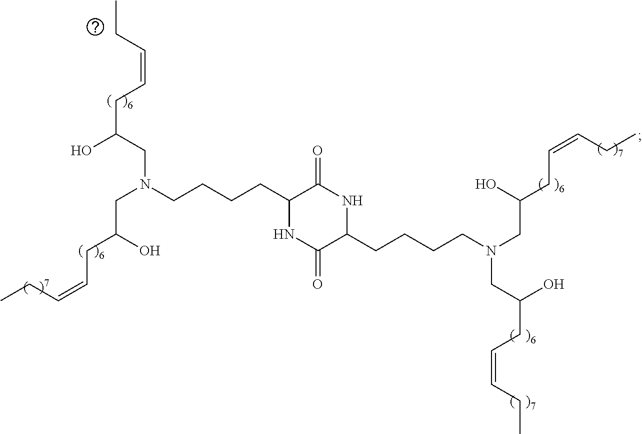 |
| 14 | 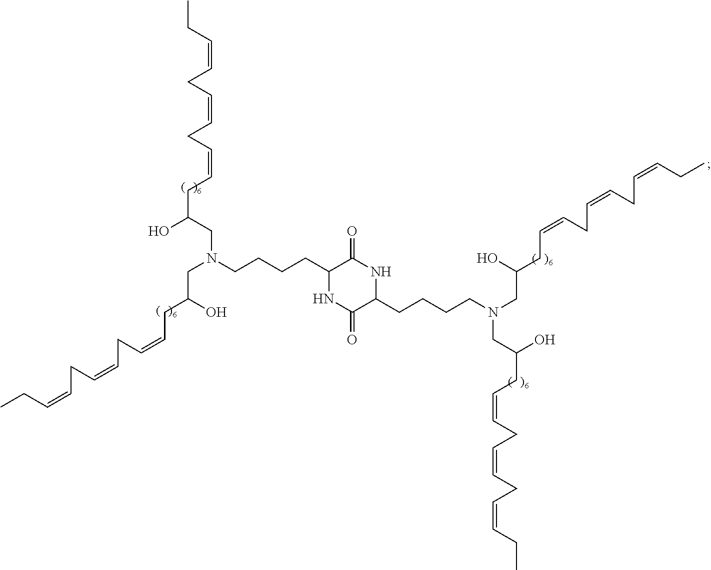 |
| 15 | 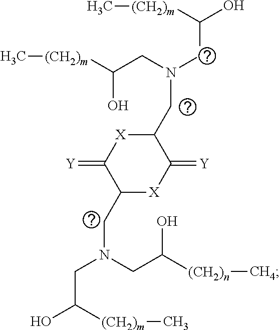 |
| 16 | 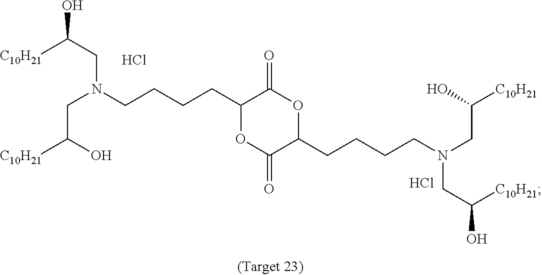 |
| 17 | 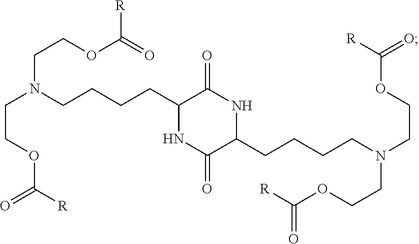 |
| 18 | 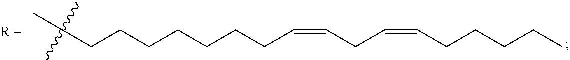 [SMILES: *CCCCCCC/C=C\C/C=C\CCCCC] |
| 19 | 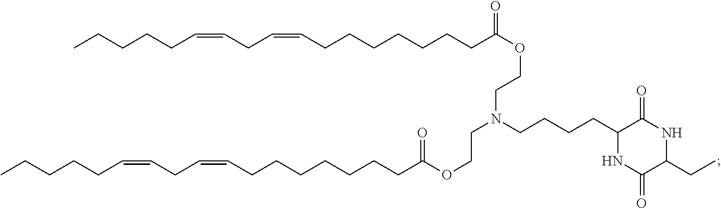 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC] |
| 20 | 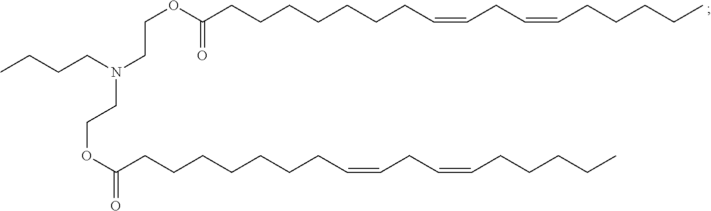 [SMILES: CCCCC/C=C\C/C=C\CCCCCCCC(=O)OCCN(CCCC)CCOC(=O)CCCCCCC/C=C\C/C=C\CCCCC ; ";"] |
| 21 | 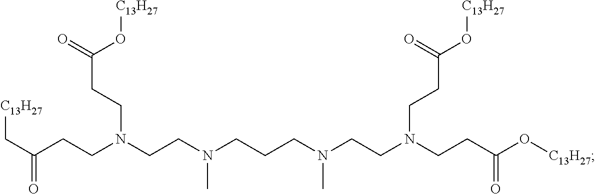 |
| 22 | 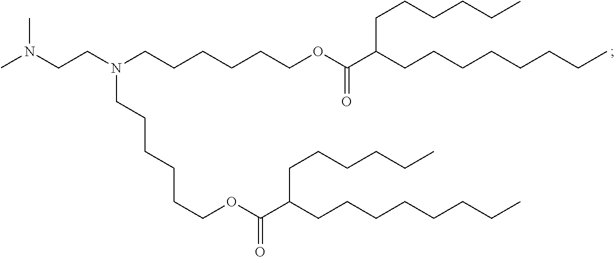 [SMILES: CCCCCCCCC(CCCCCC)C(=O)OCCCCCCN(CCCCCCOC(=O)C(CCCCCC)CCCCCCCC)CCN(C)C] |
| 23 | 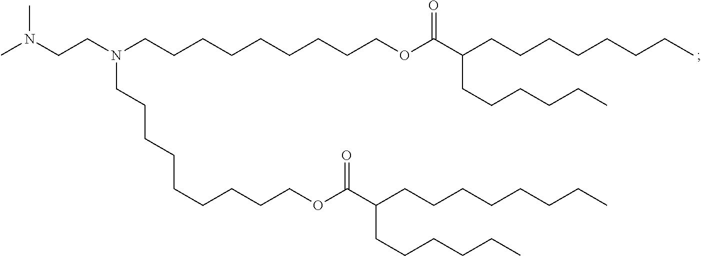 [SMILES: CCCCCCCCC(CCCCCC)C(=O)OCCCCCCCCCN(CCCCCCCCCOC(=O)C(CCCCCC)CCCCCCCC)CCN(C)C] |
| 24 | 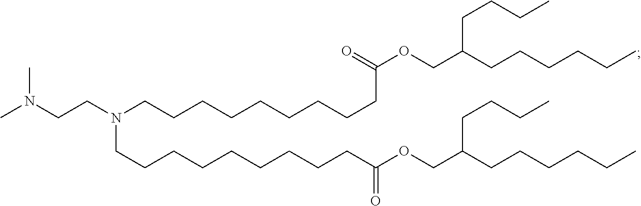 [SMILES: CCCCCCC(CCCC)COC(=O)CCCCCCCCCN(CCCCCCCCCC(=O)OCC(CCCC)CCCCCC)CCN(C)C ; ";"] |
| 25 | 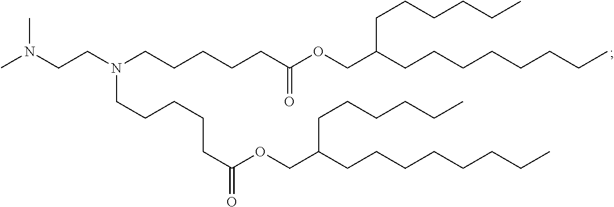 [SMILES: CCCCCCCCC(CCCCCC)COC(=O)CCCCCN(CCCCCC(=O)OCC(CCCCCC)CCCCCCCC)CCN(C)C] |
| 26 | 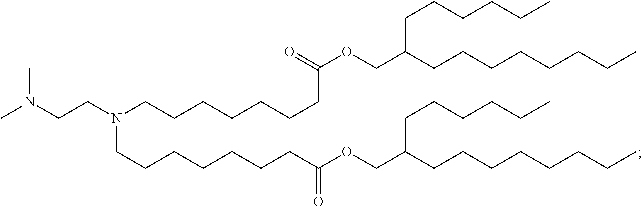 [SMILES: CCCCCCCCC(CCCCCC)COC(=O)CCCCCCCN(CCCCCCCC(=O)OCC(CCCCCC)CCCCCCCC)CCN(C)C] |
| 27 | 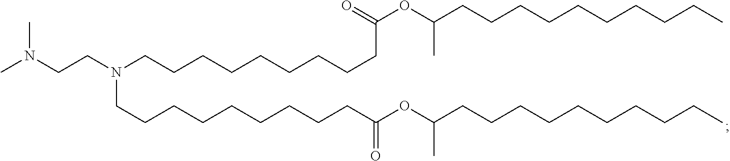 [SMILES: CCCCCCCCCCC(C)OC(=O)CCCCCCCCCN(CCCCCCCCCC(=O)OC(C)CCCCCCCCCC)CCN(C)C] |
| 28 | 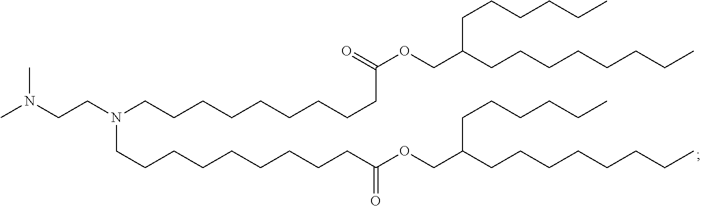 [SMILES: CCCCCCCCC(CCCCCC)COC(=O)CCCCCCCCCN(CCCCCCCCCC(=O)OCC(CCCCCC)CCCCCCCC)CCN(C)C] |
| 29 | 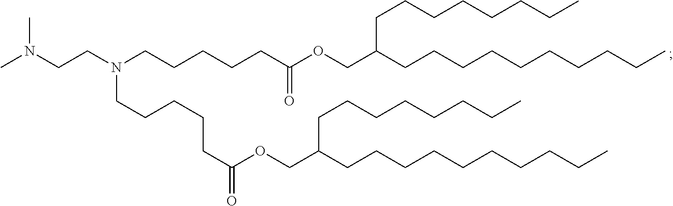 [SMILES: CCCCCCCCCCC(CCCCCCCC)COC(=O)CCCCCN(CCCCCC(=O)OCC(CCCCCCCC)CCCCCCCCCC)CCN(C)C] |
| 30 | 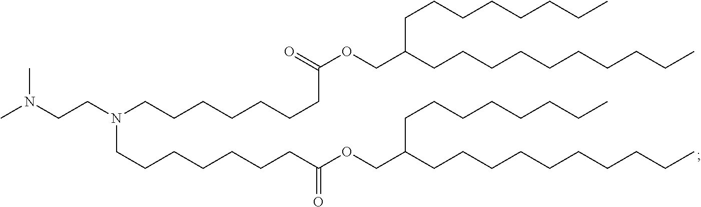 [SMILES: CCCCCCCCCCC(CCCCCCCC)COC(=O)CCCCCCCN(CCCCCCCC(=O)OCC(CCCCCCCC)CCCCCCCCCC)CCN(C)C] |
| 31 | 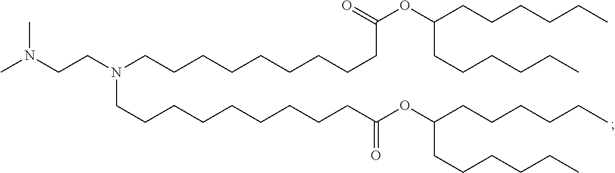 [SMILES: CCCCCCC(CCCCCC)OC(=O)CCCCCCCCCN(CCCCCCCCCC(=O)OC(CCCCCC)CCCCCC)CCN(C)C] |
| 32 | 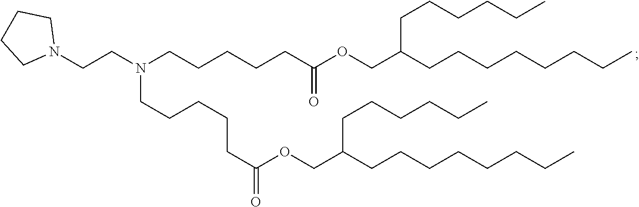 [SMILES: CCCCCCCCC(CCCCCC)COC(=O)CCCCCN(CCCCCC(=O)OCC(CCCCCC)CCCCCCCC)CCN1CCCC1] |
| 33 | 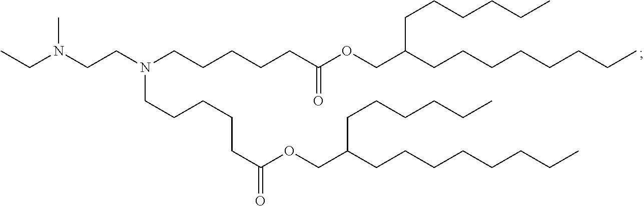 [SMILES: CCCCCCCCC(CCCCCC)COC(=O)CCCCCN(CCCCCC(=O)OCC(CCCCCC)CCCCCCCC)CCN(C)CC] |
| 34 | 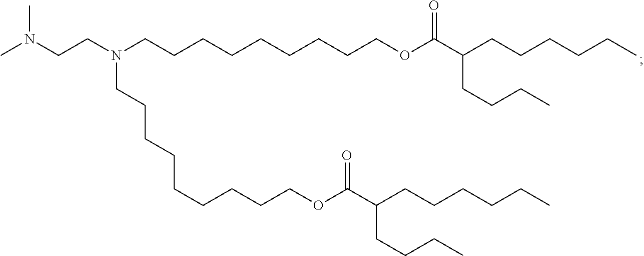 [SMILES: CCCCCCC(CCCC)C(=O)OCCCCCCCCCN(CCCCCCCCCOC(=O)C(CCCC)CCCCCC)CCN(C)C] |
| 35 | 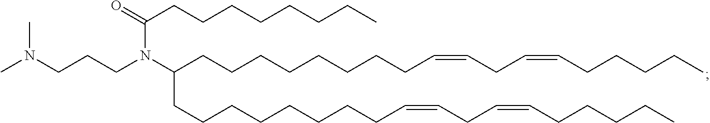 [SMILES: CCCCC/C=C\C/C=C\CCCCCCCCC(CCCCCCCC/C=C\C/C=C\CCCCC)N(CCCN(C)C)C(=O)CCCCCCCC] |
| 36 | 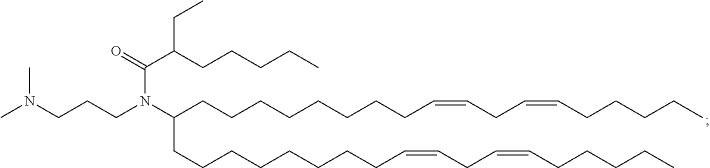 [SMILES: CCCCC/C=C\C/C=C\CCCCCCCCC(CCCCCCCC/C=C\C/C=C\CCCCC)N(CCCN(C)C)C(=O)C(CC)CCCCC] |
| 37 | 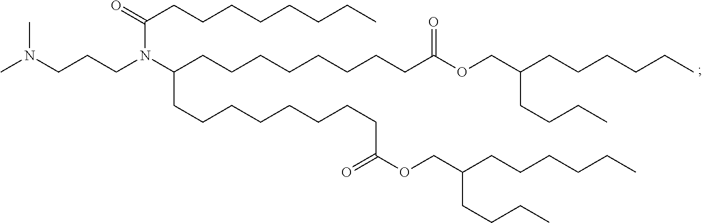 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| 38 | 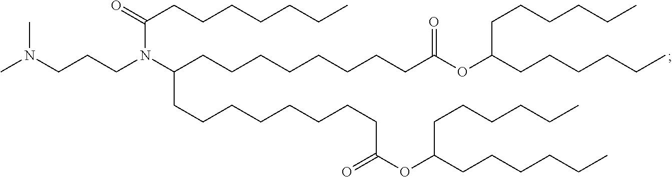 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |
| 39 | 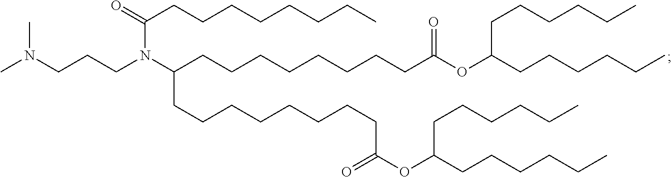 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] |
| 40 | 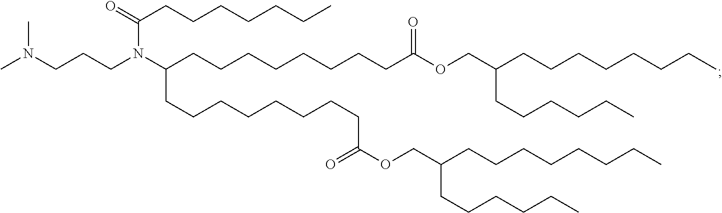 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] |
| 41 | 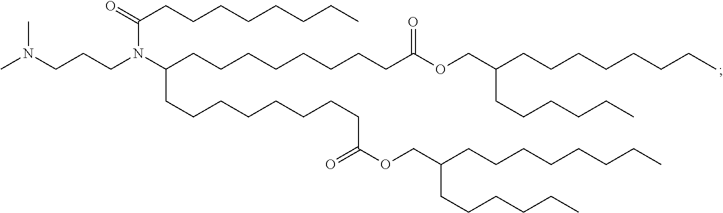 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] |
| 42 | 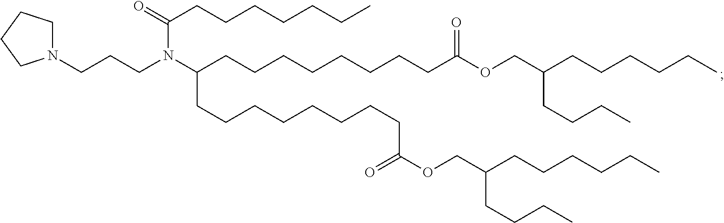 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] |
| 43 | 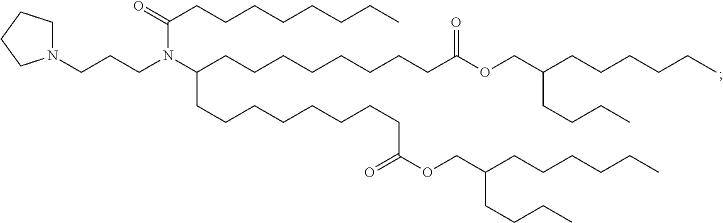 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] |
| 44 | 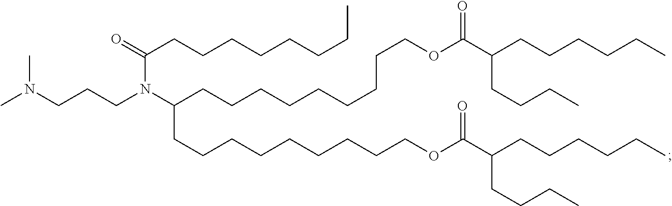 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] |
| 45 | 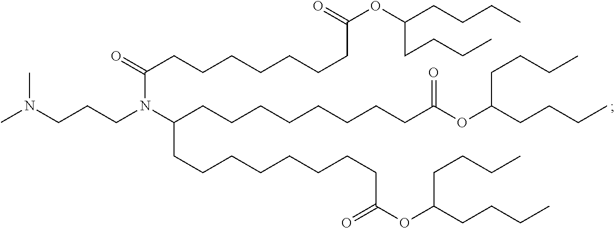 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] |
| 46 | 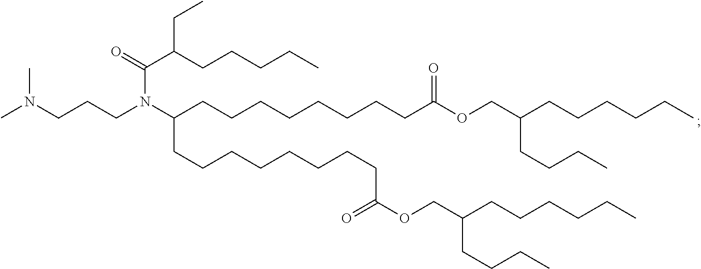 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC] |
| 47 | 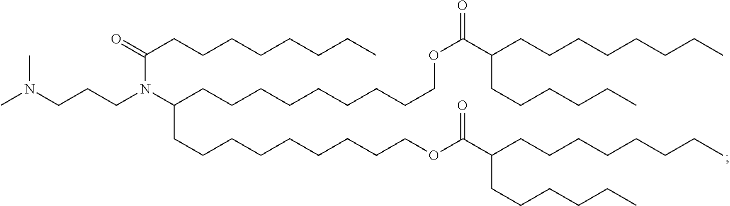 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC] |
| 48 | 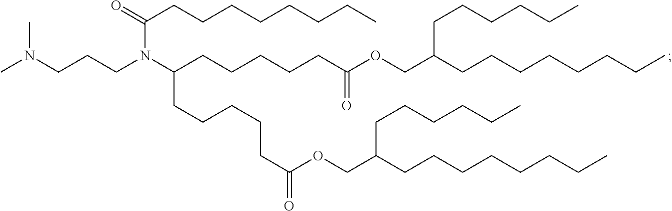 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] |
| 49 | 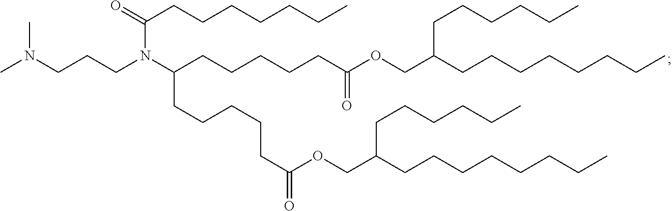 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC] |
| 50 | 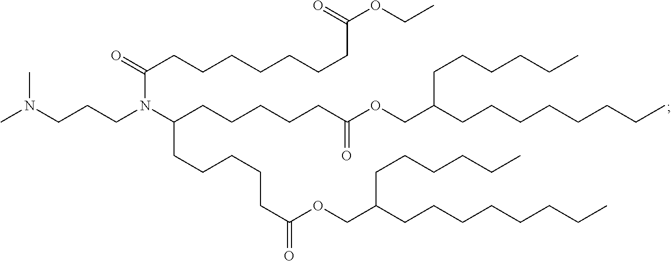 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC] |
| 51 | 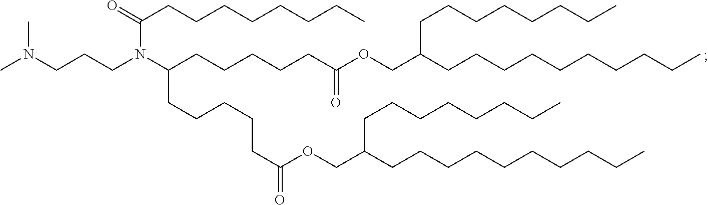 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC] |
| 52 | 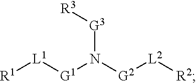 |
| 53 | 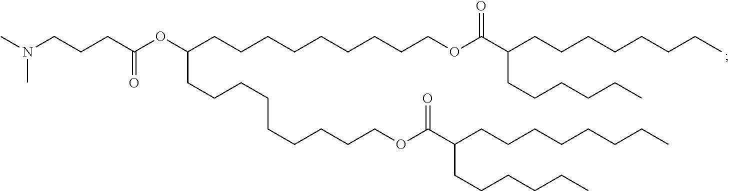 [SMILES: CCCCCCCCC(CCCCCC)C(=O)OCCCCCCCCCC(CCCCCCCCCOC(=O)C(CCCCCC)CCCCCCCC)OC(=O)CCCN(C)C] |
| 54 | 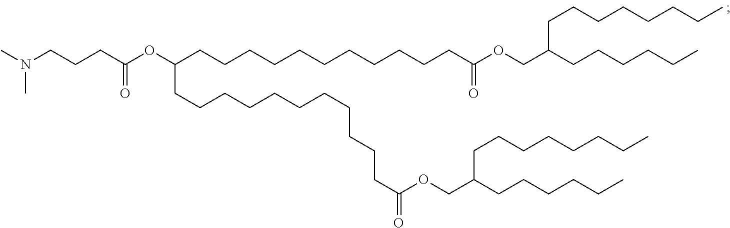 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C] |
| 55 | 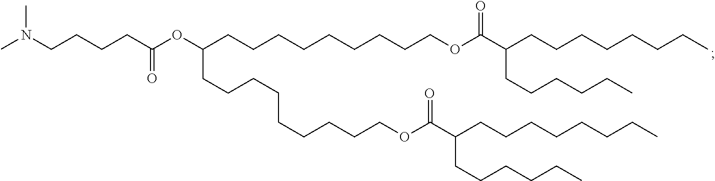 [SMILES: CCCCCCCCC(CCCCCC)C(=O)OCCCCCCCCCC(CCCCCCCCCOC(=O)C(CCCCCC)CCCCCCCC)OC(=O)CCCCN(C)C] |
| 56 | 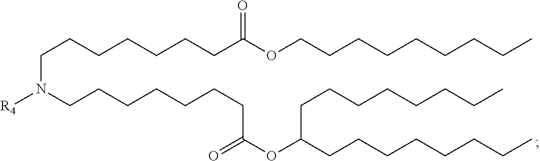 |
| 57 | 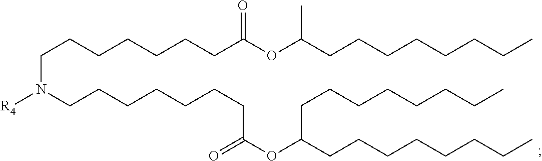 |
| 58 | 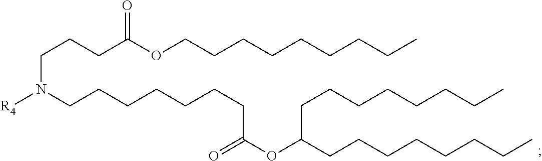 |
| 59 | 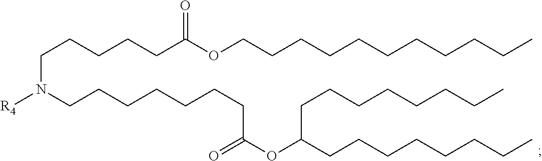 |
| 60 | 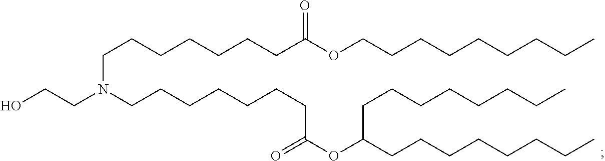 [SMILES: CCCCCCCCCOC(=O)CCCCCCCN(CCO)CCCCCCCC(=O)OC(CCCCCCCC)CCCCCCCC] |
| 61 | 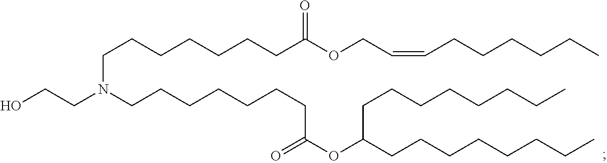 [SMILES: CCCCCC/C=C\COC(=O)CCCCCCCN(CCO)CCCCCCCC(=O)OC(CCCCCCCC)CCCCCCCC] |
| 62 | 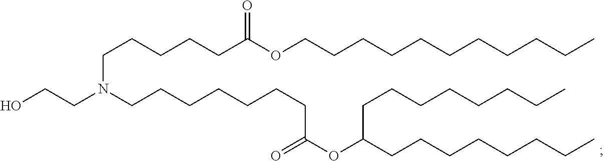 [SMILES: CCCCCCCCCCCOC(=O)CCCCCN(CCO)CCCCCCCC(=O)OC(CCCCCCCC)CCCCCCCC] |
| 63 | 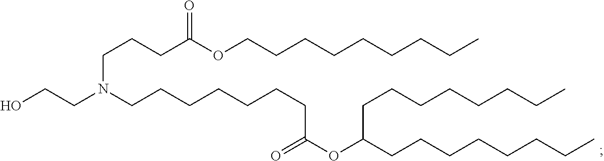 [SMILES: CCCCCCCCCOC(=O)CCCN(CCO)CCCCCCCC(=O)OC(CCCCCCCC)CCCCCCCC] |
| 64 | 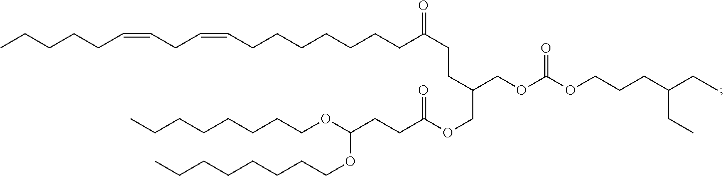 [SMILES: CCCCC/C=C\C/C=C\CCCCCCCC(=O)CCC(COC(=O)CCC(OCCCCCCCC)OCCCCCCCC)COC(=O)OCCCC(CC)CC] |
| 65 | 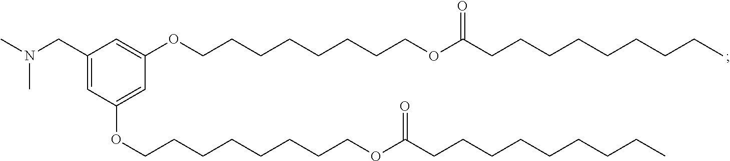 [SMILES: CCCCCCCCCC(=O)OCCCCCCCCOc1cc(CN(C)C)cc(OCCCCCCCCOC(=O)CCCCCCCCC)c1] |
| 66 | 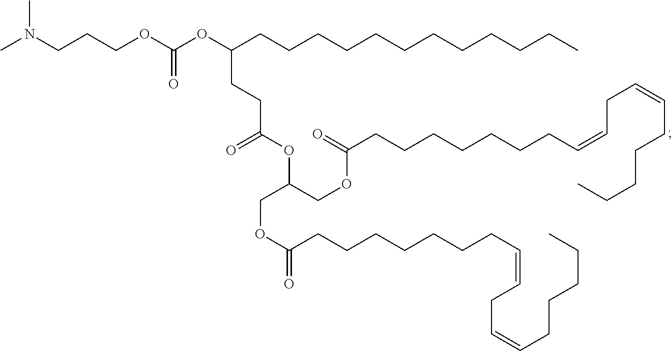 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C] |
| 67 | 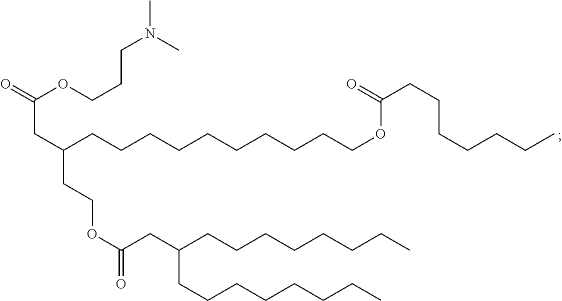 [SMILES: CCCCCCCCC(CCCCCCCC)CC(=O)OCCC(CCCCCCCCCCOC(=O)CCCCCCC)CC(=O)OCCCN(C)C] |
| 68 | 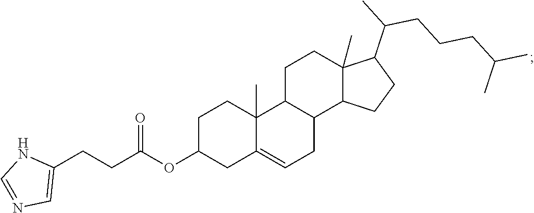 [SMILES: CC(C)CCCC(C)C1CCC2C3CC=C4CC(OC(=O)CCc5cnc[nH]5)CCC4(C)C3CCC12C] |
| 69 | 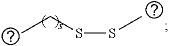 |
| 70 | 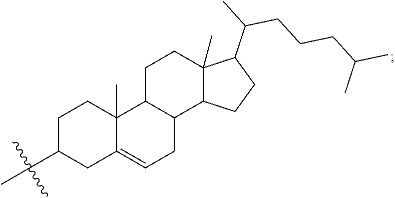 [SMILES: *C1CCC2(C)C(=CCC3C2CCC2(C)C(C(C)CCCC(C)C)CCC32)C1] |
| 71 | 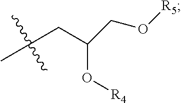 |
| 72 | 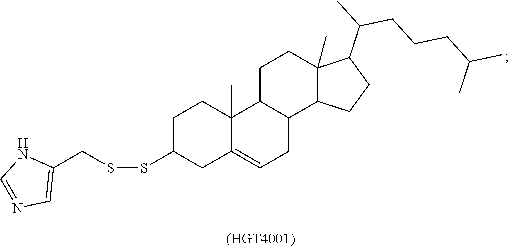 [SMILES: CC(C)CCCC(C)C1CCC2C3CC=C4CC(SSCc5cnc[nH]5)CCC4(C)C3CCC12C] |
| 73 | 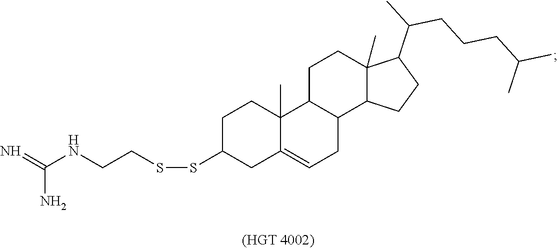 [SMILES: CC(C)CCCC(C)C1CCC2C3CC=C4CC(SSCCNC(=N)N)CCC4(C)C3CCC12C] |
| 74 | 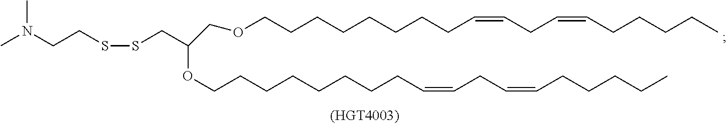 [SMILES: CCCCC/C=C\C/C=C\CCCCCCCCOCC(CSSCCN(C)C)OCCCCCCCC/C=C\C/C=C\CCCCC] |
| 75 | 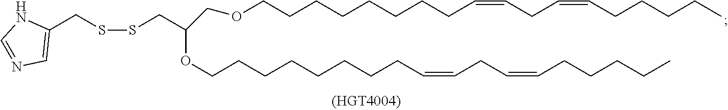 [SMILES: CCCCC/C=C\C/C=C\CCCCCCCCOCC(CSSCc1cnc[nH]1)OCCCCCCCC/C=C\C/C=C\CCCCC] |
| 76 | 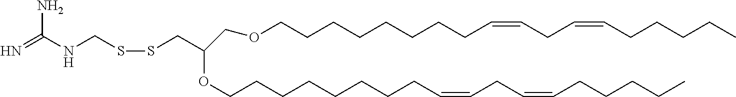 [SMILES: CCCCC/C=C\C/C=C\CCCCCCCCOCC(CSSCNC(=N)N)OCCCCCCCC/C=C\C/C=C\CCCCC] (HGT40 [symbol] |

[symbol]

indicates data missing or illegible when filed

In some embodiments of the lipid composition of the present application, the ionizable cationic lipid is present in an amount from about from about 20 to about 23. In some embodiments, the molar or weight percentage is from about 20, 20.5, 21, 21.5, 22, 22.5, to about 23 or any range derivable therein. In other embodiments, the molar or weight percentage is from about 7.5 to about 20. In some embodiments, the molar or weight percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the ionizable cationic lipid at a molar or weight percentage from about 5% to about 30%. In some embodiments of the lipid composition of the present application, the lipid composition comprises the ionizable cationic lipid at a molar or weight percentage from about 10% to about 25%. In some embodiments of the lipid composition of the present application, the lipid composition comprises the ionizable cationic lipid at a molar or weight percentage from about 15% to about 20%. In some embodiments of the lipid composition of the present application, the lipid composition comprises the ionizable cationic lipid at a molar or weight percentage from about 10% to about 20%. In some embodiments of the lipid composition of the present application, the lipid composition comprises the ionizable cationic lipid at a molar or weight percentage from about 20% to about 30%. In some embodiments of the lipid composition of the present application, the lipid composition comprises the ionizable cationic lipid at a molar or weight percentage of at least (about) 5%, at least (about) 10%, at least (about) 15%, at least (about) 20%, at least (about) 25%, or at least (about) 30%. In some embodiments of the lipid composition of the present application, the lipid composition comprises the ionizable cationic lipid at a molar or weight percentage of at most (about) 5%, at most (about) 10%, at most (about) 15%, at most (about) 20%, at most (about) 25%, or at most (about) 30%.

### Selective Organ Targeting (SORT) Lipids

In some embodiments of the lipid composition of the present application, the lipid (e.g., nanoparticle) composition is preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments of the lipid composition, the lipid composition comprises one or more selective organ targeting (SORT) lipid which leads to the selective delivery of the composition to a particular organ. In some embodiments, the SORT lipid may have two or more alkyl or alkenyl chains of C₆-C₂₄.

In some embodiments of the lipid compositions, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the lipid compositions, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid

may comprise a structural formula:

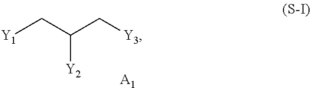

wherein:
Y₁, Y₂, or Y₃ are each independently X₁C(O)R₁ or X₂N⁺R₃R₄R₅;
provided at least one of Yi, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NRₐ, wherein Rₐ is hydrogen, C₁-C₄ alkyl, or C₁-C₄ substituted alkyl;
X₂ is C₁-C₆ alkanediyl or C₁-C₆ substituted alkanediyl;
R₃, R₄, and R₅ are each independently C₁-C₂₄ alkyl, C₁-C₂₄ substituted alkyl, C₁-C₂₄ alkenyl, C₁-C₂₄ substituted alkenyl; and
A₁ is an anion with a charge equal to the number of X₂N⁺R₃R₄R₅ groups in the compound.

In some embodiments of the SORT lipids, the permanently cationic SORT lipid has a structural formula:

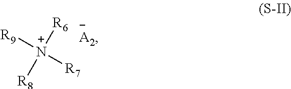

wherein:
R₆-R₉ are each independently C₁-C₂₄ alkyl, C₁-C₂₄ substituted alkyl, C₁-C₂₄ alkenyl, C₁-C₂₄ substituted alkenyl; provided at least one of R₆-R₉ is a group of C₈-C₂₄; and
A₂ is a monovalent anion.

In some embodiments of the lipid compositions, the SORT lipid is ionizable cationic lipid (i.e., comprising one or more hydrophobic components and an ionizable cationic group). The ionizable positively charged moiety may be positively charged at a physiological pH. One ionizable cationic group that may be used in the ionizable cationic lipid is a tertiary ammine group. In some embodiments of the lipid compositions, the SORT lipid has a structural formula:

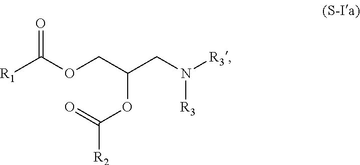

wherein:
R₁ and R₂ are each independently alkyl_{(C8-C24)}, alkenyl_{(C8-C24)}, or a substituted version of either group; and
R₃ and R₃′ are each independently alkyl_{(C≤6)} or substituted alkyl_{(C≤6)}.

In some embodiments of the lipid compositions, the SORT lipid comprises a head group of a particular structure. In some embodiments, the SORT lipid comprises a headgroup having a structural formula:

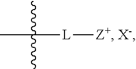

wherein L is a linker; Z⁺ is positively charged moiety and X⁻ is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the lipid compositions, the SORT lipid has a structural formula:

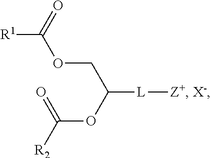

wherein R¹ and R² are each independently an optionally substituted C₆-C₂₄ alkyl, or an optionally substituted C₆-C₂₄ alkenyl.

In some embodiments of the lipid compositions, the SORT lipid has a structural formula:

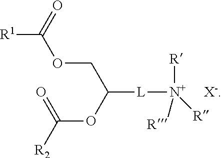

In some embodiments of the lipid compositions, the SORT lipid comprises a Linker (L). In some embodiments, L is

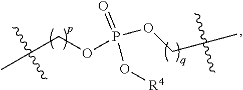

wherein:
p and q are each independently 1, 2, or 3; and
R⁴ is an optionally substituted C₁-C₆ alkyl

In some embodiments of the lipid compositions, the SORT lipid has a structural

formula:

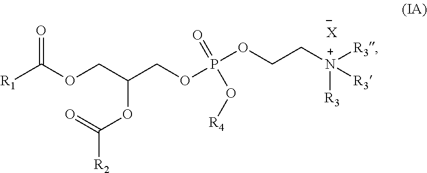

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

In some embodiments of the lipid compositions, the SORT lipid is a phosphatidylcholine (e.g., 14:0 EPC). In some embodiments, the phosphatidylcholine compound is further defined as:

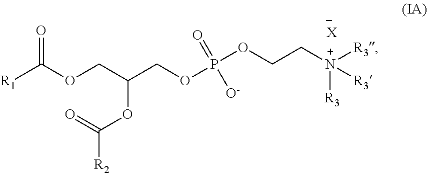

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In some embodiments of the lipid compositions, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the lipid compositions, the SORT lipid has a structural formula:

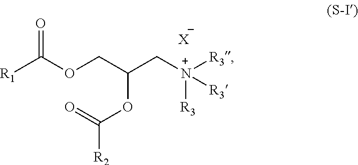

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
X⁻ is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the lipid compositions, the SORT lipid has a structural formula:

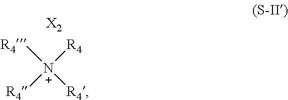

wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid has a structural formula:

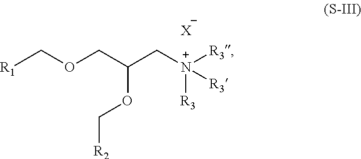

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

By way of example, and without being limited thereto, a SORT lipid of the structural formula of the immediately preceding paragraph is N-[1-(2, 3-dioleyloxy)propyl]-N,N,N-trimethylammonium chloride (DOTMA).

In some embodiments of the lipid compositions, the SORT lipid is an anionic lipid. In some embodiments of the lipid compositions, the SORT lipid has a structural formula:

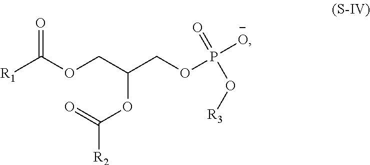

wherein:
R₁ and R₂ are each independently alkyl_{(C8-C24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
   Y₁ is alkanediyl_{(C≤6)} or substituted alkanediyl_{(C≤6)}; and
R₄ is acyloxy_{(C≤8-24)} or substituted acyloxy_{(C≤8-24)}.

In some embodiments of the lipid compositions, the SORT lipid comprises one or more selected from the lipids set forth in Table 6.

TABLE 6
Example SORT lipids

| Lipid Name | Structure |
|---|---|
| 1,2-Dioleoyl-3- dimethylammonium-propane (18:1 DODAP) | 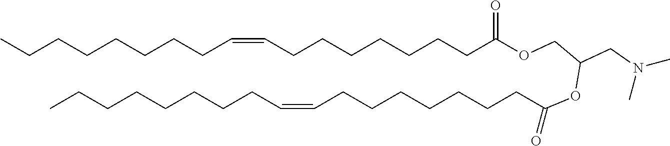 [SMILES: CCCCCCCC/C=C\CCCCCCCC(=O)OCC(CN(C)C)OC(=O)CCCCCCC/C=C\CCCCCCCC] |
| 1,2-dimyristoyl-3- trimethylammonium-propane (14:0 TAP) (e.g., chloride salt) | 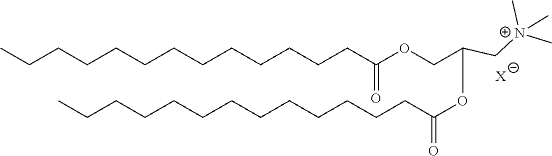 |
| 1,2-dipalmitoyl-3- trimethylammonium-propane (16:0 TAP) (e.g., chloride salt) | 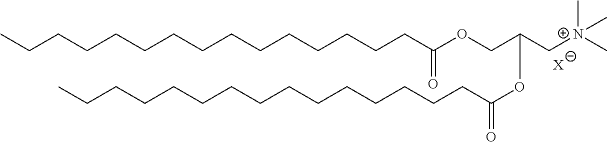 |
| 1,2-stearoyl-3- trimethylammonium-propane (18:0 TAP) (e.g., chloride salt) | 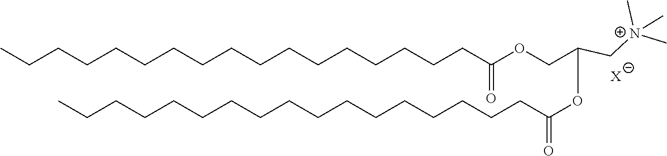 |
| 1,2-Dioleoy1-3- trimethylammonium-propane (18:1 DOTAP) (e.g., chloride salt) | 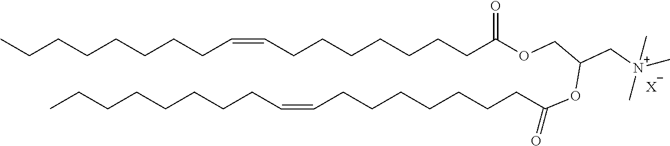 |
| 1,2-Di-O-octadecenyl-3- trimethylammonium propane (DOTMA) (e.g., chloride salt) | 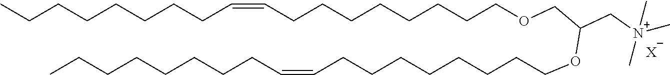 |
| Dimethyldioctadecylammonium (DDAB) (e.g., bromide salt) | 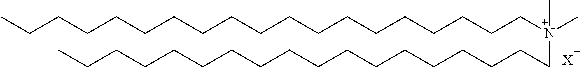 |
| 1,2-dilauroyl-sn-glycero-3- ethylphosphocholine (12:0 EPC) (e.g., chloride salt) | 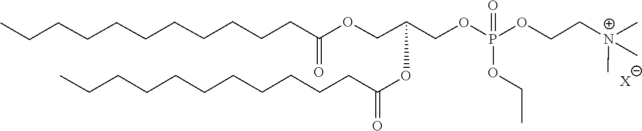 |
| 1,2-Dioleoyl-sn-glycero-3- ethylphosphocholine (14:0 EPC) (e.g., chloride salt) | 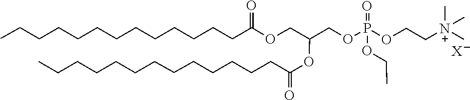 |
| 1,2-dimyristoleoyl-sn-glycero- 3-ethylphosphocholine (14:1 EPC) (e.g., triflate salt) | 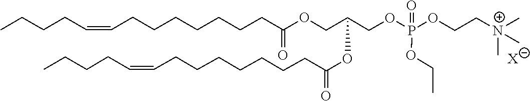 |
| 1,2-dipalmitoyl-sn-glycero-3- ethylphosphocholine (16:0 EPC) (e.g., chloride salt) | 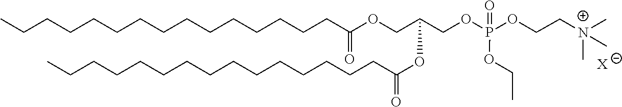 |
| 1,2-distearoyl-sn-glycero-3- ethylphosphocholine (18:0 EPC) (e.g., chloride salt) | 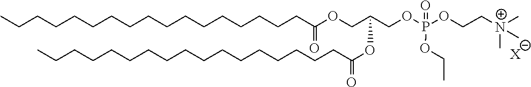 |
| 1,2-dioleoyl-sn-glycero-3- ethylphosphocholine (18:1 EPC) (e.g., chloride salt) | 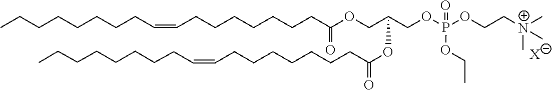 |
| 1-palmitoyl-2-oleoyl-sn- glycero-3- ethylphosphocholine (16:0-18:1 EPC) (e.g., chloride salt) | 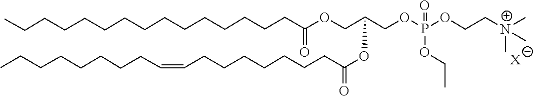 |
| 1,2-di-O-octadeceny1-3- trimethylammonium propane (18:1 DOTMA) (e.g., chloride salt) | 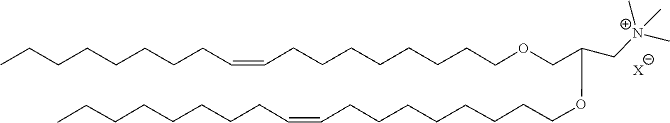 |
| 1,2-dioleoyl-sn-glycero-3- phosphate (18.1 PA) | 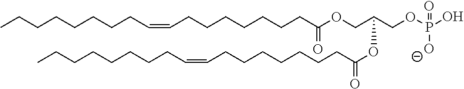 [SMILES: CCCCCCCC/C=C\CCCCCCCC(=O)OC[C@@H](COP(=O)([O-])O)OC(=O)CCCCCCC/C=C\CCCCCCCC] |

X⁻ is a counterion (e.g., Cl⁻, Br⁻, etc.)

In some embodiments of the lipid composition of the present application, the lipid composition comprises the SORT lipid at a molar or weight percentage from about 5% to about 65%, from about 10% to about 65%, from about 15% to about 65%, or from about 20% to about 65%. In some embodiments of the lipid composition of the present application, the lipid composition comprises the SORT lipid at a molar or weight percentage from about 5% to about 60%, from about 10% to about 60%, from about 15% to about 60%, from about 20% to about 60%, or from about 25% to about 60%. In some embodiments of the lipid composition of the present application, the lipid composition comprises the SORT lipid at a molar or weight percentage from about 5% to about 55%, from about 10% to about 55%, from about 15% to about 55%, from about 20% to about 55%, from about 25% to about 55%, or from about 30% to about 55%. In some embodiments of the lipid composition of the present application, the lipid composition comprises the SORT lipid at a molar or weight percentage from about 5% to about 50%, from about 10% to about 50%, from about 15% to about 50%, from about 20% to about 50%, from about 25% to about 50%, from about 30% to about 50%, from about 35% to about 50%, or from about 40% to about 50%. In some embodiments of the lipid composition of the present application, the lipid composition comprises the SORT lipid at a molar or weight percentage from about 30% to about 60%. In some embodiments of the lipid composition of the present application, the lipid composition comprises the SORT lipid at a molar or weight percentage from about 25% to about 60%. In some embodiments of the lipid composition of the present application, the lipid composition comprises the SORT lipid at a molar or weight percentage from about 5% to about 20%, from about 5% to about 25%, from about 5% to about 30%, from about 5% to about 35%, or from about 5% to about 40%. In some embodiments of the lipid composition of the present application, the lipid composition comprises the SORT lipid at a molar or weight percentage of at least (about) 5%, at least (about) 10%, at least (about) 15%, at least (about) 20%, at least (about) 25%, at least (about) 30%, at least (about) 35%, at least (about) 40%, at least (about) 45%, at least (about) 50%, at least (about) 55%, at least (about) 60%, or at least (about) 65%. In some embodiments of the lipid composition of the present application, the lipid composition comprises the SORT lipid at a molar or weight percentage of at most (about) 5%, at most (about) 10%, at most (about) 15%, at most (about) 20%, at most (about) 25%, at most (about) 30%, at most (about) 35%, at most (about) 40%, at least (about) 45%, at most (about) 50%, at most (about) 55%, at most (about) 60%, or at most (about) 65%. In some embodiments of the lipid composition of the present application, the lipid composition comprises the SORT lipid at a molar or weight percentage of (about) 5%, 6%, 7%, 8%, 9%, 10%, 15%, 20%, 25%, 30%, 35%, 40%, 45%, 50%, 55%, 60%, or 65%, or of a range between (inclusive) any two of the foregoing values.

In some embodiments of the method, the SORT lipid effects delivery of the nucleic acid editing system to the cell of the subject characterized by a greater therapeutic effect compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the SORT lipid achieves about 1.1-fold to about 20-fold therapeutic effect compared to that achieved with a reference lipid composition.

In some embodiments of the method, the SORT lipid achieves about 1.1-fold to about 10-fold therapeutic effect compared to that achieved with a reference lipid composition. In some embodiments of the method, the SORT lipid achieves about 1.1-fold to about 5-fold therapeutic effect compared to that achieved with a reference lipid composition. In some embodiments of the method, the SORT lipid achieves about 5-fold to about 10-fold therapeutic effect compared to that achieved with a reference lipid composition. In some embodiments of the method, the SORT lipid achieves about 10-fold to about 20-fold therapeutic effect compared to that achieved with a reference lipid composition. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition.

In some embodiments of the method, the SORT lipid achieves about 1.1-fold to about 20-fold therapeutic effect compared to that achieved with a reference lipid composition in cells selected from basal cell, secretory cell such as goblet cell and club cell, ciliated cell and any combination thereof. In some embodiments of the method, the SORT lipid achieves about 1.1-fold to about 10-fold greater therapeutic effect compared to that achieved with a reference lipid composition in cells selected from basal cell, secretory cell such as goblet cell and club cell, ciliated cell and any combination thereof. In some embodiments of the method, the SORT lipid achieves about 1.1-fold to about 5-fold greater therapeutic effect compared to that achieved with a reference lipid composition in cells selected from basal cell, secretory cell such as goblet cell and club cell, ciliated cell and any combination thereof. In some embodiments of the method, the SORT lipid achieves about 10-fold to about 20-fold greater therapeutic effect compared to that achieved with a reference lipid composition in cells selected from basal cell, secretory cell such as goblet cell and club cell, ciliated cell and any combination thereof. In some embodiments of the method, the SORT lipid achieves at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold therapeutic effect compared to that achieved with a reference lipid composition in cells selected from basal cell, secretory cell such as goblet cell and club cell, ciliated cell and any combination thereof.

In some embodiments of the method, the SORT lipid effects delivery of the nucleic acid editing system to cells of the subject characterized by a therapeutic effect in a greater plurality of cells compared to that achieved with a reference lipid composition. In some embodiments, the reference lipid composition does not comprise the SORT lipid. In some embodiments, the reference lipid composition does not comprise the amount of the SORT lipid. In some embodiments, the reference lipid comprises 13,16,20-tris(2-hydroxydodecyl)-13,16,20,23-tetraazapentatricontane-11,25-diol (“LF92”), a phospholipid, cholesterol, and a PEG-lipid.

In some embodiments of the method, the SORT lipid achieves therapeutic effect in about 1.1-fold to about 20-fold cells compared to that achieved with a reference lipid composition. In some embodiments of the method, the SORT lipid achieves therapeutic effect in about 1.1-fold to about 10-fold cells compared to that achieved with a reference lipid composition. In some embodiments of the method, the SORT lipid achieves therapeutic effect in about 1.1-fold to about 5-fold cells compared to that achieved with a reference lipid composition. In some embodiments of the method, the SORT lipid achieves therapeutic effect in about 10-fold to about 20-fold cells compared to that achieved with a reference lipid composition. In some embodiments of the method, the SORT lipid achieves therapeutic effect in at least about 1.1-fold, at least about 5-fold, at least about 10-fold, at least about 20-fold, at least about 50-fold, or at least about 100-fold cells compared to that achieved with a reference lipid composition.

In some embodiments of the method, the SORT lipid achieves therapeutic effect in about 1.1-fold to about 20-fold cells compared to that achieved with a reference lipid composition, wherein the cells are selected from basal cell, secretory cell such as goblet cell and club cell, ciliated cell and any combination thereof. In some embodiments of the method, the SORT lipid achieves therapeutic effect in about 1.1-fold to about 10-fold cells compared to that achieved with a reference lipid composition, wherein the cells are selected from basal cell, secretory cell such as goblet cell and club cell, ciliated cell and any combination thereof. In some embodiments of the method, the SORT lipid achieves therapeutic effect in about 5-fold to about 10-fold more cells compared to that achieved with a reference lipid composition, wherein the cells are selected from basal cell, secretory cell such as goblet cell and club cell, ciliated cell and any combination thereof. In some embodiments of the method, the SORT lipid achieves therapeutic effect in about 10-fold to about 20-fold more cells compared to that achieved with a reference lipid composition, wherein the cells are selected from basal cell, secretory cell such as goblet cell and club cell, ciliated cell and any combination thereof. In some embodiments of the method, the SORT lipid achieves therapeutic effect in at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, or at least about 20-fold more cells compared to that achieved with a reference lipid composition, wherein the cells is selected from basal cell, secretory cell such as goblet cell and club cell, ciliated cell and any combination thereof.

### Additional Lipids

In some embodiments of the lipid composition of the present application, the lipid composition further comprises an additional lipid including but not limited to a steroid or a steroid derivative, a PEG lipid, and a phospholipid.

### Phospholipids or Other Zwitterionic Lipids

In some embodiments of the lipid composition of the present application, the lipid composition further comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments of the lipid compositions, the phospholipid is not an ethylphosphocholine.

In some embodiments of the lipid composition of the present application, the compositions may further comprise a molar or weight percentage of the phospholipid to the total lipid composition from about 20 to about 23. In some embodiments, the molar percentage is from about 20, 20.5, 21, 21.5, 22, 22.5, 23, 24, 25, 26, 27, 28, 29, to about 30 or any range derivable therein. In other embodiments, the molar or weight percentage is from about 7.5 to about 60. In some embodiments, the molar or weight percentage is from about 5, 6, 7, 7.5, 8, 9, 10, 11, 12, 13, 14, 15, 16, 17, 18, 19, to about 20 or any range derivable therein. In other embodiments, the molar or weight percentage is from about 7.5 to about 60. In some embodiments, the molar or weight percentage is from about 5, 6, 7, 7.5, 8, 9, 10, 11, 12, 13, 14, 15, 16, 17, 18, 19, 20, 20.5, 21, 21.5, 22, 22.5, 23, 24, 25, 26, 27, 28, 29, to about 30 or any range derivable therein.

In some embodiments of the lipid composition of the present application, the lipid composition comprises the phospholipid at a molar or weight percentage from about 8% to about 23%. In some embodiments of the lipid composition of the present application, the lipid composition comprises the phospholipid at a molar or weight percentage from about 10% to about 20%. In some embodiments of the lipid composition of the present application, the lipid composition comprises the phospholipid at a molar or weight percentage from about 15% to about 20%. In some embodiments of the lipid composition of the present application, the lipid composition comprises the phospholipid at a molar or weight percentage from about 8% to about 15%. In some embodiments of the lipid composition of the present application, the lipid composition comprises the phospholipid at a molar or weight percentage from about 10% to about 15%. In some embodiments of the lipid composition of the present application, the lipid composition comprises the phospholipid at a molar or weight percentage from about 12% to about 18%. In some embodiments of the lipid composition of the present application, the lipid composition comprises the phospholipid at a molar or weight percentage of at least (about) 8%, at least (about) 10%, at least (about) 12%, at least (about) 15%, at least (about) 18%, at least (about) 20%, or at least (about) 23%. In some embodiments of the lipid composition of the present application, the lipid composition comprises the phospholipid at a molar or weight percentage of at most (about) 8%, at most (about) 10%, at most (about) 12%, at most (about) 15%, at most (about) 18%, at most (about) 20%, or at most (about) 23%.

### Steroids or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

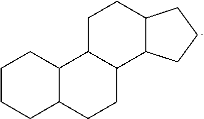 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

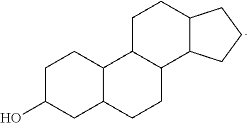 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

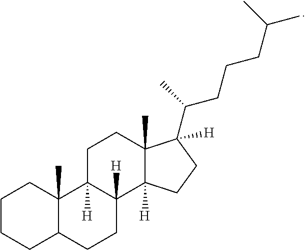

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar or weight percentage of the steroid to the total lipid composition from about 40 to about 46. In some embodiments, the molar or weight percentage is from about 40, 41, 42, 43, 44, 45, to about 46 or any range derivable therein. In other embodiments, the molar or weight percentage of the steroid relative to the total lipid composition is from about 15 to about 40, or from about 15 to about 45. In some embodiments, the molar or weight percentage is about 15, 16, 18, 20, 22, 24, 26, 28, 30, 32, 34, 36, 38, or 40, or any range derivable therein. In some embodiments, the molar or weight percentage is about 15, 16, 18, 20, 22, 24, 26, 28, 30, 32, 34, 36, 38, 40, 41, 42, 43, 44, 45, 46, 47, 48, 49, to about 50, or any range derivable therein.

In some embodiments of the lipid composition of the present application, the lipid composition comprises the steroid or steroid derivative at a molar or weight percentage from about 15% to about 46%. In some embodiments of the lipid composition of the present application, the lipid composition comprises the steroid or steroid derivative at a molar or weight percentage from about 20% to about 40%. In some embodiments of the lipid composition of the present application, the lipid composition comprises the steroid or steroid derivative at a molar or weight percentage from about 25% to about 35%. In some embodiments of the lipid composition of the present application, the lipid composition comprises the steroid or steroid derivative at a molar or weight percentage from about 30% to about 40%. In some embodiments of the lipid composition of the present application, the lipid composition comprises the steroid or steroid derivative at a molar or weight percentage from about 20% to about 30%. In some embodiments of the lipid composition of the present application, the lipid composition comprises the steroid or steroid derivative at a molar or weight percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, the lipid composition comprises the steroid or steroid derivative at a molar or weight percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

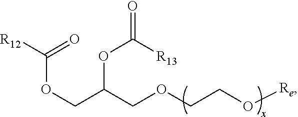

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

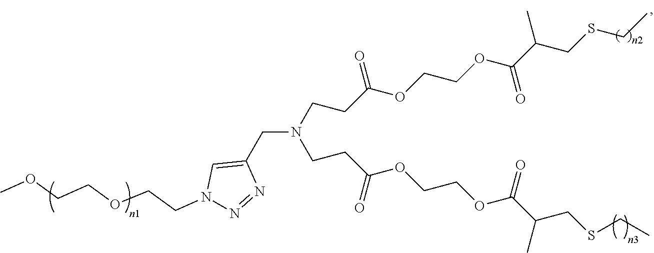

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar or weight percentage of the PEG lipid to the total lipid composition from about 4.0 to about 4.6. In some embodiments, the molar or weight percentage is from about 4.0, 4.1, 4.2, 4.3, 4.4, 4.5, to about 4.6 or any range derivable therein. In other embodiments, the molar or weight percentage is from about 1.5 to about 4.0. In some embodiments, the molar or weight percentage is from about 1, 1.5, 1.75, 2, 2.25, 2.5, 2.75, 3, 3.25, 3.5, 3.75, to about 4.0 or any range derivable therein. In some embodiments, the molar or weight percentage is from about 1, 1.5, 1.75, 2, 2.25, 2.5, 2.75, 3, 3.25, 3.5, 3.75, 4, 4.0, 4.5, 5.0, 5.5, 6.0, 6.5, 7.0, 7.5, 8.0, 8.5, 9.0, 9.5, to about 10 or any range derivable therein.

In some embodiments of the lipid composition of the present application, the lipid composition comprises the polymer-conjugated lipid at a molar or weight percentage from about 0.5% to about 10%. In some embodiments of the lipid composition of the present application, the lipid composition comprises the polymer-conjugated lipid at a molar or weight percentage from about 1% to about 8%. In some embodiments of the lipid composition of the present application, the lipid composition comprises the polymer-conjugated lipid at a molar percentage from about 1% to about 10%. In some embodiments of the lipid composition of the present application, the lipid composition comprises the polymer-conjugated lipid at a molar percentage from about 2% to about 10%. In some embodiments of the lipid composition of the present application, the lipid composition comprises the polymer-conjugated lipid at a molar or weight percentage from about 2% to about 7%. In some embodiments of the lipid composition of the present application, the lipid composition comprises the polymer-conjugated lipid at a molar or weight percentage from about 3% to about 5%. In some embodiments of the lipid composition of the present application, the lipid composition comprises the polymer-conjugated lipid at a molar or weight percentage from about 5% to about 10%. In some embodiments of the lipid composition of the present application, the lipid composition comprises the polymer-conjugated lipid at a molar or weight percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, the lipid composition comprises the polymer-conjugated lipid at a molar or weight percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Methods for Enhancing CFTR Expression or Activity

Disclosed herein includes a method for enhancing an expression or activity of cystic fibrosis transmembrane conductance regulator (CFTR) protein in cell(s). The method may comprise: (a) contacting the cell with a nucleic acid editing system assembled with lipid composition(s), which nucleic acid editing system comprises (i) a guide nucleic acid, (ii) a heterologous polypeptide comprising an endonuclease or a heterologous polynucleotide encoding the heterologous polypeptide, and (iii) a donor template nucleic acid, to yield a complex of the heterologous endonuclease with the guide nucleic acid in the cell; (b) cleaving a CFTR gene or transcript in the cell with the complex at a cleavage site to yield a cleaved CFTR gene or transcript; and (c) using the donor template nucleic acid to repair the cleaved CFTR gene or transcript to yield a repaired CFTR gene or transcript encoding a functional CFTR protein in the cell, thereby enhancing the expression or activity of CFTR protein in the cell. The CFTR gene or transcript cleaved in (b) or a cleavage event may be an endogenous CFTR gene or transcript. The CFTR gene or transcript cleaved in (b) or a cleavage event may be a mutant CFTR gene or transcript. The CFTR gene or transcript cleaved in (b) or a cleavage event may be an endogenous mutant CFTR gene or transcript. The repaired CFTR gene or transcript may be generated via homology directed repair (HDR) pathway or event(s). The functional CFTR protein encoded by the repaired CFTR gene or transcript may be a wild-type CFTR protein. The nucleic acid editing system may be one described herein, for example, in the “NUCLEIC ACID EDITING SYSTEMS” section. The method may comprise using said donor template nucleic acid to repair said cleaved CFTR gene or transcript via a homology directed repair (HDR) pathway or event(s).

In some embodiments of the method for enhancing the expression or activity of CFTR protein, the lipid composition comprises: an ionizable cationic lipid; and a selective organ targeting (SORT) lipid separate from the ionizable cationic lipid. The lipid composition may further comprise a phospholipid separate from the SORT lipid. The lipid composition may be one described herein, for example, in the “LIPID COMPOSITIONS” section.

In some embodiments of the method for enhancing the expression or activity of CFTR protein, the contacting or (a) is ex vivo. In some embodiments, the contacting or (a) is in vitro. In some embodiments, the contacting or (a) is in vivo. In some embodiments, the contacting or (a) is repeated. In some embodiments, the cell is a cell described herein, for example, in the “CELLS” section. In some embodiments, the cell is a lung cell, e.g., a lung basal cell. In some embodiments, the lung basal cell exhibits or is determined to exhibit p63. In some embodiments, the cell is an airway epithelial cell, e.g., a bronchial epithelial cell. In some embodiments, the cell is undifferentiated. In some embodiments, the cell is differentiated. In some embodiments, the contacting or (a) comprises contacting a plurality of cells (e.g., lung cells, such as lung basal cells) that comprise the cell (e.g., lung cell, such as lung basal cell). The repairing or (c) may yield a functional (e.g., wild-type) CFTR gene, transcript or protein, e.g., in at least about 10%, 15%, 20%, 25%, 30%, 35%, 40%, 45%, 50%, 60%, 65%, or 70% of the plurality of cells.

In some embodiments of the method for enhancing the expression or activity of CFTR protein, a cleavage event or (b) comprises cleaving a (e.g., endogenous) (e.g., mutant) CFTR gene or transcript that comprises a mutation relative to a corresponding wild type counterpart. The mutation may be a loss-of-function mutation, such as a nonsense or frameshift mutation. The mutation may be present in an exon selected from exons 9-27 (e.g., exon 10, exon 12) of CFTR. The mutation may be F508del or G542X. The mutation may be F508del. The mutation may be G542X. The (e.g., loss-of-function) mutation may be associated with cystic fibrosis, hereditary emphysema, or chronic obstructive pulmonary disease (COPD). The method may alter a mutant CFTR gene or transcript in cell(s) to a functional CFTR gene or transcript, for example, by inserting one or more nucleotides at or near the cleavage site.

In some embodiments of the method for enhancing the expression or activity of CFTR protein, a repair event or (c) is characterized by an off-target insertion or/and deletion (indel) rate, for example, of no more than about 50%, 49%, 48%, 47%, 46%, 45%, 44%, 43%, 42%, 41%, or 40%. The off-target indel rate may be associated with or characteristic of non-homologous end-joining (NHEJ) pathway or event(s) in the repair event or (c). The off-target indel rate may comprise a ratio of (1) a sum of test cells detected to have an incorrectly altered CFTR gene or transcript relative to (2) a sum of total test cells. The incorrectly altered CFTR gene or transcript may encode a non-functional CFTR protein. The incorrectly altered CFTR gene or transcript may comprise an insertion or/and deletion (indel) relative to an endogenous (e.g., mutant) CFTR gene or transcript in cell(s) at or near a cleavage site of the nucleic acid editing system. In some embodiments, a repair event or (c) is characterized by an on-target repair rate, for example, of at least about 5%, 6%, 7%, 8%, 9%, 10%, 11%, 12%, 13%, 14%, 15%, 16%, 17%, 18%, 19%, or 20%. The on-target repair rate may be associated with or characteristic of homology directed repair (HDR) pathway or event(s) in the repair event or (c). The on-target repair rate may comprise a ratio of (1) a sum of test cells detected to have the (e.g., correctly) repaired CFTR gene or transcript relative to (2) a sum of total test cells. The (e.g., correctly) repaired CFTR gene or transcript may encode a functional (e.g., wild-type) CFTR protein. Accurate specificity to a target sequence as well as the prevention or reduction of the number of off-target insertion and/or deletions may be performed by designing oligo using in silio prediction algorithms, or other design methods of oligos, such to analyze potential targets and off-targets in a given sequence.

In some embodiments of the method for enhancing the expression or activity of CFTR protein, the method increases an amount of a functional CFTR gene, transcript or protein in the cell (e.g., by at least about 1.1-fold) relative to a corresponding control. In some embodiments, the method yields a therapeutically effective amount of a functional of CFTR gene, transcript or protein in the cell. In some embodiments, the method yields at least about 10%, 15%, 20%, 25%, or 30%, by mole or by weight, among all detected or detectable CFTR gene, transcript or protein. The functional CFTR gene, transcript or protein may be a wild-type CFTR gene, transcript or protein. The corresponding control may be a corresponding cell absent the contacting or (a). The corresponding control may be a corresponding cell absent the contacting or (a).

In some embodiments of the method for enhancing the expression or activity of CFTR protein, the method enhances (e.g., chloride) ion transport in cell(s) (e.g., by at least about 1.1-fold) relative to a corresponding control. The method may reduce defective export from or import to cell(s) of chloride, such as chloride anion or in the form of a chloride salt or other chloride-containing compound. The method may enhance or stimulate ion (e.g., chloride) transport in cell(s). The enhanced or stimulated ion (e.g., chloride) transport may result in secretion or absorption of (e.g., chloride) ions. The corresponding control may be a corresponding cell absent the contacting. Enhanced (e.g., chloride) ion transport may be determined by evaluating CFTR-mediated currents across cell(s) by employing standard Ussing chamber (see Ussing and Zehrahn, Acta. Physiol. Scand. 23:110-127, 1951) or nasal potential difference measurements (see Knowles et al., Hum. Gene Therapy 6:445-455, 1995). The enhanced chloride transport may be determined by the Ieq (equivalen current) assay using the TECC-24 system as described in Vu et al., J. Med. Chem. 2017, 60, 458-473, which is hereby incorporated by reference in its entirety.

The enhanced (e.g., chloride) ion transport may be determined by CFTR-dependent whole-cell current measurement(s), as described in International Patent Application No. PCT/US2017/032967, published as WO2017201091, which is hereby incorporated by reference in its entirety.

In some embodiments, the method further comprises deriving (e.g., by cell culturing) a cell composition (e.g., a lung cell composition) from the cell.

### Methods for Lung Cell Editing

Disclosed herein includes a method for genetic correction of cystic fibrosis transmembrane conductance regulator (CFTR) in a lung (e.g., basal) cell, comprising: contacting the lung (e.g., basal) cell with a composition that comprises a nucleic acid editing system assembled with a lipid composition, thereby delivering the nucleic acid editing system to the lung (e.g., basal) cell.

Disclosed herein includes a method for genetic correction of cystic fibrosis transmembrane conductance regulator (CFTR) in a cell composition, comprising: contacting the cell composition comprising a plurality of lung (e.g., basal) cells with a composition that comprises a nucleic acid editing system assembled with a lipid composition, thereby delivering the nucleic acid editing system, e.g., to at least about 10%, 15%, 20%, 25%, 30%, 35%, 40%, 45%, 50%, 60%, 65%, or 70% of the plurality of lung (e.g., basal) cells.

Disclosed herein includes a method for genetic correction of cystic fibrosis transmembrane conductance regulator (CFTR) in a cell composition, comprising: contacting the cell composition with a composition that comprises a nucleic acid editing system assembled with a lipid composition, which cell composition comprise a lung (e.g., basal) cell and a lung non-basal cell, thereby delivering the nucleic acid editing system to the lung (e.g., basal) cell in a greater amount than that delivered to the lung non-basal cell. The non-basal cell may be an ionocyte (e.g., exhibiting or determined to exhibit to FOXI1), a ciliated cell, or a secretory cell (such as goblet cell and club cell).

In some embodiments of any one of the methods for genetic correction of CFTR of this section, the lung (e.g., basal) cell or the plurality of lung (e.g., basal) cells is/are determined to exhibit a mutation in CFTR gene. In some embodiments of any one of the methods for genetic correction of CFTR of this section, the lung (e.g., basal) cell or the plurality of lung (e.g., basal) cells exhibit(s) a mutation in CFTR gene.

In some embodiments of any one of the methods for genetic correction of CFTR of this section, the lung (e.g., basal) cell or the plurality of lung (e.g., basal) cells is/are from a subject. The subject may be determined to exhibit a mutation in CFTR gene. The subject may exhibit a mutation in CFTR gene.

In some embodiments of any one of the methods for genetic correction of CFTR of this section, the contacting is ex vivo. In some embodiments of any one of the methods for genetic correction of CFTR of this section, the contacting is in vitro. In some embodiments of any one of the methods for genetic correction of CFTR of this section, the contacting is in vivo.

In some embodiments of the method, a cell or plurality of cells is isolated from the subject. The compositions as described elsewhere here may be contacted with the cell outside of the subject. Upon administration of the composition or therapeutic, the cell may be re-injected or other wise re-introduced into the subject. In some embodiments of the method, the cell is a cell line. In some embodiments of the method, the cell is a lung cell. In some embodiments, the lung cell is a lung airway cell. Examples of lung airway cells that can be targeted by the delivery of the present application includes but is not limited to basal cell, secretory cell such as goblet cell and club cell, ciliated cell and any combination thereof.

### Methods of Treatment

Disclosed herein includes a method for treating a subject having or suspected of having a cystic fibrosis transmembrane conductance regulator (CFTR)-associated condition, the method comprising administering to the subject a composition comprising a nucleic acid editing system (such as one described herein in the “NUCLEIC ACID EDITING SYSTEMS” section) assembled with a lipid composition (such as one described herein in the “LIPID COMPOSITIONS” section). The CFTR-associated condition may be cystic fibrosis, hereditary emphysema, chronic obstructive pulmonary disease (COPD), or a combination thereof. The subject may be a mammal. The subject may be a non-human species (e.g., mouse, rat, rabbit, dog, monkey, gibbon, chimp, ape, baboon, cow, pig, horse, sheep, cat and other species). The subject may be a human. The subject may be determined to exhibit a mutation (e.g., F508del or G542X) in CFTR gene. In some embodiments, the administering comprises systemic (e.g., intravenous) administration. In some embodiments, the subject is selected from the group consisting of mouse, rat, monkey, and human. In some embodiments, the subject is a human.

### Engineered Cells

Disclosed herein includes an engineered cell composition comprising or derived from a cell, the cell comprising a heterologous cystic fibrosis transmembrane conductance regulator (CFTR) gene, transcript or protein produced by a composition described herein, e.g., in the “NUCLEIC ACID EDITING SYSTEMS” section.

Disclosed herein includes an engineered cell composition comprising or derived from a cell having an expression or activity of cystic fibrosis transmembrane conductance regulator (CFTR) protein enhanced by a method described herein, e.g., in the “METHODS FOR ENHANCING CFTR EXPRESSION OR ACTIVITY” section.

Disclosed herein includes an engineered cell composition comprising or derived from a lung (e.g., basal) cell or a plurality of lung (e.g., basal) cells brought in contact with a composition that comprises a nucleic acid editing system assembled with a lipid composition as described herein, e.g., in the “METHODS FOR LUNG CELL EDITING” section.

In some embodiments, the engineered cell composition is derived in vitro. In some embodiments, the engineered cell composition is derived ex vivo. CELLS Basal cells

Basal cells are derived from undifferentiated columnar epithelium in the developing airway. They are characterized by basal position in the columnar epithelium, the presence of hemidesmosomes (characterized by alpha 6 beta 4 integrins), cytokeratins 5 and 14, NGFR, and the nuclear protein p63. The distribution of basal cells varies by airway level and animal species. Airways that are larger in diameter have more basal cells than airways with smaller diameters. As the airway decreases in diameter, the number of basal cells also decreases, and none are present in the terminal bronchioles.

In another aspect, provided herein is a method for (e.g., lung) basal cell delivery of a nucleic acid editing system, comprising: contacting said (e.g., lung) basal cell with a composition comprising said nucleic acid editing system assembled with a lipid composition, thereby delivering said nucleic acid editing system to said (e.g., lung) basal cell. In some embodiments, the contacting is ex vivo. For example, cells may be isolated from a patient and contacted with the composition. The cells may then be reintroduced to the subject. In some embodiments, the contacting is in vitro. In some embodiments, the contacting is in vivo. The cells may be derived are be from the subject. The cells may be in the subject. The subject may be a subject as described elsewhere herein. For example, the subject may be determined to exhibit a mutation in the CFTR gene.

In another aspect, provided herein is a method for (e.g., lung) basal cell delivery of a nucleic acid editing system, comprising: contacting a (e.g., lung) cell composition comprising a plurality of (e.g., lung) basal cells with a composition that comprises said nucleic acid editing system assembled with a lipid composition, thereby delivering said nucleic acid editing system to at least 15% of said plurality of (e.g., lung) basal cells. The lung cell composition may also comprise other lung cells, as described elsewhere herein, for example, an ionocyte, a ciliated cell, a secretory cell, or a combination thereof. The (e.g. lung) cell composition may comprise a first cell of a first CFTR genotype and a second cell of a second CFTR genotype. The (e.g. lung) cells may comprises a variety of genotypes or a variety of CFTR alleles.

In another aspect, provided herein is a method for (e.g., lung) basal cell-targeted delivery of a nucleic acid editing system, comprising: contacting a plurality of (e.g., lung) cells of a plurality of cell types with a composition that comprises said nucleic acid editing system assembled with a lipid composition, which plurality of cells comprise a (e.g., lung) basal cell and a (e.g., lung) non-basal cell, thereby delivering said nucleic acid editing system to said basal cell in a greater amount than that delivered to said non-basal cell. The lung cell composition may also comprise other lung cells, as described elsewhere herein, for example, an ionocyte, a ciliated cell, a secretory cell, or a combination thereof. The (e.g. lung) cell composition may comprise a first cell of a first CFTR genotype and a second cell of a second CFTR genotype. The (e.g. lung) cells may comprises a variety of genotypes or a variety of CFTR alleles.

In another aspect, provided herein is a method for delivery to basal cells of a subject, comprising administrating to the subject the pharmaceutical composition as described in the present application. In some embodiments of the method, the pharmaceutical composition comprises a nucleic acid editing system assembled with a lipid composition as described in the present application, wherein the lipid composition comprises (i) an ionizable cationic lipid; and (ii) a selective organ targeting (SORT) lipid separate from the ionizable cationic lipid. The lipid composition may further comprise a phospholipid. In some embodiments, the basal cell is a lung basal cell.

In some embodiments of the method, the pharmaceutical composition is administrated to the subject through any suitable delivery. In some embodiment, the pharmaceutical composition is administrated to the subject through inhalation. In some embodiments, the pharmaceutical composition is administrated to the subject through systemic administration such as intravenous administration.

### Ciliated Cells

Ciliated cells are those cells with cilia structures on the cell surface. Examples of ciliated cells include but are not limited to respiratory tract ciliated cells, oviduct ciliated cell, uterine endometrial ciliated cells, rete testis ciliated cells, ductulus efferens ciliated cells, and/or ciliated ependymal cells. Human respiratory tract ciliated cells bear 200 to 300 cilia on their surface. Cilia are elongated motile cylindrical projections from the apical cell membrane, about 0.25 mm in diameter, that contain microtubules and cytoplasm in continuity with that of the cell. Human tracheal cilia are 5 to 8 mm long, becoming shorter in more distal airways.

The structure of a cilium is complex and consists of an axoneme, anchored by a basal body and a rootlet to the cell, and it possesses some smaller claw-like formations on its tip. The direction in which the basal body points defines the orientation of the cilium and the direction of the effective beat. The axoneme contains nine pairs of microtubules which surround a central pair of microtubules, as well as radial spokes and peripheral nexin links, which to a great extent maintain the wheel-like arrangement of the cilium. Inner and outer arms attach to the microtubules. The main structural protein of the doublets is tubulin. The arms (inner and outer) contain dynein, which is a protein classified as an ATPase. Dynein generates the force that results in a sliding movement of the microtubules, responsible for ciliary movement. It is generally accepted that the outer dynein arms are mostly responsible for beating frequency whereas the inner dynein arms together with the radial spokes and nexin links have a role in the waveform of the beating. Changes in the structural integrity of the axoneme can result in abnormal movement that ranges from stillness to aberrant patterns of hyperactivity.

### Secretory Cell

“Secretory cell” refers to cells specialized for secretion. These cells are usually epithelial in origin and have characteristic, well developed rough endoplasmic reticulum or, in the case of cells secreting lipids or lipid-derived products have well developed smooth endoplasmic reticulum. Examples of secretory cells include: salivary gland cells, mammary gland cells, lacrimal gland cells, creuminous gland cells, eccrine sweat gland cells, apocrine sweat gland cells, sebaceous gland cells, Bowman's gland cells, Brunner's gland cells, seminal vesicle cells, prostate gland cells, bulbourethral gland cells, Bartholin's gland cells, gland of Littre cells, endometrial cells, goblet cells of the respiratory and digestive tracts, mucous cells of the stomach, zymogenic cells of gastric glands, oxyntic cells of gastric glands, acinar cells of the pancreas, paneth cells of the small intestine, type II pneumocytes of the lung, club cells of the lung, anterior pituitary cells, cells of the intermediate pituitary, cells of the posterior pituitary, cells of the gut and respiratory tract, cells of the thyroid gland, cells of the parathyroid gland, cells of the adrenal gland, cells of the testes, cells of the ovaries, cells of the juxtaglomerular apparatus of the kidney, cells secreting extracellular matrix (e.g., epithelial cells, nonepithelial cells (such as fibroblasts, chondrocytes, osteoblasts/osteocytes, osteoprogenitor cells), and secretory cells of the immune system (e.g., Ig producing B cells, cytokine producing T cells, etc.).

The following are examples of compositions and evaluations of compositions of the disclosure. It is understood that various other embodiments may be practiced, given the general description provided above.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for enhancing an expression or activity of cystic fibrosis transmembrane conductance regulator (CFTR) protein in a cell, the method comprising: (a) contacting said cell with a nucleic acid editing system assembled with a lipid composition, which nucleic acid editing system comprises (i) a guide nucleic acid, (ii) a heterologous polypeptide comprising an endonuclease or a heterologous polynucleotide encoding said heterologous polypeptide, and (iii) a donor template nucleic acid, to yield a complex of said heterologous endonuclease with said guide nucleic acid in said cell; (b) cleaving a CFTR gene or transcript in said cell with said complex at a cleavage site to yield a cleaved CFTR gene or transcript; and (c) using said donor template nucleic acid to repair said cleaved CFTR gene or transcript to yield a repaired CFTR gene or transcript encoding a functional CFTR protein in said cell, thereby enhancing said expression or activity of CFTR protein in said cell.

Embodiment 2. The method of Embodiment 1, wherein (c) is characterized by an off-target insertion or/and deletion (indel) rate of no more than about 50%, 49%, 48%, 47%, 46%, 45%, 44%, 43%, 42%, 41%, or 40%.

Embodiment 3. The method of Embodiment 2, wherein said off-target indel rate comprises a ratio of (1) a sum of test cells detected to have an incorrectly altered CFTR gene or transcript relative to (2) a sum of total test cells.

Embodiment 4. The method of any one of Embodiments 1-3, wherein (c) is characterized by an on-target repair rate of at least about 5%, 6%, 7%, 8%, 9%, 10%, 11%, 12%, 13%, 14%, 15%, 16%, 17%, 18%, 19%, or 20%.

Embodiment 5. The method of Embodiment 4, wherein said on-target repair rate comprises a ratio of (1) a sum of test cells detected to have said repaired CFTR gene or transcript relative to (2) a sum of total test cells.

Embodiment 6. The method of any one of Embodiments 1-5, wherein the method increases an amount of a functional CFTR gene, transcript or protein in said cell (e.g., by at least about 1.1-fold) relative to a corresponding control, optionally, wherein said corresponding control is a corresponding cell absent said contacting.

Embodiment 7. The method of any one of Embodiments 1-6, wherein the method yields a therapeutically effective amount of a functional of CFTR gene, transcript or protein in said cell (e.g., at least about 10%, 15%, 20%, 25%, or 30% among all detectable CFTR gene, transcript or protein).

Embodiment 8. The method of any one of Embodiments 1-7, wherein the method enhances (e.g., chloride) ion transport in said cell (e.g., by at least about 1.1-fold) relative to a corresponding control, optionally, wherein said corresponding control is a corresponding cell absent said contacting.

Embodiment 9. The method of any one of Embodiments 1-8, wherein said cell is a lung cell.

Embodiment 10. The method of Embodiment 9, wherein said cell is a lung basal cell.

Embodiment 11. The method of any one of Embodiments 1-10, wherein said cell is an airway epithelial cell (e.g., a bronchial epithelial cell).

Embodiment 12. The method of any one of Embodiments 1-11, wherein said cell is undifferentiated.

Embodiment 13. The method of any one of Embodiments 1-11, wherein said cell is differentiated.

Embodiment 14. The method of any one of Embodiments 1-13, wherein (b) comprises cleaving a CFTR gene or transcript that comprises a loss-of-function mutation.

Embodiment 15. The method of Embodiment 14, wherein said loss-of-function mutation comprises a mutation in an exon selected from exons 9-27 of CFTR.

Embodiment 16. The method of Embodiment 14 or 15, wherein said loss-of-function mutation is F508del or G542X.

Embodiment 17. The method of any one of Embodiments 14-16, wherein said loss-of-function mutation is associated with cystic fibrosis, hereditary emphysema, or chronic obstructive pulmonary disease (COPD).

Embodiment 18. The method of any one of Embodiments 1-17, wherein said contacting is ex vivo.

Embodiment 19. The method of any one of Embodiments 1-17, wherein said contacting is in vitro.

Embodiment 20. The method of any one of Embodiments 1-17, wherein said contacting is in vivo.

Embodiment 21. The method of any one of Embodiments 1-20, wherein said contacting is repeated.

Embodiment 22. The method of any one of Embodiments 1-21, wherein said contacting comprises contacting a plurality of cells that comprise said cell.

Embodiment 23. The method of Embodiment 22, wherein said repairing yields a functional CFTR gene, transcript or protein in at least about 10%, 15%, 20%, 25%, 30%, 35%, 40%, 45%, 50%, 60%, 65%, or 70% of said plurality of cells, optionally wherein said plurality of cells are a plurality of (e.g., lung) basal cells.

Embodiment 24. The method of any one of Embodiments 1-23, wherein said lipid composition comprises: an ionizable cationic lipid; and a selective organ targeting (SORT) lipid separate from said ionizable cationic lipid.

Embodiment 25. The method of Embodiment 24, wherein said lipid composition comprises a phospholipid separate from said SORT lipid.

Embodiment 26. The method of any one of Embodiments 1-25, wherein said therapeutic effect is characterized by an amount or activity of said agent detectable in said at least about 10% (e.g., at least about 15%) basal cells in said organ or tissue of said subject.

Embodiment 27. The method of any one of Embodiments 1-26, wherein said lipid composition comprises said SORT lipid at a molar percentage from about 20% to about 65%.

Embodiment 28. The method of any one of Embodiments 1-27, wherein said lipid composition comprises said ionizable cationic lipid at a molar percentage from about 5% to about 30%.

Embodiment 29. The method of any one of Embodiments 1-28, wherein said lipid composition comprises said phospholipid at a molar percentage from about 8% to about 23%.

Embodiment 30. The method of any one of Embodiments 1-29, wherein said phospholipid is not an ethylphosphocholine.

Embodiment 31. The method of any one of Embodiments 1-30, wherein said lipid composition further comprises a steroid or steroid derivative.

Embodiment 32. The method of Embodiment 31, wherein said lipid composition comprises said steroid or steroid derivative at a molar percentage from about 15% to about 46%.

Embodiment 33. The method of any one of Embodiments 1-32, wherein said lipid composition further comprises a polymer-conjugated lipid (e.g., poly(ethylene glycol) (PEG)-conjugated lipid).

Embodiment 34. The method of Embodiment 33, wherein said lipid composition comprises said polymer-conjugated lipid at a molar percentage from about 0.5% to about 10%, from about 1% to about 10%, or from about 2% to about 10%.

Embodiment 35. The method of any one of Embodiments 1-34, wherein said therapeutic agent is a polynucleotide; and wherein a molar ratio of nitrogen in said lipid composition to phosphate in said polynucleotide (N/P ratio) is no more than about 20:1.

Embodiment 36. The method of Embodiment 35, wherein said N/P ratio is from about 5:1 to about 20:1.

Embodiment 37. The method of any one of Embodiments 1-36, wherein a molar ratio of said therapeutic agent to total lipids of said lipid composition is no more than about 1:1, 1:10, 1:50, or 1:100.

Embodiment 38. The method of any one of Embodiments 1-37, wherein at least about 85% of said therapeutic agent is encapsulated in particles of said lipid compositions.

Embodiment 39. The method of any one of Embodiments 1-38, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

Embodiment 40. The method of any one of Embodiments 1-39, wherein said lipid composition has an apparent ionization constant (pKa) outside a range of 6 to 7.

Embodiment 41. The method of Embodiment 40, wherein said apparent pKa of said lipid composition is of about 7 or higher.

Embodiment 42. The method of Embodiment 40, wherein said apparent pKa of said lipid composition is of about 8 or higher.

Embodiment 43. The method of Embodiment 42, wherein said apparent pKa of said lipid composition is from about 8 to about 13.

Embodiment 44. The method of any one of Embodiments 1-43, wherein said SORT lipid comprises a permanently positively charged moiety (e.g., a quaternary ammonium ion).

Embodiment 45. The method of Embodiment 44, wherein said SORT lipid comprises a counterion.

Embodiment 46. The method of any one of Embodiments 1-45, wherein said SORT lipid is a phosphocholine lipid (e.g., saturated or unsaturated).

Embodiment 47. The method of any one of Embodiments 46, wherein said SORT lipid is an ethylphosphocholine.

Embodiment 48. The method of any one of Embodiments 1-47, wherein said SORT lipid comprises a headgroup having a structural formula:

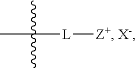

wherein L is a (e.g., biodegradable) linker; Z⁺ is positively charged moiety (e.g., a quaternary ammonium ion); and X⁻ is a counterion.

Embodiment 49. The method of Embodiment 48, wherein said SORT lipid has a structural formula:

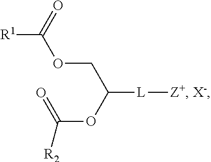

wherein R¹ and R² are each independently an optionally substituted C₆-C₂₄ alkyl, or an optionally substituted C₆-C₂₄ alkenyl.

Embodiment 50. The method of Embodiment 48, wherein said SORT lipid has a structural formula:

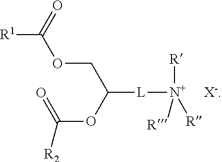

Embodiment 51. The method of any one of Embodiments 48-50, wherein L is

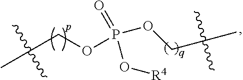

wherein: p and q are each independently 1, 2, or 3; and R⁴ is an optionally substituted C₁-C₆ alkyl.

Embodiment 52. The method of Embodiment 48, wherein said SORT lipid has a structural formula:

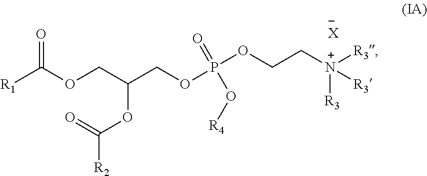

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

Embodiment 53. The method of any one of Embodiments 1-47, wherein said SORT lipid has a structural formula:

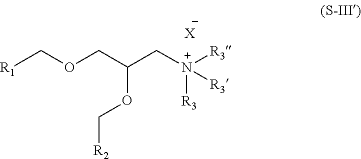

wherein:
   R₁ and R₂ are each independently alkyl_{(C8-C24)}, alkenyl_{(C8-C24)}, or a substituted version of either group;
   R₃, R₃′, and R₃″ are each independently alkyl_{(C≤6)} or substituted alkyl_{(C≤6)};
   X⁻ is a monovalent anion.

Embodiment 54. The method of any one of Embodiments 1-47, wherein said SORT lipid has a structural formula:

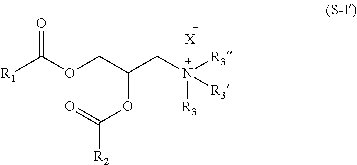

wherein:
   R₁ and R₂ are each independently alkyl_{(C8-C24)}, alkenyl_{(C8-C24)}, or a substituted version of either group;
   R₃, R₃′, and R₃″ are each independently alkyl_{(C≤6)} or substituted alkyl_{(C≤6)};
   X⁻ is a monovalent anion.

Embodiment 55. The method of any one of Embodiments 1-47, wherein said SORT lipid has a structural formula:

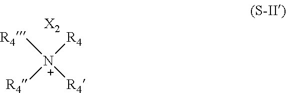

wherein:
   R₄ and R₄′ are each independently alkyl_{(C6-C24)}, alkenyl_{(C6-C24)}, or a substituted version of either group;
   R₄″ is alkyl_{(C≤24)}, alkenyl_{(C≤24)}, or a substituted version of either group;
   R₄′″ is alkyl_{(C1-C8)}, alkenyl_{(C2-C8)}, or a substituted version of either group; and
   X₂ is a monovalent anion.

Embodiment 56. The method of any one of Embodiments 1-47, wherein said SORT lipid is selected from those set forth in Table 6, or pharmaceutically acceptable salts thereof, or a subset of the lipids and the pharmaceutically acceptable salts thereof.

Embodiment 57. The method of any one of Embodiments 1-56, wherein the ionizable cationic lipid is a dendrimer or dendron of a generation (g) having a structural formula:

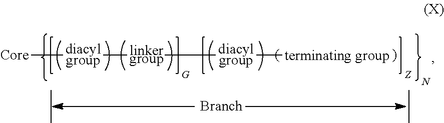

or a pharmaceutically acceptable salt thereof, wherein:
(a) the core comprises a structural formula (X_{Core}):

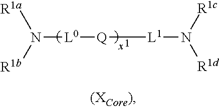

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

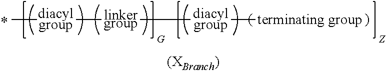

wherein:
   * indicates a point of attachment of the branch to the core;
   g is 1, 2, 3, or 4;

$$\\ Z = {2}^{{}_{} ( g - 1 )} ;$$

G=0, when g=1; or G=Σᵢ₌₀^{i=g-2}2ⁱ, when g≠1;
(c) each diacyl group independently comprises a structural formula

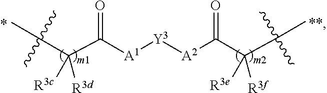

wherein:
   * indicates a point of attachment of the diacyl group at the proximal end thereof;
   ** indicates a point of attachment of the diacyl group at the distal end thereof;
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl; m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

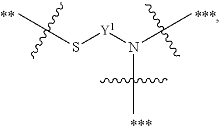

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 58. The method of Embodiment 57, wherein x¹ is 0, 1, 2, or 3.

Embodiment 59. The method of Embodiment 57 or 58, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl (e.g.,

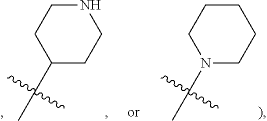 [SMILES: *C1CCNCC1 ; *N1CCCCC1]

N—(C₁-C₃ alkyl)-piperidinyl (e.g.,

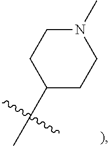 [SMILES: *C1CCN(C)CC1]

piperazinyl (e.g.,

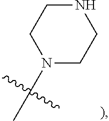 [SMILES: *N1CCNCC1]

N—(C₁-C₃ alkyl)-piperadizinyl (e.g.,

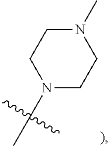 [SMILES: *N1CCN(C)CC1]

morpholinyl (e.g.,

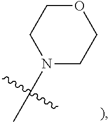 [SMILES: *N1CCOCC1]

### N-pyrrolidinyl (e.g.,

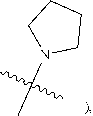 [SMILES: *N1CCCC1]

pyrrolidinyl (e.g.,

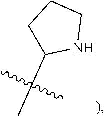 [SMILES: *C1CCCN1]

or N—(C₁-C₃ alkyl)-pyrrolidinyl (e.g.,

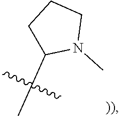 [SMILES: *C1CCCN1C]

(e.g., C₆-C₁₀) aryl, and C₃-C₅ heteroaryl (e.g., imidazolyl (e.g.,

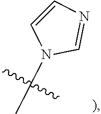 [SMILES: *n1ccnc1]

or pyridinyl (e.g.,

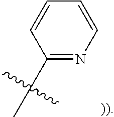 [SMILES: *c1ccccn1]

Embodiment 60. The method of Embodiment 59, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 61. The method of any one of Embodiments 57-60, wherein R^{3a} and R^{3b} are each independently at each occurrence hydrogen.

Embodiment 62. The method of any one of Embodiments 57-61, wherein the plurality (N) of branches comprises at least 3 (e.g., at least 4, or at least 5) branches.

Embodiment 63. The method of any one of Embodiments 57-62, wherein g=1; G=0; and Z=1.

Embodiment 64. The method of Embodiment 63, wherein each branch of the plurality of branches comprises a structural formula)

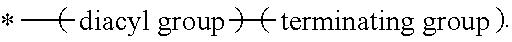

Embodiment 65. The method of any one of Embodiments 57-62, wherein g=2; G=1; and Z=2.

Embodiment 66. The method of Embodiment 65, wherein each branch of the plurality of branches comprises a structural formula

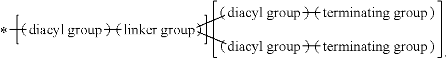

Embodiment 67. The method of any one of Embodiments 57-66, wherein the core comprises a structural formula:

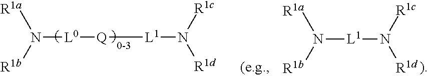

Embodiment 68. The method of any one of Embodiments 57-66, wherein the core comprises a structural formula:

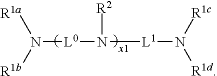

Embodiment 69. The method of Embodiment 68, wherein the core comprises a structural formula:

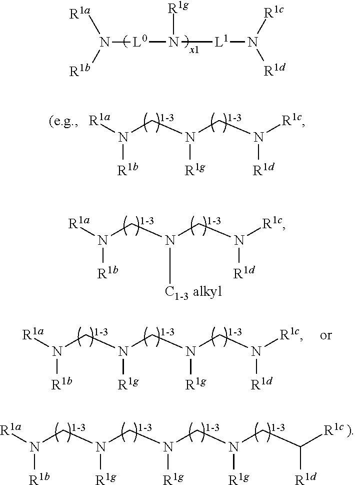

Embodiment 70. The method of Embodiment 68, wherein the core comprises a structural formula:

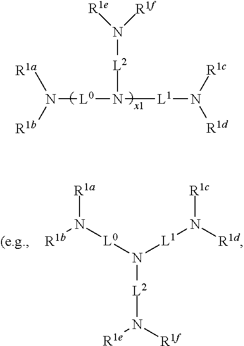

such as

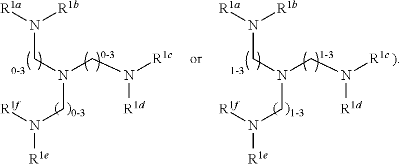

Embodiment 71. The method of any one of Embodiments 57-66, wherein the core comprises a structural formula:

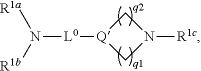

wherein Q′ is —NR²— or —CR^{3a}R^{3b}—; q¹ and q² are each independently 1 or 2.

Embodiment 72. The method of Embodiment 71, wherein the core comprises a structural formula:

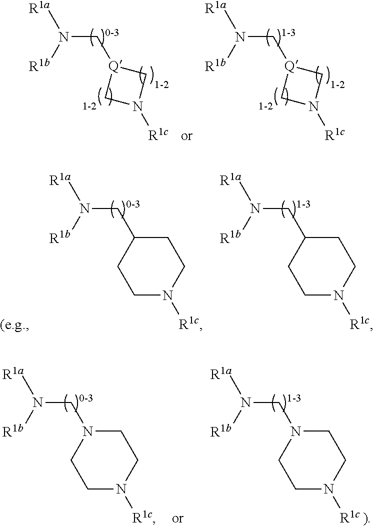

Embodiment 73. The method of any one of Embodiments 57-66, wherein the core comprises a structural formula

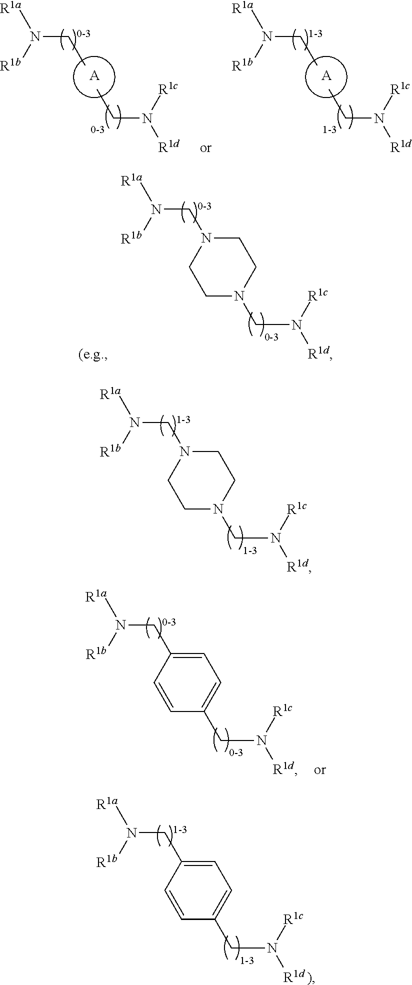

wherein ring A is an optionally substituted aryl or an optionally substituted (e.g., C₃-C₁₂, such as C₃-C₅) heteroaryl.

Embodiment 74. The method of any one of Embodiments 57-66, wherein the core comprises has a structural formula

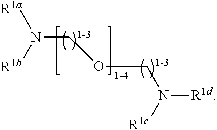

Embodiment 75. The method of any one of Embodiments 57-66, wherein the core is selected from those set forth in Table 1 or a subset thereof.

Embodiment 76. The method of any one of Embodiments 57-66, wherein the core comprises a structural formula selected from the group consisting of:

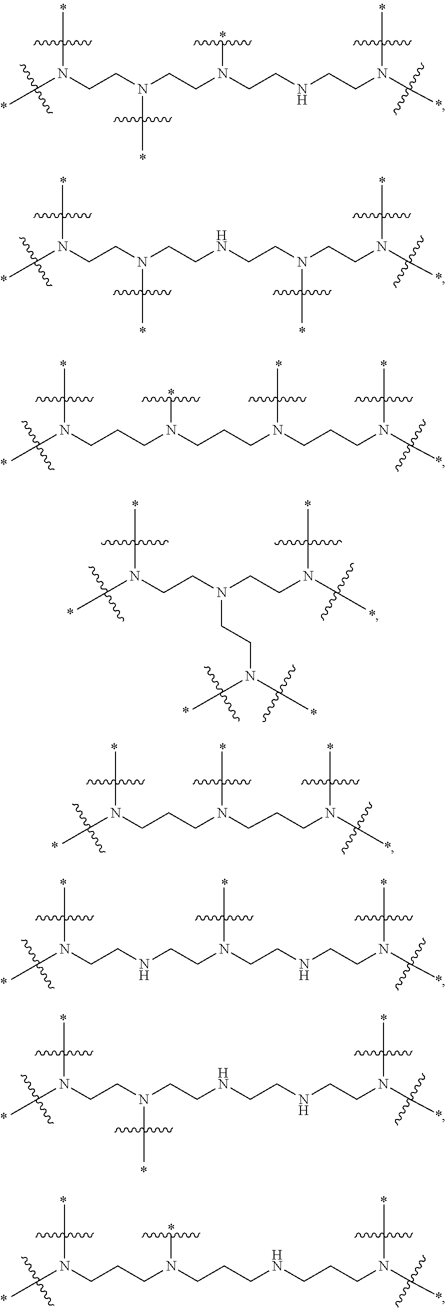

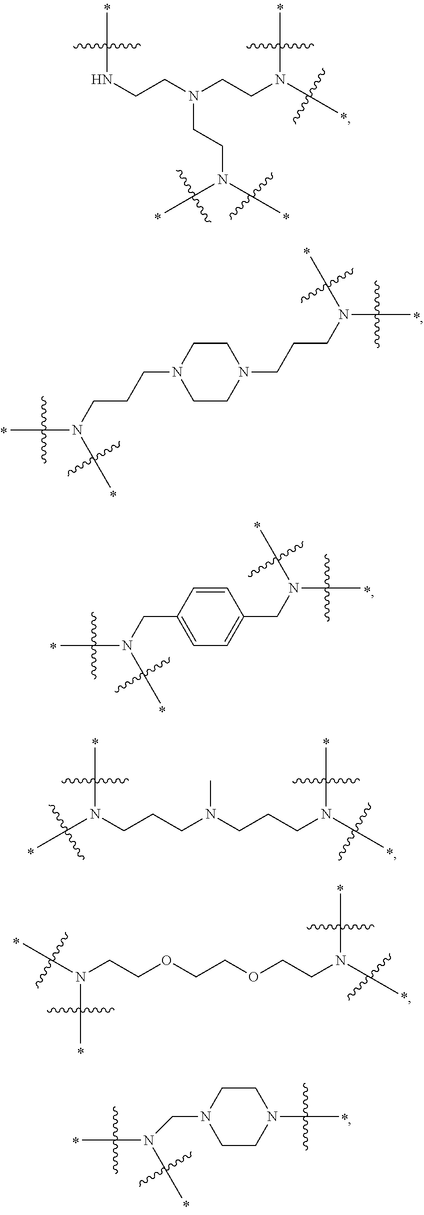 [SMILES: *NCCN(CCN(*)*)CCN(*)* ; *N(*)CCCN1CCN(CCCN(*)*)CC1 ; *N(*)Cc1ccc(CN(*)*)cc1 ; *N(*)CCCN(C)CCCN(*)* ; *N(*)CCOCCOCCN(*)* ; *N(*)CN1CCN(*)CC1]

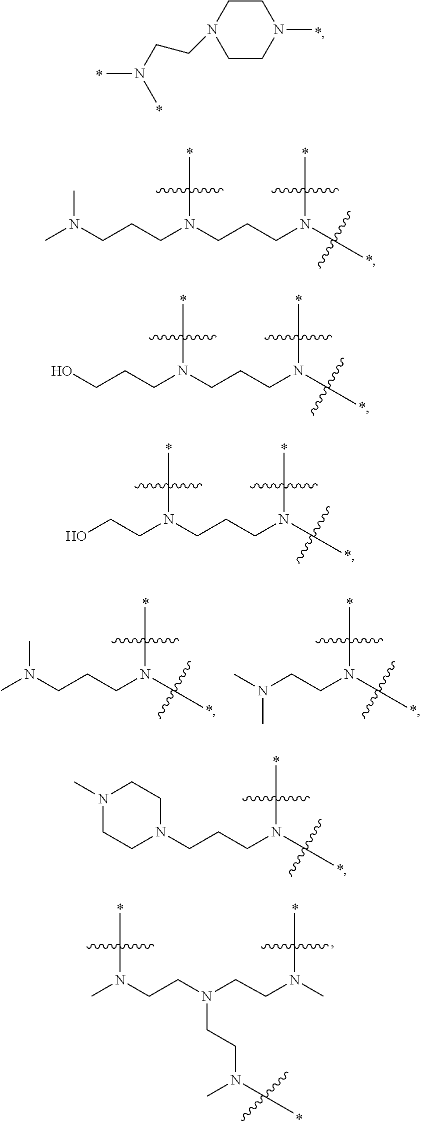

and pharmaceutically acceptable salts thereof, wherein * indicates a point of attachment of the core to a branch of the plurality of branches.

Embodiment 77. The method of any one of Embodiments 57-66, wherein the core comprises a structural formula selected from the group consisting of:

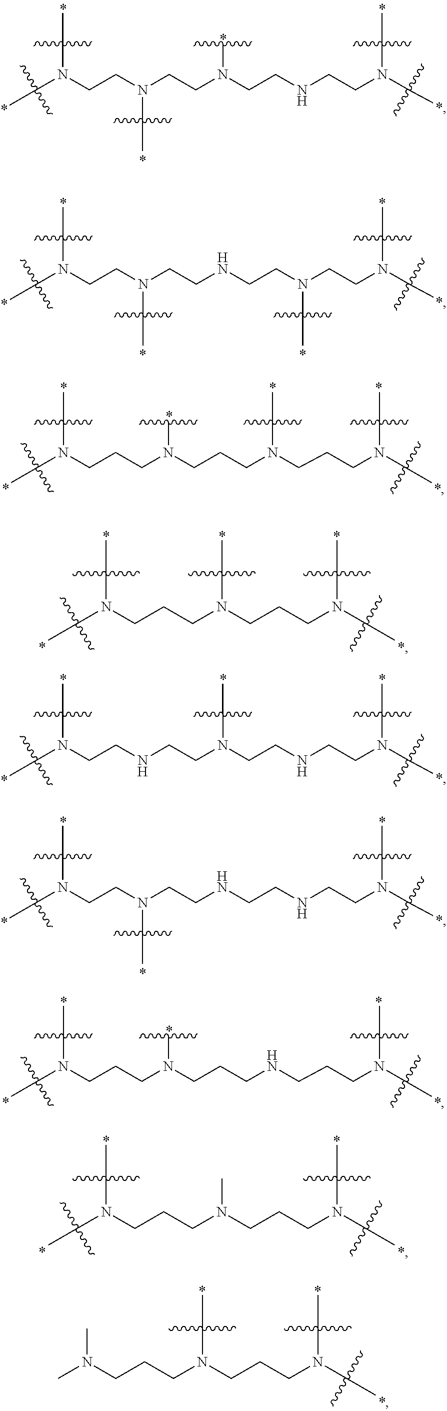

and pharmaceutically acceptable salts thereof, wherein * indicates a point of attachment of the core to a branch of the plurality of branches.

Embodiment 78. The method of any one of Embodiments 57-66, wherein the core has the structure

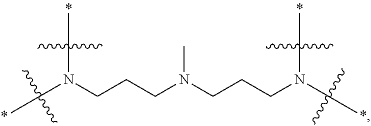 [SMILES: *N(*)CCCN(C)CCCN(*)*]

wherein * indicates a point of attachment of the core to a branch of the plurality of branches or H.

Embodiment 79. The method of Embodiment 78, wherein at least 2 branches are attached to the core.

Embodiment 80. The method of Embodiment 78, wherein at least 3 branches are attached to the core.

Embodiment 81. The method of Embodiment 78, wherein at least 4 branches are attached to the core.

Embodiment 82. The method of any one of Embodiments 57-66, wherein the core has the structure

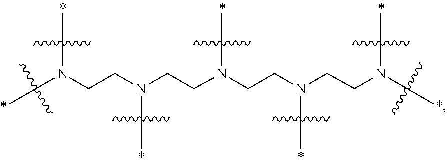 [SMILES: *N(*)CCN(*)CCN(*)CCN(*)CCN(*)*]

wherein * indicates a point of attachment of the core to a branch of the plurality of branches or H.

Embodiment 83. The method of Embodiment 82, wherein at least 4 branches are attached to the core.

Embodiment 84. The method of Embodiment 82, wherein at least 5 branches are attached to the core.

Embodiment 85. The method of Embodiment 82, wherein at least 6 branches are attached to the core.

Embodiment 86. The method of any one of Embodiments 57-85, wherein A¹ is —O— or —NH—.

Embodiment 87. The method of Embodiment 86, wherein A¹ is —O—.

Embodiment 88. The method of any one of Embodiments 57-87, wherein A² is —O— or —NH—.

Embodiment 89. The method of any Embodiment 88, wherein A² is —O—.

Embodiment 90. The method of any one of Embodiments 57-89, wherein Y³ is C₁-C₁₂ (e.g., C₁-C₆, such as C₁-C₃) alkylene.

Embodiment 91. The method of any one of Embodiments 57-90, wherein the diacyl group independently at each occurrence comprises a structural formula

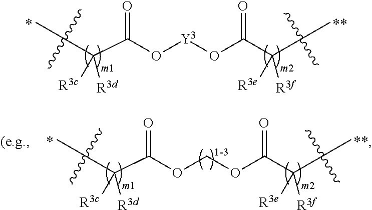

such as

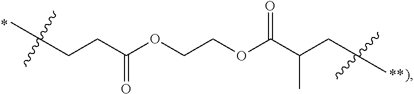 [SMILES: *CCC(=O)OCCOC(=O)C(C)C*]

optionally wherein R^{3c}, R^{3d}, R^{3e}and R^{3f} are each independently at each occurrence hydrogen or C₁-C₃ alkyl.

Embodiment 92. The method of any one of Embodiments 57-91, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] (e.g.,

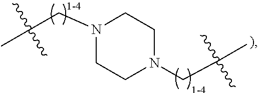

and [(C₁-C₄) alkylene]-phenylene-[(C₁-C₄) alkylene](e.g.,

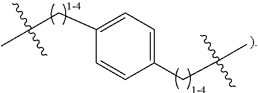

Embodiment 93. The method of Embodiment 92, wherein L⁰, L¹, and L² are each independently at each occurrence selected from C₁-C₆ alkylene (e.g., C₁-C₃ alkylene), —(C₁-C₃ alkylene-O)₁-₄-(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 94. The method of Embodiment 92, wherein L⁰, L¹, and L² are each independently at each occurrence C₁-C₆ alkylene (e.g., C₁-C₃ alkylene).

Embodiment 95. The method of Embodiment 92, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 96. The method of Embodiment 92, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 97. The method of any one of Embodiments 57-96, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

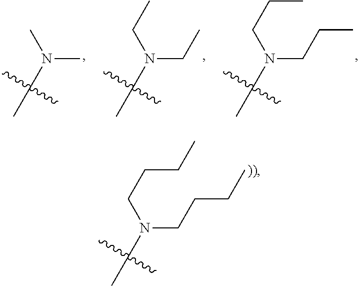

C₄-C₆ N-heterocycloalkyl (e.g., N-pyrrolidinyl

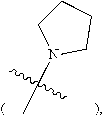 [SMILES: *N1CCCC1]

### N-piperidinyl

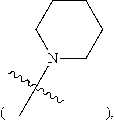 [SMILES: *N1CCCCC1]

### N-azepanyl

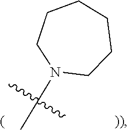 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

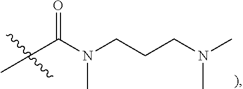 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

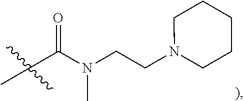 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl) (e.g.,

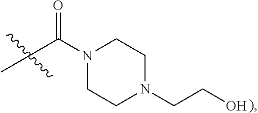 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 98. The method of Embodiment 97, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

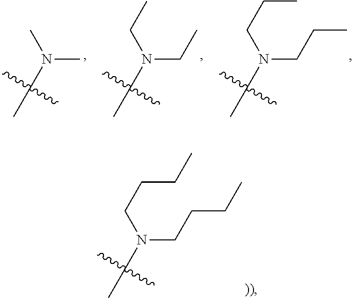

C₄-C₆ N-heterocycloalkyl (e.g., N-pyrrolidinyl

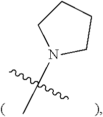 [SMILES: *N1CCCC1]

### N-piperidinyl

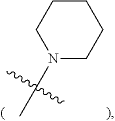 [SMILES: *N1CCCCC1]

### N-azepanyl

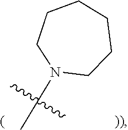 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

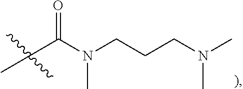 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

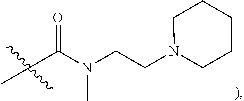 [SMILES: *C(=O)N(C)CCN1CCCCC1]

and —C(O)—(C₄-C₆ N-heterocycloalkyl) (e.g.,

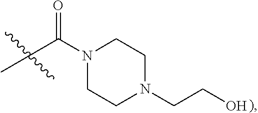 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 99. The method of Embodiment 98, wherein each terminating group is independently C₁-C₁₈ (e.g., C₄-C₁₈) alkylthiol, wherein the alkyl moiety is optionally substituted with one substituent —OH.

Embodiment 100. The method of Embodiment 98, wherein each terminating group is independently C₁-C₁₈ (e.g., C₄-C₁₅) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

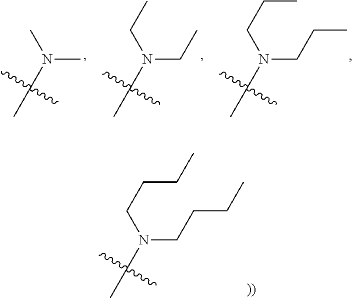

and C₄-C₆ N-heterocycloalkyl (e.g., N-pyrrolidinyl

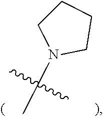 [SMILES: *N1CCCC1]

### N-piperidinyl

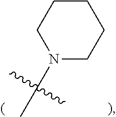 [SMILES: *N1CCCCC1]

### N-azepanyl

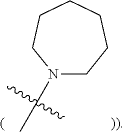 [SMILES: *N1CCCCCC1]

Embodiment 101. The method of Embodiment 97, wherein each terminating group is independently C₁-C₁₈ (e.g., C₄-C₁₈) alkenylthiol or C₁-C₁₈ (e.g., C₄-C₁₈) alkylthiol.

Embodiment 102. The method of Embodiment 101, wherein each terminating group is independently C₁-C₁₈ (e.g., C₄-C₁₈) alkylthiol.

Embodiment 103. The method of Embodiment 102, wherein each terminating group is independently selected from the group consisting of:

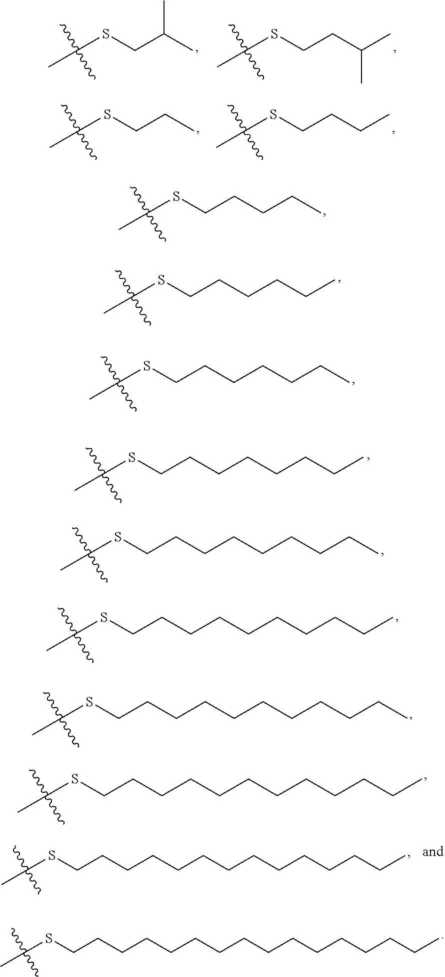

Embodiment 104. The method of any one of Embodiments 57-96, wherein each terminating group is independently selected from those set forth in Table 3 or a subset

thereof.

Embodiment 105. The method of any one of Embodiments 1-56, wherein the ionizable cationic lipid is selected from those set forth in Table 4, or pharmaceutically acceptable salts thereof, or a subset of the lipids and the pharmaceutically acceptable salts thereof.

Embodiment 106. The method of any one of Embodiments 1-56, wherein the ionizable cationic lipid is selected from those set forth in Table 4 or Table 5, or pharmaceutically acceptable salts thereof, or a subset of the lipids and the pharmaceutically acceptable salts thereof.

Embodiment 107. The method of any one of Embodiments 1-106, further comprising deriving a cell composition from said cell.

Embodiment 108. An engineered cell composition comprising or derived from a cell having an expression or activity of cystic fibrosis transmembrane conductance regulator (CFTR) protein enhanced by a method of any one of Embodiments 1-106.

Embodiment 109. A composition comprising a nucleic acid editing system assembled with a lipid composition, wherein said nucleic acid editing system comprises:
(i) a guide nucleic acid comprising a targeting sequence that is complementary with a target sequence of a cystic fibrosis transmembrane conductance regulator (CFTR) gene or transcript;
(ii) a polypeptide comprising an endonuclease or a polynucleotide encoding said polypeptide, which endonuclease is configured to (1) form a complex with said guide nucleic acid and (2) cleave said CFTR gene or transcript in a cell in a cleavage event; and
(iii) a donor template nucleic acid configured to alter said CFTR gene or transcript, subsequent to said cleavage event, to provide a functional CFTR gene, transcript or protein in said cell.

Embodiment 110. The composition of Embodiment 109, wherein said guide nucleic acid comprises a nucleotide sequence selected from those set forth in Table A and complementary sequences thereof.

Embodiment 111. The composition of Embodiment 109 or 110, wherein said donor template nucleic acid comprises a nucleotide sequence selected from those set forth in Table B and complementary sequences thereof.

Embodiment 112. The composition of any one of Embodiments 109-111, wherein said donor template nucleic acid comprises a 5′ homology arm.

Embodiment 113. The composition of any one of Embodiments 109-112, wherein said donor template nucleic acid comprises a 3′ homology arm.

Embodiment 114. The composition of any one of Embodiments 109-113, wherein (ii) is a messenger ribonucleic acid (mRNA) encoding said polypeptide comprising said endonuclease.

Embodiment 115. The composition of any one of Embodiments 109-113, wherein (ii) is said polypeptide comprising said endonuclease.

Embodiment 116. The composition of any one of Embodiments 109-115, wherein said endonuclease is a CRISPR-associated (Cas) polypeptide or a modification thereof.

Embodiment 117. The composition of Embodiment 116, wherein said endonuclease is Cas9.

Embodiment 118. The composition of any one of Embodiments 109-117, wherein (i) and (iii) are present on two different molecules.

Embodiment 119. The composition of any one of Embodiments 109-118, wherein (i), (ii), and (iii) are present on three different molecules.

Embodiment 120. The composition of any one of Embodiments 109-118, wherein at least two of (i), (ii) and (iii) are present on one molecule.

Embodiment 121. The composition of any one of Embodiments 109-120, wherein (i) and (ii) are present in said composition at a molar or weight ratio from 1:1 to 1:20.

Embodiment 122. The composition of any one of Embodiments 109-121, wherein (i) and (iii) are present in said composition at a molar or weight ratio from 1:1 to 1:30.

Embodiment 123. The composition of any one of Embodiments 109-122, wherein said lipid composition comprises: an ionizable cationic lipid; and a selective organ targeting (SORT) lipid separate from said ionizable cationic lipid.

Embodiment 124. The composition of Embodiment 123, wherein said therapeutic effect is characterized by an amount or activity of said agent detectable in said at least about 10% (e.g., at least about 15%) basal cells in said organ or tissue of said subject.

Embodiment 125. The composition of any one of Embodiments 109-124, wherein said lipid composition further comprises (iii) a phospholipid.

Embodiment 126. The composition of any one of Embodiments 109-125, wherein said lipid composition comprises said SORT lipid at a molar percentage from about 20% to about 65%.

Embodiment 127. The composition of any one of Embodiments 109-126, wherein said lipid composition comprises said ionizable cationic lipid at a molar percentage from about 5% to about 30%.

Embodiment 128. The composition of any one of Embodiments 109-127, wherein said lipid composition comprises said phospholipid at a molar percentage from about 8% to about 23%.

Embodiment 129. The composition of any one of Embodiments 109-128, wherein said phospholipid is not an ethylphosphocholine.

Embodiment 130. The composition of any one of Embodiments 109-129, wherein said lipid composition further comprises a steroid or steroid derivative.

Embodiment 131. The composition of Embodiment 130, wherein said lipid composition comprises said steroid or steroid derivative at a molar percentage from about 15% to about 46%.

Embodiment 132. The composition of any one of Embodiments 109-131, wherein said lipid composition further comprises a polymer-conjugated lipid (e.g., poly(ethylene glycol) (PEG)-conjugated lipid).

Embodiment 133. The composition of Embodiment 132, wherein said lipid composition comprises said polymer-conjugated lipid at a molar percentage from about 0.5% to about 10%.

Embodiment 134. The composition of Embodiment 132, wherein said lipid composition comprises said polymer-conjugated lipid at a molar percentage from about 1% to about 10%.

Embodiment 135. The composition of Embodiment 132, wherein said lipid composition comprises said polymer-conjugated lipid at a molar percentage from about 2% to about 10%.

Embodiment 136. The composition of any one of Embodiments 109-135, wherein said therapeutic agent is a polynucleotide; and wherein a molar ratio of nitrogen in said lipid composition to phosphate in said polynucleotide (N/P ratio) is no more than about 20:1.

Embodiment 137. The composition of Embodiment 136, wherein said N/P ratio is from about 5:1 to about 20:1.

Embodiment 138. The composition of any one of Embodiments 109-137, wherein a molar ratio of said therapeutic agent to total lipids of said lipid composition is no more than about 1:1, 1:10, 1:50, or 1:100.

Embodiment 139. The composition of any one of Embodiments 109-138, wherein at least about 85% of said therapeutic agent is encapsulated in particles of said lipid compositions.

Embodiment 140. The composition of any one of Embodiments 109-139, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

Embodiment 141. The composition of any one of Embodiments 109-140, wherein said lipid composition has an apparent ionization constant (pKa) outside a range of 6 to 7.

Embodiment 142. The composition of Embodiment 141, wherein said apparent pKa of said lipid composition is of about 7 or higher.

Embodiment 143. The composition of Embodiment 141, wherein said apparent pKa of said lipid composition is of about 8 or higher.

Embodiment 144. The composition of Embodiment 143, wherein said apparent pKa of said lipid composition is from about 8 to about 13.

Embodiment 145. The composition of any one of Embodiments 109-144, wherein said SORT lipid comprises a permanently positively charged moiety (e.g., a quaternary ammonium ion).

Embodiment 146. The composition of Embodiment 145, wherein said SORT lipid comprises a counterion.

Embodiment 147. The composition of any one of Embodiments 109-146, wherein said SORT lipid is a phosphocholine lipid (e.g., saturated or unsaturated).

Embodiment 148. The composition of any one of Embodiments 147, wherein said SORT lipid is an ethylphosphocholine.

Embodiment 149. The composition of any one of Embodiments 109-148, wherein said SORT lipid comprises a headgroup having a structural formula:

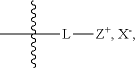

wherein L is a (e.g., biodegradable) linker; Z⁺ is positively charged moiety (e.g., a quaternary ammonium ion); and X⁻ is a counterion.

Embodiment 150. The composition of Embodiment 149, wherein said SORT lipid has a structural formula:

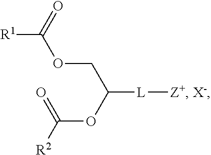

wherein R¹ and R² are each independently an optionally substituted C₆-C₂₄ alkyl, or an optionally substituted C₆-C₂₄ alkenyl.

Embodiment 151. The composition of Embodiment 149, wherein said SORT lipid has a structural formula:

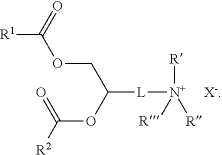

Embodiment 152. The composition of any one of Embodiments 149-151, wherein L is

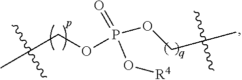

wherein: p and q are each independently 1, 2, or 3; and R⁴ is an optionally substituted C₁-C₆ alkyl.

Embodiment 153. The composition of Embodiment 149, wherein said SORT lipid has a structural formula:

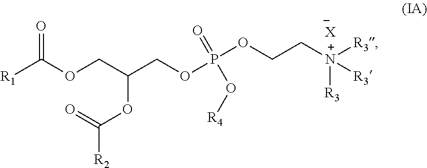

wherein:
R₁ and R₂ are each independently alkyl_{(C8-C24)}, alkenyl_{(C8-C24)}, or a substituted version of either group;
R₃, R₃′, and R₃″ are each independently alkyl_{(C6)} or substituted alkyl(_{C≤6});
R₄ is alkyl_{(C≤6)} or substituted alkyl_{(C≤6)}; and
X⁻ is a monovalent anion.

Embodiment 154. The composition of any one of Embodiments 109-146, wherein said SORT lipid has a structural formula:

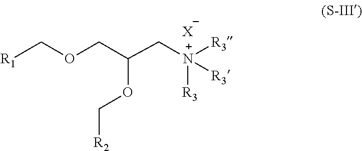

wherein:
   R₁ and R₂ are each independently alkyl_{(C8-C24)}, alkenyl_{(C8-C24)}, or a substituted version of either group;
   R₃, R₃′, and R₃″ are each independently alkyl_{(C≤6)} or substituted alkyl(C≤6);
   X⁻ is a monovalent anion.

Embodiment 155. The composition of any one of Embodiments 109-146, wherein said SORT lipid has a structural formula:

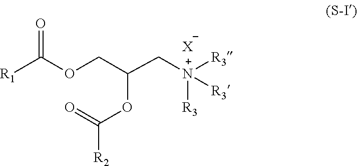

wherein:
   R₁ and R₂ are each independently alkyl_{(C8-C24)}, alkenyl_{(C8-C24)}, or a substituted version of either group;
   R₃, R₃′, and R₃″ are each independently alkyl_{(C6)} or substituted alkyl(_{C6});
   X⁻ is a monovalent anion.

Embodiment 156. The composition of any one of Embodiments 109-146, wherein said SORT lipid has a structural formula:

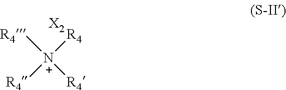

wherein:
   R₄ and R₄′ are each independently alkyl_{(C6-C24)}, alkenyl_{(C6-C24)}, or a substituted version of either group;
   R₄″ is alkyl_{(C≤24)}, alkenyl_{(C≤24)}, or a substituted version of either group;
   R₄′″ is alkyl_{(C1-C8)}, alkenyl_{(C2-C8)}, or a substituted version of either group; and
   X₂ is a monovalent anion.

Embodiment 157. The composition of any one of Embodiments 109-144, wherein said SORT lipid is selected from those set forth in Table 6, or pharmaceutically acceptable salts thereof, or a subset of the lipids and the pharmaceutically acceptable salts thereof.

Embodiment 158. The composition of any one of Embodiments 109-157, wherein the ionizable cationic lipid is a dendrimer or dendron of a generation (g) having a structural formula:

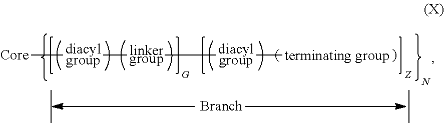

or a pharmaceutically acceptable salt thereof, wherein:
(a) the core comprises a structural formula (X_{Core}):

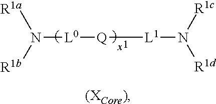

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

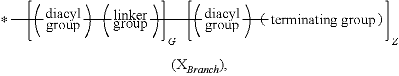

wherein:
   * indicates a point of attachment of the branch to the core;
   g is 1, 2, 3, or 4;

$$\\ Z = {2}^{{}_{} ( g - 1 )} ;$$

G=0, when g=1; or G=Σᵢ₌₀^{i=g-2}2ⁱ, when g≠1;
(c) each diacyl group independently comprises a structural formula

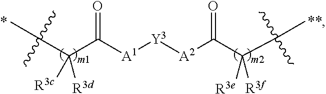

wherein:
   * indicates a point of attachment of the diacyl group at the proximal end thereof;
   ** indicates a point of attachment of the diacyl group at the distal end thereof;
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₆) alkyl; and
(d) each linker group independently comprises a structural formula

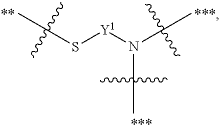

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 159. The composition of Embodiment 158, wherein x¹ is 0, 1, 2, or 3.

Embodiment 160. The composition of Embodiment 158 or 159, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl (e.g.,

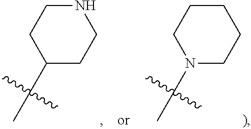 [SMILES: *C1CCNCC1 ; *N1CCCCC1]

N—(C₁-C₃ alkyl)-piperidinyl (e.g.,

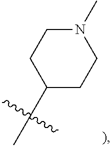 [SMILES: *C1CCN(C)CC1]

piperazinyl (e.g.,

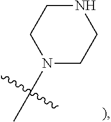 [SMILES: *N1CCNCC1]

N—(C₁-C₃ alkyl)-piperadizinyl (e.g.,

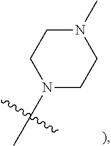 [SMILES: *N1CCN(C)CC1]

morpholinyl (e.g.,

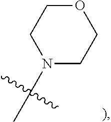 [SMILES: *N1CCOCC1]

### N-pyrrolidinyl (e.g.,

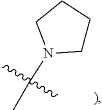 [SMILES: *N1CCCC1]

pyrrolidinyl (e.g.,

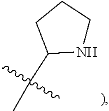 [SMILES: *C1CCCN1]

or N—(C₁-C₃ alkyl)-pyrrolidinyl (e.g.,

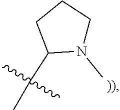 [SMILES: *C1CCCN1C]

(e.g., C₆-C₁₀) aryl, and C₃-C₅ heteroaryl (e.g., imidazolyl (e.g.,

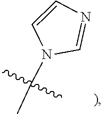 [SMILES: *n1ccnc1]

or pyridinyl (e.g.,

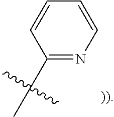 [SMILES: *c1ccccn1]

Embodiment 161. The composition of Embodiment 160, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 162. The composition of any one of Embodiments 158-161, wherein R^{3a} and R^{3b} are each independently at each occurrence hydrogen.

Embodiment 163. The composition of any one of Embodiments 158-162, wherein the plurality (N) of branches comprises at least 3 (e.g., at least 4, or at least 5) branches.

Embodiment 164. The composition of any one of Embodiments 158-163, wherein g=1; G=0; and Z=1.

Embodiment 165. The composition of Embodiment 164, wherein each branch of the plurality of branches comprises a structural formula

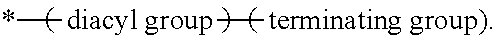

Embodiment 166. The composition of any one of Embodiments 158-163, wherein g=2; G=1; and Z=2.

Embodiment 167. The composition of Embodiment 166, wherein each branch of the plurality of branches comprises a structural formula

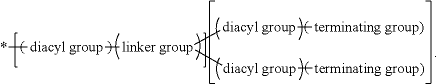

Embodiment 168. The composition of any one of Embodiments 158-167, wherein the core comprises a structural formula:

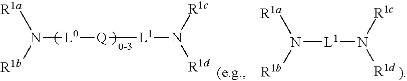

Embodiment 169. The composition of any one of Embodiments 158-167, wherein the core comprises a structural formula:

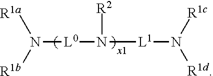

Embodiment 170. The composition of Embodiment 169, wherein the core comprises a structural formula:

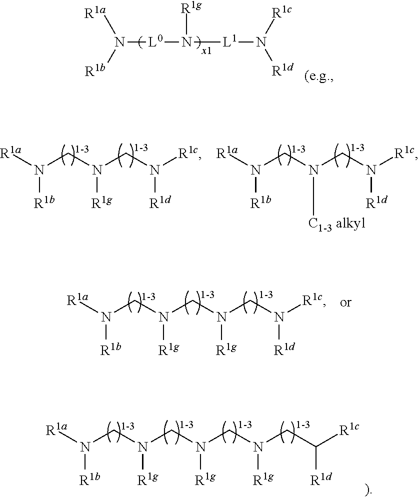

Embodiment 171. The composition of Embodiment 169, wherein the core comprises a structural formula:

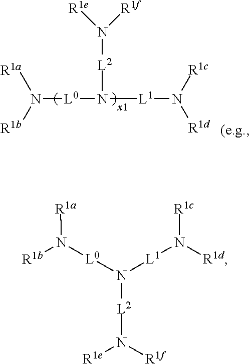

such as

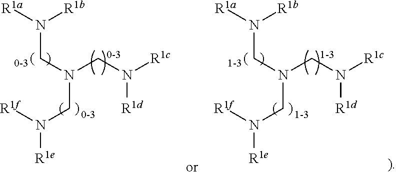

Embodiment 172. The composition of any one of Embodiments 158-167, wherein the core comprises a structural formula:

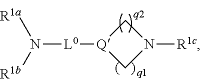

wherein Q′ is —NR²— or —CR^{3a}R^{3b}—; q¹ and q² are each independently 1 or 2.

Embodiment 173. The composition of Embodiment 172, wherein the core comprises a structural formula:

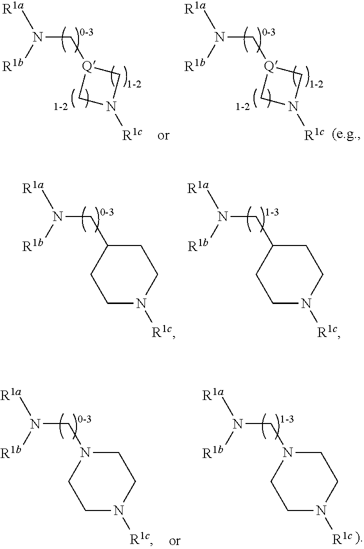

Embodiment 174. The composition of any one of Embodiments 158-167, wherein the core comprises a structural formula

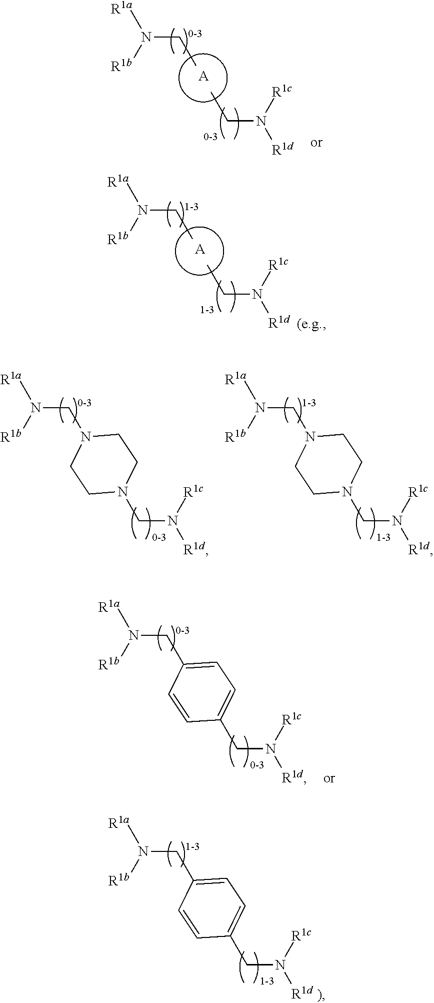

wherein ring A is an optionally substituted aryl or an optionally substituted (e.g., C₃-C₁₂, such as C₃-C₅) heteroaryl.

Embodiment 175. The composition of any one of Embodiments 158-167, wherein the core comprises has a structural formula

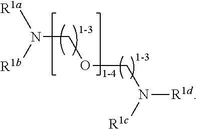

Embodiment 176. The composition of any one of Embodiments 158-167, wherein the core is selected from those set forth in Table 1 or a subset thereof.

Embodiment 177. The composition of any one of Embodiments 158-167, wherein the core comprises a structural formula selected from the group consisting of:

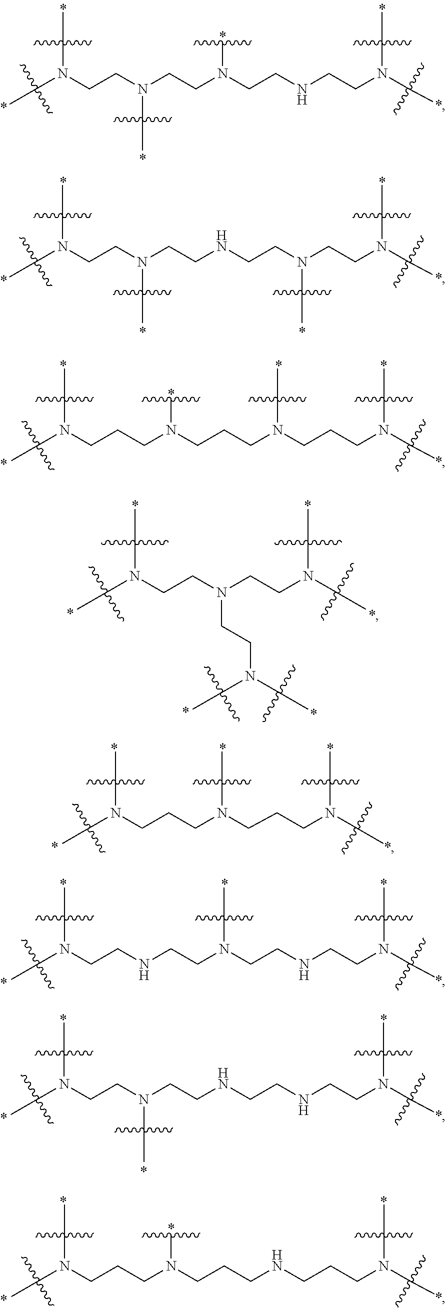

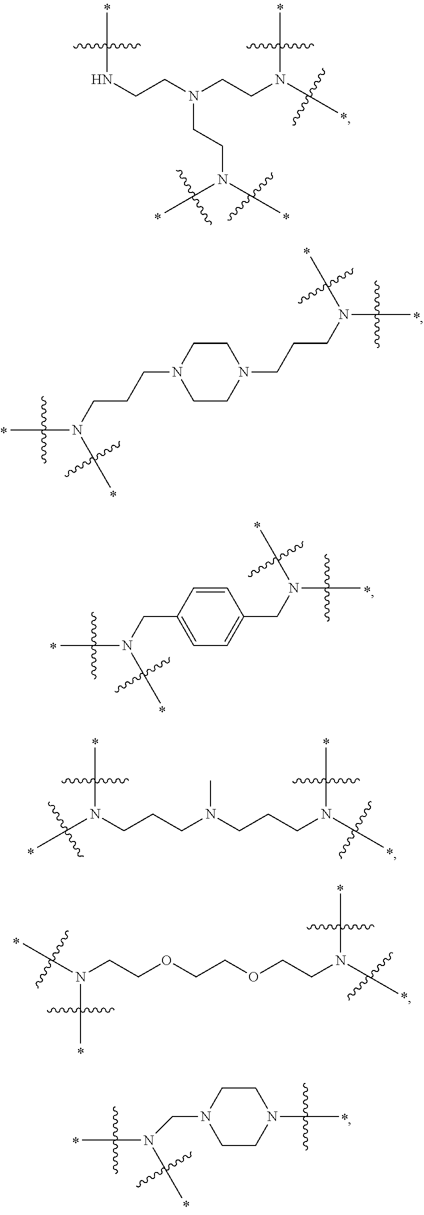 [SMILES: *NCCN(CCN(*)*)CCN(*)* ; *N(*)CCCN1CCN(CCCN(*)*)CC1 ; *N(*)Cc1ccc(CN(*)*)cc1 ; *N(*)CCCN(C)CCCN(*)* ; *N(*)CCOCCOCCN(*)* ; *N(*)CN1CCN(*)CC1]

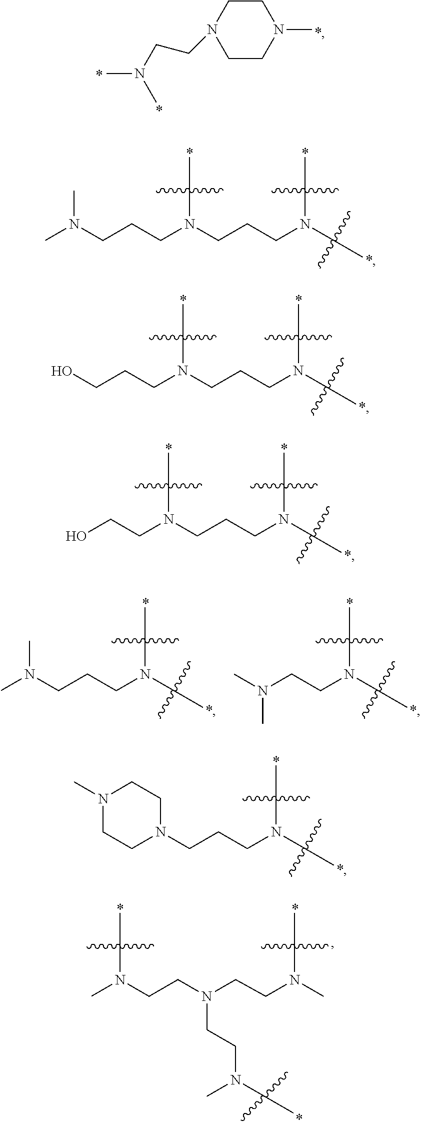

and pharmaceutically acceptable salts thereof, wherein * indicates a point of attachment of the core to a branch of the plurality of branches.

Embodiment 178. The composition of any one of Embodiments 158-167, wherein the core comprises a structural formula selected from the group consisting of:

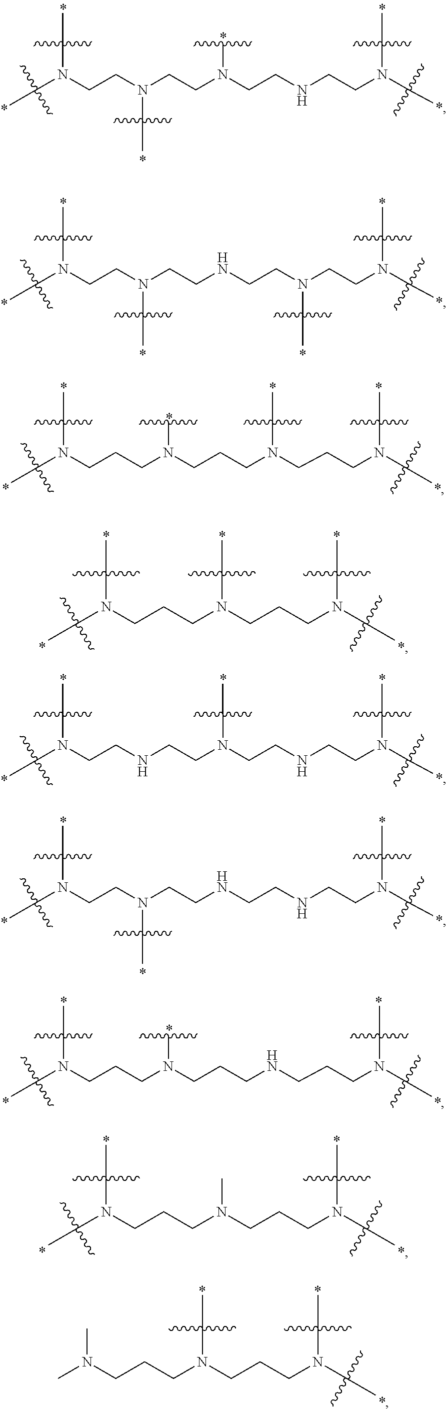

and pharmaceutically acceptable salts thereof, wherein * indicates a point of attachment of the core to a branch of the plurality of branches.

Embodiment 179. The composition of any one of Embodiments 158-167, wherein the core has the structure

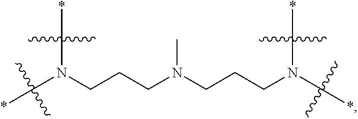

wherein * indicates a point of attachment of the core to a branch of the plurality of branches or H.

Embodiment 180. The composition of Embodiment 179, wherein at least 2 branches are attached to the core.

Embodiment 181. The composition of Embodiment 179, wherein at least 3 branches are attached to the core.

Embodiment 182. The composition of Embodiment 179, wherein at least 4 branches are attached to the core.

Embodiment 183. The composition of any one of Embodiments 158-167, wherein the core has the structure

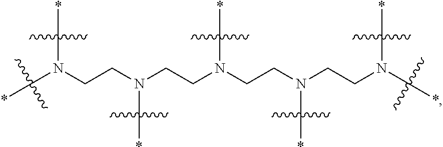

wherein * indicates a point of attachment of the core to a branch of the plurality of branches or H.

Embodiment 184. The composition of Embodiment 183, wherein at least 4 branches are attached to the core.

Embodiment 185. The composition of Embodiment 183, wherein at least 5 branches are attached to the core.

Embodiment 186. The composition of Embodiment 183, wherein at least 6 branches are attached to the core.

Embodiment 187. The composition of any one of Embodiments 158-186, wherein A¹ is —O— or —NH—.

Embodiment 188. The composition of Embodiment 187, wherein A¹ is —O—.

Embodiment 189. The composition of any one of Embodiments 158-188, wherein A² is —O— or —NH—.

Embodiment 190. The composition of any Embodiment 189, wherein A² is —O—.

Embodiment 191. The composition of any one of Embodiments 158-190, wherein Y³ is C₁-C₁₂ (e.g., C₁-C₆, such as C₁-C₃) alkylene.

Embodiment 192. The composition of any one of Embodiments 158-191, wherein the diacyl group independently at each occurrence comprises a structural formula

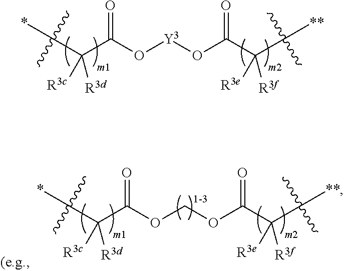

such as

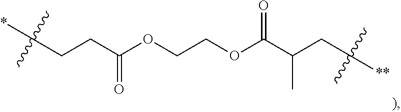

optionally wherein R^{3c}, R^{3d}, R^{3e}, and R^{3f} are each independently at each occurrence hydrogen or C₁-C₃ alkyl.

Embodiment 193. The composition of any one of Embodiments 158-192, wherein L⁰, L¹, and L² are each independently at each occurrence selected from a covalent bond, C₁-C₆alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] (e.g.,

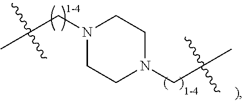

and [(C₁-C₄) alkylene]-phenylene-[(C₁-C₄) alkylene] (e.g.,

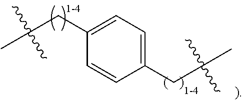

Embodiment 194. The composition of Embodiment 193, wherein L⁰, Lt, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 195. The composition of Embodiment 193, wherein L⁰, Lt, and L² are each independently at each occurrence C₁-C₆ alkylene (e.g., C₁-C₃ alkylene).

Embodiment 196. The composition of Embodiment 193, wherein L⁰, Lt, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 197. The composition of Embodiment 193, wherein L⁰, Lt, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 198. The composition of any one of Embodiments 158-197, wherein each terminating group is independently C₁-C₁₈ (e.g., C₄-C₁₈) alkenylthiol or C₁-C₁₈ (e.g., C₄-Cis) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

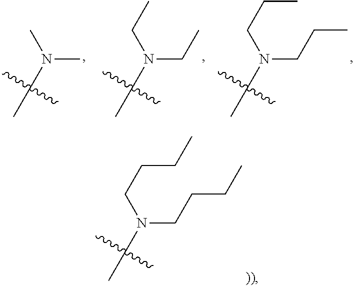

C₄-C₆ N-heterocycloalkyl (e.g., N-pyrrolidinyl

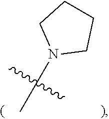 [SMILES: *N1CCCC1]

### N-piperidinyl

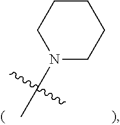 [SMILES: *N1CCCCC1]

### N-azepanyl

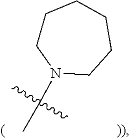 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

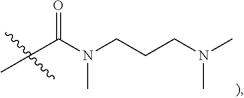 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

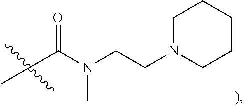 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl) (e.g.,

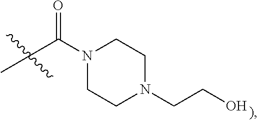 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 199. The composition of Embodiment 198, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₅) alkylamino (e.g., C₁-C₆ mono-alkylamino (such as —NHCH₂CH₂CH₂CH₃) or C₁-C₈ di-alkylamino (such as

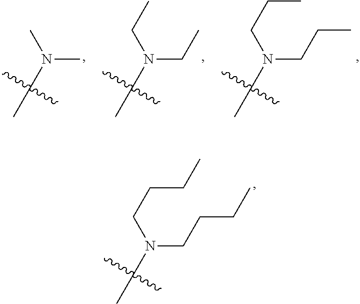

C₄-C₆ N-heterocycloalkyl (e.g., N-pyrrolidinyl

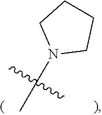 [SMILES: *N1CCCC1]

### N-piperidinyl

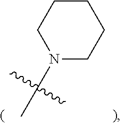 [SMILES: *N1CCCCC1]

### N-azepanyl

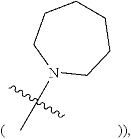 [SMILES: *N1CCCCCC1]

—C(O)OH, —C(O)N(C₁-C₃ alkyl)-(C₁-C₆ alkylene)-(C₁-C₁₂ alkylamino (e.g., mono- or di-alkylamino)) (e.g.,

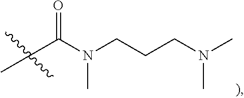 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

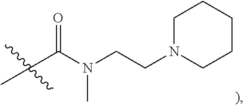 [SMILES: *C(=O)N(C)CCN1CCCCC1]

and —C(O)—(C₄-C₆ N-heterocycloalkyl) (e.g.,

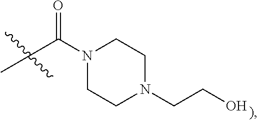 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 200. The composition of Embodiment 199, wherein each terminating group is independently C₁-C₁₈ (e.g., C₄-C₁₈) alkylthiol, wherein the alkyl moiety is optionally substituted with one substituent —OH.

Embodiment 201. The composition of Embodiment 199, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

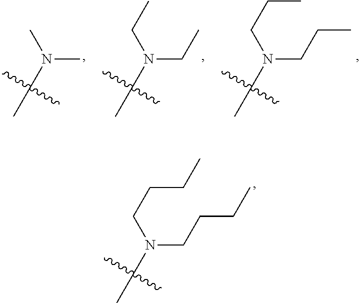

and C₄-C₆ N-heterocycloalkyl (e.g., N-pyrrolidinyl

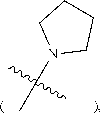 [SMILES: *N1CCCC1]

### N-piperidinyl

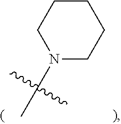 [SMILES: *N1CCCCC1]

### N-azepanyl

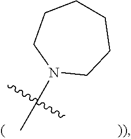 [SMILES: *N1CCCCCC1]

Embodiment 202. The composition of Embodiment 198, wherein each terminating group is independently C₁-C₁₈ (e.g., C₄-C₁₈) alkenylthiol or C₁-C₁₈ (e.g., C₄-C₁₈) alkylthiol.

Embodiment 203. The composition of Embodiment 202, wherein each terminating group is independently C₁-C₁₈ (e.g., C₄-C₁₈) alkylthiol.

Embodiment 204. The composition of Embodiment 203, wherein each terminating group is independently selected from the group consisting of:

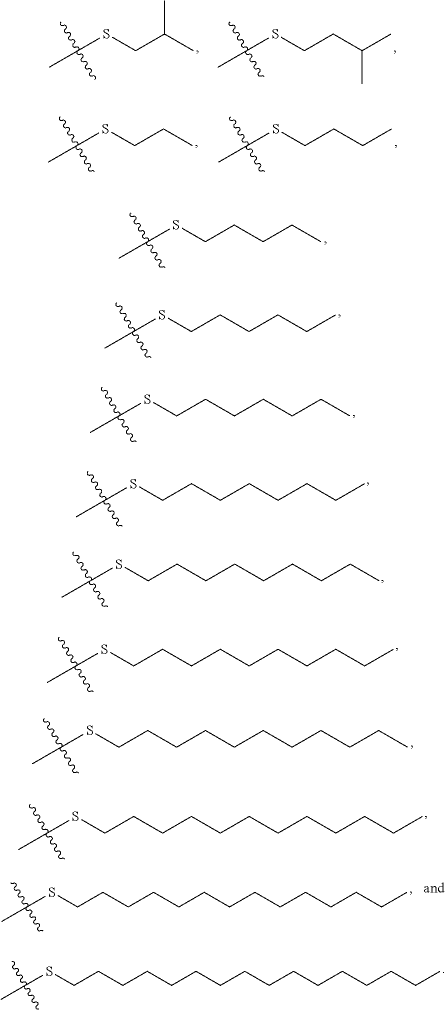

Embodiment 205. The composition of any one of Embodiments 158-197, wherein each terminating group is independently selected from those set forth in Table 3 or a subset thereof.

Embodiment 206. The composition of any one of Embodiments 109-157, wherein the ionizable cationic lipid is selected from those set forth in Table 4, or pharmaceutically acceptable salts thereof, or a subset of the lipids and the pharmaceutically acceptable salts thereof.

Embodiment 207. The composition of any one of Embodiments 109-157, wherein the ionizable cationic lipid is selected from those set forth in Table 4 or Table 5, or pharmaceutically acceptable salts thereof, or a subset of the lipids and the pharmaceutically acceptable salts thereof.

Embodiment 208. The composition of any one of Embodiments 109-207, wherein said composition is formulated for pharmaceutical (e.g., systemic) administration.

Embodiment 209. An engineered cell composition comprising or derived from a cell, which cell comprises a heterologous cystic fibrosis transmembrane conductance regulator (CFTR) gene, transcript or protein produced by a composition of any one of Embodiments 109-208.

Embodiment 210. A method for genetic correction of cystic fibrosis transmembrane conductance regulator (CFTR) in a lung basal cell, comprising: contacting said lung basal cell with a composition that comprises a nucleic acid editing system assembled with a lipid composition, thereby delivering said nucleic acid editing system to said lung basal cell.

Embodiment 211. A method for genetic correction of cystic fibrosis transmembrane conductance regulator (CFTR) in a cell composition, comprising: contacting said cell composition comprising a plurality of lung basal cells with a composition that comprises a nucleic acid editing system assembled with a lipid composition, thereby delivering said nucleic acid editing system to at least about 10%, 15%, 20%, 25%, 30%, 35%, 40%, 45%, 50%, 60%, 65%, or 70% of said plurality of lung basal cells.

Embodiment 212. A method for genetic correction of cystic fibrosis transmembrane conductance regulator (CFTR) in a cell composition, comprising: contacting said cell composition with a composition that comprises a nucleic acid editing system assembled with a lipid composition, which cell composition comprise a lung basal cell and a lung non-basal cell, thereby delivering said nucleic acid editing system to said lung basal cell in a greater amount than that delivered to said lung non-basal cell.

Embodiment 213. The method of Embodiment 212, wherein said non-basal cell is an ionocyte, a ciliated cell, or a secretory cell.

Embodiment 214. The method of any one of Embodiments 210-213, wherein said lung basal cell or said plurality of lung basal cells is/are determined to exhibit a mutation in CFTR gene.

Embodiment 215. The method of any one of Embodiments 210-213, wherein said lung basal cell or said plurality of lung basal cells exhibit(s) a mutation in CFTR gene.

Embodiment 216. The method of any one of Embodiments 210-215, wherein said lung basal cell or said plurality of lung basal cells is/are from a subject

Embodiment 217. The method of Embodiment 216, wherein said subject is determined to exhibit a mutation in CFTR gene.

Embodiment 218. The method of Embodiment 216, wherein said subject exhibits a mutation in CFTR gene.

Embodiment 219. The method of any one of Embodiments 210-218, wherein said contacting is ex vivo.

Embodiment 220. The method of any one of Embodiments 210-218, wherein said contacting is in vitro.

Embodiment 221. The method of any one of Embodiments 210-218, wherein said contacting is in vivo.

Embodiment 222. A method for treating a subject having or suspected of having a cystic fibrosis transmembrane conductance regulator (CFTR)-associated condition, the method comprising administering to said subject a composition comprising a nucleic acid editing system assembled with a lipid composition.

Embodiment 223. The method of Embodiment 222, wherein said CFTR-associated condition is cystic fibrosis, hereditary emphysema, chronic obstructive pulmonary disease (COPD), or a combination thereof.

Embodiment 224. The method of Embodiment 222 or 223, wherein said subject is a mammal.

Embodiment 225. The method of Embodiment 224, wherein said subject is a human.

Embodiment 226. The method of any one of Embodiments 222-225, wherein said subject is determined to exhibit a mutation (e.g., F508del or G542X) in CFTR gene.

Embodiment 227. The method of any one of Embodiments 222-226, wherein said administering comprises systemic administration.

Embodiment 228. The method of any one of Embodiments 208-227, wherein said lipid composition is according to any one of embodiments 24-106 or 123-207.

### EXAMPLES

### Example 1: Editing of CFTR

Human bronchial epithelial cells are thawed and plated. 5×10⁵ undifferentiated HBE cells were seeded into each well of 6-well plates overnight before treatment. At day1, 100 uL of LNP sample solution was added into each well containing 2 mL of fresh medium (1 ug total nucleic acid per well). 3 wells of the plate are treated with liponanoparticles comprising Cas9 mRNA, gRNA specific for CFTR, and a donor nucleic acid specific for F508del mutant allele, and 1 well is subject to a control formulation. After 4-5 days cells are passaged in to a T-75 flask and allowed to grow for 7-10 days. A sample of 500,000 cells is removed and lyse. The lyse cells are subjected to sequencing and the CFTR gene is analyzed to determine correction. Corrected cells are passage again and plated on HTS transwell filter plates to and allowed to differentiate. The cells are then subjected to analysis to determine the presence of protein expression and properly functioning cells.

### Quantification Methods of Mature CFTR

Cells are lysed directly in 2× Sample Buffer ((Tris-HCL 250 mM, pH 6.8, 20% Glycerol, 2.5% SDS, 0.1% Bromophenol blue). Cell lysate proteins were separated by electrophoresis on 7%/10% step (wt/vol) polyacrylamide gels using a Tris-glycine buffering system and transferred to polyvinylidene fluoride Immobilon membranes (ENID Millipore).

Western blot analysis was performed using primary CFTR antibody (596) (University of North Carolina School of Medicine, Chapel Hill, NC) actin antibody (EMD Millipore), and secondary antibody IRdye-680RD (Li-Cor) and imaged/quantified using a Li-Cor Odyssey CLx (Li-Cor). Data was plotted using Prism 6 (Graphpad).

CFTR-Dependent Whole-Cell Current detection

Whole-cell configuration of the patch-clamp technique was used to measure the Cl— current. The pipette solution contained 145 mM NMDG⁺-Cl⁻, 1 mM MgCk, 2 mM EGTA, 5 mM ATP, and 10 mM HEPES (pH 7.3 with Tris), The bath solution was 145 mM NMDG⁺-Cl⁻, 1 mM MgCl₂, 1 mM CaCl₂, 10 mM HEPES and 10 mM glucose (pH 7.4 with Tris). The current was recorded with an Axopatch 200B patch-clamp amplifier and digitized at 2 kHz.

The membrane conductance was probed by stepping the membrane potential from a holding potential of 0 mV to membrane potentials −40 and +40 mV steps for 200 ms. Whole-cell current responses were measured in response to 10 μM forskolin plus 100 μM IBMX and 10 μM CFTRInh-172 (Inh-172). Pipettes had resistances between 3 and 5 M-? when filled with pipette solution and seal resistance exceeded 8 GU. Current recording and analysis was performed with pClamp 9.2 software and analyzed with Origin 8 software.

### Example 2: Detection of CFTR Editing

Undifferentiated Human bronchial epithelial cells (unHBE) were thawed and plated. 465,000 undifferentiated HBE cells were seeded into each well of 6-well plates overnight before treatment. After 3 days the cells were lysed. The lyse cells were subjected to sequencing and the CFTR gene are analyzed to determine correction. FIG. 1A shows a chart of indel % for different LNP compositions. The chart indicates a successful introduction of indels into the CFTR gene. For example, “sgF508Del-1” (SEQ ID NO. 1) and “SgF508-1-NTS-60” (SEQ ID NO: 6) were used for H1BE treatment.

In another plate, 1 ug of total RNA (including Cas9 mRNA, gRNA specific for CFTR, and a donor nucleic acid specific for F508del mutant allele) was added to each well. FIG. 1B shows sets of a chart of the editing of hBEs using different LNP compositions, with corresponding numbers shown in FIG. 1B.

In another set of example experiments, 5×10⁵ undifferentiated HBE cells were seeded into each well of 6-well plates overnight before treatment. At day1, 100 μL of LNP sample solution was added into each well containing 2 mL of fresh medium (1 ug total nucleic acid per well), changed to fresh medium at day 3 and collected cells for PCR and sequencing at day 4. FIG. 1D shows an additional chart of the editing of hBEs using different LNP compositions. The % of indels and the knockout and knock-in scores were analyzed for different donor nucleic acids. The compositions are able to knock-in a correction to the cells, as well as knock-out a sequence of the gene when delivered without a donor nucleic acid. FIG. 1D illustrates Indel % and Knock-in % after treated HBE cells with DOTAP 10 LNPs containing different I-DR templates. HDR groups involved co-delivery of Cas9 mRNA, sgF508del, and the respective HDR template in DOTAP 10 LNPs. DOTAP 10 LNPs only (“LNP ONLY”) and DOTAP 10 LNPs encapsulating Cas9 mRNA and sgF508del (“INDEL ONLY”) were used as controls. These data were analyzed with TIDER analysis based on Sanger DNA sequencing data. Indels (%) refers to insertions and deletions by non-homologous end joining (NHEJ) (leading to gene knockout), Knockin (%) refers to gene correction by homology directed repair (HDR)).

Example tested lipid compositions generally include 40-50% DOTAP 5A2-SC8 SORT LNP formulations (for in vivo lung editing) and 10% DOTAP 5A2-SC8 SORT LNP formulations (for in vitro gene editing). For example, in F508del HBEs treatment experiments, lung SORT formulations “DOTAP 10” (that comprise 10 mol % DOTAP), and the functional variations with slightly tweaked molar ratios, were used. An example formulation used to aid in vitro transfection and F508del HBE editing was 5A2-SC8:DOPE:Cholesterol:PEG2000-DMG:DOTAP=32.4:18:36:3.6:10 (mol/mol), where total lipids: total nucleic acids=40:1 (wt/wt). Cas9 mRNA, sgRNA, and ssDNA template (Weight ratio among Cas9 mRNA:sgRNA:HDR=0.5:1:6) were assembled with the lipid delivery composition.

“DOTAP 40” LNPs were also explored for effective in vivo gene editing in lungs. For example, 5A2-SC8:DOPE:Cholesterol:DMG-PEG:DOTAP=21.6:12:24:214:40 (molar ratio) was utilized. Total lipid:total nucleic acid=20:1 (mass ratio):Weight ratio among Cas9 mRNA:sgR:NA:HDR=0.5:1:6.

### Example 3: Gene Correction of CFTR-G542X

Hela-G542X cells are thawed and plated, 1 ug of total RNA (including Cas9 mRNA, gRNA specific for CFTR, and a donor nucleic acid specific for G542X mutant allele).

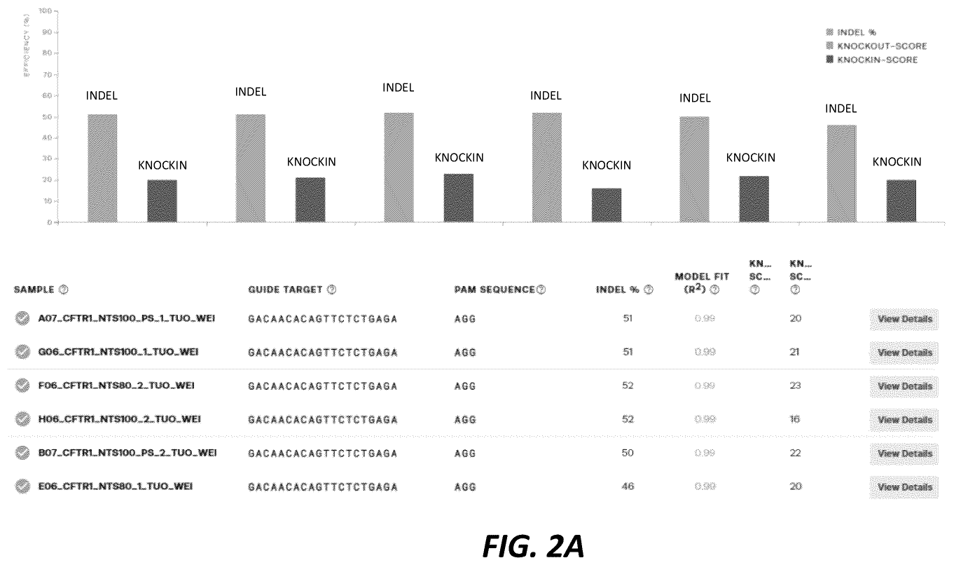

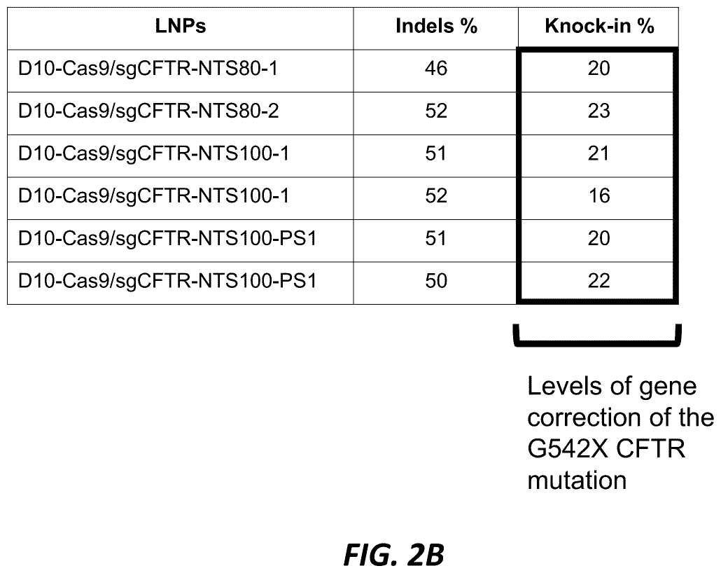

After 3 days the cells were lysed. The lyse cells were subjected to sequencing and the CFTR gene are analyzed to determine correction. FIG. 2A shows sets of a chart of the editing of Hela-G542X cells, with corresponding numbers shown in FIG. 2B. The % of indels and the knockout and knock-in scores were analyzed for different donor nucleic acids. The compositions are able to knock-in a correction to the cells at a rate of around 20%.

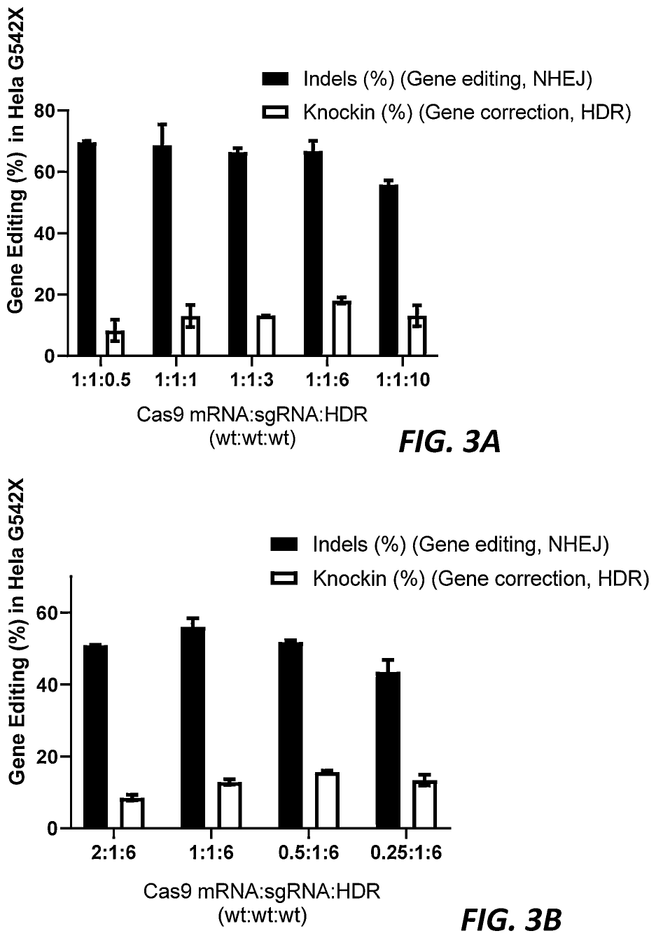

1.5×10⁵ Hela-G542X cells were seeded in 12-well plate overnight. Then the cells were treated with 100 ul of “DOTAP 10” (10 mol % DOTAP) LNPs with different weight ratios among three components (together with 500 uL of fresh medium), cells were added with 1 mL of fresh medium after treated for 2 days and cells were collected for PCR and DNA sequencing after treated for 3 days. TIDER analysis was used based on Sanger DNA sequencing data. Additionally, the weight ratio of the individually components in the LNP were analyzed FIGS. 3A and 3B show charts of the indel % and the Knockin % of the for varying ration of the Cas9 mRNA, sgRNA, and donor nucleic acid (HDR).

### Example 4: Delivery to Basal Lung Cells of Mice

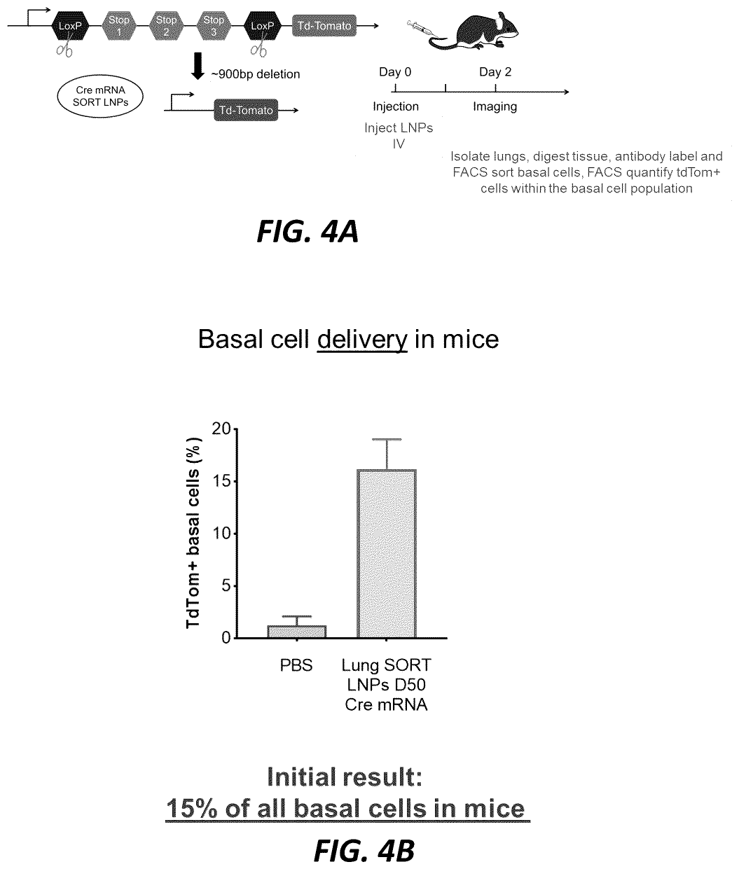

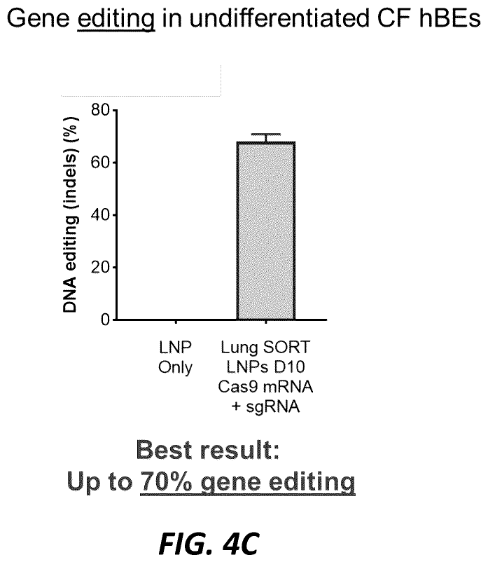

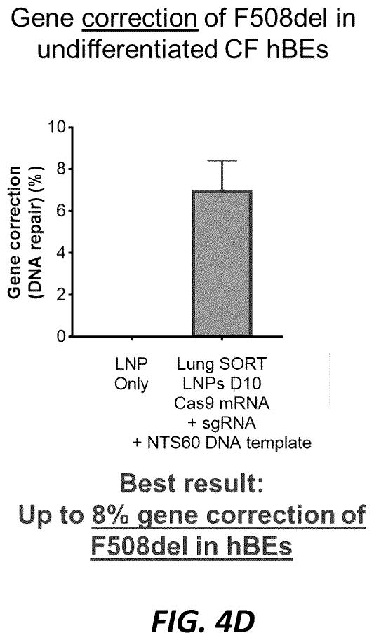

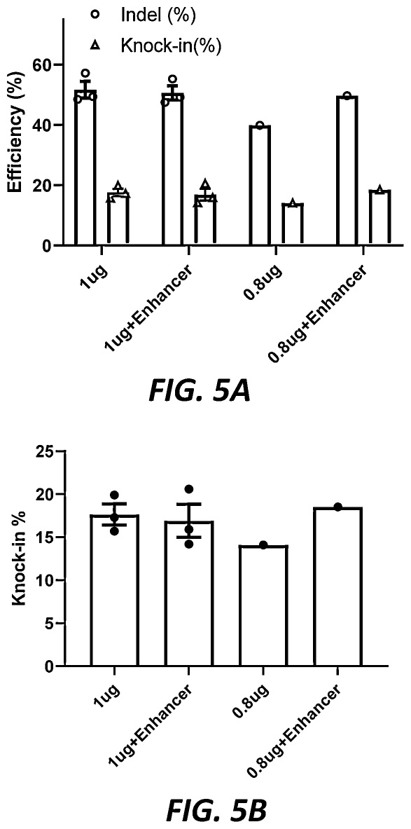

B6.Cg-Gt(ROSA)26Sortm9(CAG-tdTomato)Hze/J mice (also known as Ai9 or Ai9(RCL-tdT) mice) were obtained from The Jackson Laboratory (007909) and bred to maintain homozygous expression of the Cre reporter allele that has a loxP-flanked STOP cassette preventing transcription of a CAG promoter-driven red fluorescent tdTomato protein. Gene editing deletes the stop and turns on red fluorescent tdTomato protein expression. Lipid nanoparticles comprising 50% DOTAP (“DOTAP 50”) LNPs, for example, 5A2-SC8:DOPE: Cholesterol:PEG2000-DMG:DOTAP=18:10:20:2:50 (molar ratio), and a Cre mRNA were delivered to mice using intravenous delivery. The lungs of the mice were isolated and digested. The cells were labeled with Ghost Red 780 to distinguish live and dead cells and anti-p75 NGF receptor antibody to define basal cells and then analyzed using FACS. FIG. 4B shows quantification result of tdTOM+ cells of whole basal cell population in mouse lungs (n=4) (DOTAP 50 LNPs, 0.5 mg/kg Cre mRNA, 2 days). FACS gating for analysis of tdTOM+ expression in lung basal cells included Ghost Red 780 was used to distinguish live and dead cells and anti-p75 NGF receptor antibody to define basal cells. PBS group was used as negative control. Analysis was performed 2 days after IV injection. Total lipid:Cre mRNA=40:1 (mass ratio); Dose: 0.5 mg/kg of Cre mRNA; Injection route: Intravenous (IV). Gene editing deleted the stop and turns on red fluorescent tdTomato protein expression, as indicated in FIG. 4A. In related experiments, LNP comprising Cas9 mRNA and a gRNA were administered to undifferentiated HBE cells. DNA was isolated and sequenced. FIG. 4B illustrates that the delivery technology described herein effectuated at least (about) 15% of all basal cells in the tested subject (mice).

FIG. 4C shows the generation of indels from the Cas9 and gRNA in human epitehial cells frown at an air-liquid interface. LNPs comprising Cas9 mRNA, a gRNA and a donor nucleic acid were administered to undifferentiated HBE cells to enable homology-directed repair. DNA from the cells were isolated and sequenced. FIG. 4C illustrates gene editing effective in at least (about) 70% of the tested undifferentiated CF hBEs achieved by the technology described herein.

FIG. 4D shows the % of gene repair in the cells. Particularly, the technology described herein achieved at least (about) 8% gene correction of F508del in undifferentiated CF hBEs.

### Example 5: Treatment of a Subject with CFTR Mutations

A subject having or suspected of having a CFTR-associated condition is given a treatment by administering a composition of LNP, Cas9 mRNA, a gRNA, and a donor nucleic acid. The subject is monitored at regular intervals for expression of CFTR in the lungs. A sample of lung tissue from the subject is taken comprising lung cells. The cells are harvested and prepared for DNA sequencing. The cells are sequence and the CFTR allele is analyzed for any mutations.

Alternately or additionally, a subject having or suspected of having a CFTR-associated condition may have some lung cells extracted. The bronchial epithelial cells are subjected to a treatment by administering a composition of LNP, Cas9 mRNA, a gRNA, and a donor nucleic acid. The cells are then transplanted back into the subject lungs and allowed to propogate. The subject is monitored at regular intervals for expression of CFTR in the lungs. A sample of lung tissue from the subject is taken comprising lung cells. The cells are harvested and prepared for DNA sequencing. The cells are sequence and the CFTR allele is analyzed for any mutations.

### Example 6: TIDER Analysis

HBE cells (e.g., passage one (P1)) were seeded in 6-well plate (150 k/well) and cultured for 4 days. The cells were then treated with 100 μL of Cas9 mRNA:sgRNA:HDR (0.5:1:6 wt/wt/wt), assembled with lipid composition(s) described herein (e.g., 5A2-SC8 10% DOTAP), in 2 mL fresh medium. The HDR template as set forth in Table B (“NTS60”) was used. Fresh medium was changed at day 2 after treatment; and the treated cells were subsequently collected for sequencing at day 3. Part of the treatment comprises adding an HDR enhancer (2 μL per well) that was expected to increase usage of HDR versus NHEJ for repair of double strand breaks (DSBs). Unexpectedly, the data indicate no significant enhancement with the usage of the enhancer.

### Example 7: HDR Mediated CRISPR/Cas Genome Editing in Cystic Fibrosis Models

Although the majority of Cystic Fibrosis (CF) patients are eligible for effective modulator drug therapy, roughly −10% of patients still have no effective medicine to take. This issue is especially acute for individuals with nonsense mutations in the Cystic Fibrosis Transmembrane Conductance Regulator (CFTR) gene. Gene correction through delivery of the CRISPR/Cas system to CF-relevant organs/cells would be a significant advance towards new therapies for CF. However, this strategy is severely limited by the lack of efficient genome editor delivery carriers. Provided herein are improved Lung Selective Organ Targeting (SORT) Lipid Nanoparticles (LNPs) that can efficiently deliver Cas9 mRNA, sgRNA, and donor ssDNA templates for precise homology-directed repair (HDR)-mediated gene correction in ex vivo and in vivo CF models. Importantly, optimized lung targeting SORT LNPs can deliver mRNA to lung basal stem cells with high potency, which can contribute to long-lasting expression of CFTR proteins in epithelial cells derived from basal cells. Optimized Lung SORT LNPs successfully corrected the G542X CFTR mutation in genetically engineered mice harboring homozygous G542X mutations and in patient-derived human bronchial epithelial (HBE) cells harboring homozygous F508del mutations. This led to restored expression of CFTR proteins and chloride transport function.

Although effective therapies have been developed for patients with gating, conduction defects, or two copies of the F508del mutation in the Cystic Fibrosis Transmembrane Conductance Regulator (CFTR) gene, most patients with two copies of nonsense mutations remain untreatable with current approaches. CRISPR/Cas is a revolutionary genome editing technology that if successfully developed to correct CFTR mutations could be a transformative advance resulting in long lasting therapies for all Cystic Fibrosis (CF) patients, including those with nonsense mutations.

A key bottleneck is the lack of delivery strategies required to enable targeted editing in specific cells, especially cells in lungs. To date, successful in vivo editing has been mediated mainly by viral vectors, which present challenges for clinical translation due to potential immunogenicity, concerns about rare but dangerous integration events, and inability to re-dose. Intravenous non-viral lipid nanoparticle (LNP) delivery can offer advantages in those respects, targeting is often limited to the liver. Selective Organ Targeting (SORT) LNPs can enable intravenous delivery of nucleic acids and proteins to the lungs, liver, and spleen, plus local cell-specific delivery to the muscle and brain. Importantly, Lung SORT LNPs can enable genome editing in cells across the lungs, from the endothelium to the epithelium in reporter mice.

A series of permanently cationic Lung SORT lipids were examined wherein the molar ratio of lipids components within LNPs was modified, and improved LNP formulations for precise HDR genome editing ex vivo (DOTAP10 LNPs) and in vivo (DOTAP40 LNPs) were identified. The HDR correction efficiency in BFP/GFP switchable HEK293 cells with a Y66H mutation achieved as high as 50% and turned on bright GFP fluorescence in treated cells. Strikingly, optimized lung targeting LNPs (DOTAP40) could transfect −60% of lung basal cell population after systemic IV administration, which is very promising for CF therapy as sufficient CFTR gene correction in lung basal stem cells could contribute to long-term functional restoration of the airway epithelium. Optimized SORT LNPs can successfully correct the G542X CFTR mutation in mouse lungs of G542X CF mice and restore CFTR function in intestinal organoids derived from these animals. Additionally, SORT LNPs efficiently corrected the F508del mutation in patient-derived human bronchial epithelial (HBE) cells and restored the expression of CFTR protein and relative CFTR function after propagation.

Optimization of SORT LNPs Enabled Enhanced mRNA Delivery to Mouse Lungs with Low Toxicity.

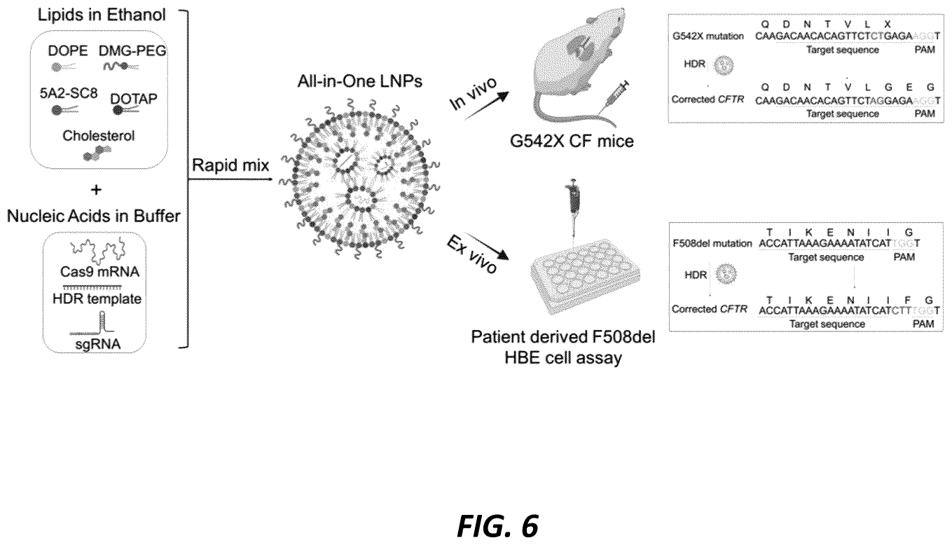

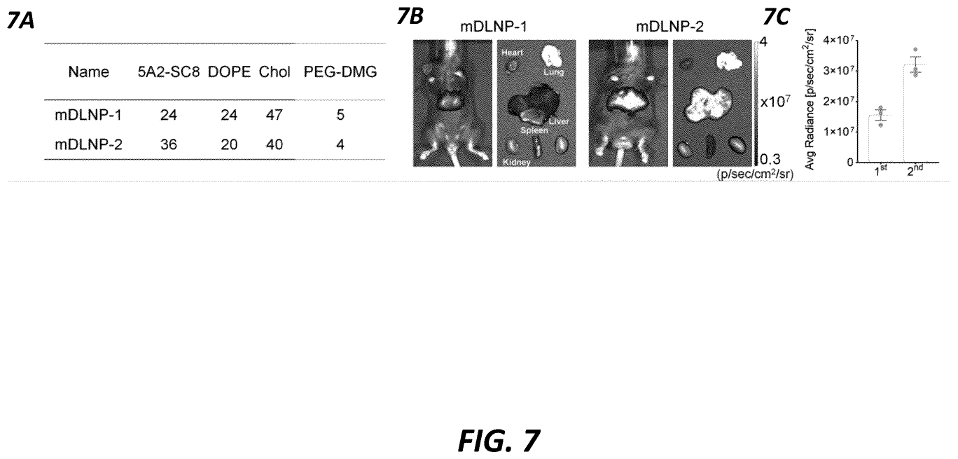

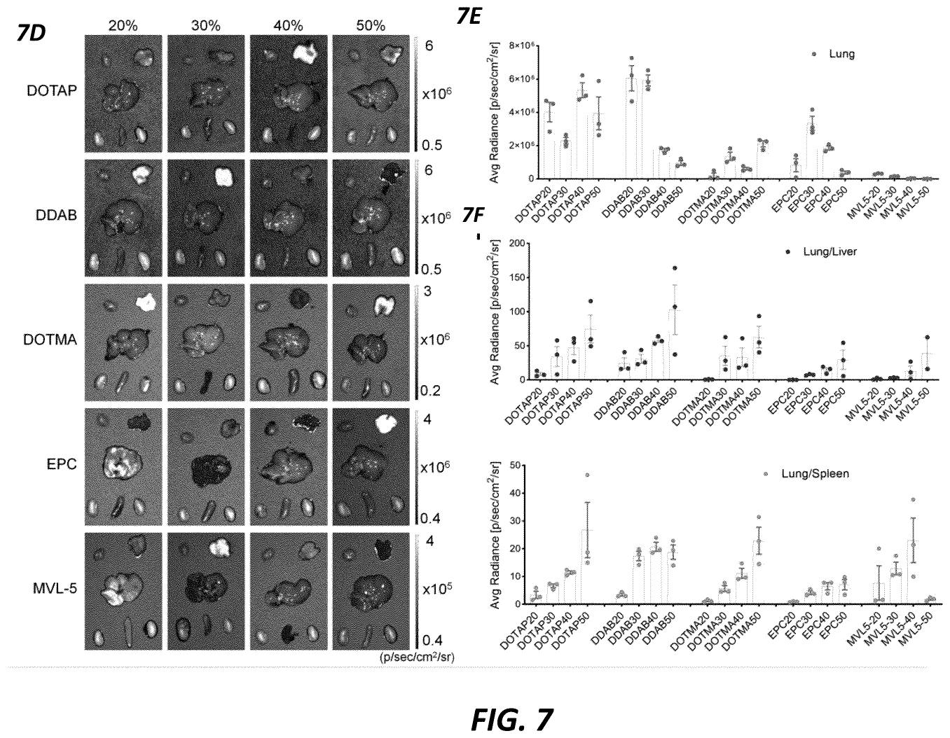

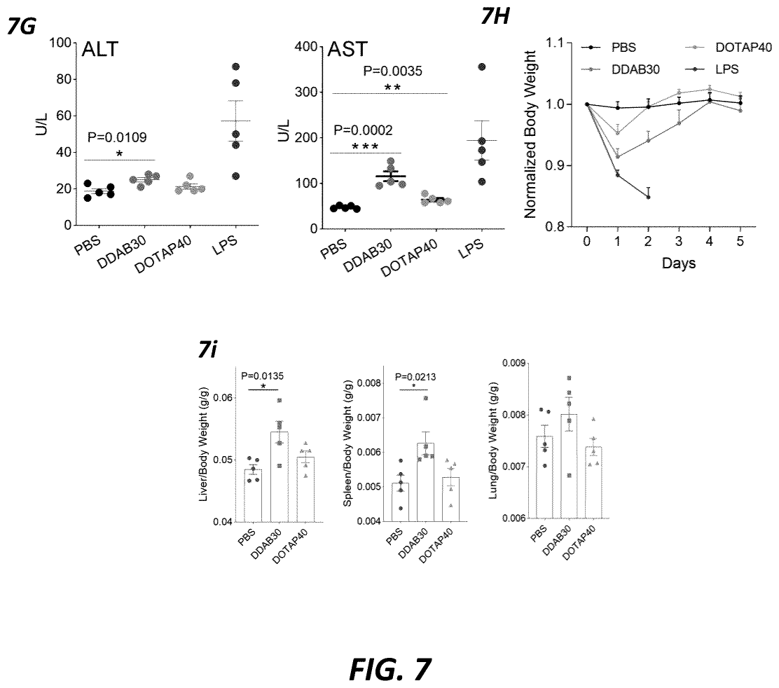

Development of a CRISPR/Cas HDR approach requires delivery of multiple components, including different nucleic acid types (Cas9 mRNA, sgRNA, and ssDNA) (FIG. 6). To improve delivery efficacy, the molar ratios of lipids used in LNPs were modified to optimize for higher lung-specific activity. In some instances, increasing the proportion of the ionizable amino lipid (5A2-SC8) in a previously identified mDLNP formulation (termed mDLNP-1) significantly enhanced mRNA delivery in vivo. Using the SORT lipid mixing strategy, lipid ratios were modified to identify second generation mDLNPs (termed mDLNP-2) with a molar ratio of 5A2-SC8/DOPE/Chol/PEG-DMG fixed at 36/20/40/4 (FIG. 7A).

LNP formulations were examined for IV delivery of Luciferase (Luc) encoding mRNA in C₅₇BL/6 mice. Luc activity was quantified 6 hours after injection using in vivo luminescence imaging. mDLNP-2 enabled higher luminescence activity compared to mDLNP-1, indicating higher mRNA delivery efficiency (FIG. 7B, FIG. 7C).

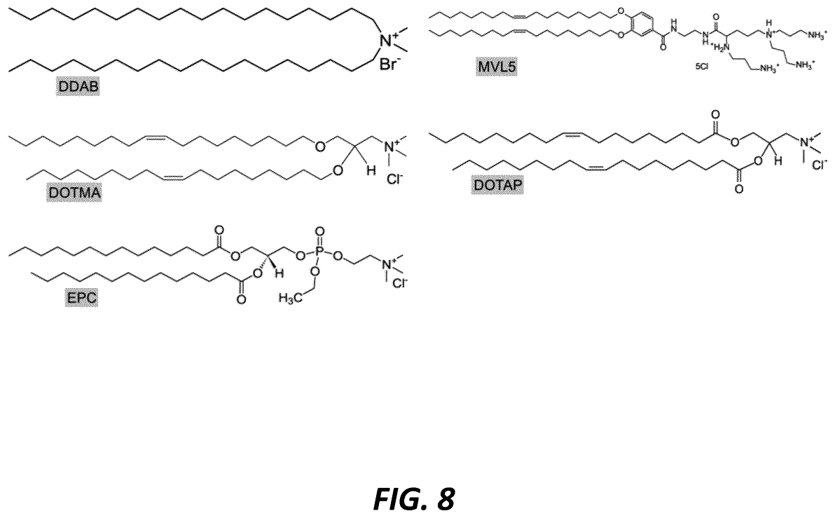

Since SORT molecules for lung targeting fall into a generalizable class, a series of permanently cationic lipids with different chemical structures, including DOTAP, DDAB, DOTMA, EPC, and MVL5 were screened (FIG. 8). These cationic lipids were incorporated into mDLNP-2 with different percentages (fraction of total lipids). By delivering Luc mRNA, the lung-targeting efficacy of these LNP carriers in vivo was compared. Incorporation of DOTAP and DDAB in mDLNP-2 exhibited higher luminescence activity than others (FIG. 7D, FIG. 7E). To evaluate the lung targeting specificity, the relative luciferase expression of Lung/Liver and Lung/Spleen of different LNP carriers was calculated based on in vivo imaging data (FIG. 7F). All identified Lung SORT LNPs targeted the lungs effectively and hold promise for further development and optimization. Following an analysis of both lung delivery efficacy and specificity, DOTAP40 LNPs and DDAB30 LNPs were selected for further consideration.

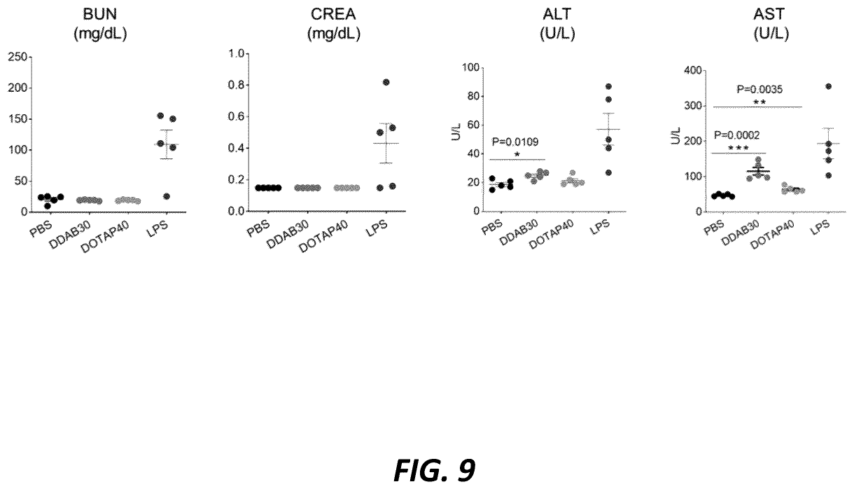

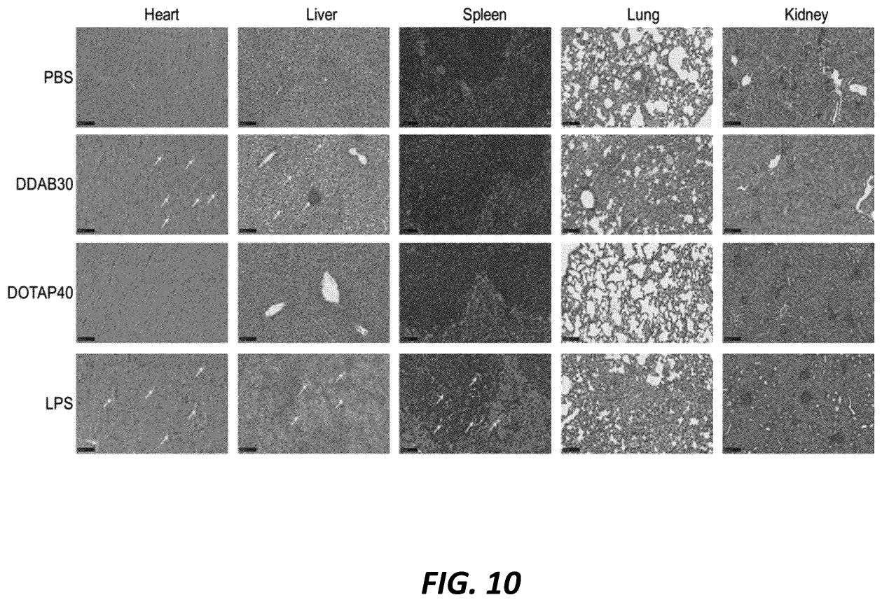

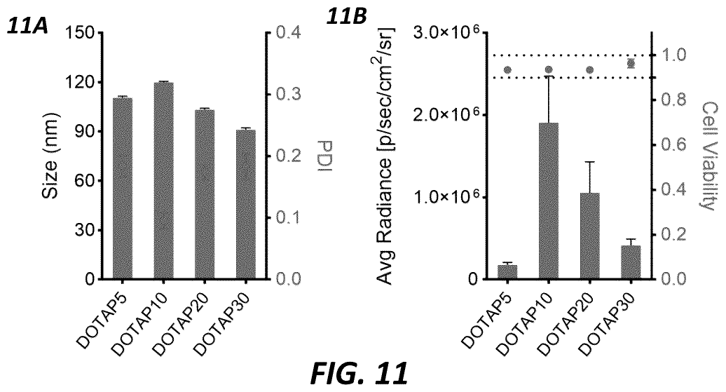

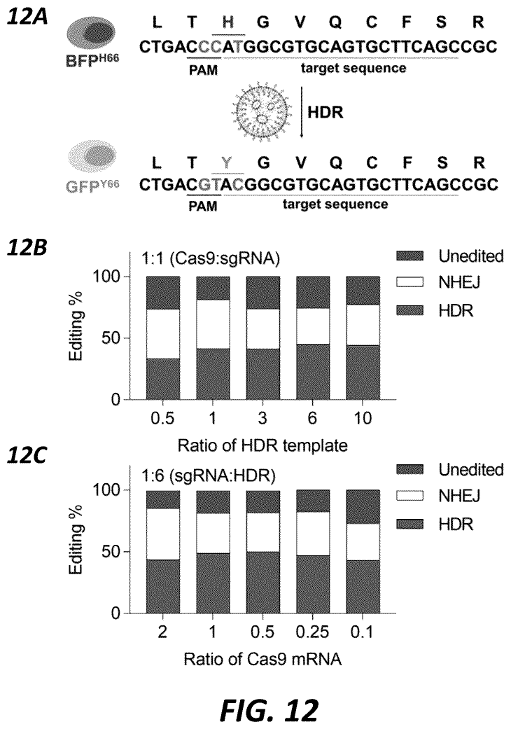

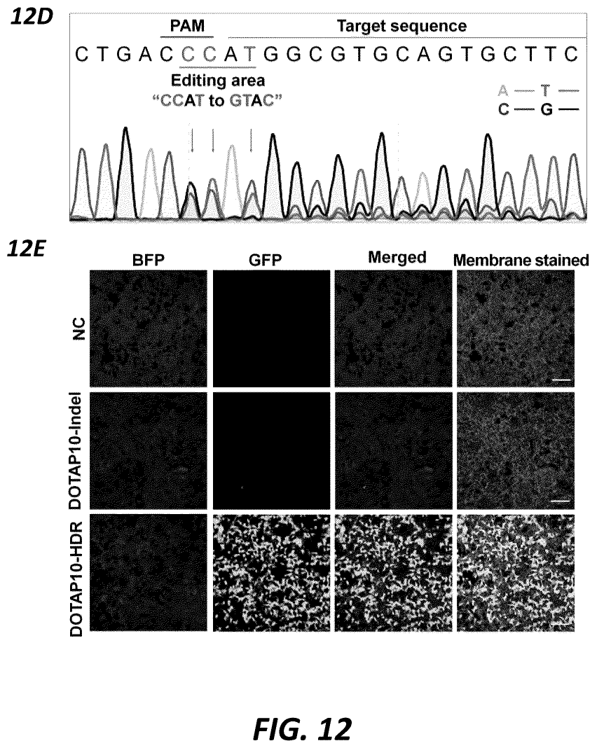

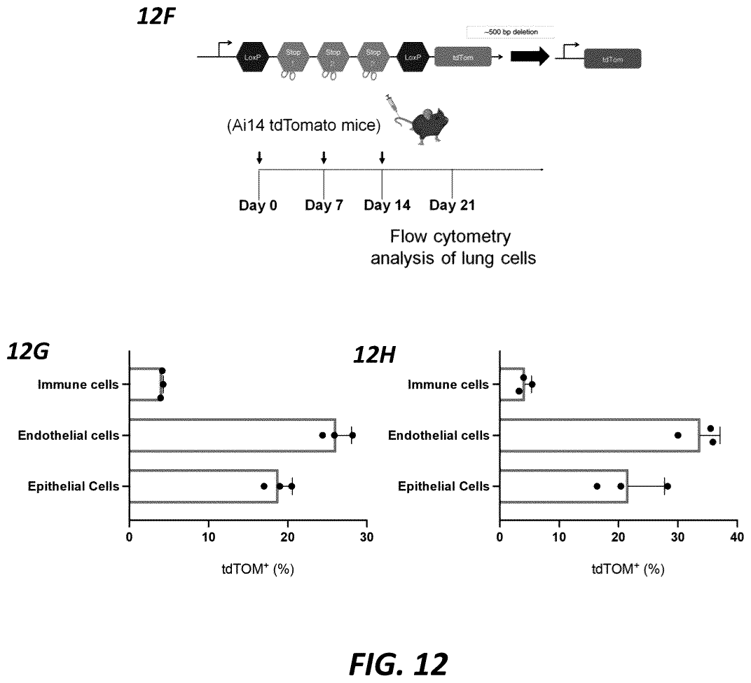

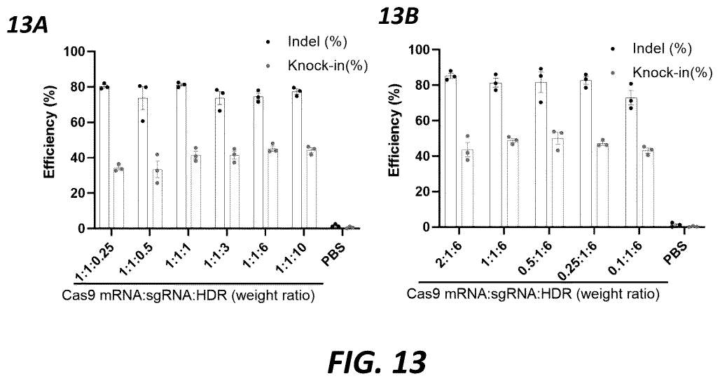

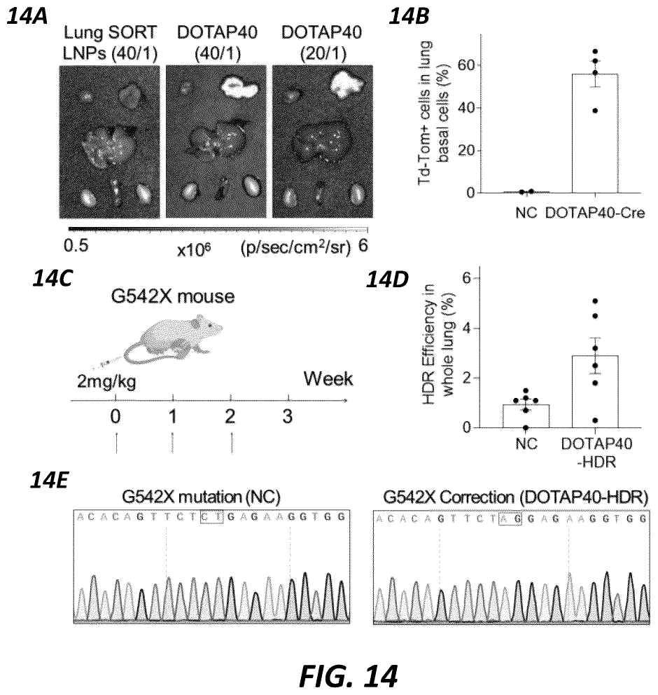

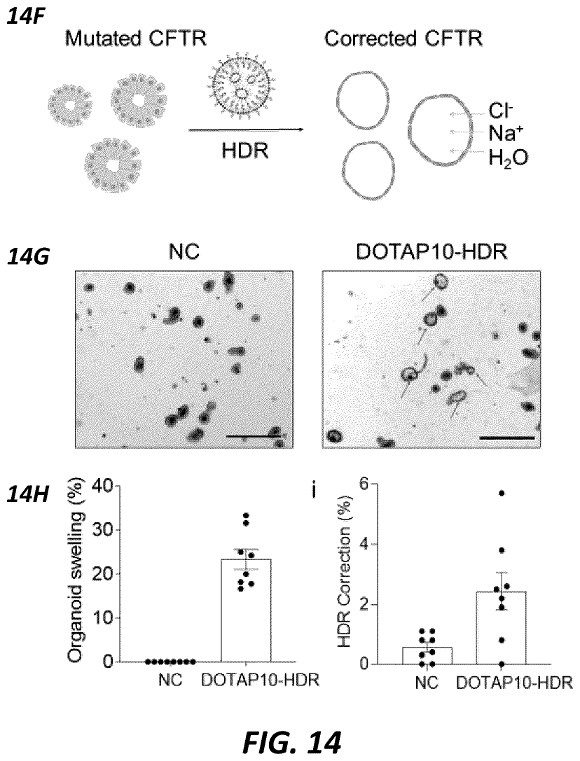

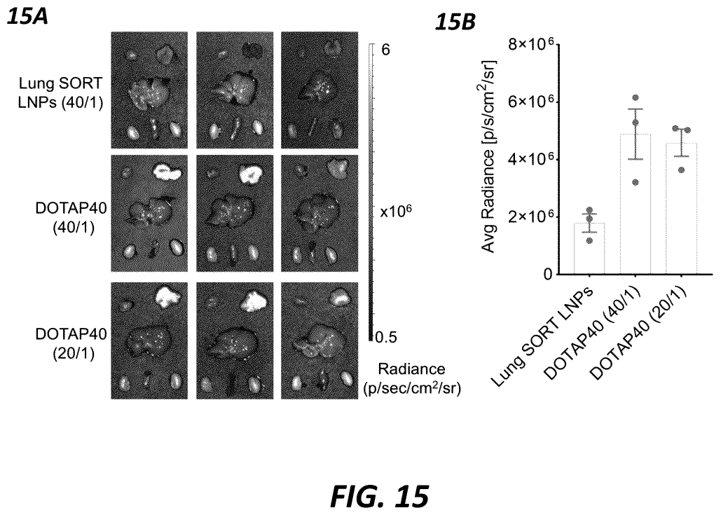

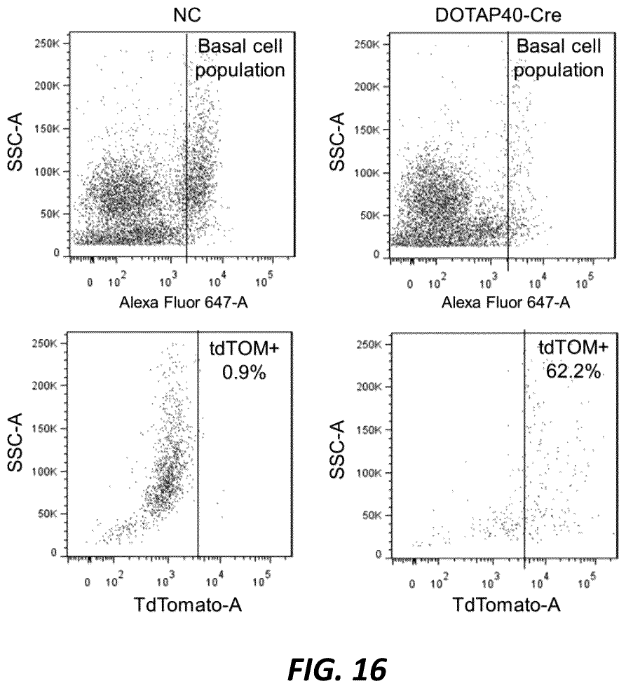

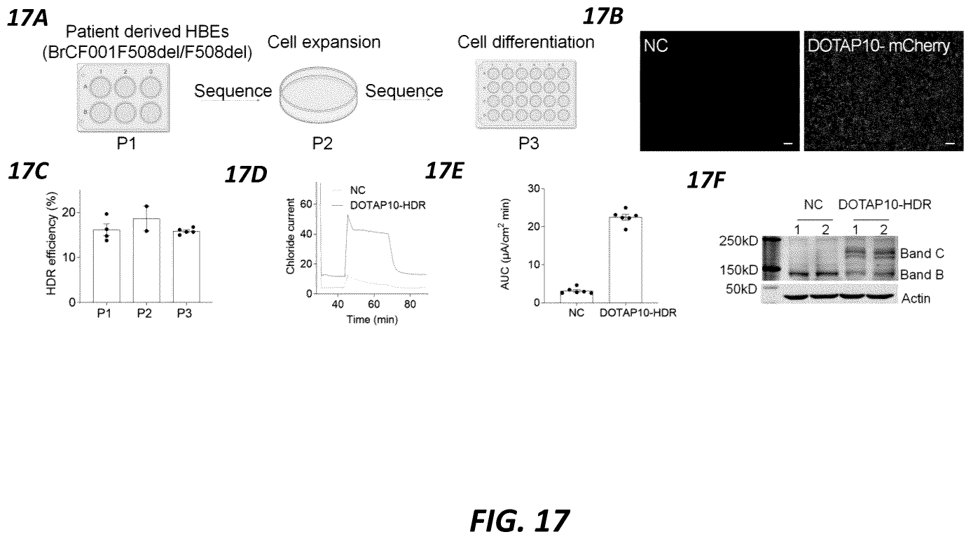

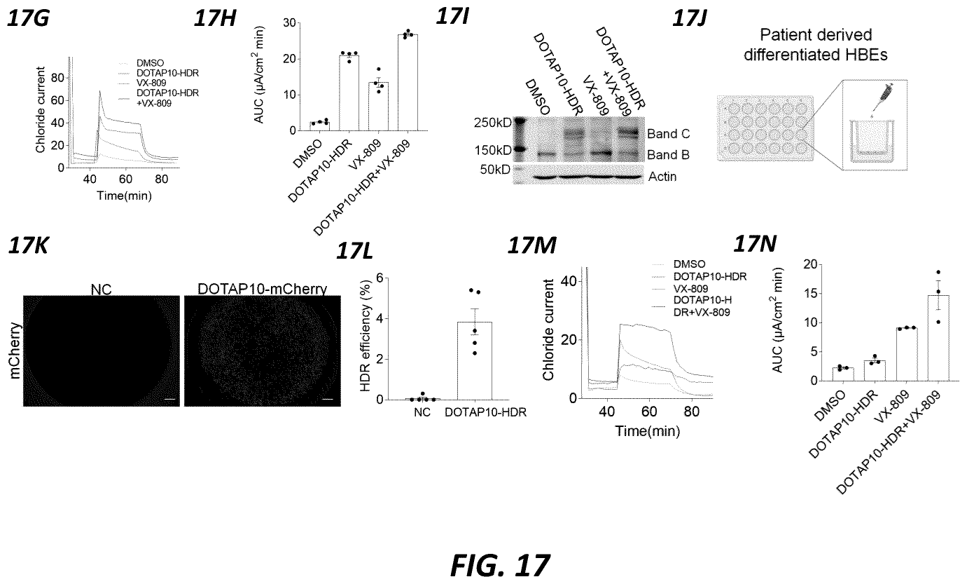

During the evaluation process, it was determined that DDAB LNPs may exhibit some toxicity after treatment. To further confirm this, in vivo toxicity of DOTAP40 and DDAB30 LNPs treatments were evaluated by measuring serum biomarkers relating to liver function (AST and ALT) and kidney function (BUN and CREA). Higher toxicity in the liver was observed in the DDAB30 LNP treatment group after 24h (FIG. 7G, FIG. 9). Body weight changes and noted increased weight loss in mice treated with DDAB30 LNPs were observed, suggesting higher acute toxicity (FIG. 7H). At the end of the study, mice were sacrificed and organs (liver, spleen, and lung) were excised and weighed. The liver/body weight ratio and spleen/body weight ratio were increased with DDAB30 LNP treatment, suggesting higher toxicity (FIG. 7I). Tissue damage was also detectable in immunohistochemistry (IHC) images after treatment of DDAB30 LNPs (FIG. 10). Based on these findings comparing DDAB and DOTAP based SORT LNPs, DOTAP40 SORT LNPs were selected for continued study.

Subsequent studies were conducted to determine whether there are optimal DOTAP LNPs for in vitro cell studies or intramuscular injection. To optimize DOTAP based SORT LNPs for in vitro utility, a series of mDLNP-2 SORT LNPs with 5%-30% DOTAP were prepared and Luc mRNA delivery to HeLa cells was examined. The result showed that DOTAP10 was the most efficacious, producing the highest luminescence intensity after 24h treatment (FIG. 11).

DOTAP10 LNPs Enabled Efficient Gene Correction in BFP/GFP Switchable HEK293 Cells with Y66H Mutation.

LNPs have been previously reported to efficiently edit cells by co-delivery of Cas9 mRNA and sgRNA in vivo, leading to gene knock-out by forming insertions and deletions (indels) at the double strand break (DSB) site. However, this approach is not useful for genome correction. For CRISPR/Cas, precise gene correction at the mutated site by HDR is required to restore function. To evaluate the ability of DOTAP LNPs to deliver Cas9 mRNA, sgRNA, and ssDNA HDR templates for precise gene correction, BFP/GFP switchable reporter HEK293 cells were selected. These cells expressing a GFP sequence have a single amino acid mutation (Y66H) in their GFP sequencing (TAC mutated to CAT), which alters the fluorescence from green (GFP) to blue (BFP). Once the mutation is corrected, the GFP function will be restored and the fluorescence will return to green (FIG. 12A).

Because the internal ratio among multiple CRISPR/Cas9 nucleic acid components is critical for HDR correction, a series of DOTAP10 LNPs with different weight ratios among Cas9 mRNA, sgRNA, and HDR template were prepared. First, the weight ratio of Cas9 mRNA:sgRNA was fixed at 1:1, and adjusted the ratio of HDR template added. Sequencing results suggested that Cas9 mRNA:sgRNA:HDR at 1:1:6 had the highest HDR correction efficiency (FIG. 12B, FIG. 13A). Next, the weight ratio of sgRNA:HDR was fixed at 1:6 and the ratio of Cas9 mRNA added was adjusted. The second round of screening showed that the optimal weight ratio among Cas9 mRNA:sgRNA:HDR was 0.5:1:6, and the HDR correction efficiency reached as high as 50% (FIG. 12C, FIG. 12D, FIG. 13B). Using the optimal internal ratio, DOTAP10 LNPs encapsulating Cas9 mRNA, sgRNA, and HDR templates (termed as DOTAP10-HDR) successfully corrected the mutated GFP gene and switched on bright GFP fluorescence in HEK293 cells, while little green signal was detected with treatment of DOTAP 10 LNPs encapsulating Cas9 mRNA and sgRNA (named DOTAP10-Indel) (FIG. 12E). Recognizing that inclusion of HDR templates could affect SORT LNP formation, whether DOTAP40 LNPs could induce indels in lox-stop-lox tdTomato mice²⁰ was examined following IV administration of Cas9 mRNA and sgRNA targeting the lox-stop-lox site (sgTOM1) with or without sacrificial ssDNA donor template (FIG. 12F). Similar levels of editing were reached in endothelial and epithelial lung cells of mice that received mRNA:sgRNA (FIG. 12G) or mRNA:sgRNA:ssDNA (FIG. 12H), indicating that triple loaded SORT LNPs retain editing capacity in vivo.

Lung SORT LNPs could Reach Basal Stem Cells in Mouse Lungs.

As a result of the high HDR correction efficiency in HEK293 cells, CF mouse model harboring G542X mutation were treated. To maximize the dose of LNPs for in vivo study, the weight ratio between total lipid/total NA of DOTAP40 LNPs was changed from 40:1 to 20:1 maintain efficacy, thereby improving tolerability of DOTAP containing LNPs. Comparable delivery efficiency in mouse lungs was observed after decreasing weight ratio from 40:1 to 20:1 (FIG. 14A, FIG. 15), indicating that less lipids could be used. The total lipid/total NA of DOTAP 40 LNPs was fixed at 20:1 for future in vivo studies.

Airway basal cells are stem cells that can differentiate into different cell types of airway epithelium. Correcting sufficient gene mutations in basal stem cells could contribute to long-term functional restoration of the airway epithelium. Given this, development of a delivery system that could deliver CRISPR/Cas9 system to basal cells would be a critical and meaningful advance for CF therapy. Previously, a single administration of Lung SORT LNPs (5A2-SC8/DOTAP/DOPE/Cholesterol/PEG-DMG=11.9/50/11.9/23.8/2.4 (mol/mol)) transfected ˜60% of all endothelial cells and ˜40% of all epithelial cells following a single IV injection of 0.3 mg kg⁻¹ Cre recombinase (Cre) mRNA to lox-stop-lox tdTomato mice. Given that Lung SORT LNPs access epithelial cells from the blood side, Lung SORT LNPs may also reach basal cells. To determine this, the transfection efficiency of optimized DOTAP40 LNPs encapsulating Cre mRNA (DOTAP40-Cre) to NGFR⁺ basal cells was quantified using flow cytometry of cells extracted from edited mouse lungs following IV injection in tdTOM reporter mice (twice 2 mg kg⁻¹ Cre mRNA). DOTAP40-Cre transfected −60% of NGFR⁺ basal cell populations after treatment (FIG. 14B, FIG. 16). This result encouraged further evaluation of the possibility of the delivery system to correct CFTR mutations in lungs of a G542X CF mouse model.

DOTAP LNPs Successfully Corrected G542X Mutation in CF Models and Restored CFTR Function in Intestinal Organoids with G542X Mutation.

DOTAP40 LNPs were evaluated for treatment of the CF mouse model with homozygous G542X nonsense mutation. DOTAP40 LNPs encapsulating Cas9 mRNA, sgRNA, and HDR templates targeting G542X mutation (DOTAP40-HDR) were administered IV by tail vein into CF mice every week for total three times. One week after the last injection, whole mouse lungs were collected and homogenized for genome DNA extraction and DNA sequencing (FIG. 14C). TIDER analysis result based on DNA sequencing showed that DOTAP40-HDR treatment successfully corrected G542X mutation in mouse lungs. About ˜4% of CFTR gene extracted from whole lung tissue was corrected (FIG. 14D). Because of the analysis of all cells from the lungs, the rate of editing may be diluted through isolation of cells assumed to be irrelevant for CF treatment (e.g., immune cells that are transfected less than other cell types in the lungs by SORT LNPs) (FIG. 14G, FIG. 14H). This result is promising, as there are few dividing cell populations that can be corrected through HDR-mediated strategy. This requirement of CRISPR/Cas9 HDR is likely a limiting factor here, which may be overcome in future studies using next generation gene editors that do not require double strand breaks. The TA clone result further confirmed that gene correction at G542X mutation site was observed with DOTAP40-HDR treatment (FIG. 14E).

Although the G542X mouse model contains G542X mutation in their genomic DNA, the lungs of these mice do not show CF-related pathological features. Therefore, this model cannot be used to evaluate restoration of CFTR function. To overcome this limitation, cells were isolated from the intestines of these mice and formed intestinal organoids containing G542X mutation as an ex vivo model to evaluate CFTR function after DOTAP10-HDR treatment. The CFTR activator, forksolin, can stimulate intracellular pathways and phosphorylate CFTR, finally opening the CFTR channel and leading to ion/water uptake and organoid swelling. If the mutated CFTR gene is corrected, organoid swelling should be observed using the forksolin-induced swelling (FIS) assay, while uncorrected organoids (negative control, NC) will remain at the baseline volume (FIG. 14F). Using the FIS assay, organoid swelling after treatment of DOTAP10-HDR was successfully observed (FIG. 14G). Quantitative results suggested that over 20% of organoids swelled after DOTAP10-HDR treatment, demonstrating CFTR gene correction occurred which led to functional CFTR expression (FIG. 14H). TIDER analysis based on DNA sequencing further confirmed the result (FIG. 14I).

DOTAP LNPs Corrected CFTR Mutation and Partially Restored CFTR Function in Patient-Derived Human Bronchial Epithelial (HBE) Cells with F508del CFTR Mutation.

The F508del mutation is the most common CF disease allele, which accounts for 70% of all CF mutations. Even though there are CFTR modulators on the market that can help relieve disease related symptoms, there is still no way to cure CF disease permanently. Primary human bronchial epithelial (HBE) cells from CF patients with F508del mutation (F508del/F508del CF HBEs) are widely recognized as the gold standard assay for preclinical CF studies most predictive of clinical benefit. Because F508del HBEs are more commonly available than G542X HBEs, this study focused on correcting the F508del mutation in the HBE assay. Therefore, whether the LNP formulations could deliver RNA cargos to homozygous F508del/F508del HBEs (FIG. 17A) was examined. DOTAP10 LNPs encapsulating mCherry mRNA (DOTAP10-mCherry, 0.5 μg mL⁻¹) were transfected to F508del/F508del CF HBEs and their cellular uptake was evaluated using fluorescence microscopy. Bright mCherry fluorescence was observed after transfection, compared to NC group (FIG. 17B) across the entire field of view. This demonstrated that the LNP formulation could effectively encapsulate and deliver RNA cargoes to HBE cells, providing potential for genome editing of CF mutations.

The possibility of DOTAP10 LNPs to correct F508del mutation in HBE cells was analyzed. Patient-derived undifferentiated HBE cells with homozygous F508del mutation (passage 1, P1) were treated with DOTAP10 LNPs encapsulating Cas9 mRNA, sgRNA, and HDR template targeting the F508del mutation (DOTAP10-HDR) for 4 days before cell expansion (passage 2, P2). One week after cell expansion, these cells were transferred to transwell inserts for cell differentiation (passage 3, P3) (FIG. 17A). During cell differentiation processing, the HDR correction efficiency maintained at as high as 16% with DOTAP10-HDR treatment (FIG. 17C). At the end of the study, Cl⁻ channel activity mediated by DOTAP10-HDR in differentiated HBE cells (P3) was evaluated. DOTAP10-HDR treatment successfully restored CFTR mediated Cl⁻ transport, compared to untreated group (NC) (FIG. 17D, FIG. 17E). The western blot result further confirmed the expression of CFTR protein after gene correction by DOTAP10-HDR, while no CFTR protein (at band C) was detected with NC group (FIG. 17F). Meanwhile, HDR efficacy of DOTAP10-HDR, VX-809 (a CFTR modulator) and their co-treatment on restoration of CFTR function were compared. DOTAP10-HDR showed better effect than VX-809 (FIG. 17G). Co-treatment of DOTAP10-HDR and VX-809 significantly enhanced Cl⁻ transport activity, suggesting possible advantage of combinational therapy in future CF studies (FIG. 17H). These results were further confirmed by increased expression of CFTR protein after single treatment of DOTAP10-HDR and co-treatment of DOTAP10-HDR/VX-809 (FIG. 17I).

Compared to undifferentiated HBEs, fully differentiated HBE cells with F508del mutations secrete thicker mucus on the air interface, which is a more challenging barrier for LNP transport. Additionally, these cells have reached a steady state with slow cell division that likely hinders CRISPR/Cas HDR correction that requires cell division. Nevertheless, this model can be a useful addition to broader consideration of in vitro, ex vivo, and in vivo genome correction efforts (FIG. 17J). To pursue this goal, DOTAP10 LNP delivery of mCherry mRNA to fully differentiated F508del HBE cells was examined. Bright mCherry fluorescence was observed after treatment (FIG. 17K), indicating that DOTAP10 LNPs can transfect fully differentiated HBE cells. Next, DOTAP10-HDR LNPs delivering CRISPR/Cas components were directly added onto the apical surface of differentiated HBE cells with F508del mutation in transwell inserts. Around 4% of HDR correction was detected after three days based on DNA sequencing and TIDER analysis (FIG. 17L). Although lower than the correction rate observed in undifferentiated cells, this may be due to cell cycle states of slowly dividing cells that restrict HDR, as extensive LNP and assay optimization did not increase correction rates (data not shown). Co-treatment of DOTAP10-HDR and VX-809 significantly enhanced Cl⁻ transport and improved CFTR function in differentiated F508del HBE cells (FIG. 17M). Quantitative data from values of area under the curve (AUC) additionally confirmed this result (FIG. 17N). These results demonstrated that DOTAP LNPs can effectively transport through mucus, internalize into HBE cells, and release encapsulated cargoes, providing potential for future efforts in genome editing of CF mutations.

### Discussion

Development of Lung SORT LNPs capable of correcting CFTR in mouse models of CF and human CF HBEs represents key advances on the path to genome correction therapies. At present, synthetic carriers that can mediate nucleic acid delivery to lung basal cells are limited. Moreover, synthetic carriers that demonstrate high levels of CRISPR/Cas-mediated gene correction of CFTR in vivo are limited. Provided herein are second generation Lung SORT LNPs (DOTAP40 LNPs), which significantly enhanced lung-targeting efficacy through screening a series of permanently cationic lipids and adjusting their internal ratios among lipid components. It was determined that DOTAP40 LNPs could deliver mRNA to lung basal stem cells with high potency. LNPs that can reach lung basal cells following IV administration is a critical step likely required for durable genome therapies in humans. Although correction of CFTR various airway cell types is expected to transiently restore CFTR activity, it is now understood that correction of airway basal and stem cells (progenitor cells) would likely be required to enable long-lasting expression of CFTR in epithelial cells derived from progenitor cells. Direct, in vivo editing of these cell populations may represent the best opportunity for durable “cures” of CF.

Gene correction of CFTR in the lungs of G542X mice is a significant advance. The approval of Trikafta presents an advance—although the majority of CF patients now have a medicine to take, those with nonsense mutations do not. Since G542X is the most common nonsense mutation of CFTR gene, thus this mutation was selected for in vivo gene correction. SORT LNPs may enable successful gene correction in mouse lungs of CF model harboring G542X mutation. In addition to G542X mutation, SORT LNPs can also be extended to other mutations, both rare and common.

In addition, SORT LNPs successfully corrected CFTR and partially restored CFTR function in patient-derived F508del HBE cells. These trans-well function assays have proven to be highly predictive of clinical benefit and have supported the approval of CFTR modulators that are now used to treat CF patients that harbor specific CFTR mutations such as F508del or G551D.

### Materials and Methods

### Materials

5A2-SC8 were synthesized and purified by following published protocols. 1,2-dioleoyl-sn-glycero-3-phosphoethanolamine (DOPE), 1,2-dioleoyl-3-trimethylammonium-propane (DOTAP), dimethyldioctadecylammonium (DDAB), 1,2-dimyristoyl-sn-glycero-3-ethylphosphocholine (EPC), 1,2-di-O-octadecenyl-3 trimethylammonium propane (chloride salt) (DOTMA), and N1-[2-((1S)-1-[(3-aminopropyl)amino]-4-[di(3-amino-propyl)amino]butylcarboxamido)ethyl]-3,4-di[oleyloxy]-benzamide (MVL5) were purchased from Avanti Polar Lipids. Cholesterol was purchased from Sigma-Aldrich. 1,2-Dimyristoyl-sn-glycerol-methoxy(poly((ethylene glycol) MW 2000) (DMG-PEG2000) was purchased from NOF America Corporation. Pur-A-Lyzer Midi Dialysis Kits (WMCO, 3.5 kDa) were purchased from Sigma-Aldrich. Lab-Tek chambered cover glass units were purchased from Thermo Fisher Scientific. Luc mRNA and mCherry mRNA were purchased from TriLink BioTechnologies. Cre mRNA and Cas9 mRNA were produced using in vitro transcription (IVT). D-Luciferin (sodium salt) was purchased from Gold Biotechnology. Anti-p75 NGF Receptor antibody (ab8875) and Donkey Anti-Rabbit IgG H&L (Alexa Fluor® 647) (ab150075) was purchased from Abcam. Anti-CFTR antibody (UNC 596) was purchased from University of North Carolina and anti-Actin antibody (MAB1501) was purchased from Millipore. VX-809 (S1565) was purchased from Selleckchem. End-modified sgRNAs (Table 7) were purchased from Synthego. All primers (Table 9) and end-modified HDR templates (Table 8) were synthesized by Integrated DNA Technologies (IDT).

### LNP Formulations

5A2-SC8, DOPE, cholesterol, DMG-PEG, and permanently cationic lipids (DOTAP, DDAB, DOTMA, EPC, or MVL5) were dissolved in ethanol at given molar ratios. mRNA or total nucleic acids (NA, including Cas9 mRNA, sgRNA and HDR template) were dissolved in Citrate buffer (pH 4). Then the acidic buffer was pipette mixed rapidly into the lipid solution in ethanol at a volume ratio of 3:1, whereas the weight ratio between total lipids and total NA was at 40:1 or 20:1. Afterward, the solution was incubated for 15 min at room temperature to finish assembly. The fresh formulations were directly characterized and used for in vitro assays. For animal experiments, the formulations were dialyzed (Pur-A-Lyzer Midi Dialysis Kits, WMCO 3.5 kDa) against 1×PBS for 3 h to remove acidic buffer and ethanol before in vivo injections.

TABLE 7

| | Target Sequences | SEQ | PAM |
|---|---|---|---|
| Name | (5′ to 3′) | ID NO: | (5′ to 3′) |
| sgBGFP | GCTGAAGCACTGCACGCCAT | 15 | GGG |
| sgG542X | GACAACACAGTTCTCTGAGA | 16 | AGG |
| sgF508del | ACCATTAAAGAAAATATCAT | 17 | TGG |

TABLE 8

| | | SEQ ID |
|---|---|---|
| Name | ssDNA HDR Template (5′ to 3′) | NO: |
| BGFP | GCCACCTACGGCAAGCTGACCCTGAAGTTCATCTGCACC | 18 |
| | ACCGGCAAGCTGCCCGTGCCCTGGCCCACCCTCGTGACC | |
| | ACCCTGACGTACGGCGTGCAGTGCTTCAGCCGCTACCCC | |
| | GACCACATGA | |
| G542X | TTTCAATTTTGGATTGTGCATGCTAAATTTTATTTCTGGT | 19 |
| | GTTATGCTTTGGATAATAGGACATCACCAAGTTTGCAGA | |
| | ACAAGACAACACAGTTCTAGGAGAAGGTGGAGTCACAC | |
| | TGAGTGGAGGTCAGCGTGCAAGGATTTCTTTAGCAAGGT | |
| | AAATATTTAACTGTTGGTCTTGTGAGCACTTGCTGTAAAT | |
| | ACTA | |
| F508del | AAGAATTTCATTCTGTTCTCAGTTTTCCTGGATTATGCCT | 20 |
| | GGCACCATTAAAGAAAATATCATCTTTGGTGTTTCCTAT | |
| | GATGAATATAGATACAGAAGCGTCATCAAAGCATGCCA | |
| | ACT | |

TABLE 9

| | Forward Primers | SEQ ID | Reverse Primers | SEQ ID |
|---|---|---|---|---|
| Name | (5′ to 3′) | NO: | (5′ to 3′) | NO: |
| BGFP | AAGCTTGGTACCGA | 21 | TTGTGGCGGATCTTGAAG | 24 |
| | GCTCG | | TTCAC | |
| G542X | TGAATGGCACTTGA | 22 | GAAATTCAAATCAGAGGC | 25 |
| | GTTTATATGATGGG | | ATCTGGGGA | |
| F508del | TGGGGGCAAGTGAA | 23 | CATTGAGGACGTTTGTCT | 26 |
| | TCCTGAGC | | CACTAATGAGT | |

In Vitro Gene Correction in HEK293 Cells with Y66H GFP Mutation

HEK293 cells with Y66H GFP mutation were cultured in DMEM containing 10% FBS and 1% penicillin/streptomycin at 37° C./ 5% CO₂. Briefly, HEK293 cells were seeded into 12-well plates at a cell density of 1.5×10⁵ cells per well and incubated overnight. Then, the medium was replaced with 0.5 mL of fresh completed DiVEM and 100 μL of DOTAPIO LNPs with different weight ratios of Cas9 mnRNA/sgRNA/HDR template were added (total nucleic acid at 0.8 ng L⁻¹). Two days after, 1 mL of fresh medium was added into each well, to maintain enough nutrition. After three days of treatment, cells of each well were collected, washed, and lysed using 50 μL of 1×passive lysis buffer (Promega) containing 2 μL of proteinase K (Thermofisher), through a PCR program (65° C. for 15 min, 95° C. for 10 min). Afterwards, the gene correction sequence was amplified using the following PCR amplification program (95° C. for 5 min; (95° C. for 30s; 64° C. for 30s; 72° C. for 30s) for 35 cycles; 72° C. for 7 min and then keep at 4° C.). Cell lysates were used as DNA templates. PCR amplicons were then purified using PCR purification kits/gel purification kit. Purified PCR amplicons were then sequenced. The sequencing data was analyzed using the TIDER webtool (http://shinyapps.datacurators.nl/tider/), to calculate HDR correction efficiency.

Confocal Imaging of HEK293 Cells with Y66H GFP Mutation

6×10⁴ HEK293 cells with Y66H GFP mutation were seeded in 8-well confocal dish overnight. Afterwards, DOTAP10-HDR LNPs encapsulating Cas9 mRNA, sgRNA, and HDR template (0.5:1:6, weight ratio) were added into cells and incubated for two days. NC group (PBS treatment) and DOTAP10-NHEJ LNPs encapsulating Cas9 mRNA and sgRNA group were used as controls. The dose of total nucleic acid per well was at 0.4 ng L⁻¹. After two days, each well was stained with cell mask deep red stain solution (1:1000 dilution) at 37° C. for 10 min and washed carefully 3 times with PBS buffer. Then cells were observed using confocal microscopy (Zeiss LSM 700).

### Animal Experiments

C₅₇BL/6 mice were obtained from the UTSW Mouse Breeding Core Facility. B6.Cg-Gt(ROSA)26Sortm9(CAG-tdTomato)Hze/J mice (also known as Ai9 tdTOM mice) were purchased from The Jackson Laboratory (007909) and bred to maintain homozygous expression of the Cre reporter allele that has a loxP-flanked STOP cassette preventing transcription of a CAG promoter-driven red fluorescent tdTomato protein. Following Cre-mediated recombination, Ai9 mice will express tdTomato fluorescence. Ai9 tdTOM mice are congenic on the C₅₇BL/6J genetic background.

The creation of the G542X mouse model was previously described. Mice homozygous for these mutations were created by breeding heterozygous males and females. Genotyping was completed by PCR analysis using DNA extracts from ear biopsies. To detect the G542X allele (319 bp) primers P1 (5′-ACAAGACAACACAGTTCTCT-3′ (SEQ ID NO: 27)) and P2 (5′ TCCATGCACCATAACAACAAGT-3′ (SEQ ID NO: 28)) were used. To detect the wildtype (WT) allele (319 bp) P2 and P3 (5′-ACAAGACAACACAGTTCTTG-3′ (SEQ ID NO: 29)) were used in a separate reaction. PCR reactions were completed for 40 cycles of 95° C. for 30 seconds, 58° C. for 30 seconds and 72° C. for 30 seconds and products were run out on 2% agarose gels. All mice were allowed unrestricted access to water and solid chow (Harlan Teklad 7960; Harlan Teklad Global Diets). All animals were maintained on a 12-h light, 12-h dark schedule at a mean ambient temperature of 22° C. and were housed in standard polysulfone microisolator cages in ventilated units with corncob bedding.

In Vivo Luc mRNA Delivery

C₅₇BL/6 mice with weight at around 20 g were i.v. injected with various Luc mRNA formulations (0.1 mg kg⁻¹ of Luc mRNA, n=3 per group). After 6 hours, these mice were injected with D-Luciferin (150 mg kg⁻¹, intraperitoneal) and imaged immediately using an IVIS Lumina system (Perkin Elmer). Afterwards, these mice were killed, and tissues were excised for ex vivo imaging using IVIS Lumina system.

### In Vivo Toxicity Evaluation

C₅₇BL/6 mice with weights at 18-20 g, were randomly divided into four groups: PBS group, DDAB30 LNPs group, DOTAP40 LNPs group, and Lipopolysaccharide (LPS) group (n=5 per group). Briefly, DDAB30 LNPs and DOTAP40 LNPs were injected into mouse tail veil, with mCherry mRNA dose at 1.5 mg kg⁻¹ (total lipid/total RNA at 40:1). PBS treated group was used as negative control. Intraperitoneal injection (I.P.) of LPS (5 mg kg⁻¹) was used as positive control. After treatment of 24h, the whole blood was collected, and the serum was separated to measure the liver function (AST and ALT) and kidney function (BUN and CREA). Mice in LPS group were sacrificed after treated for two days and other groups were sacrificed at day 5. During the period, body weight changes of these mice were monitored. At day 5, mice were sacrificed and tissue weights (liver, spleen and lung) of each mouse were recorded. All tissue (heart, liver, spleen, lung and kidney) sections with H&E staining were then prepared and analyzed.

Basal Stem Cells Editing in tdTOM Mice with DOTAP40-Cre LNPs Treatment

tdTOM mice were treated with DOTAP40 LNPs (total lipid/total NA at 20:1) encapsulating Cre recombinase mRNA twice by I.V. injection (2 mg kg⁻¹ Cre mRNA). Two days after the second injection, mouse lungs were collected, and lung cells were isolated and stained. Briefly, lung tissue was minced using A blade and transferred to an EP tube containing 250 μL of 2×digestion medium (90 units L⁻¹ collagenase I, 50 units L⁻¹ DNase I and 60 units L⁻¹ hyaluronidase, 1% FBS) and homogenized with tissue grinder. Afterwards, tissue solution was transferred to a 15 mL centrifuge tube containing 10 mL of 2×digestion medium and incubated at 37° C. for 1 hour with shaking. Next, the lung solution was filtered using a 70-μm filter and washed once with 1×PBS. A cell pellet was obtained by centrifuging for 10 min at a speed of 500×g at 4° C. The supernatant was removed, and the cell pellet was resuspended in 2 mL of 1×red blood cell lysis buffer (BioLegend, 420301) and incubated on ice for 5 min. After incubation, 4 mL of cell staining buffer (BioLegend) was added to stop red blood cell lysis. The solution was then centrifuged again at 500×g for 10 min to obtain cell pellet. The single cells were resuspended in cell staining buffer to make cell solution at density of 1-5×10⁶ cells mL⁻¹. 100 uL of cell solution was transferred to a new EP tube to incubate with Anti-p75 NGF receptor antibody (1:100 dilution) for 30 mins on ice, to stain lung basal cells. Afterwards, the cells were washed with 1×PBS buffer twice and incubated with Donkey Anti-Rabbit IgG H&L (Alexa Fluor® 647) (1:2000) for 30 mins on ice, protecting from light (total volume 100 μL). The stained cells were washed twice with 1 mL of 1×PBS, then resuspended in 500 μL 1×PBS for flow cytometry analysis using a LSRForessa SORP (version 8.0.1, BD Biosciences) in the Moody Foundation Flow Cytometry Facility. Ghost Dye Red 780 (Tonbo Biosciences, 13-0865-T500) was used to discriminate live cells. Nanoparticle only treatment group (NC) was used as negative control. The data of flow cytometry were analyzed using FLOWJO software version 7.6 (FLOWJO).

Gene Correction in CF Mouse Model with G542X Mutation

DOTAP40-HDR LNPs delivering Cas9 mRNA, sgRNA and HDR template were I.V. injected into CF mouse model containing G542X mutation every week for total three times (total NA at 2 mg kg⁻¹, total lipid/total NA at 20:1, n=6). One week after the last injection, mouse whole lungs were collected and homogenized for genome DNA extraction. Using genome DNA as DNA template, the gene correction sequence was amplified using a PCR amplification program (95° C. for 5 min; (95° C. for 30s; 64° C. for 30s; 72° C. for 45s) for 35 cycles; 72° C. for 7 min and then keep at 4° C.). PCR amplicons were then purified using PCR purification kits. Purified PCR amplicons were then sequenced. The sequencing data was analyzed using the TIDER webtool (http://shinyapps.datacurators.nl/tider/), to calculate HDR correction efficiency. NP only treatment group was used as negative control (NC).

### Crypt Harvest and Intestinal Organoid Culture

Intestinal organoids were cultured similar to previously described methods. Mice were sacrificed by CO₂ asphyxiation, and 20 cm of intestine measured from the stomach were removed. Fecal matter was flushed from the intestine with Ca²⁺ and Mg²⁺-free PBS, and the intestine was flayed using dissecting scissors. The villi were scraped from the small intestine using a microscope slide, and the intestine was cut into ˜1 cm segments, which were suspended in 2 mM EDTA in Ca²⁺ and Mg²⁺-free PBS. The intestinal segments were incubated on a shaker for 30 minutes at room temperature. The segments were then vortexed at for 10 seconds, allowed to settle, and then the supernatant was removed and stored in a 10 cm dish. This process was repeated until four supernatant fractions were produced. The fractions were inspected under a microscope, and the fraction which was most enriched for crypts was passed through a 70 μm cell strainer. The crypts were pelleted at 1,000×G for 10 minutes, then resuspended in 1:1 mixture of Intesticult Organoid Growth Media (OGM; STEMCELL Technologies) and MatriGel (Corning) at a concentration of 10 crypts/μl. The organoids were seeded to 12-well plates, with 70 μl Matrigel:OGM added to each well in 4-5 droplets. The plate was placed in a 37° C./5% CO₂ incubator for 15 minutes to allow the MatriGel to harden. The MatriGel domes were then immersed in 1 mL OGM and returned to the 37° C./5% CO₂ incubator. OGM was changed every 3-4 days, and the organoids were passaged once every 5-7 days.

### G542X Correction in Intestinal Organoids Using LNPs

Organoids were grown in Matrigel droplets in a 12 well plate to approximately 75% confluency with Mouse Intesticult OGM containing 10 uM Y-27632 (STEMCELL Technologies) and 5 uM CHIR 99021 (Sigma-Aldrich). Organoids were released from Matrigel using PBS and centrifugation. Pelleted organoids are resuspended in 1 ml of Acutase (Life Technologies) and incubated at 37° C. for 5 minutes to digest organoids into single cells. Digestion was halted by quenching the Acutase with 2 ml of DMEM media containing 10% FBS. Cells were then placed in an Eppendorf tube with 200 μL of OGM containing (2.5 ng/μL of total NA) and incubated at 37° C./5% CO₂ for 4 hours. Cells and media were then placed in one well of a Matrigel coated 96-well plate. Cells were grown at 37° C./5% CO₂ for approximately 4-5 days until full organoids develop from the surviving single stem cells. Forskolin-induced swelling of the resulting intestinal organoids were carried out as previously described with small modifications. 200 μl OGM containing 20 μM forskolin was added to each well, creating a 10 μM final concentration. Kinetic brightfield images of FIS were acquired under live cell conditions with a Lionheart FX Automated Microscope (Biotek Instruments, Winooski, Vermont). After one hour of FIS, each organoid was scored as either corrected for CFTR activity, if the organoid swelled, or not corrected for CFTR activity, if the organoid did not swell. 8 96-wells per treatment group were used for each experiment. DNA was isolated from each well for sequencing of the G542X locus.

### Uptake of DOTAP10-mCherry in Undifferentiated HBE Cells

Cystic fibrosis patient derived undifferentiated HBE cells with homozygous F508del mutation were thawed and seeded at 150K/well on 6-well plates. Cultures were maintained every day in Lonza BEGM for 4 days prior to treatment with DOTAP10 LNPs encapsulating mCherry mRNA (DOTAP10-mCherry) for 24 hours (mCherry mRNA dose at 1 g well⁻¹). Afterwards, the cellular uptake of DOTAP10-mCherry was imaged using Keyence microscope (BZ-X Analyzer software version 1.0.0, Keyence Corporation).

LNPs Treatment in Undifferentiated HBE Cells with F508del Mutation.

Cystic fibrosis patient derived HBE cells with homozygous F508del mutation (passage 1, P1) were thawed and seeded at 150K/well on 6-well plates on day 0. Cultures were maintained every day in Lonza BEGM for 4 days prior to treatment. At day 4, P1 cells were treated with DOTAP10-HDR LNPs (at total NA concentration of 800 ng/well) for 4 days before transferring into 10 cm² dish and T-75 flask for cell expansion (passage 2, P2). Untreated group was used as negative control (NC). One week after, these cells were established on Transwell Inserts for cell differentiation (passage 3, P3). Cultures were maintained on a 3 days/week feeding routine with Vertex ALI media containing Ultroser G serum for 4 weeks before following function studies. Several wells from P1, P2 and P3 were collected and lysed using 1×passive lysis buffer (Promega) containing proteinase K (Thermofisher), through a PCR program (65° C. for 15 min, 95° C. for 10 min). Afterwards, the gene correction sequence was amplified using the following PCR amplification program (95° C. for 5 min; (95° C. for 30s; 64° C. for 30s; 72° C. for 40s) for 35 cycles; 72° C. for 7 min and then keep at 4° C.). Cell lysates were used as DNA templates. PCR amplicons were then purified using gel purification kit. Purified PCR amplicons were then sequenced. The sequencing data was analyzed using the TIDER webtool (http://shinyapps.datacurators.nl/tider/), to calculate HDR correction efficiency.

LNPs Treatment in Differentiated HBE Cells with F508del Mutation

Patient derived HBE cells with homozygous F508del mutation (passage 1, P1) were thawed and seeded at 150K/well on 6-well plates. Cultures were maintained everyday in Lonza BEGM for one week and then transferred to a T-75 flask for cell expansion (passage 2, P2). Cultures were maintained every day in Lonza BEGM for another one week. Next, Passage 3 (P3) cultures were established on HTS Transwell Inserts and airlifted; cultures were then maintained on a 3 days/week feeding routine with Vertex ALI media containing Ultroser G serum for cell differentiation. Afterwards, P3 HBEs were washed according to ReCode wash protocol and 12 μg of DOTAP10-HDR formulation were directly administered to apical surface of cultures in liquid bolus and cultured for another four weeks before following function studies. Several wells were collected after treated for 3 days and lysed using 30 μL of 1×passive lysis buffer (Promega) containing 2 μL of proteinase K (Thermofisher), through a PCR program (65° C. for 15 min, 95° C. for 10 min). Afterwards, the gene correction sequence was amplified using the following PCR amplification program (95° C. for 5 min; (95° C. for 30s; 64° C. for 30s; 72° C. for 40s) for 35 cycles; 72° C. for 7 min and then keep at 4° C.). Cell lysates were used as DNA templates. PCR amplicons were then purified using gel purification kit. Purified PCR amplicons were then sequenced. The sequencing data was analyzed using the TIDER webtool (http://shinyapps.datacurators.nl/tider/), to calculate HDR correction efficiency.

### Functional Studies in Differentiated HBE Cells.

CFTR function was measured as transepithelial chloride secretion across fully differentiated P3 F508del/F508del HBEs using a Transepithelial Current Clamp (TECC) and 24-well electrode manifold (EP Devices). Selected wells were pretreated for 24 hours with 3 μM VX-809 or 0.2% DMSO. To prepare the plate for functional analysis, differentiation media was replaced with HEPES buffered F-12 assay medium (pH 7.4) on both the apical and basolateral sides. After a 45 minute incubation in a 37° C. incubator without CO₂, the 24-well transwell was mounted onto a 37° C. heated platform and transepithelial resistance (Rt) and voltage (Vt) were continuously measured. Baseline values were measured for ˜30 minutes, then Rt and Vt were measured for ˜15 minutes after apical addition of benzamil (6 μM final concentration), for ˜30 minutes after simultaneous apical/basolateral addition of forskolin (10 uM final concentration)/VX-770 (1 μM final concentration), and for ˜15 minutes after basolateral addition of bumetanide (20 μM final concentration). Benzamil inhibits sodium transport by the epithelial sodium channel, forskolin increases intracellular cAMP which results in the downstream activation of the CFTR channel, VX-770 is a potentiator for the F508del-CFTR channel, and bumetanide inhibits the basolateral membrane NaCl₂ cotransporter which ultimately blocks Cl₂ transport. CFTR functional results are presented as equivalent chloride current (I_{eq}) which was calculated using Ohm's law, I_{eq}=Vt/Rt.

### Western Blot

P3 F508del HBE cells were lysed immediately following TECC-24 functional analysis. After gently washing HBEs with PBS, 35 μl of RIPA buffer (150 mM NaCl, 50 mM Tris-HCl (pH 8), 1% IGEPAL, 0.5% sodium deoxycholate, 0.1% SDS, 1% protease inhibitor) was added directly to transwell insert, rocked for 1 hour at 4° C., then collected in 1.5 ml tubes on ice. Insoluble fractions were separated by centrifugation at 1600×g for 5 minutes at 4° C. The supernatant was then collected in a separate 1.5 ml tube and Laemmli sample buffer was added at 1:6 dilution. Entire lysate was loaded, and protein was separated on a 7-10% hand-cast SDS-PAGE gel at 100V for 1.5-2 hours. Next, protein was transferred to an Immobilon-FL PVDF (Millipore, #IPFL00010) membrane for 1 hour at 100 V at 4° C. using the Mini Trans-Blot Electrophoretic Transfer Cell system. The membrane was then blocked with 5% milk in TBS for 1 hour at room temperature. Anti-CFTR primary antibody was incubated overnight at 1:15,000 dilution in 1% Tween 20 in TBS (TBST) at 4° C. The following day, anti-actin was added at 1:50,000 dilution for 30 minutes in TBST at room temperature. The membrane was then washed 3 times with TBST and incubated with IRDye 680RD Goat anti-Mouse IgG secondary antibody (LI-COR, 926-68070) at 1:10,000 dilution in TBST for 1 hour at room temperature. The membrane was washed again three times with TBST and once with TBS, and then finally imaged using the LI-COR Odyssey CLx imaging system.

In Vitro Delivery of Luc mRNA and Cell Viability Study

Hela cells were seeded into white 96-well plates at a density of 1×10⁴ cells per well the day before transfection. The culture was treated with 5-30% of DOTAP incorporated LNP formulations containing Luc mRNA (20 ng of Luc mRNA per well). ONE-Glo+Tox kits were used to detect luciferase expression and evaluate cytotoxicity 24 hr after the treatment by following Promega's standard protocol.

Gene Editing (Cas9 mRNA/sgRNA and Cas9/sgRNA/ssDNA) in the tdTOM Mice Model

To evaluate in vivo gene editing by using a two-component system (Cas9 mRNA/sgRNA) against a three-component system (Cas9 mRNA/sgRNA/ssDNA), Ai14 tdTOM reporter mice of comparable weight and same sex were selected. The formulations were prepared as previously described, with a total lipid to total nucleic acid weight ratio of 20:1. tdTOM mice were treated once a week with DOTAP40-NHEJ (Cas9 mRNA:sgTOM1=2:1, wt/wt) and DOTAP40-HDR (Cas9 mRNA: sgTOM1: ssDNA HDR template for CF G542X=2:1:3, wt/wt/wt) formulations respectively with a total RNA dosage of 1 mg kg⁻¹ by intravenous administrations. One week following the third injection, mice (n=3 per group) were sacrificed, and the lungs cells were isolated and stained for cytometry analysis to determine the proportion of tdTOM+ cells in each different cell type.

Briefly, mouse lungs were resected; and the tissues were placed into ice cold PBS. The lung tissue was then cut into small pieces and transferred into a 50 mL tube containing 10 mL of 1× lung digestion media [RPMI dissociation medium (1:1 vol/vol) RPMI supplemented with 2% wt/vol BSA, 300 U/mL collagenase, 100 U/mL hyaluronidase]. The 50 mL tube was then incubated at 37° C. for 1 hr while shaking at 180 rpm. After incubation, the homogenized lung cell solution was pipetted up and down several times to remove cell clumps and finally filtered through a 70-micron cell strainer into a new 50 mL falcon tube. The filter was washed with 10 mL wash buffer consisting of cold PBS and 2% fetal bovine serum (FBS). The sample was then centrifuged at 1200 rpm for 5 minutes. The supernatant was removed, and the cell pellet was resuspended in 10 ml of cold wash buffer. Next, the red blood cells were lysed by resuspending the cell pellet in 5 mL of 1×RBC lysis buffer (BioLegend) at room temperature for 5 minutes. After 5 minutes, 10 mL of wash media was added to the sample. The sample was centrifuged at 1200 rpm for 5 minutes. Finally, the RBC free cell pellet was resuspended in 5 mL of cell staining buffer (BioLegend) and proceeded with antibody staining for flowcytometry.

Single-cell suspensions obtained from the mouse lungs were pre-blocked with mouse Fc-receptor blocker (BioLegend) for 15 minutes. Subsequently cells were labeled with an Alexa fluor 488-conjugated anti mouse CD31, Pacific, blue-conjugated anti mouse CD45 and Alexa fluor 647-conjugated anti mouse EpCAM antibodies (all from BioLegend) by incubating 100 μL of cell suspension with antibodies for 15 minutes on ice. Ghost dye red (BioLegend) was used to identify the dead cells. Next, the cell pellet was washed 3-times with cell staining buffer to remove excess antibodies. Finally, the cell pellet was resuspended in 500 μL of cold cell staining buffer and kept on ice until analysis by flow cytometer. Cells were then analyzed by Becton Dickenson (BD) LSR Fortessa flow cytometer. Data obtained from the flow cytometer were finally analyzed by flowjo software (BD).

### TA Cloning for Sequencing

Taq polymerase-amplified PCR products from DOTAP40-HDR treated G542× mice lungs was inserted into a plasmid by using the TOPO® TA Cloning® Kits following the standard protocol. 50 single colonies were picked and cultured overnight in LB medium containing 50 ug/mL ampicillin. Plasmid DNA from each single colony was extracted using QIAprep Spin Miniprep Kit and later sequenced to detect SORT-LNP mediated gene correction.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for enhancing an expression or activity of cystic fibrosis transmembrane conductance regulator (CFTR) protein in a cell, the method comprising:
(a) contacting said cell with a nucleic acid editing system assembled with a lipid composition, which nucleic acid editing system comprises (i) a guide nucleic acid, (ii) a heterologous polypeptide comprising an endonuclease or a heterologous polynucleotide encoding said heterologous polypeptide, and (iii) a donor template nucleic acid, to yield a complex of said heterologous endonuclease with said guide nucleic acid in said cell;
(b) cleaving a CFTR gene or transcript in said cell with said complex at a cleavage site to yield a cleaved CFTR gene or transcript; and
(c) using said donor template nucleic acid to repair said cleaved CFTR gene or transcript to yield a repaired CFTR gene or transcript encoding a functional CFTR protein in said cell, thereby enhancing said expression or activity of CFTR protein in said cell.

2. The method of claim 1, wherein (c) is characterized by an off-target insertion or/and deletion (indel) rate of no more than about 50%, 49%, 48%, 47%, 46%, 45%, 44%, 43%, 42%, 41%, or 40%.

3. The method of claim 2, wherein said off-target indel rate comprises a ratio of (1) a sum of test cells detected to have an incorrectly altered CFTR gene or transcript relative to (2) a sum of total test cells.

4. The method of any one of claims 1-3, wherein (c) is characterized by an on-target repair rate of at least about 5%, 6%, 7%, 8%, 9%, 10%, 11%, 12%, 13%, 14%, 15%, 16%, 17%, 18%, 19%, or 20%.

5. The method of claim 4, wherein said on-target repair rate comprises a ratio of (1) a sum of test cells detected to have said repaired CFTR gene or transcript relative to (2) a sum of total test cells.

6. The method of any one of claims 1-5, wherein the method increases an amount of a functional CFTR gene, transcript or protein in said cell (e.g., by at least about 1.1-fold) relative to a corresponding control, optionally, wherein said corresponding control is a corresponding cell absent said contacting.

7. The method of any one of claims 1-6, wherein the method yields a therapeutically effective amount of a functional of CFTR gene, transcript or protein in said cell (e.g., at least about 10%, 15%, 20%, 25%, or 30% among all detectable CFTR gene, transcript or protein).

8. The method of any one of claims 1-7, wherein the method enhances (e.g., chloride) ion transport in said cell (e.g., by at least about 1.1-fold) relative to a corresponding control, optionally, wherein said corresponding control is a corresponding cell absent said contacting.

9. The method of any one of claims 1-8, wherein said cell is a lung cell.

10. The method of claim 9, wherein said cell is a lung basal cell.

11. The method of any one of claims 1-10, wherein said cell is an airway epithelial cell (e.g., a bronchial epithelial cell).

12. The method of any one of claims 1-11, wherein said cell is undifferentiated.

13. The method of any one of claims 1-11, wherein said cell is differentiated.

14. The method of any one of claims 1-13, wherein (b) comprises cleaving a CFTR gene or transcript that comprises a loss-of-function mutation.

15. The method of claim 14, wherein said loss-of-function mutation comprises a mutation in an exon selected from exons 9-27 of CFTR.

16. The method of claim 14 or 15, wherein said loss-of-function mutation is F508del or G542X.

17. The method of any one of claims 14-16, wherein said loss-of-function mutation is associated with cystic fibrosis, hereditary emphysema, or chronic obstructive pulmonary disease (COPD).

18. The method of any one of claims 1-17, wherein said contacting is ex vivo.

19. The method of any one of claims 1-17, wherein said contacting is in vitro.

20. The method of any one of claims 1-17, wherein said contacting is in vivo.

21. The method of any one of claims 1-20, wherein said contacting is repeated.

22. The method of any one of claims 1-21, wherein said contacting comprises contacting a plurality of cells that comprise said cell.

23. The method of claim 22, wherein said repairing yields a functional CFTR gene, transcript or protein in at least about 10%, 15%, 20%, 25%, 30%, 35%, 40%, 45%, 50%, 60%, 65%, or 70% of said plurality of cells, optionally wherein said plurality of cells are a plurality of (e.g., lung) basal cells.

24. The method of any one of claims 1-23, wherein said lipid composition comprises:
an ionizable cationic lipid; and
a selective organ targeting (SORT) lipid separate from said ionizable cationic lipid.

25. The method of claim 24, wherein said lipid composition comprises a phospholipid separate from said SORT lipid.

26. The method of any one of claims 1-25, further comprising deriving a cell composition from said cell.

27. An engineered cell composition comprising or derived from a cell having an expression or activity of cystic fibrosis transmembrane conductance regulator (CFTR) protein enhanced by a method of any one of claims 1-25.

28. A composition comprising a nucleic acid editing system assembled with a lipid composition, wherein said nucleic acid editing system comprises:
(i) a guide nucleic acid comprising a targeting sequence that is complementary with a target sequence of a cystic fibrosis transmembrane conductance regulator (CFTR) gene or transcript;
(ii) a polypeptide comprising an endonuclease or a polynucleotide encoding said polypeptide, which endonuclease is configured to (1) form a complex with said guide nucleic acid and (2) cleave said CFTR gene or transcript in a cell in a cleavage event; and
(iii) a donor template nucleic acid configured to alter said CFTR gene or transcript, subsequent to said cleavage event, to provide a functional CFTR gene, transcript or protein in said cell.

29. The composition of claim 28, wherein said guide nucleic acid comprises a nucleotide sequence selected from those set forth in Table A and complementary sequences thereof.

30. The composition of claim 28 or 29, wherein said donor template nucleic acid comprises a nucleotide sequence selected from those set forth in Table B and complementary sequences thereof.

31. The composition of any one of claims 28-30, wherein said donor template nucleic acid comprises a 5′ homology arm.

32. The composition of any one of claims 28-31, wherein said donor template nucleic acid comprises a 3′ homology arm.

33. The composition of any one of claims 28-32, wherein (ii) is a messenger ribonucleic acid (mRNA) encoding said polypeptide comprising said endonuclease.

34. The composition of any one of claims 28-32, wherein (ii) is said polypeptide comprising said endonuclease.

35. The composition of any one of claims 28-34, wherein said endonuclease is a CRISPR-associated (Cas) polypeptide or a modification thereof.

36. The composition of claim 35, wherein said endonuclease is Cas9.

37. The composition of any one of claims 28-36, wherein (i) and (iii) are present on two different molecules.

38. The composition of any one of claims 28-37, wherein (i), (ii), and (iii) are present on three different molecules.

39. The composition of any one of claims 28-37, wherein at least two of (i), (ii) and (iii) are present on one molecule.

40. The composition of any one of claims 28-39, wherein (i) and (ii) are present in said composition at a molar or weight ratio from 1:1 to 1:20.

41. The composition of any one of claims 28-40, wherein (i) and (iii) are present in said composition at a molar or weight ratio from 1:1 to 1:30.

42. The composition of any one of claims 28-41, wherein said composition is formulated for pharmaceutical (e.g., systemic) administration.

43. An engineered cell composition comprising or derived from a cell, which cell comprises a heterologous cystic fibrosis transmembrane conductance regulator (CFTR) gene, transcript or protein produced by a composition of any one of claims 28-42.

44. A method for genetic correction of cystic fibrosis transmembrane conductance regulator (CFTR) in a lung basal cell, comprising:
contacting said lung basal cell with a composition that comprises a nucleic acid editing system assembled with a lipid composition, thereby delivering said nucleic acid editing system to said lung basal cell.

45. A method for genetic correction of cystic fibrosis transmembrane conductance regulator (CFTR) in a cell composition, comprising:
contacting said cell composition comprising a plurality of lung basal cells with a composition that comprises a nucleic acid editing system assembled with a lipid composition, thereby delivering said nucleic acid editing system to at least about 10%, 15%, 20%, 25%, 30%, 35%, 40%, 45%, 50%, 60%, 65%, or 70% of said plurality of lung basal cells.

46. A method for genetic correction of cystic fibrosis transmembrane conductance regulator (CFTR) in a cell composition, comprising:
contacting said cell composition with a composition that comprises a nucleic acid editing system assembled with a lipid composition, which cell composition comprise a lung basal cell and a lung non-basal cell, thereby delivering said nucleic acid editing system to said lung basal cell in a greater amount than that delivered to said lung non-basal cell.

47. The method of claim 46, wherein said non-basal cell is an ionocyte, a ciliated cell, or a secretory cell.

48. The method of any one of claims 44-47, wherein said lung basal cell or said plurality of lung basal cells is/are determined to exhibit a mutation in CFTR gene.

49. The method of any one of claims 44-47, wherein said lung basal cell or said plurality of lung basal cells exhibit(s) a mutation in CFTR gene.

50. The method of any one of claims 44-49, wherein said lung basal cell or said plurality of lung basal cells is/are from a subject.

51. The method of claim 50, wherein said subject is determined to exhibit a mutation in CFTR gene.

52. The method of claim 50, wherein said subject exhibits a mutation in CFTR gene.

53. The method of any one of claims 44-52, wherein said contacting is ex vivo.

54. The method of any one of claims 44-52, wherein said contacting is in vitro.

55. The method of any one of claims 44-52, wherein said contacting is in vivo.

56. A method for treating a subject having or suspected of having a cystic fibrosis transmembrane conductance regulator (CFTR)-associated condition, the method comprising administering to said subject a composition comprising a nucleic acid editing system assembled with a lipid composition.

57. The method of claim 56, wherein said CFTR-associated condition is cystic fibrosis, hereditary emphysema, chronic obstructive pulmonary disease (COPD), or a combination thereof.

58. The method of claim 56 or 57, wherein said subject is a mammal.

59. The method of claim 58, wherein said subject is a human.

60. The method of any one of claims 56-59, wherein said subject is determined to exhibit a mutation (e.g., F508del or G542X) in CFTR gene.

61. The method of any one of claims 56-60, wherein said administering comprises systemic administration.

62. A composition comprising a lipid composition assembled with a nucleic acid editing system, wherein the nucleic acid editing system comprises:
(a) a guide nucleic acid;
(b) a heterologous polypeptide comprising an endonuclease or a heterologous polynucleotide encoding said heterologous polypeptide; and
(c) a donor template nucleic acid,
wherein the lipid composition comprises a selective organic targeting (SORT) lipid, wherein said SORT lipid has a structural formula (S-I′): 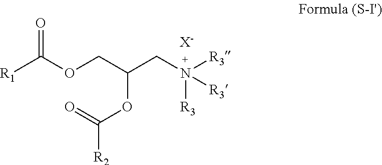
wherein:
R₁ and R₂ are each independently alkyl(C₈-C₂₄), alkenyl(C₈-C₂₄), or a substituted version of either group;
R₃, R₃′, and R₃″ are each independently alkyl(C_{≤6}) or substituted alkyl(C≤₆); and
X⁻ is a monovalent anion,
wherein said composition is configured to repair a cleaved cystic fibrosis transmembrane conductance regulator (CFTR) gene or transcript to yield a repaired CFTR gene or transcript encoding a functional CFTR protein when said composition is delivered to a cell, thereby enhancing an expression or activity of said functional CFTR protein in said cell.

63. The composition of claim 62, wherein said SORT lipid is 1,2-dioleoyl-3-trimethylammonium-propane (DOTAP).

64. The composition of any one of claims 62-63, wherein said lipid composition comprises about 10 mole percent (mol %) to about 40 mol % of said SORT lipid (e.g., DOTAP).

65. The composition of any one of claims 62-64, wherein said lipid composition comprises an ionizable cationic lipid separate from said SORT lipid.

66. The composition of any one of claims 62-65, wherein said donor template nucleic acid is configured to alter a gene or transcript in a homology directed repair (HDR) pathway.

67. The composition of any one of claims 62-66, wherein said endonuclease is a CRISPR-associated (Cas) polypeptide or a modification thereof.

68. The composition of any one of claims 62-67, wherein said endonuclease is Cas9.
